(19) United States
(12) Patent Application Publication
(10) Pub. No.: US 2016/0002221 A1
(43) Pub. Date: Jan. 7, 2016

(54) Substituted Pyridopyrazines as Syk Inhibitors

(71) Applicant: HUTCHISON MEDIPHARMA LIMITED, Shanghai (CN)

(72) Inventors: Wei-Guo SU, Shanghai (CN); Wei DENG, Shanghai (CN)

(21) Appl. No.: 14/650,281

(22) PCT Filed: Dec. 6, 2013
(86) PCT No.: PCT/CN2013/088817

§ 371 Date: Jun. 5, 2015

(30) Foreign Application Priority Data
Dec. 7, 2012 (CN) PCT/CN2012/086144

(51) Int. Cl. C07D 471/04

(52) U.S. Cl. CPC C07D 471/04

(57) ABSTRACT

The present invention relates to pyridopyrazine compounds of formula (I), pharmaceutical compositions thereof and methods of use therefore, wherein R¹, R², R³, L, m, p and W are as defined in the specification.

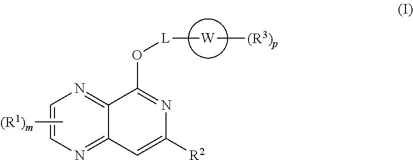

### TECHNICAL FIELD

The present invention relates to novel pyridopyrazine compounds, pharmaceutical compositions thereof and methods of use therefore.

### BACKGROUND OF THE INVENTION

Protein kinases, the largest family of human enzymes, encompass well over 500 proteins. Spleen Tyrosine Kinase (Syk) is a member of the Syk family of tyrosine kinases, and is a regulator of early B-cell development as well as mature B-cell activation, signaling, and survival.

Syk is a non-receptor tyrosine kinase that plays critical roles in immunoreceptor- and integrin-mediated signaling in a variety of cell types, including B cells, macrophages, monocytes, mast cells, eosinophils, basophils, neutrophils, dendritic cells, T cells, natural killer cells, platelets, and osteoclasts. Immunoreceptors as described herein include classical immunoreceptors and immunoreceptor-like molecules. Classical immunoreceptors include B-cell and T-cell antigen receptors as well as various immunoglobulin receptors (Fc receptors). Immunoreceptor-like molecules are either structurally related to immunoreceptors or participate in similar signal transduction pathways, and are primarily involved in non-adaptive immune functions, including, for example, neutrophil activation, natural killer cell recognition, and osteoclast activity. Integrins are cell surface receptors that play key roles in the control of leukocyte adhesion and activation in both innate and adaptive immunity.

Ligand binding leads to activation of both immunoreceptors and integrins, which results in Src family kinases being activated, and phosphorylation of immunoreceptor tyrosine-based activation motifs (ITAMs) in the cytoplasmic face of receptor-associated transmembrane adaptors. Syk binds to the phosphorylated ITAM motifs of the adaptors, leading to activation of Syk and subsequent phosphorylation and activation of downstream signaling pathways.

Syk is essential for B-cell activation through B-cell receptor (BCR) signaling. SYK becomes activated upon binding to phosphorylated BCR and thus initiates the early signaling events following BCR activation. B-cell signaling through BCR can lead to a wide range of biological outputs, which in turn depend on the developmental stage of the B-cell. The magnitude and duration of BCR signals must be precisely regulated. Aberrant BCR-mediated signaling can cause disregulated B-cell activation and/or the formation of pathogenic auto-antibodies leading to multiple autoimmune and/or inflammatory diseases. Mice lacking Syk show impaired maturation of B-cells, diminished immunoglobulin production, compromised T-cell-independent immune responses, and marked attenuation of the sustained calcium sign upon BCR stimulation.

A large body of evidence supports the role of B-cells and the humoral immune system in the pathogenesis of autoimmune and/or inflammatory diseases. Protein-based therapeutics (such as Rituxan) developed to deplete B-cells represent an approach to the treatment of a number of autoimmune and inflammatory diseases. Auto-antibodies and their resulting immune complexes are known to play pathogenic roles in autoimmune disease and/or inflammatory disease. The pathogenic response to these antibodies is dependent on signaling through Fc Receptors, which is, in turn, dependent upon Syk. Because of Syk's role in B-cell activation, as well as FcR dependent signaling, inhibitors of Syk can be useful as inhibitors of B-cell mediated pathogenic activity, including autoantibody production. Therefore, inhibition of Syk enzymatic activity in cells is proposed as a treatment for autoimmune disease through its effects on autoantibody production.

Syk also plays a key role in FCεRI mediated mast cell degranulation and eosinophil activation. Thus, Syk is implicated in allergic disorders including asthma. Syk binds to the phosphorylated gamma chain of FCεRI via its SH2 domains and is essential for downstream signaling. Syk deficient mast cells demonstrate defective degranulation, and arachidonic acid and cytokine secretion. This also has been shown for pharmacologic agents that inhibit Syk activity in mast cells. Syk antisense oligonucleotides inhibit antigen-induced infiltration of eosinophils and neutrophils in an animal model of asthma. Syk deficient eosinophils also show impaired activation in response to FCεRI stimulation. Therefore, small molecule inhibitors of Syk may be useful for treatment of allergy-induced inflammatory diseases including asthma.

Syk is also expressed in mast cells and monocytes and has been shown to be important for the function of these cells. For example, Syk deficiency in mice is associated with impaired IgE-mediated mast cell activation, which causes marked diminution of TNF-alpha and other inflammatory cytokine release. Additionally, Syk inhibitors have been shown to inhibit antigen-induced passive cutaneous anaphylaxsis, bronchoconstriction and bronchial edema in rats.

Thus, the inhibition of Syk activity can be useful for the treatment of allergic disorders, autoimmune diseases, and inflammatory diseases, such as: SLE, rheumatoid arthritis, multiple vasculitides, idiopathic thrombocytopenic purpura (ITP), myasthenia gravis, allergic rhinitis, chronic obstructive pulmonary disease (COPD), adult respiratory distress syndrome (ARDS) and asthma. In addition, Syk has been reported to play an important role in ligand-independent tonic signaling through the B-cell receptor, known to be an important survival signal in B-cells. Thus, inhibition of Syk activity may be useful in treating certain types of cancer, including B-cell lymphoma and leukemia.

Vascular endothelial growth factor (VEGF)-A, a major regulator for angiogenesis, binds and activates two tyrosine kinase receptors, VEGFR-1 (Flt-1) and VEGFR-2 (KDR). VEGFR-1 (Flt-1) and VEGFR-2 (KDR) play different roles in physiological and pathological angiogenesis. VEGFR-2 (KDR) has strong tyrosine kinase activity, and mostly uses the Phospholipase-Cy-Protein kinaseC pathway to activate MAP-kinase and DNA synthesis. VEGFR-2 (KDR) is the major positive signal transducer for both physiological and pathological angiogenesis including cancer and diabetic retinopathy. Thus, VEGFR-2 (KDR) kinase inhibitors are being used in the treatment of a wide variety of cancers. Recent studies have shown that patients will likely require long-term treatment with these agents. Hypertension has emerged as a frequent side effect associated with agents that block signaling through the VEGF pathway (Pankaj Bhargava, Am. J. Physiol. Regul. Integr. Comp. Physiol. 297:R1-R5, 2009). Several studies results indicate that the vasodilation and hypotensive effect of VEGF may involve its both receptors, but VEGFR-2 (KDR) is the predominant receptor mediating this effect (Bing Li, et al., Hypertension. 39:1095-1100, 2002).

Fms-like tyrosine kinase 3 (Flt-3) or receptor-type tyrosine-protein kinase Flt3 (also known as Cluster of differentiation antigen 135, CD135) is a cytokine receptor which belongs to the receptor tryrosin kinase class III. Flt-3 is normally expressed by hematopoietic stem/progenitor cells. Signaling through Flt-3 plays a role in cell survival, proliferation, and differentiation. Flt-3 is important for lymphocyte (B cell and T cell) development, but not for the development of other blood cells (myeloid development). Flt-3 knockout mice have a subtle hematopoietic stem/progenitor cells deficit. Thus, targeted disruption of the Flt-3 gene leads to deficiencies in primitive hematopoietic progenitors.

WO 2012/123312 (GLAXO GROUP LIMITED), titled as “PYRIDO[3,4-B]PYRAZINE DERIVATIVES AS SYK INHIBITORS” and filed on Mar. 8, 2012, discloses noval pyrido[3,4-b]pyrazines which have SYK inhibitory activity.

### SUMMARY OF THE INVENTION

Provided is at least one compound of formula (I):

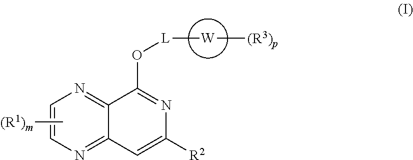

and/or its racemic mixture, enantiomers, diastereomers, tautomers, or mixtures of optional ratio, or at least one pharmaceutically acceptable salt, or solvate thereof, wherein

R¹ is independently chosen from hydrogen, halo, —CN, —OH, optionally substituted C₁-C₆alkyl, optionally substituted C₁-C₆alkoxy, —NH₂, —NH(C₁-C₄alkyl), and —N(C₁-C₄alkyl)(C₁-C₄alkyl),

R² is aryl, or heteroaryl, each of which is optionally substituted by one or more groups selected from halo, —NR⁵R⁶, —S(O)ₙR⁸, —C(O)R⁹, —C(O)OR⁷, —CN, —C(O)NR⁵R⁶, —NR⁵C(O)R⁹, —NR⁵S(O)ₙR⁸, —NR⁵S(O)ₙNR¹⁰R¹¹, —NR⁵C(O)OR⁷, NR⁵C(O)NR¹⁰R¹¹, —NO₂, —S(O)ₙNR⁵R⁶, optionally substituted lower alkyl, optionally substituted cycloalkyl, optionally substituted heterocycle, optionally substituted heteroaryl, optionally substituted aryl, optionally substituted alkenyl, and optionally substituted alkynyl,

L is a bond, or optionally substituted C₁-C₆alkylene,

W is cycloalkyl, heterocycle, aryl, or heteroaryl,

R³ is independently selected from hydrogen, -Lx-halo, -Lx-R⁴, -Lx-NR⁵R⁶, -Lx-OR⁷, -Lx-S(O)ₙR⁸, -Lx-C(O)R⁹, —S(O)ₙ-Lx-R⁸, —C(O)-Lx-R⁹, -Lx-CN, -Lx-NR⁵C(O)R⁹, -Lx-NR⁵S(O)ₙR⁸, -Lx-NR⁵C(O)NR¹⁰R¹¹, -Lx-NR⁵S(O)ₙNR¹⁰R¹¹, Lx-NR⁵C(O)OR⁷, -Lx-NR⁵S(O)ₙOR⁷, —NO₂, -Lx-C(O)NR⁵R⁶, -Lx-S(O)ₙNR⁵R⁶, oxo(═O), optionally substituted cycloalkyl, optionally substituted heterocycle, optionally substituted heteroaryl, and optionally substituted aryl,

provided when L is methylene and W is 5- or 6-membered heterocycle, R³ is independently selected from -Lx-NR⁵R⁶, -Lx-OR⁷, -Lx-S(O)ₙR⁸, -Lx-C(O)R⁹, —S(O)ₙ-Lx-R⁸, —C(O)-Lx-R⁹, -Lx-CN, -Lx-NR⁵C(O)R⁹, -Lx-NR⁵S(O)ₙR⁸, -Lx-NR⁵C(O)NR¹⁰R¹¹, -Lx-NR⁵S(O)ₙNR¹⁰R¹¹, -Lx-NR⁵C(O)OR⁷, -Lx-NR⁵S(O)ₙOR⁷, —NO₂, -Lx-C(O)NR⁵R⁶, -Lx-S(O)ₙNR⁵R⁶, oxo(═O), optionally substituted cycloalkyl, optionally substituted heterocycle, optionally substituted heteroaryl, and optionally substituted aryl,

R⁴ is C₁-C₆alkyl, C₂-C₆alkenyl, or C₂-C₆alkynyl, each of which is optionally substituted,

R⁵, R⁶, R⁷, R⁸, R⁹, R¹⁰, and R¹¹ are independently selected from hydrogen, alkyl, cycloalkyl, aryl, heteroaryl, and heterocycle, each of which except for hydrogen, is optionally substituted with one or more groups selected from halo, —OH, —O(C₁-C₄alkyl), —CN, C₁-C₄alkyl, —NH₂, —NH(C₁-C₄alkyl), —N(C₁-C₄alkyl)(C₁-C₄alkyl), —C(O)NH₂, —C(O)NH(C₁-C₄alkyl), —C(O)N(C₁-C₄alkyl)(C₁-C₄alkyl), —C(O)(C₁-C₄alkyl), —NHC(O)(C₁-C₄alkyl), —N(C₁-C₄alkyl)C(O)(C₁-C₄alkyl), —S(O)ₙNH₂, —S(O)ₙNH(C₁-C₄alkyl), —S(O)ₙN(C₁-C₄alkyl)(C₁-C₄alkyl), —S(O)ₙ(C₁-C₄alkyl), —NHS(O)ₙ(C₁-C₄alkyl), —N(C₁-C₄alkyl)S(O)ₙ(C₁-C₄alkyl) optionally substituted C₃-C₈cycloalkyl, and optionally substituted 3-8 membered heterocycle, wherein C₁-C₄alkyl is optionally substituted by halo, —OH, —OMe, —CN,

or R⁵ and R⁶, R⁵ and R⁷, R⁵ and R⁸, R⁵ and R⁹, and R⁵ and R¹⁰ together with the atom(s) to which they are attached can form a ring, which is optionally substituted with one or more groups selected from halo, —OH, —O(C₁-C₄alkyl), —CN, C₁-C₄alkyl, —NH₂, —NH(C₁-C₄alkyl), —N(C₁-C₄alkyl)(C₁-C₄alkyl), —C(O)NH₂, —C(O)NH(C₁-C₄alkyl), —C(O)N(C₁-C₄alkyl)(C₁-C₄alkyl), —C(O)(C₁-C₄alkyl), —NHC(O)(C₁-C₄alkyl), —N(C₁-C₄alkyl)C(O)(C₁-C₄alkyl), —S(O)ₙNH₂, —S(O)ₙNH(C₁-C₄alkyl), —S(O)ₙN(C₁-C₄alkyl)(C₁-C₄alkyl), —S(O)ₙ(C₁-C₄alkyl), —NHS(O)ₙ(C₁-C₄alkyl), —N(C₁-C₄alky)S(O)ₙ(C₁-C₄alkyl), optionally substituted C₃-C₈cycloalkyl, and optionally substituted 3-8 membered heterocycle, wherein C₁-C₄alkyl is optionally substituted by halo, —OH, —OMe, or —CN,

Lx is a bond, or optionally substituted C₁-C₆alkylene,

wherein each optionally substituted group above for which the substituent(s) is (are) not specifically designated, can be unsubstituted or independently substituted with, for example, one or more, such as one, two, or three, substituents independently chosen from C₁-C₄ alkyl, cycloalkyl, aryl, heterocycle, heteroaryl, aryl-C₁-C₄ alkyl-, heteroaryl-C₁-C₄ alkyl-, C₁-C₄ haloalkyl-, —OC₁—C₄ alkyl, —OC₁—C₄ alkylphenyl, —C₁-C₄ alkyl-OH, —C₁-C₄ alkyl-O—C₁-C₄ alkyl, —OC₁—C₄ haloalkyl, halo, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo, —CO₂H, —C(O)OC₁—C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl, —C(O)(C₃-C₈cycloalkyl), —C(O)(C₅-C₁₀aryl), —C(O)(C₃-C₅heterocycle), —C(O)(C₅-C₁₀heteroaryl), —C(O)(C₁-C₄alkyl)-(C₃-C₈cycloalkyl), —C(O)(C₁-C₄alkyl)-(C₅-C₁₀aryl), —C(O)(C₁-C₄alkyl)-(C₃-C₈heterocycle), —C(O)(C₁-C₄alkyl)-(C₅-C₁₀heteroaryl), —C(O)C₁-C₄ haloalkyl, —OC(O)C₁-C₄ alkyl, —SO₂(C₁-C₄ alkyl), —SO₂(C₃-C₈cycloalkyl), —SO₂(C₅-C₁₀aryl), —SO₂(C₃-C₅heterocycle), —SO₂(C₅-C₁₀heteroaryl), —SO₂(C₁-C₄alkyl)-(C₃-C₈cycloalkyl), —SO₂(C₁-C₄alkyl)-(C₅-C₁₀aryl), —SO₂(C₁-C₄alkyl)-(C₃-C₈heterocycle), —SO₂(C₁-C₄alkyl)-(C₅-C₁₀heteroaryl), —SO₂(C₁-C₄ haloalkyl), —SO₂NH₂, —SO₂NH(C₁-C₄ alkyl), —SO₂N(C₁-C₄ alkyl)(C₁-C₄ alkyl), —SO₂NH(phenyl), —SO₂N(C₁-C₄ alkyl) (phenyl), —NHSO₂(C₁-C₄ alkyl), —NHSO₂(phenyl), and —NHSO₂(C₁-C₄ haloalkyl), in which each of alkyl, cycloalkyl, aryl, heterocycle, and heteroaryl is optionally substituted by one or more groups chosen from halo, cycloalkyl, heterocycle, C₁-C₄ alkyl, C₁-C₄ haloalkyl-, —OC₁—C₄ alkyl, C₁-C₄ alkyl-OH, —C₁-C₄ alkyl-O—C₁-C₄ alkyl, —OC₁—C₄ haloalkyl, cyano, nitro, —NH₂, —OH, —CO₂H, —C(O)OC₁—C₄ alkyl, —CON(C₁-C₄ alkyl)(C₁-C₄ alkyl), —CONH(C₁-C₄ alkyl), —CONH₂, —NHC(O)(C₁-C₄ alkyl), —N(C₁-C₄ alkyl)C(O)(C₁-C₄ alkyl), —SO₂(C₁-C₄ alkyl), —SO₂(phenyl), —SO₂(C₁-C₄ haloalkyl), —SO₂NH₂, —SO₂NH(C₁-C₄ alkyl), —SO₂N(C₁-C₄ alkyl) (C₁-C₄ alkyl), —SO₂NH(phenyl), —SO₂N(C₁-C₄ alkyl)(phenyl), —NHSO₂(C₁-C₄ alkyl), —N(C₁-C₄ alkyl)SO₂(C₁-C₄ alkyl), —NHSO₂(phenyl), —N(C₁-C₄ alkyl)SO₂(phenyl), —NHSO₂(C₁-C₄ haloalkyl), and —N(C₁-C₄ alkyl)SO₂(C₁-C₄ haloalkyl),

m is 0, 1 or 2,

n is 1 or 2,

p is 1, 2 or 3.

Compounds described herein are useful as inhibitors of SYK. Compounds of the present invention were also found to exhibit good kinase selectivity on SYK against other kinases such as VEGFR-2 (KDR) or Flt-3.

Also provided is a pharmaceutical composition comprising at least one compound and/or at least one pharmaceutically acceptable salt thereof described herein and at least one pharmaceutically acceptable carrier.

Also provided is a method of inhibiting the activity of Syk kinase comprising inhibiting said activity with an effective amount of at least one compound and/or at least one pharmaceutically acceptable salt thereof described herein.

Also provided is a method of treating a subject with a recognized inflammatory disease responsive to inhibition of Syk comprising administering to said subject in recognized need thereof an effective amount to treat said disease of at least one compound and/or at least one pharmaceutically acceptable salt thereof described herein.

As used in the present specification, the following words, phrases and symbols are generally intended to have the meanings as set forth below, except to the extent that the context in which they are used indicates otherwise. The following abbreviations and terms have the indicated meanings throughout:

A dash (“-”) that is not between two letters or symbols is used to indicate a point of attachment for a substituent. For example, —CONH₂ is attached through the carbon atom.

The term “alkyl” herein refers to a straight or branched hydrocarbon, containing 1-18, preferably 1-12, more preferably 1-6 carbon atoms. Examples of alkyl groups include, but are not limited to, methyl, ethyl, n-propyl, i-propyl, n-butyl, i-butyl, and t-butyl. “Lower alkyl” refers to a straight or branched hydrocarbon, containing 1-6, preferably 1-4 carbon atoms.

By “alkoxy” is meant a straight or branched alkyl group containing 1-18, preferably 1-12, more preferably 1-6 carbon atoms attached through an oxygen bridge such as, for example, methoxy, ethoxy, propoxy, isopropoxy, n-butoxy, sec-butoxy, tert-butoxy, pentoxy, 2-pentyloxy, isopentoxy, neopentoxy, hexoxy, 2-hexoxy, 3-hexoxy, 3-methylpentoxy, and the like. Alkoxy groups will usually have from 1 to 6 carbon atoms attached through the oxygen bridge. “Lower alkoxy” refers to a straight or branched alkoxy, wherein the alkyl portion contains 1-6, preferably 1-4 carbon atoms.

The term “alkenyl” herein refers to a straight or branched hydrocarbon, containing one or more C═C double bonds and 2-10, preferably 2-6 carbon atoms. Examples of alkenyl groups include, but are not limited to, vinyl, 2-propenyl, and 2-butenyl.

The term “alkynyl” herein refers to a straight or branched hydrocarbon, containing one or more C≡C triple bonds and 2-10, preferably 2-6 carbon atoms. Examples of alkynyl groups include, but are not limited to, ethynyl, 2-propynyl, and 2-butynyl.

The term “alkylene” herein refers to branched and unbranched alkylene groups with 1 to 6 carbon atoms. Alkylene groups with 1 to 4 carbon atoms are preferred. Examples of these include, but are not limited to: methylene, ethylene, propylene, 1-methylethylene, butylene, 1-methylpropylene, 1,1-dimethylethylene, 1,2-dimethylethylene, pentylene, 1,1-dimethylpropylene, 2,2-dimethylpropylene, 1,2-dimethylpropylene, 1,3-dimethylpropylene or hexylene. Unless stated otherwise, the definitions propylene, butylene, pentylene and hexylene include all the possible isomeric forms of the groups in question with the same number of carbons. Thus, for example, propylene includes also 1-methylethylene and butylene includes 1-methylpropylene, 1,1-dimethylethylene, 1,2-dimethylethylene.

The term “cycloalkyl” refers to saturated and partially unsaturated cyclic hydrocarbon groups having 3 to 12, preferably 3 to 8 carbon atoms. Examples of cycloalkyl groups include, but are not limited to, cyclopropyl, cyclobutyl, cyclopentyl, cyclopentenyl, cyclohexyl, cyclohexenyl, cycloheptyl, and cyclooctyl. The ring may be saturated or have one or more double bonds (i.e. partially unsaturated), but not fully conjugated, and not aryl, as defined herein.

“Aryl” encompasses:
5- and 6-membered carbocyclic aromatic rings, for example, benzene;
8- and 12-membered bicyclic ring systems wherein at least one ring is carbocyclic and aromatic, for example, naphthalene, indane, indoline, indolin-2-one, 2,3-dihydrobenzofuran, benzo[d][1,3]dioxole, and 1,2,3,4-tetrahydroquinoline, chroman, 2,3-dihydrobenzo[b][1,4]dioxine, 3,4-dihydro-2H-benzo[b][1,4]oxazine, isochroman, 1,3-dihydroisobenzofuran, 1H-benzo[d][1,3]oxazin-2(4H)-one and
11- and 14-membered tricyclic ring systems wherein at least one ring is carbocyclic and aromatic, for example, fluorene.
   For example, aryl includes 5- and 6-membered carbocyclic aromatic rings fused to a 5- to 7-membered heterocyclic ring containing one or more heteroatoms selected from N, O, and S, provided that the point of attachment is at the carbocyclic aromatic ring. Bivalent radicals formed from substituted benzene derivatives and having the free valences at ring atoms are named as substituted phenylene radicals. Bivalent radicals derived from univalent polycyclic hydrocarbon radicals whose names end in “-yl” by removal of one hydrogen atom from the carbon atom with the free valence are named by adding “-idene” to the name of the corresponding univalent radical, e.g., a naphthyl group with two points of attachment is termed naphthylidene. Aryl, however, does not encompass or overlap in any way with heteroaryl, separately defined below. Hence, if one or more carbocyclic aromatic rings are fused with a heterocyclic aromatic ring, the resulting ring system is heteroaryl, not aryl, as defined herein.

The term “halo” includes fluoro, chloro, bromo, and iodo, and the term “halogen” includes fluorine, chlorine, bromine, and iodine.

The term “heteroaryl” refers to

5- to 8-membered aromatic, monocyclic rings containing one or more, for example, from 1 to 4, or, in some embodiments, from 1 to 3, heteroatoms selected from N, O, and S, with the remaining ring atoms being carbon;

8- to 12-membered bicyclic rings containing one or more, for example, from 1 to 4, or, in some embodiments, from 1 to 3, heteroatoms selected from N, O, and S, with the remaining ring atoms being carbon and wherein at least one heteroatom is present in an aromatic ring; and

11- to 14-membered tricyclic rings containing one or more, for example, from 1 to 4, or in some embodiments, from 1 to 3, heteroatoms selected from N, O, and S, with the remaining ring atoms being carbon and wherein at least one heteroatom is present in an aromatic ring.

For example, heteroaryl includes a 5- to 7-membered heterocyclic aromatic ring fused to a 5- to 7-membered cycloalkyl ring. For such fused, bicyclic heteroaryl ring systems wherein only one of the rings contains one or more heteroatoms, the point of attachment is at the heteroaromatic ring.

When the total number of S and O atoms in the heteroaryl group exceeds 1, those heteroatoms are not adjacent to one another. In some embodiments, the total number of S and O atoms in the heteroaryl group is not more than 2. In some embodiments, the total number of S and O atoms in the aromatic heterocycle is not more than 1.

Examples of heteroaryl groups include, but are not limited to, (as numbered from the linkage position assigned priority 1), 2-pyridyl, 3-pyridyl, 4-pyridyl, 2,3-pyrazinyl, 3,4-pyrazinyl, 2,4-pyrimidinyl, 3,5-pyrimidinyl, 1-pyrazolyl, 2,3-pyrazolyl, 2,4-imidazolinyl, isoxazolyl, oxazolyl, thiazolyl, thiadiazolyl, tetrazolyl, thienyl, benzothienyl, furyl, benzofuryl, benzoimidazolinyl, indolinyl, pyridizinyl, triazolyl, quinolinyl, pyrazolyl, and 5,6,7,8-tetrahydroisoquinoline.

Bivalent radicals derived from univalent heteroaryl radicals whose names end in “-yl” by removal of one hydrogen atom from the atom with the free valence are named by adding “-idene” to the name of the corresponding univalent radical, e.g., a pyridyl group with two points of attachment is a pyridylidene. Heteroaryl does not encompass or overlap with aryl as defined above.

Substituted heteroaryl also includes ring systems substituted with one or more oxide (—O⁻) substituents, such as pyridinyl N-oxides.

By “heterocycle” is meant a 3- to 12-membered (preferably 3- to 8-membered) monocyclic, bicyclic or tricyclic saturated or partially unsaturated ring containing at least 2 carbon atoms in addition to 1-3 heteroatoms independently selected from oxygen, sulfur, and nitrogen. “Heterocycle” also refers to 5- to 7-membered heterocyclic ring containing one or more heteroatoms selected from N, O, and S fused with 5-, 6-, and/or 7-membered cycloalkyl, heterocyclic, carbocyclic aromatic or heteroaromatic ring, provided that the point of attachment is at the heterocyclic ring. “Heterocycle” also refers to an aliphatic spirocyclic ring containing one or more heteroatoms selected from N, O, and S, provided that the point of attachment is at the heterocyclic ring. The rings may be saturated or have one or more double bonds (i.e. partially unsaturated). The heterocycle can be substituted by oxo. The point of the attachment may be carbon or heteroatom in the heterocyclic ring. A heterocyle is not a heteroaryl as defined herein.

Suitable heterocycles include, for example (as numbered from the linkage position assigned priority 1), 1-pyrrolidinyl, 2-pyrrolidinyl, 2,4-imidazolidinyl, 2,3-pyrazolidinyl, 1-piperidinyl, 2-piperidinyl, 3-piperidinyl, 4-piperidinyl, and 2,5-piperazinyl. Morpholinyl groups are also contemplated, including 2-morpholinyl and 3-morpholinyl (numbered wherein the oxygen is assigned priority 1). Substituted heterocycle also includes ring systems substituted with one or more oxo moieties, such as piperidinyl N-oxide, morpholinyl-N-oxide, 1-oxo-1-thiomorpholinyl and 1,1-dioxo-1-thiomorpholinyl.

By “optional” or “optionally” is meant that the subsequently described event or circumstance may or may not occur, and that the description includes instances where the event or circumstance occurs and instances in which it does not. For example, “optionally substituted alkyl” encompasses both “unsubstituted alkyl” and “substituted alkyl” as defined below. It will be understood by those skilled in the art, with respect to any group containing one or more substituents, that such groups are not intended to introduce any substitution or substitution patterns that are sterically impractical, synthetically non-feasible and/or inherently unstable.

The term “substituted”, as used herein, means that any one or more hydrogens on the designated atom or group is replaced with a selection from the indicated group, provided that the designated atom's normal valence is not exceeded. When a substituent is oxo (i.e., ═O) then 2 hydrogens on the atom are replaced. Combinations of substituents and/or variables are permissible only if such combinations result in stable compounds or useful synthetic intermediates. A stable compound or stable structure is meant to imply a compound that is sufficiently robust to survive isolation from a reaction mixture, and subsequent formulation as an agent having at least practical utility. Unless otherwise specified, substituents are named into the core structure. For example, it is to be understood that when (cycloalkyl)alkyl is listed as a possible substituent, the point of attachment of this substituent to the core structure is in the alkyl portion.

In some embodiments, “substituted with one or more groups” refers to two hydrogens on the designated atom or group being independently replaced with two selections from the indicated group of substituents. In some embodiments, “substituted with one or more groups” refers to three hydrogens on the designated atom or group being independently replaced with three selections from the indicated group of substituents. In some embodiments, “substituted with one or more groups” refers to four hydrogens on the designated atom or group being independently replaced with four selections from the indicated group of substituents.

Compounds described herein include, but are not limited to, when possible, to the extent that they can be made by one of ordinary skill without undue experimentation, their regioisomers, their N-oxide derivatives, their optical isomers, such as enantiomers and diastereomers, mixtures of enantiomers, including racemates, mixtures of diastereomers, and other mixtures thereof, to the extent they can be made by one of ordinary skill in the art by routine experimentation. In those situations, the single enantiomers or diastereomers, i.e., optically active forms, can be obtained by asymmetric synthesis or by resolution of the racemates or mixtures of enantiomers or diastereomers. Resolution of the racemates or mixtures of diastereomers, if possible, can be accomplished, for example, by conventional methods such as crystallization in the presence of a resolving agent, or chromatography, using, for example a chiral high-pressure liquid chromatography (HPLC) column. In addition, when possible, such compounds include Z- and E-forms (or cis- and trans-forms) of compounds with carbon-carbon double bonds. Where compounds described herein exist in various tautomeric forms, the term “compound” is intended to include, to the extent they can be made without undue experimentation, all tautomeric forms of the compound. Such compounds also include crystal forms including polymorphs and clathrates, to the extent they can be made by one of ordinary skill in the art without undue experimentation. Similarly, the term “salt” is intended to include all isomers, racemates, other mixtures, Z- and E-forms, tautomeric forms and crystal forms of the salt of the compound, to the extent they can be made by one of ordinary skill in the art without undue experimentation.

“Pharmaceutically acceptable salts” include, but are not limited to salts with inorganic acids, such as hydrochlorate, phosphate, diphosphate, hydrobromate, sulfate, sulfinate, nitrate, and like salts; as well as salts with an organic acid, such as malate, maleate, fumarate, tartrate, succinate, citrate, acetate, lactate, methanesulfonate, p-toluenesulfonate, 2-hydroxyethylsulfonate, benzoate, salicylate, stearate, and alkanoate such as acetate, salts with HOOC—(CH₂)ₙ—COOH where n is 0-4, and like salts. Similarly, pharmaceutically acceptable cations include, but are not limited to sodium, potassium, calcium, aluminum, lithium, and ammonium.

In addition, if a compound described herein is obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used without undue experimentation to prepare non-toxic pharmaceutically acceptable addition salts.

A “solvate,” such as a “hydrate,” is formed by the interaction of a solvent and a compound. The term “compound” is intended to include solvates, including hydrates, of compounds, to the extent they can be made by one of ordinary skill in the art by routine experimentation. Similarly, “salts” includes solvates, such as hydrates, of salts, to the extent they can be made by one of ordinary skill in the art by routine experimentation. Suitable solvates are pharmaceutically acceptable solvates, such as hydrates, including monohydrates and hemi-hydrates, to the extent they can be made by one of ordinary skill in the art by routine experimentation.

As used herein the terms “group”, “radical” or “fragment” are synonymous and are intended to indicate functional groups or fragments of molecules attachable to a bond or other fragments of molecules.

The term “active agent” is used to indicate a chemical substance which has biological activity. In some embodiments, an “active agent” is a chemical substance having pharmaceutical utility.

“Treating,” “treat,” or “treatment” or “alleviation” refers to administering at least one compound and/or at least one pharmaceutically acceptable salt thereof described herein to a subject that has a disease or disorder, or has a symptom of a disease or disorder, or has a predisposition toward a disease or disorder, with the purpose to cure, heal, alleviate, relieve, alter, remedy, ameliorate, improve, or affect cancer, the symptoms of the disease or disorder, or the predisposition toward the disease or disorder. In some embodiments, the disease or disorder may be cancer. In some embodiments, the disease or disorder may be an inflammatory disease.

The term “effective amount” refers to an amount of at least one compound and/or at least one pharmaceutically acceptable salt thereof described herein effective to “treat”, as defined above, a disease or disorder in a subject responsive to the inhibition of Syk. The effective amount may cause any of the changes observable or measurable in a subject as described in the definition of “treating,” “treat,” “treatment” and “alleviation” above. For example, in the case of cancer, the effective amount can reduce the number of cancer or tumor cells; reduce the tumor size; inhibit or stop tumor cell infiltration into peripheral organs including, for example, the spread of tumor into soft tissue and bone; inhibit and stop tumor metastasis; inhibit and stop tumor growth; relieve to some extent one or more of the symptoms associated with the cancer, reduce morbidity and mortality; improve quality of life; or a combination of such effects. An effective amount may be an amount sufficient to decrease the symptoms of a disease responsive to inhibition of Syk kinase

The term “effective amount” may also refer to an amount of at least one compound and/or at least one pharmaceutically acceptable salt described herein effective to inhibit the activity of Syk in a subject responsive to the inhibition of Syk.

The term “inhibition” indicates a decrease in the baseline activity of a biological activity or process. “Inhibition of Syk” refers to a decrease in the activity of Syk kinase as a direct or indirect response to the presence of at least one compound and/or at least one pharmaceutically acceptable salt thereof described herein, relative to the activity of Syk kinase in the absence of the at least one compound and/or the at least one pharmaceutically acceptable salt thereof. The decrease in activity may be due to the direct interaction of the at least one compound and/or at least one pharmaceutically acceptable salt thereof described herein with the Syk kinase, or due to the interaction of the at least one compound and/or at least one pharmaceutically acceptable salt thereof described herein, with one or more other factors that in turn affect the at least one kinase activity. For example, the presence of at least one compound and/or at least one pharmaceutically acceptable salt thereof described herein, may decrease the at least one kinase activity by directly binding to the Syk kinase, by causing (directly or indirectly) another factor to decrease the at least one kinase activity, or by (directly or indirectly) decreasing the amount of the at least one kinase present in the cell or organism.

### DETAILED DESCRIPTION OF THE INVENTION

Provided is at least one compound of formula (I):

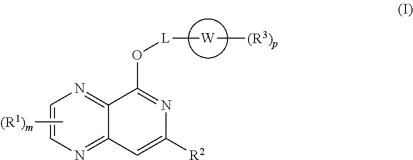

and/or its racemic mixture, enantiomers, diastereomers, tautomers, or mixtures of optional ratio, or at least one pharmaceutically acceptable salt, or solvate thereof, wherein
R¹ is independently chosen from hydrogen, halo, —CN, —OH, optionally substituted C₁-C₆ alkyl, optionally substituted C₁-C₆ alkoxy, —NH₂, —NH(C₁-C₄alkyl), and —N(C₁-C₄alkyl)(C₁-C₄alkyl),
R² is aryl, or heteroaryl, each of which is optionally substituted by one or more groups selected from halo, —NR⁵R⁶, —OR⁷, —S(O)ₙR⁸, —C(O)R⁹, —C(O)OR⁷, —CN, —C(O)NR⁵R⁶, —NR⁵C(O)R⁹, —NR⁵S(O)ₙR⁸, —NR⁵S(O)ₙNR¹⁰R¹¹, —NR⁵C(O)OR⁷, —NR⁵C(O)NR¹⁰R¹¹, —NO₂, —S(O)ₙNR⁵R⁶, optionally substituted lower alkyl, optionally substituted cycloalkyl, optionally substituted heterocycle, optionally substituted heteroaryl, optionally substituted aryl, optionally substituted alkenyl, and optionally substituted alkynyl,
L is a bond, or optionally substituted C₁-C₆alkylene,
W is cycloalkyl, heterocycle, aryl, or heteroaryl
R³ is independently selected from hydrogen, -Lx-halo, -Lx-R⁴, -Lx-NR⁵R⁶, -Lx-OR⁷, -Lx-S(O)ₙR⁸, -Lx-C(O)R⁹, —S(O)ₙ-Lx-R⁸, —C(O)-Lx-R⁹, -Lx-CN, -Lx-NR⁵C(O)R⁹, -Lx-NR⁵S(O)ₙR⁸, -Lx-NR⁵C(O)NR¹⁰R¹¹, -Lx-NR⁵S(O)ₙNR¹⁰R¹¹, -Lx-NR⁵C(O)OR⁷, -Lx-NR⁵S(O)ₙOR⁷, —NO₂, -Lx-C(O)NR⁵R⁶, -Lx-S(O)ₙNR⁵R⁶, oxo(═O), optionally substituted cycloalkyl, optionally substituted heterocycle, optionally substituted heteroaryl, and optionally substituted aryl,
provided when L is methylene and W is 5- or 6-membered heterocycle, R³ is independently selected from -Lx-NR⁵R⁶, -Lx-OR⁷, -Lx-S(O)ₙR⁸, -Lx-C(O)R⁹, —S(O)ₙ-Lx-R⁸, —C(O)-Lx-R⁹, -Lx-CN, -Lx-NR⁵C(O)R⁹, -Lx-NR⁵S(O)ₙR⁸, -Lx-NR⁵C(O)NR¹⁰R¹¹, -Lx-NR⁵S(O)ₙNR¹⁰R¹¹, -Lx-NR⁵C(O)OR⁷, -Lx-NR⁵S(O)ₙOR⁷, —NO₂, -Lx-C(O)NR⁵R⁶, -Lx-S(O)ₙ NR⁵R⁶, oxo(═O), optionally substituted cycloalkyl, optionally substituted heterocycle, optionally substituted heteroaryl, and optionally substituted aryl
R⁴ is C₁-C₆alkyl, C₂-C₆alkenyl, or C₂-C₆alkynyl, each of which is optionally substituted,
R⁵, R⁶, R⁷, R⁸, R⁹, R¹⁰, and R¹¹ are independently selected from hydrogen, alkyl, cycloalkyl, aryl, heteroaryl, and heterocycle, each of which except for hydrogen, is optionally substituted with one or more groups selected from halo, —OH, —O(C₁-C₄alkyl), —CN, C₁-C₄alkyl, —NH₂, —NH(C₁-C₄alkyl), —N(C₁-C₄alkyl)(C₁-C₄alkyl), —C(O)NH₂, —C(O)NH(C₁-C₄alkyl), —C(O)N(C₁-C₄alkyl)(C₁-C₄alkyl), —C(O)(C₁-C₄alkyl), —NHC(O)(C₁-C₄alkyl), —N(C₁-C₄alkyl)C(O)(C₁-C₄alkyl), —S(O)ₙNH₂, —S(O)ₙNH(C₁-C₄alkyl), —S(O)ₙN(C₁-C₄alkyl)(C₁-C₄alkyl), —S(O)ₙ(C₁-C₄alkyl), —NHS(O)ₙ(C₁-C₄alkyl), —N(C₁-C₄alky)S(O)ₙ(C₁-C₄alkyl), optionally substituted C₃-C₈ cycloalkyl, and optionally substituted 3-8 membered heterocycle, wherein C₁-C₄alkyl is optionally substituted by halo, —OH, —OMe, or —CN,
or R⁵ and R⁶, R⁵ and R⁷, R⁵ and R⁸, R⁵ and R⁹, and R⁵ and R¹⁰ together with the atom(s) to which they are attached can form a ring, which is optionally substituted with one or more groups selected from halo, —OH, —O(C₁-C₄alkyl), —CN, C₁-C₄alkyl, —NH₂, —NH(C₁-C₄alkyl), —N(C₁-C₄alkyl)(C₁-C₄alkyl), —C(O)NH₂, —C(O)NH(C₁-C₄alkyl), —C(O)N(C₁-C₄alkyl)(C₁-C₄alkyl), —C(O)(C₁-C₄alkyl), —NHC(O)(C₁-C₄alkyl), —N(C₁-C₄alkyl)C(O)(C₁-C₄alkyl), —S(O)ₙNH₂, —S(O)ₙNH(C₁-C₄alkyl), —S(O)ₙN(C₁-C₄alkyl)(C₁-C₄alkyl), —S(O)ₙ(C₁-C₄alkyl), —NHS(O)ₙ(C₁-C₄alkyl), —N(C₁-C₄alky)S(O)ₙ(C₁-C₄alkyl), optionally substituted C₃-C₈ cycloalkyl, and optionally substituted 3-8 membered heterocycle, wherein C₁-C₄alkyl is optionally substituted by halo, —OH, —OMe, or —CN,
Lx is a bond, or optionally substituted C₁-C₆alkylene,
wherein each optionally substituted group above for which the substituent(s) is (are) not specifically designated, can be unsubstituted or independently substituted with, for example, one or more, such as one, two, or three, substituents independently chosen from C₁-C₄ alkyl, cycloalkyl, aryl, heterocycle, heteroaryl, aryl-C₁-C₄ alkyl-, heteroaryl-C₁-C₄ alkyl-, C₁-C₄ haloalkyl-, —OC₁—C₄ alkyl, —OC₁—C₄ alkylphenyl, —C₁-C₄ alkyl-OH, —C₁-C₄ alkyl-O—C₁-C₄ alkyl, —OC₁—C₄ haloalkyl, halo, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo, —CO₂H, —C(O)OC₁—C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl, —C(O)(C₃-C₈cycloalkyl), —C(O)(C₅-C₁₀aryl), —C(O)(C₃-C₈heterocycle), —C(O)(C₅-C₁₀heteroaryl), —C(O) (C₁-C₄alkyl)-(C₃-C₈cycloalkyl), —C(O)(C₁-C₄alkyl)-(C₅-C₁₀aryl), —C(O)(C₁-C₄alkyl)-(C₃-C₈heterocycle), —C(O)(C₁-C₄alkyl)-(C₅-C₁₀heteroaryl), —C(O)C₁-C₄ haloalkyl, —OC(O)C₁-C₄ alkyl, —SO₂(C₁-C₄ alkyl), —SO₂(C₃-C₈cycloalkyl), —SO₂(C₅-C₁₀aryl), —SO₂(C₃-C₈heterocycle), —SO₂(C₅-C₁₀heteroaryl), —SO₂(C₁-C₄alkyl)-(C₃-C₈cycloalkyl), —SO₂(C₁-C₄alkyl)-(C₅-C₁₀aryl), —SO₂(C₁-C₄alkyl)-(C₃-C₈heterocycle), —SO₂(C₁-C₄alkyl)-(C₅-C₁₀heteroaryl), —SO₂(C₁-C₄ haloalkyl), —SO₂NH₂, —SO₂NH(C₁-C₄ alkyl), —SO₂N(C₁-C₄ alkyl)(C₁-C₄ alkyl), —SO₂NH(phenyl), —SO₂N(C₁-C₄ alkyl) (phenyl), —NHSO₂(C₁-C₄ alkyl), —NHSO₂(phenyl), and —NHSO₂(C₁-C₄ haloalkyl), in which each of alkyl, cycloalkyl, aryl, heterocycle, and heteroaryl is optionally substituted by one or more groups chosen from halo, cycloalkyl, heterocycle, C₁-C₄ alkyl, C₁-C₄ haloalkyl-, —OC₁—C₄ alkyl, C₁-C₄ alkyl-OH, —C₁-C₄ alkyl-O—C₁-C₄ alkyl, —OC₁—C₄ haloalkyl, cyano, nitro, —NH₂, —OH, —CO₂H, —C(O)OC₁—C₄ alkyl, —CON(C₁-C₄ alkyl)(C₁-C₄ alkyl), —CONH(C₁-C₄ alkyl), —CONH₂, —NHC(O)(C₁-C₄ alkyl), —N(C₁-C₄ alkyl)C(O)(C₁-C₄ alkyl), —SO₂(C₁-C₄ alkyl), —SO₂(phenyl), —SO₂(C₁-C₄ haloalkyl), —SO₂NH₂, —SO₂NH(C₁-C₄ alkyl), —SO₂N(C₁-C₄ alkyl) (C₁-C₄ alkyl), —SO₂NH(phenyl), —SO₂N(C₁-C₄ alkyl)(phenyl), —NHSO₂(C₁-C₄ alkyl), —N(C₁-C₄ alkyl)SO₂(C₁-C₄ alkyl), —NHSO₂(phenyl), —N(C₁-C₄ alkyl)SO₂(phenyl), —NHSO₂(C₁-C₄ haloalkyl), and —N(C₁-C₄ alkyl)SO₂(C₁-C₄ haloalkyl),

m is 0, 1 or 2,

n is 1 or 2,

p is 1, 2 or 3.

In some embodiments, R¹ is independently chosen from hydrogen, halo, —OH, —CN, optionally substituted C₁-C₆alkyl, and optionally substituted C₁-C₆alkoxy, —NH₂, —NH(C₁-C₄ alkyl), and —N(C₁-C₄ alkyl)(C₁-C₄ alkyl).

In some embodiments, R¹ is independently chosen from hydrogen, halo, —CN, hydroxyl; or is chosen from methyl, ethyl, n-propyl, i-propyl, —NH₂, N-methylamino, N,N-dimethylamino, N-ethylamino, N-n-propylamino, N-i-propylamino, methoxy, ethoxy, propoxy, and isopropoxy, each of which is optionally substituted.

In some embodiments, R¹ is hydrogen.

In some embodiments, m is 1.

In some embodiments, p is 1, or 2.

In some embodiments, R² is C₅-C₁₀aryl, or 5-10 membered heteroaryl, each of which is optionally substituted by one or more groups selected from halo, —NR⁵R⁶, —OR⁷, —S(O)ₙR⁸, —C(O)R⁹, —C(O)OR⁷, —CN, —C(O)NR⁵R⁶, —NR⁵C(O)R⁹, —NR⁵S(O)ₙR⁸, —NR⁵S(O)ₙNR¹⁶R¹¹, —NR⁵C(O)OR⁷, —NR⁵C(O)NR¹⁰R¹¹, —NO₂, —S(O)ₙNR⁵R⁶, optionally substituted C₁-C₆ alkyl, optionally substituted C₃-C₈ cycloalkyl, optionally substituted 3-8 membered heterocycle, optionally substituted 5-10 membered heteroaryl, optionally substituted C₅-C₁₀ aryl, optionally substituted C₂-C₆ alkenyl, and optionally substituted C₂-C₆ alkynyl,

R⁵, R⁶, R⁷, R⁸, R⁹, R¹⁰, and R¹¹ are independently selected from hydrogen, alkyl, cycloalkyl, aryl, heteroaryl, and heterocycle, each of which except for hydrogen, is optionally substituted with one or more groups selected from halo, —OH, —O(C₁-C₄ alkyl), —CN, C₁-C₄ alkyl, —NH₂, —NH(C₁-C₄ alkyl), —N(C₁-C₄ alkyl)(C₁-C₄ alkyl), —C(O)NH₂, —C(O)NH(C₁-C₄ alkyl), —C(O)N(C₁-C₄ alkyl)(C₁-C₄ alkyl), —C(O)(C₁-C₄ alkyl), —NHC(O)(C₁-C₄ alkyl), —N(C₁-C₄ alkyl)C(O)(C₁-C₄ alkyl), —S(O)ₙNH₂, —S(O)ₙNH(C₁-C₄ alkyl), —S(O)ₙN(C₁-C₄ alkyl)(C₁-C₄ alkyl), —S(O)ₙ(C₁-C₄ alkyl), —NHS(O)ₙ(C₁-C₄ alkyl), —N(C₁-C₄ alky)S(O)ₙ(C₁-C₄ alkyl), optionally substituted C₃-C₈ cycloalkyl, and optionally substituted 3-8 membered heterocycle, wherein C₁-C₄ alkyl is optionally substituted by halo, —OH, —OMe, or —CN,

or R⁵ and R⁶, R⁵ and R⁷, R⁵ and R⁸, R⁵ and R⁹, and R⁵ and R¹⁰ together with the atom(s) to which they are attached can form a ring, which is optionally substituted with one or more groups selected from halo, —OH, —O(C₁-C₄ alkyl), —CN, C₁-C₄ alkyl, —NH₂, —NH(C₁-C₄ alkyl), —N(C₁-C₄ alkyl)(C₁-C₄ alkyl), —C(O)NH₂, —C(O)NH(C₁-C₄ alkyl), —C(O)N(C₁-C₄ alkyl)(C₁-C₄ alkyl), —C(O)(C₁-C₄ alkyl), —NHC(O)(C₁-C₄ alkyl), —N(C₁-C₄ alkyl)C(O)(C₁-C₄ alkyl), —S(O)ₙNH₂, —S(O)ₙNH(C₁-C₄ alkyl), —S(O)ₙN(C₁-C₄ alkyl)(C₁-C₄ alkyl), —S(O)ₙ(C₁-C₄ alkyl), —NHS(O)ₙ(C₁-C₄ alkyl), —N(C₁-C₄ alky)S(O)ₙ(C₁-C₄ alkyl), optionally substituted C₃-C₈ cycloalkyl, and optionally substituted 3-8 membered heterocycle, wherein C₁-C₄ alkyl is optionally substituted by halo, —OH, —OMe, or —CN.

In some embodiments, R² is independently chosen from phenyl, naphthyl, pyridyl, pyrimidinyl, pyrazinyl, pyridazinyl, pyrrolyl pyrazolyl, imidazolinyl, oxazolyl, isoxazolyl, thiazolyl, thienyl, furyl, benzofuryl, benzothienyl, benzoimidazolinyl, indolyl, indazolyl, quinolinyland, indanyl, indolinyl, indolin-2-one, 2,3-dihydrobenzofuryl, benzo[d][1,3]dioxolyl, and 1,2,3,4-tetrahydroquinolinyl, chroman, 2,3-dihydrobenzo[b][1,4]dioxinyl, 3,4-dihydro-2H-benzo[b][1,4]oxazinyl, isochroman, 1,3-dihydroisobenzofuryl, 1H-benzo[d][1,3]oxazin-2(4H)-onyl, each of which is optionally substituted by one or more groups selected from halo, —NR⁵R⁶, —OR⁷, —S(O)ₙR⁸, —C(O)R⁹, —C(O)OR⁷, —CN, —C(O)NR⁵R⁶, —NR⁵C(O)R⁹, —NR⁵S(O)ₙR⁸, —NR⁵S(O)ₙNR¹⁰R¹¹, —NR⁵C(O)OR⁷, —NR⁵C(O)NR¹⁰R¹¹, —NO₂, and —S(O)ₙNR⁵R⁶; or selected from methyl, ethyl, n-propyl, i-propyl, n-butyl, i-butyl, and t-butyl, cyclopropyl, cyclobutyl, cyclopentyl, cyclopentenyl, cyclohexyl, cyclohexenyl, pyrrolidinyl, tetrahydrofuryl, tetrahydropyranyl, piperidinyl, piperazinyl, morpholinyl, homomorpholinyl, thiomorpholinyl, diazepanyl, oxazepanyl, pyridyl, pyrimidinyl, pyrazinyl, pyridazinyl, pyrroly, l pyrazolyl, imidazolinyl, oxazolyl, isoxazolyl, thiazolyl, thienyl, furyl, benzofuryl, benzothienyl, benzoimidazolinyl, indolyl, indazolyl, quinolinyl, phenyl, and naphthyl, each of which is optionally substituted by one or more groups selected from halo, —NR⁵R⁶, —OR⁷, —S(O)ₙR⁸, —C(O)R⁹, —C(O)OR⁷, —CN, —C(O)NR⁵R⁶, —NR⁵C(O)R⁹, —NR⁵S(O)ₙR⁸, —NR⁵S(O)ₙNR¹⁰R¹¹, —NR⁵C(O)OR⁷, —NR⁵C(O)NR¹⁰R¹¹, —NO₂, —S(O)ₙNR⁵R⁶, optionally substituted lower alkyl, optionally substituted cycloalkyl, optionally substituted heterocycle, optionally substituted heteroaryl, optionally substituted aryl, optionally substituted alkenyl, and optionally substituted alkynyl,

R⁵, R⁶, R⁷, R⁸, R⁹, R¹⁰, and R¹¹ are independently selected from hydrogen, alkyl, cycloalkyl, aryl, heteroaryl, and heterocycle, each of which except for hydrogen, is optionally substituted with one or more groups selected from halo, —OH, —O(C₁-C₄ alkyl), —CN, C₁-C₄ alkyl, —NH₂, —NH(C₁-C₄ alkyl), —N(C₁-C₄ alkyl)(C₁-C₄ alkyl), —C(O)NH₂, —C(O)NH(C₁-C₄ alkyl), —C(O)N(C₁-C₄ alkyl)(C₁-C₄ alkyl), —C(O)(C₁-C₄ alkyl), —NHC(O)(C₁-C₄ alkyl), —N(C₁-C₄ alkyl)C(O)(C₁-C₄ alkyl), —S(O)ₙNH₂, —S(O)ₙNH(C₁-C₄ alkyl), —S(O)ₙN(C₁-C₄ alkyl)(C₁-C₄ alkyl), —S(O)ₙ(C₁-C₄ alkyl), —NHS(O)ₙ(C₁-C₄ alkyl), —N(C₁-C₄ alky)S(O)ₙ(C₁-C₄ alkyl), optionally substituted C₃-C₈ cycloalkyl, and optionally substituted 3-8 membered heterocycle, wherein C₁-C₄ alkyl is optionally substituted by halo, —OH, —OMe, or —CN,

or R⁵ and R⁶, R⁵ and R⁷, R⁵ and R⁸, R⁵ and R⁹, and R⁵ and R¹⁰ together with the atom(s) to which they are attached can form a ring, which is optionally substituted with one or more groups selected from halo, —OH, —O(C₁-C₄ alkyl), —CN, C₁-C₄ alkyl, —NH₂, —NH(C₁-C₄ alkyl), —N(C₁-C₄ alkyl)(C₁-C₄ alkyl), —C(O)NH₂, —C(O)NH(C₁-C₄ alkyl), —C(O)N(C₁-C₄ alkyl)(C₁-C₄ alkyl), —C(O)(C₁-C₄ alkyl), —NHC(O)(C₁-C₄ alkyl), —N(C₁-C₄ alkyl)C(O)(C₁-C₄ alkyl), —S(O)ₙNH₂, —S(O)ₙNH(C₁-C₄ alkyl), —S(O)ₙN(C₁-C₄ alkyl)(C₁-C₄ alkyl), —S(O)ₙ(C₁-C₄ alkyl), —NHS(O)ₙ(C₁-C₄ alkyl), —N(C₁-C₄ alky)S(O)ₙ(C₁-C₄ alkyl), optionally substituted C₃-C₈ cycloalkyl, and optionally substituted 3-8 membered heterocycle, wherein C₁-C₄ alkyl is optionally substituted by halo, —OH, —OMe, or —CN.

In some embodiments, R² is chosen from

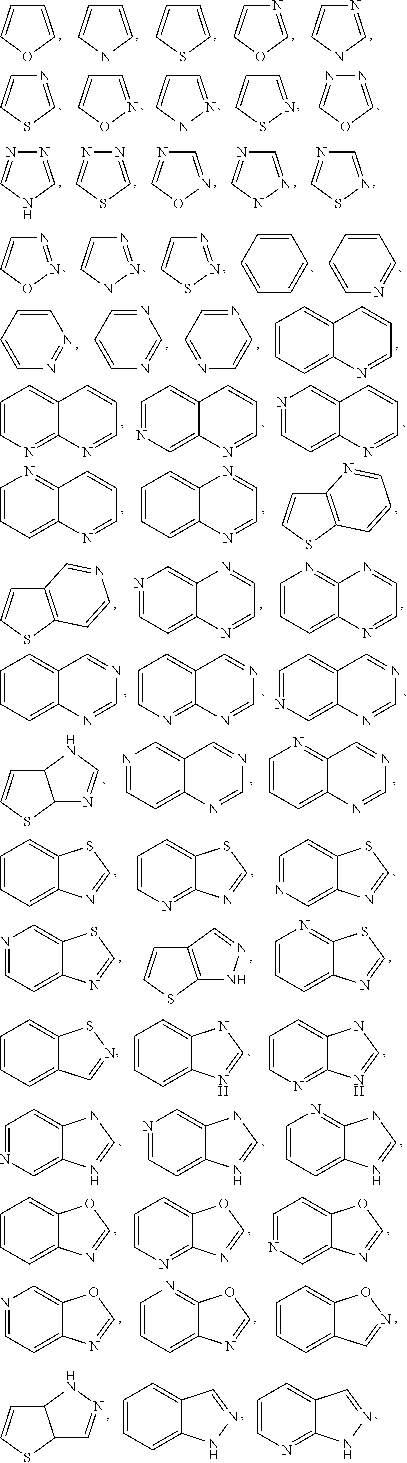

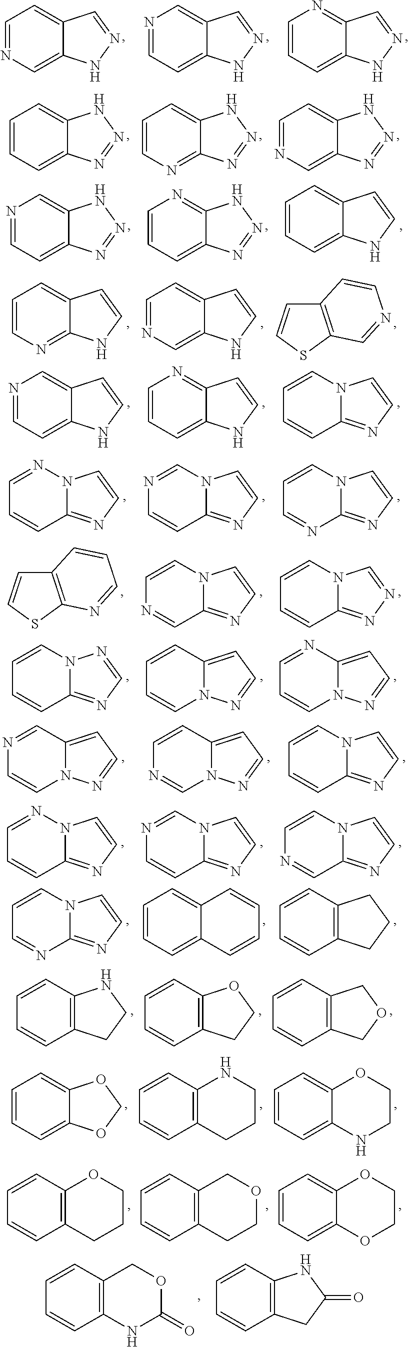

each of which is optionally substituted by one or more groups selected from halo, —NR⁵R⁶, —OR⁷, —S(O)ₙR⁸, —C(O)R⁹, —C(O)OR⁷, —CN, —C(O)NR⁵R⁶, —NR⁵C(O)R⁹, —NR⁵S(O)ₙR⁸, —NR⁵S(O)ₙNR¹⁰R¹¹, —NR⁵C(O)OR⁷, —NR⁵C(O)NR¹⁰R¹¹, —NO₂, and —S(O)ₙNR⁵R⁶; or selected from methyl, ethyl, n-propyl, i-propyl, n-butyl, i-butyl, and t-butyl, cyclopropyl, cyclobutyl, cyclopentyl, cyclopentenyl, cyclohexyl, cyclohexenyl, pyrrolidinyl, tetrahydrofuryl, tetrahydropyranyl, piperidinyl, piperazinyl, morpholinyl, homomorpholinyl, thiomorpholinyl, diazepanyl, oxazepanyl, pyridyl, pyrimidinyl, pyrazinyl, pyridazinyl, pyrroly, l pyrazolyl, imidazolinyl, oxazolyl, isoxazolyl, thiazolyl, thienyl, furyl, benzofuryl, benzothienyl, benzoimidazolinyl, indolyl, indazolyl, quinolinyl, phenyl, and naphthyl, each of which is optionally substituted by one or more groups selected from halo, —NR⁵R⁶, —OR⁷, —S(O)ₙR⁵, —C(O)R⁹, —C(O)OR⁷, —CN, —C(O)NR⁵R⁶, —NR⁵C(O)R⁹, —NR⁵S(O)ₙR⁵, —NR⁵S(O)ₙNR¹⁰R¹¹, —NR⁵C(O)OR⁷, —NR⁵C(O)NR¹⁰R¹¹, —NO₂, —S(O)ₙNR⁵R⁶, optionally substituted lower alkyl, optionally substituted cycloalkyl, optionally substituted heterocycle, optionally substituted heteroaryl, optionally substituted aryl, optionally substituted alkenyl, and optionally substituted alkynyl,

R⁵, R⁶, R⁷, R⁸, R⁹, R¹⁰, and R¹¹ are independently selected from hydrogen, alkyl, cycloalkyl, aryl, heteroaryl, and heterocycle, each of which except for hydrogen, is optionally substituted with one or more groups selected from halo, —OH, —O(C₁-C₄ alkyl), —CN, C₁-C₄ alkyl, —NH₂, —NH(C₁-C₄ alkyl), —N(C₁-C₄ alkyl)(C₁-C₄ alkyl), —C(O)NH₂, —C(O)NH(C₁-C₄ alkyl), —C(O)N(C₁-C₄ alkyl)(C₁-C₄ alkyl), —C(O)(C₁-C₄ alkyl), —NHC(O)(C₁-C₄ alkyl), —N(C₁-C₄ alkyl)C(O)(C₁-C₄ alkyl), —S(O)ₙNH₂, —S(O)ₙNH(C₁-C₄ alkyl), —S(O)ₙN(C₁-C₄ alkyl)(C₁-C₄ alkyl), —S(O)ₙ(C₁-C₄ alkyl), —NHS(O)ₙ(C₁-C₄ alkyl), —N(C₁-C₄ alky)S(O)ₙ(C₁-C₄ alkyl), optionally substituted C₃-C₈ cycloalkyl, and optionally substituted 3-8 membered heterocycle, wherein C₁-C₄ alkyl is optionally substituted by halo, —OH, —OMe, or —CN,

or R⁵ and R⁶, R⁵ and R⁷, R⁵ and R⁸, R⁵ and R⁹, and R⁵ and R¹⁰ together with the atom(s) to which they are attached can form a ring, which is optionally substituted with one or more groups selected from halo, —OH, —O(C₁-C₄ alkyl), —CN, C₁-C₄ alkyl, —NH₂, —NH(C₁-C₄ alkyl), —N(C₁-C₄ alkyl)(C₁-C₄ alkyl), —C(O)NH₂, —C(O)NH(C₁-C₄ alkyl), —C(O)N(C₁-C₄ alkyl)(C₁-C₄ alkyl), —C(O)(C₁-C₄ alkyl), —NHC(O)(C₁-C₄ alkyl), —N(C₁-C₄ alkyl)C(O)(C₁-C₄ alkyl), —S(O)ₙNH₂, —S(O)ₙNH(C₁-C₄ alkyl), —S(O)ₙN(C₁-C₄ alkyl)(C₁-C₄ alkyl), —S(O)ₙ(C₁-C₄ alkyl), —NHS(O)ₙ(C₁-C₄ alkyl), —N(C₁-C₄ alky)S(O)ₙ(C₁-C₄ alkyl), optionally substituted C₃-C₈ cycloalkyl, and optionally substituted 3-8 membered heterocycle, wherein C₁-C₄ alkyl is optionally substituted by halo, —OH, —OMe, or —CN.

In some embodiments, R² is chosen from

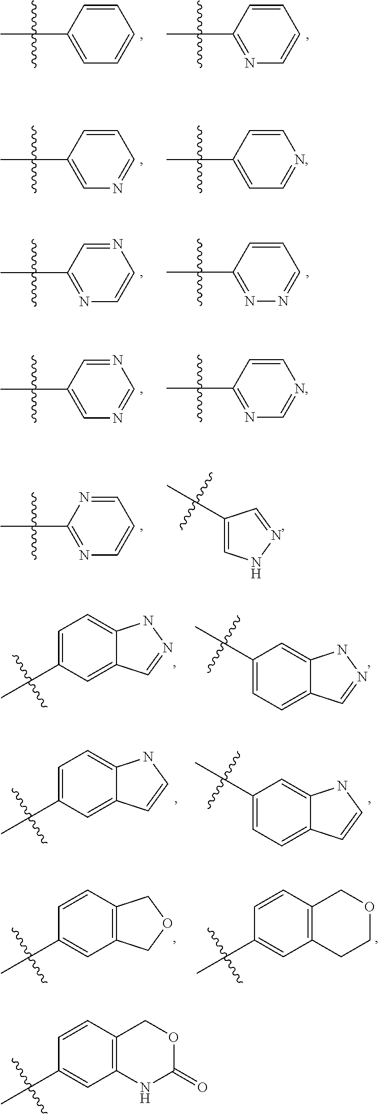

each of which is optionally substituted by one or more groups selected from halo, —NR⁵R⁶, —OR⁷, —S(O)ₙR⁸, —C(O)R⁹, —C(O)OR⁷, —CN, —C(O)NR⁵R⁶, —NR⁵C(O)R⁹, —NR⁵S(O)ₙR⁸, —NR⁵S(O)ₙNR¹⁰R¹¹, —NR⁵C(O)OR⁷, —NR⁵C(O)NR¹⁰R¹¹, —NO₂, and —S(O)ₙNR⁵R⁶; or selected from methyl, ethyl, n-propyl, i-propyl, n-butyl, i-butyl, and t-butyl, cyclopropyl, cyclobutyl, cyclopentyl, cyclopentenyl, cyclohexyl, cyclohexenyl, pyrrolidinyl, tetrahydrofuryl, tetrahydropyranyl, piperidinyl, piperazinyl, morpholinyl, homomorpholinyl, thiomorpholinyl, diazepanyl, oxazepanyl, pyridyl, pyrimidinyl, pyrazinyl, pyridazinyl, pyrroly, l pyrazolyl, imidazolinyl, oxazolyl, isoxazolyl, thiazolyl, thienyl, furyl, benzofuryl, benzothienyl, benzoimidazolinyl, indolyl, indazolyl, quinolinyl, phenyl, and naphthyl, each of which is optionally substituted by one or more groups selected from halo, —NR⁵R⁶, —OR⁷, —S(O)ₙR⁸, —C(O)R⁹, —C(O)OR⁷, —CN, —C(O)NR⁵R⁶, —NR⁵C(O)R⁹, —NR⁵S(O)ₙR⁸, —NR⁵S(O)ₙNR¹⁰R¹¹, —NR⁵C(O)OR⁷, —NR⁵C(O)NR¹⁰R¹¹, —NO₂, —S(O)ₙNR⁵R⁶, optionally substituted lower alkyl, optionally substituted cycloalkyl, optionally substituted heterocycle, optionally substituted heteroaryl, optionally substituted aryl, optionally substituted alkenyl, and optionally substituted alkynyl,

R⁵, R⁶, R⁷, R⁸, R⁹, R¹⁰, and R¹¹ are independently selected from hydrogen, alkyl, cycloalkyl, aryl, heteroaryl, and heterocycle, each of which except for hydrogen, is optionally substituted with one or more groups selected from halo, —OH, —O(C₁-C₄ alkyl), —CN, C₁-C₄ alkyl, —NH₂, —NH(C₁-C₄ alkyl), —N(C₁-C₄ alkyl)(C₁-C₄ alkyl), —C(O)NH₂, —C(O)NH(C₁-C₄ alkyl), —C(O)N(C₁-C₄ alkyl)(C₁-C₄ alkyl), —C(O)(C₁-C₄ alkyl), —NHC(O)(C₁-C₄ alkyl), —N(C₁-C₄ alkyl)C(O)(C₁-C₄ alkyl), —S(O)ₙNH₂, —S(O)ₙNH(C₁-C₄ alkyl), —S(O)ₙN(C₁-C₄ alkyl)(C₁-C₄ alkyl), —S(O)ₙ(C₁-C₄ alkyl), —NHS(O)ₙ(C₁-C₄ alkyl), —N(C₁-C₄ alky)S(O)ₙ(C₁-C₄ alkyl), optionally substituted C₃-C₈ cycloalkyl, and optionally substituted 3-8 membered heterocycle, wherein C₁-C₄ alkyl is optionally substituted by halo, —OH, —OMe, or —CN,

or R⁵ and R⁶, R⁵ and R⁷, R⁵ and R⁸, R⁵ and R⁹, and R⁵ and R¹⁰ together with the atom(s) to which they are attached can form a ring, which is optionally substituted with one or more groups selected from halo, —OH, —O(C₁-C₄ alkyl), —CN, C₁-C₄ alkyl, —NH₂, —NH(C₁-C₄ alkyl), —N(C₁-C₄ alkyl)(C₁-C₄ alkyl), —C(O)NH₂, —C(O)NH(C₁-C₄ alkyl), —C(O)N(C₁-C₄ alkyl)(C₁-C₄ alkyl), —C(O)(C₁-C₄ alkyl), —NHC(O)(C₁-C₄ alkyl), —N(C₁-C₄ alkyl)C(O)(C₁-C₄ alkyl), —S(O)ₙNH₂, —S(O)ₙNH(C₁-C₄ alkyl), —S(O)ₙN(C₁-C₄ alkyl)(C₁-C₄ alkyl), —S(O)ₙ(C₁-C₄ alkyl), —NHS(O)ₙ(C₁-C₄ alkyl), —N(C₁-C₄ alky)S(O)ₙ(C₁-C₄ alkyl), optionally substituted C₃-C₈ cycloalkyl, and optionally substituted 3-8 membered heterocycle, wherein C₁-C₄ alkyl is optionally substituted by halo, —OH, —OMe, or —CN.

In some embodiments, R² is

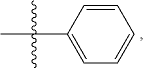 [SMILES: *c1ccccc1]

which is optionally substituted by one or more groups selected from halo, —NR⁵R⁶, —OR⁷, —S(O)ₙR⁸, —C(O)R⁹, —C(O)OR⁷, —CN, —C(O)NR⁵R⁶, —NR⁵C(O)R⁹, —NR⁵S(O)ₙR⁸, —NR⁵S(O)ₙNR¹⁰R¹¹, —NR⁵C(O)OR⁷, —NR⁵C(O)NR¹⁰R¹¹, —NO₂, and —S(O)ₙNR⁵R⁶; or selected from methyl, ethyl, n-propyl, i-propyl, n-butyl, i-butyl, and t-butyl, cyclopropyl, cyclobutyl, cyclopentyl, cyclopentenyl, cyclohexyl, cyclohexenyl, pyrrolidinyl, tetrahydrofuryl, tetrahydropyranyl, piperidinyl, piperazinyl, morpholinyl, homomorpholinyl, thiomorpholinyl, diazepanyl, oxazepanyl, pyridyl, pyrimidinyl, pyrazinyl, pyridazinyl, pyrroly, l pyrazolyl, imidazolinyl, oxazolyl, isoxazolyl, thiazolyl, thienyl, furyl, benzofuryl, benzothienyl, benzoimidazolinyl, indolyl, indazolyl, quinolinyl, phenyl, and naphthyl, each of which is optionally substituted by one or more groups selected from halo, —NR⁵R⁶, —OR⁷, —S(O)ₙR⁸, —C(O)R⁹, —C(O)OR⁷, —CN, —C(O)NR⁵R⁶, —NR⁵C(O)R⁹, —NR⁵S(O)ₙR⁸, —NR⁵S(O)ₙNR¹⁰R¹¹, —NR⁵C(O)OR⁷, —NR⁵C(O)NR¹⁰R¹¹, —NO₂, —S(O)ₙNR⁵R⁶, optionally substituted lower alkyl, optionally substituted cycloalkyl, optionally substituted heterocycle, optionally substituted heteroaryl, optionally substituted aryl, optionally substituted alkenyl, and optionally substituted alkynyl,

R⁵, R⁶, R⁷, R⁸, R⁹, R¹⁰, and R¹¹ are independently selected from hydrogen, alkyl, cycloalkyl, aryl, heteroaryl, and heterocycle, each of which except for hydrogen, is optionally substituted with one or more groups selected from halo, —OH, —O(C₁-C₄ alkyl), —CN, C₁-C₄ alkyl, —NH₂, —NH(C₁-C₄ alkyl), —N(C₁-C₄ alkyl)(C₁-C₄ alkyl), —C(O)NH₂, —C(O)NH(C₁-C₄ alkyl), —C(O)N(C₁-C₄ alkyl)(C₁-C₄ alkyl), —C(O)(C₁-C₄ alkyl), —NHC(O)(C₁-C₄ alkyl), —N(C₁-C₄ alkyl)C(O)(C₁-C₄ alkyl), —S(O)ₙNH₂, —S(O)ₙNH(C₁-C₄ alkyl), —S(O)ₙN(C₁-C₄ alkyl)(C₁-C₄ alkyl), —S(O)ₙ(C₁-C₄ alkyl), —NHS(O)ₙ(C₁-C₄ alkyl), —N(C₁-C₄ alky)S(O)ₙ(C₁-C₄ alkyl), optionally substituted C₃-C₈ cycloalkyl, and optionally substituted 3-8 membered heterocycle, wherein C₁-C₄ alkyl is optionally substituted by halo, —OH, —OMe, or —CN,

or R⁵ and R⁶, R⁵ and R⁷, R⁵ and R⁸, R⁵ and R⁹, and R⁵ and R¹⁰ together with the atom(s) to which they are attached can form a ring, which is optionally substituted with one or more groups selected from halo, —OH, —O(C₁-C₄ alkyl), —CN, C₁-C₄ alkyl, —NH₂, —NH(C₁-C₄ alkyl), —N(C₁-C₄ alkyl)(C₁-C₄ alkyl), —C(O)NH₂, —C(O)NH(C₁-C₄ alkyl), —C(O)N(C₁-C₄ alkyl)(C₁-C₄ alkyl), —C(O)(C₁-C₄ alkyl), —NHC(O)(C₁-C₄ alkyl), —N(C₁-C₄ alkyl)C(O)(C₁-C₄ alkyl), —S(O)ₙNH₂, —S(O)ₙNH(C₁-C₄ alkyl), —S(O)ₙN(C₁-C₄ alkyl)(C₁-C₄ alkyl), —S(O)ₙ(C₁-C₄ alkyl), —NHS(O)ₙ(C₁-C₄ alkyl), —N(C₁-C₄ alky)S(O)ₙ(C₁-C₄ alkyl), optionally substituted C₃-C₈ cycloalkyl, and optionally substituted 3-8 membered heterocycle, wherein C₁-C₄ alkyl is optionally substituted by halo, —OH, —OMe, or —CN.

In some embodiments, L is a bond.

In some embodiments, L is —CH₂—.

In some embodiments, L is —CH₂CH₂—.

In some embodiments, W is C₃-C₈ cycloalkyl, 3-8 membered heterocycle, C₅-C₁₀ aryl, or 5-10 membered heteroaryl.

In some embodiments, W is cyclopropyl, cyclobutyl, cyclopentyl, cyclopentenyl, cyclohexyl, cyclohexenyl, pyrrolidinyl, tetrahydrofuryl, tetrahydropyranyl, piperidinyl, piperazinyl, morpholinyl, homomorpholinyl, thiomorpholinyl, phenyl, naphthyl pyridyl, pyrimidinyl, pyrazinyl, pyridazinyl, pyrrolyl, pyrazolyl, imidazolinyl, oxazolyl, isoxazolyl, thiazolyl, thienyl, furyl, benzofuryl, benzothienyl, benzoimidazolinyl, indolyl, indazolyl, or quinolinyl.

In some embodiments, W is cyclohexyl, tetrahydrofuryl, tetrahydropyranyl, piperidinyl, morpholinyl, phenyl, or pyrazolyl.

In some embodiments, W is tetrahydrofuryl.

In some embodiments, W is

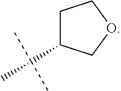

In some embodiments, W is tetrahydropyranyl.

In some embodiments, W is

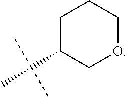

In some embodiments, W is morpholinyl.

In some embodiments, W is morpholinyl, which is substituted by R³ on nitrogen atom.

In some embodiments, W is

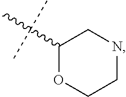

which is substituted by R³ on nitrogen atom, wherein R³ is independently selected from -Lx-S(O)ₙR⁵, -Lx-C(O)R⁹, —S(O)ₙ-Lx-R⁵, —C(O)-Lx-R⁹, -Lx-NR⁵C(O)R⁹, -Lx-NR⁵S(O)ₙR⁵, -Lx-NR⁵C(O)NR¹⁰R¹¹, -Lx-NR⁵S(O)ₙNR¹⁰R¹¹, -Lx-C(O)NR⁵R⁶, -Lx-S(O)ₙNR⁵R⁶;

R⁵, R⁶, R⁷, R⁸, and R⁹ are independently selected from hydrogen, alkyl, cycloalkyl, aryl, heteroaryl, and heterocycle, each of which except for hydrogen, is optionally substituted with one or more groups selected from halo, —OH, —O(C₁-C₄ alkyl), —CN, C₁-C₄ alkyl, —NH₂, —NH(C₁-C₄ alkyl), —N(C₁-C₄ alkyl)(C₁-C₄ alkyl), —C(O)NH₂, —C(O)NH(C₁-C₄ alkyl), —C(O)N(C₁-C₄ alkyl)(C₁-C₄ alkyl), —C(O)(C₁-C₄ alkyl), —NHC(O)(C₁-C₄ alkyl), —N(C₁-C₄ alkyl)C(O)(C₁-C₄ alkyl), —S(O)ₙNH₂, —S(O)ₙNH(C₁-C₄ alkyl), —S(O)ₙN(C₁-C₄ alkyl)(C₁-C₄ alkyl), —S(O)ₙ(C₁-C₄ alkyl), —NHS(O)ₙ(C₁-C₄ alkyl), —N(C₁-C₄ alky)S(O)ₙ(C₁-C₄ alkyl), optionally substituted C₃-C₈ cycloalkyl, and optionally substituted 3-8 membered heterocycle, wherein C₁-C₄ alkyl is optionally substituted by halo, —OH, —OMe, or —CN,

or R⁵ and R⁶, R⁵ and R⁷, R⁵ and R⁸, and R⁵ and R⁹ together with the atom(s) to which they are attached can form a ring, which is optionally substituted with one or more groups selected from halo, —OH, —O(C₁-C₄ alkyl), —CN, C₁-C₄ alkyl, —NH₂, —NH(C₁-C₄ alkyl), —N(C₁-C₄ alkyl)(C₁-C₄ alkyl), —C(O)NH₂, —C(O)NH(C₁-C₄ alkyl), —C(O)N(C₁-C₄ alkyl)(C₁-C₄ alkyl), —C(O)(C₁-C₄ alkyl), —NHC(O)(C₁-C₄ alkyl), —N(C₁-C₄ alkyl)C(O)(C₁-C₄ alkyl), —S(O)ₙNH₂, —S(O)ₙNH(C₁-C₄ alkyl), —S(O)ₙN(C₁-C₄ alkyl)(C₁-C₄ alkyl), —S(O)ₙ(C₁-C₄ alkyl), —NHS(O)ₙ(C₁-C₄ alkyl), —N(C₁-C₄ alky)S(O)ₙ(C₁-C₄ alkyl), optionally substituted C₃-C₈ cycloalkyl, and optionally substituted 3-8 membered heterocycle, wherein C₁-C₄ alkyl is optionally substituted by halo, —OH, —OMe, or —CN,

Lx is optionally substituted C₁-C₈ alkylene.

In some embodiments, W is

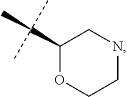

which is substituted by R³ on nitrogen atom, wherein R³ is independently selected from -Lx-S(O)ₙR⁸, -Lx-C(O)R⁹, —S(O)ₙ-Lx-R⁸, —C(O)-Lx-R⁹, -Lx-NR⁵C(O)R⁹, -Lx-NR⁵S(O)ₙR⁸, -Lx-NR⁵C(O)NR¹⁰R¹¹, -Lx-NR⁵S(O)ₙNR¹⁰R¹¹, -Lx-C(O)NR⁵R⁶, -Lx-S(O)ₙNR⁵R⁶

R⁵, R⁶, R⁷, R⁸, and R⁹ are independently selected from hydrogen, alkyl, cycloalkyl, aryl, heteroaryl, and heterocycle, each of which except for hydrogen, is optionally substituted with one or more groups selected from halo, —OH, —O(C₁-C₄ alkyl), —CN, C₁-C₄ alkyl, —NH₂, —NH(C₁-C₄ alkyl), —N(C₁-C₄ alkyl)(C₁-C₄ alkyl), —C(O)NH₂, —C(O)NH(C₁-C₄ alkyl), —C(O)N(C₁-C₄ alkyl)(C₁-C₄ alkyl), —C(O)(C₁-C₄ alkyl), —NHC(O)(C₁-C₄ alkyl), —N(C₁-C₄ alkyl)C(O)(C₁-C₄ alkyl), —S(O)ₙNH₂, —S(O)ₙNH(C₁-C₄ alkyl), —S(O)ₙN(C₁-C₄ alkyl)(C₁-C₄ alkyl), —S(O)ₙ(C₁-C₄ alkyl), —NHS(O)ₙ(C₁-C₄ alkyl), —N(C₁-C₄ alky)S(O)ₙ(C₁-C₄ alkyl), optionally substituted C₃-C₈ cycloalkyl, and optionally substituted 3-8 membered heterocycle, wherein C₁-C₄ alkyl is optionally substituted by halo, —OH, —OMe, or —CN,

or R⁵ and R⁶, R⁵ and R⁷, R⁵ and R⁸, and R⁵ and R⁹ together with the atom(s) to which they are attached can form a ring, which is optionally substituted with one or more groups selected from halo, —OH, —O(C₁-C₄ alkyl), —CN, C₁-C₄ alkyl, —NH₂, —NH(C₁-C₄ alkyl), —N(C₁-C₄ alkyl)(C₁-C₄ alkyl), —C(O)NH₂, —C(O)NH(C₁-C₄ alkyl), —C(O)N(C₁-C₄ alkyl)(C₁-C₄ alkyl), —C(O)(C₁-C₄ alkyl), —NHC(O)(C₁-C₄ alkyl), —N(C₁-C₄ alkyl)C(O)(C₁-C₄ alkyl), —S(O)ₙNH₂, —S(O)ₙNH(C₁-C₄ alkyl), —S(O)ₙN(C₁-C₄ alkyl)(C₁-C₄ alkyl), —S(O)ₙ(C₁-C₄ alkyl), —NHS(O)ₙ(C₁-C₄ alkyl), —N(C₁-C₄ alky)S(O)ₙ(C₁-C₄ alkyl), optionally substituted C₃-C₈ cycloalkyl, and optionally substituted 3-8 membered heterocycle, wherein C₁-C₄ alkyl is optionally substituted by halo, —OH, —OMe, or CN,

Lx is optionally substituted C₁-C₈ alkylene.

In some embodiments, W is

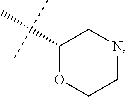

which is substituted by R³ on nitrogen atom, wherein R³ is independently selected from -Lx-S(O)ₙR⁸, -Lx-C(O)R⁹, —S(O)ₙ-Lx-R⁸, —C(O)-Lx-R⁹, -Lx-NR⁵C(O)R⁹, -Lx-NR⁵S(O)ₙR⁸, -Lx-NR⁵C(O)NR¹⁰R¹¹, -Lx-NR⁵S(O)ₙNR¹⁰R¹¹, -Lx-C(O)NR⁵R⁶, -Lx-S(O)ₙNR⁵R⁶

R⁵, R⁶, R⁷, R⁸, and R⁹ are independently selected from hydrogen, alkyl, cycloalkyl, aryl, heteroaryl, and heterocycle, each of which except for hydrogen, is optionally substituted with one or more groups selected from halo, —OH, —O(C₁-C₄ alkyl), —CN, C₁-C₄ alkyl, —NH₂, —NH(C₁-C₄ alkyl), —N(C₁-C₄ alkyl)(C₁-C₄ alkyl), —C(O)NH₂, —C(O)NH(C₁-C₄ alkyl), —C(O)N(C₁-C₄ alkyl)(C₁-C₄ alkyl), —C(O)(C₁-C₄ alkyl), —NHC(O)(C₁-C₄ alkyl), —N(C₁-C₄ alkyl)C(O)(C₁-C₄ alkyl), —S(O)ₙNH₂, —S(O)ₙNH(C₁-C₄ alkyl), —S(O)ₙN(C₁-C₄ alkyl)(C₁-C₄ alkyl), —S(O)ₙ(C₁-C₄ alkyl), —NHS(O)ₙ(C₁-C₄ alkyl), —N(C₁-C₄ alky)S(O)ₙ(C₁-C₄ alkyl), optionally substituted C₃-C₈ cycloalkyl, and optionally substituted 3-8 membered heterocycle, wherein C₁-C₄ alkyl is optionally substituted by halo, —OH, —OMe, or —CN,

or R⁵ and R⁶, R⁵ and R⁷, R⁵ and R⁸, and R⁵ and R⁹ together with the atom(s) to which they are attached can form a ring, which is optionally substituted with one or more groups selected from halo, —OH, —O(C₁-C₄ alkyl), —CN, C₁-C₄ alkyl, —NH₂, —NH(C₁-C₄ alkyl), —N(C₁-C₄ alkyl)(C₁-C₄ alkyl), —C(O)NH₂, —C(O)NH(C₁-C₄ alkyl), —C(O)N(C₁-C₄ alkyl)(C₁-C₄ alkyl), —C(O)(C₁-C₄ alkyl), —NHC(O)(C₁-C₄ alkyl), —N(C₁-C₄ alkyl)C(O)(C₁-C₄ alkyl), —S(O)ₙNH₂, —S(O)ₙNH(C₁-C₄ alkyl), —S(O)ₙN(C₁-C₄ alkyl)(C₁-C₄ alkyl), —S(O)ₙ(C₁-C₄ alkyl), —NHS(O)ₙ(C₁-C₄ alkyl), —N(C₁-C₄ alky)S(O)ₙ(C₁-C₄ alkyl), optionally substituted C₃-C₈ cycloalkyl, and optionally substituted 3-8 membered heterocycle, wherein C₁-C₄ alkyl is optionally substituted by halo, —OH, —OMe, or CN,

Lx is optionally substituted C₁-C₈ alkylene.

In some embodiments, R³ is independently selected from hydrogen, -Lx-halo, -Lx-R⁴, -Lx-NR⁵R⁶, -Lx-OR⁷, -Lx-S(O)ₙR⁸, -Lx-C(O)R⁹, —S(O)ₙ-Lx-R⁸, —C(O)-Lx-R⁹, -Lx-CN, -Lx-NR⁵C(O)R⁹, -Lx-NR⁵S(O)ₙR⁸, -Lx-NR⁵C(O)NR¹⁰R¹¹, -Lx-NR⁵S(O)ₙNR¹⁰R¹¹, -Lx-NR⁵C(O)OR⁷, -Lx-NR⁵S(O)ₙOR⁷, —NO₂, -Lx-C(O)NR⁵R⁶, -Lx-S(O)ₙNR⁵R⁶, oxo(═O), optionally substituted C₃-C₈cycloalkyl, optionally substituted 3-8 membered heterocycle, optionally substituted C₅-C₁₀aryl, and optionally substituted 5-10 membered heteroaryl, provided when L is methylene and W is 5- or 6-membered heterocycle, R³ is independently selected from -Lx-NR⁵R⁶, -Lx-OR⁷, -Lx-S(O)ₙR⁸, -Lx-C(O)R⁹, —S(O)ₙ-Lx-R⁸, —C(O)-Lx-R⁹, -Lx-CN, -Lx-NR⁵C(O)R⁹, -Lx-NR⁵S(O)ₙR⁸, -Lx-NR⁵C(O)NR¹⁰R¹¹, -Lx-NR⁵S(O)ₙNR¹⁶R¹¹, -Lx-NR⁵C(O)OR⁷, -Lx-NR⁵S(O)ₙOR⁷, —NO₂, -Lx-C(O)NR⁵R⁶, -Lx-S(O)ₙNR⁵R⁶, oxo(═O), optionally substituted C₃-C₈cycloalkyl, optionally substituted 3-8 membered heterocycle, optionally substituted C₅-C₁₀aryl, and optionally substituted 5-10 membered heteroaryl,

R⁴ is optionally substituted C₁-C₄alkyl,

R⁵, R⁶, R⁷, R⁸, R⁹, R¹⁰, and R¹¹ are independently selected from hydrogen, alkyl, cycloalkyl, aryl, heteroaryl, and heterocycle, each of which except for hydrogen, is optionally substituted with one or more groups selected from halo, —OH, —O(C₁-C₄alkyl), —CN, C₁-C₄ alkyl, —NH₂, —NH(C₁-C₄ alkyl), —N(C₁-C₄ alkyl)(C₁-C₄ alkyl), —C(O)NH₂, —C(O)NH(C₁-C₄ alkyl), —C(O)N(C₁-C₄ alkyl)(C₁-C₄ alkyl), —C(O)(C₁-C₄ alkyl), —NHC(O)(C₁-C₄ alkyl), —N(C₁-C₄ alkyl)C(O)(C₁-C₄ alkyl), —S(O)ₙNH₂, —S(O)ₙNH(C₁-C₄ alkyl), —S(O)ₙN(C₁-C₄ alkyl)(C₁-C₄ alkyl), —S(O)ₙ(C₁-C₄ alkyl), —NHS(O)ₙ(C₁-C₄ alkyl), —N(C₁-C₄ alky)S(O)ₙ(C₁-C₄ alkyl), optionally substituted C₃-C₈ cycloalkyl, and optionally substituted 3-8 membered heterocycle, wherein C₁-C₄ alkyl is optionally substituted by halo, —OH, —OMe, or —CN,

or R⁵ and R⁶, R⁵ and R⁷, R⁵ and R⁸, R⁵ and R⁹, and R⁵ and R¹⁰ together with the atom(s) to which they are attached can form a ring, which is optionally substituted with one or more groups selected from halo, —OH, —O(C₁-C₄alkyl), —CN, C₁-C₄alkyl, —NH₂, —NH(C₁-C₄alkyl), —N(C₁-C₄ alkyl)(C₁-C₄ alkyl), —C(O)NH₂, —C(O)NH(C₁-C₄ alkyl), —C(O)N(C₁-C₄ alkyl)(C₁-C₄ alkyl), —C(O)(C₁-C₄ alkyl), —NHC(O)(C₁-C₄ alkyl), —N(C₁-C₄ alkyl)C(O)(C₁-C₄ alkyl), —S(O)ₙNH₂, —S(O)ₙNH(C₁-C₄ alkyl), —S(O)ₙN(C₁-C₄ alkyl)(C₁-C₄ alkyl), —S(O)ₙ(C₁-C₄ alkyl), —NHS(O)ₙ(C₁-C₄ alkyl), —N(C₁-C₄alky)S(O)ₙ(C₁-C₄alkyl), optionally substituted C₃-C₈ cycloalkyl, and optionally substituted 3-8 membered heterocycle, wherein C₁-C₄ alkyl is optionally substituted by halo, —OH, —OMe, or —CN,

Lx is a bond, or optionally substituted C₁-C₆ alkylene.

In some embodiments, R³ is independently selected from hydrogen, -Lx-halo, -Lx-R⁴, -Lx-NR⁵R⁶, -Lx-OR⁷, -Lx-S(O)ₙR⁸, -Lx-C(O)R⁹, —S(O)ₙ-Lx-R⁸, —C(O)-Lx-R⁹, -Lx-CN, -Lx-NR⁵C(O)R⁹, -Lx-NR⁵S(O)ₙR⁸, -Lx-NR⁵C(O)NR¹⁰R¹¹, -Lx-NR⁵S(O)ₙNR¹⁶R¹¹, -Lx-NR⁵C(O)OR⁷, -Lx-NR⁵S(O)ₙOR⁷, —NO₂, -Lx-C(O)NR⁵R⁶, -Lx-S(O)ₙNR⁵R⁶, oxo(═O), or selected from cyclopropyl, cyclobutyl, cyclopentyl, cyclopentenyl, cyclohexyl, cyclohexenyl, pyrrolidinyl, tetrahydrofuryl, tetrahydropyranyl, piperidinyl, piperazinyl, morpholinyl, homomorpholinyl, thiomorpholinyl, diazepanyl, oxazepanyl, phenyl, naphthyl pyridyl, pyrimidinyl, pyrazinyl, pyridazinyl, pyrrolyl, pyrazolyl, imidazolinyl, oxazolyl, isoxazolyl, thiazolyl, thienyl, furyl, benzofuryl, benzothienyl, benzoimidazolinyl, indolyl, indazolyl, and quinolinyl, each of which is optionally substituted. Provided when L is methylene and W is 5- or 6-membered heterocycle, R³ is independently selected from -Lx-NR⁵R⁶, -Lx-OR⁷, -Lx-S(O)ₙR⁸, -Lx-C(O)R⁹, —S(O)ₙ-Lx-R⁸, —C(O)-Lx-R⁹, -Lx-CN, -Lx-NR⁵C(O)R⁹, -Lx-NR⁵S(O)ₙR⁸, -Lx-NR⁵C(O)NR¹⁰R¹¹, -Lx-NR⁵S(O)ₙNR¹⁰R¹¹, -Lx-NR⁵C(O)OR⁷, -Lx-NR⁵S(O)ₙOR⁷, —NO₂, -Lx-C(O)NR⁵R⁶, -Lx-S(O)ₙNR⁵R⁶, oxo(═O),

R⁴ is methyl, ethyl, n-propyl, i-propyl, n-butyl, i-butyl, and t-butyl, each of which is optionally substituted,

R⁵, R⁶, R⁷, R⁸, R⁹, R¹⁰, and R¹¹ are independently selected from hydrogen, alkyl, cycloalkyl, aryl, heteroaryl, and heterocycle, each of which except for hydrogen, is optionally substituted with one or more groups selected from halo, —OH, —O(C₁-C₄ alkyl), —CN, C₁-C₄ alkyl, —NH₂, —NH(C₁-C₄ alkyl), —N(C₁-C₄ alkyl)(C₁-C₄ alkyl), —C(O)NH₂, —C(O)NH(C₁-C₄ alkyl), —C(O)N(C₁-C₄ alkyl)(C₁-C₄ alkyl), —C(O)(C₁-C₄ alkyl), —NHC(O)(C₁-C₄ alkyl), —N(C₁-C₄ alkyl)C(O)(C₁-C₄ alkyl), —S(O)ₙNH₂, —S(O)ₙNH(C₁-C₄ alkyl), —S(O)ₙN(C₁-C₄ alkyl)(C₁-C₄ alkyl), —S(O)ₙ(C₁-C₄ alkyl), —NHS(O)ₙ(C₁-C₄ alkyl), —N(C₁-C₄ alky)S(O)ₙ(C₁-C₄ alkyl), optionally substituted C₃-C₈ cycloalkyl, and optionally substituted 3-8 membered heterocycle, wherein C₁-C₄ alkyl is optionally substituted by halo, —OH, —OMe, or —CN,

or R⁵ and R⁶, R⁵ and R⁷, R⁵ and R⁸, R⁵ and R⁹, and R⁵ and R¹⁰ together with the atom(s) to which they are attached can form a ring, which is optionally substituted with one or more groups selected from halo, —OH, —O(C₁-C₄ alkyl), —CN, C₁-C₄ alkyl, —NH₂, —NH(C₁-C₄ alkyl), —N(C₁-C₄ alkyl)(C₁-C₄ alkyl), —C(O)NH₂, —C(O)NH(C₁-C₄ alkyl), —C(O)N(C₁-C₄ alkyl)(C₁-C₄ alkyl), —C(O)(C₁-C₄ alkyl), —NHC(O)(C₁-C₄ alkyl), —N(C₁-C₄ alkyl)C(O)(C₁-C₄ alkyl), —S(O)ₙNH₂, —S(O)ₙNH(C₁-C₄ alkyl), —S(O)ₙN(C₁-C₄ alkyl)(C₁-C₄ alkyl), —S(O)ₙ(C₁-C₄ alkyl), —NHS(O)ₙ(C₁-C₄ alkyl), —N(C₁-C₄ alky)S(O)ₙ(C₁-C₄ alkyl), optionally substituted C₃-C₈ cycloalkyl, and optionally substituted 3-8 membered heterocycle, wherein C₁-C₄ alkyl is optionally substituted by halo, —OH, —OMe, or —CN,

Lx is a bond, or optionally substituted C₁-C₄ alkylene.

In some embodiments, R³ is independently selected from hydrogen, -Lx-OR⁷, -Lx-S(O)ₙR⁸, -Lx-C(O)R⁹, —S(O)ₙ-Lx-R⁸, —C(O)-Lx-R⁹, -Lx-NR⁵C(O)R⁹, -Lx-NR⁵S(O)ₙR⁸, -Lx-NR⁵C(O)NR¹⁰R¹¹, -Lx-NR⁵S(O)ₙNR¹⁰R¹¹, -Lx-C(O)NR⁵R⁶, -Lx-S(O)ₙNR⁵R⁶, and oxo(═O),

R⁵, R⁶, R⁷, R⁸, R⁹, R¹⁰, and R¹¹ are independently selected from hydrogen, alkyl, cycloalkyl, aryl, heteroaryl, and heterocycle, each of which except for hydrogen, is optionally substituted with one or more groups selected from halo, —OH, —O(C₁-C₄ alkyl), —CN, C₁-C₄ alkyl, —NH₂, —NH(C₁-C₄ alkyl), —N(C₁-C₄ alkyl)(C₁-C₄ alkyl), —C(O)NH₂, —C(O)NH(C₁-C₄ alkyl), —C(O)N(C₁-C₄ alkyl)(C₁-C₄ alkyl), —C(O)(C₁-C₄ alkyl), —NHC(O)(C₁-C₄ alkyl), —N(C₁-C₄ alkyl)C(O)(C₁-C₄ alkyl), —S(O)ₙNH₂, —S(O)ₙNH(C₁-C₄ alkyl), —S(O)ₙN(C₁-C₄ alkyl)(C₁-C₄ alkyl), —S(O)ₙ(C₁-C₄ alkyl), —NHS(O)ₙ(C₁-C₄ alkyl), —N(C₁-C₄ alky)S(O)ₙ(C₁-C₄ alkyl), optionally substituted C₃-C₈ cycloalkyl, and optionally substituted 3-8 membered heterocycle, wherein C₁-C₄ alkyl is optionally substituted by halo, —OH, —OMe, or —CN,

or R⁵ and R⁶, R⁵ and R⁷, R⁵ and R⁸, R⁵ and R⁹, and R⁵ and R¹⁰ together with the atom(s) to which they are attached can form a ring, which is optionally substituted with one or more groups selected from halo, —OH, —O(C₁-C₄ alkyl), —CN, C₁-C₄ alkyl, —NH₂, —NH(C₁-C₄ alkyl), —N(C₁-C₄ alkyl)(C₁-C₄ alkyl), —C(O)NH₂, —C(O)NH(C₁-C₄ alkyl), —C(O)N(C₁-C₄ alkyl)(C₁-C₄ alkyl), —C(O)(C₁-C₄ alkyl), —NHC(O)(C₁-C₄ alkyl), —N(C₁-C₄ alkyl)C(O)(C₁-C₄ alkyl), —S(O)ₙNH₂, —S(O)ₙNH(C₁-C₄ alkyl), —S(O)ₙN(C₁-C₄ alkyl)(C₁-C₄ alkyl), —S(O)ₙ(C₁-C₄ alkyl), —NHS(O)ₙ(C₁-C₄ alkyl), —N(C₁-C₄ alky)S(O)ₙ(C₁-C₄ alkyl), optionally substituted C₃-C₈ cycloalkyl, and optionally substituted 3-8 membered heterocycle, wherein C₁-C₄ alkyl is optionally substituted by halo, —OH, —OMe, or —CN,

Lx is a bond, or optionally substituted C₁-C₄ alkylene.

In some embodiments, R⁵, R⁶, R⁷, R⁸, R⁹, R¹⁰, and R¹¹ are independently selected from hydrogen, C₁-C₄alkyl, C₃-C₈cycloalkyl, C₈-C₁₀aryl, 5-10 membered heteroaryl, and 3-8 membered heterocycle, each of which except for hydrogen, is optionally substituted with one or more groups selected from halo, —OH, —O(C₁-C₄ alkyl), —CN, C₁-C₄ alkyl, —NH₂, NH(C₁-C₄ alkyl), —N(C₁-C₄ alkyl)(C₁-C₄ alkyl), —C(O)NH₂, —C(O)NH(C₁-C₄ alkyl), —C(O)N(C₁-C₄ alkyl)(C₁-C₄ alkyl), —C(O)(C₁-C₄ alkyl), —NHC(O)(C₁-C₄ alkyl), —N(C₁-C₄ alkyl)C(O)(C₁-C₄ alkyl), —S(O)ₙNH₂, —S(O)ₙNH(C₁-C₄ alkyl), —S(O)ₙN(C₁-C₄ alkyl)(C₁-C₄ alkyl), —S(O)ₙ(C₁-C₄ alkyl), —NHS(O)ₙ(C₁-C₄ alkyl), —N(C₁-C₄ alky)S(O)ₙ(C₁-C₄ alkyl), optionally substituted C₃-C₈ cycloalkyl, and optionally substituted 3-8 membered heterocycle, wherein C₁-C₄ alkyl is optionally substituted by halo, —OH, —OMe, or —CN.

In some embodiments, R⁵, R⁶, R⁷, R⁸, R⁹, R¹⁰, and R¹¹ are independently selected from hydrogen, methyl, ethyl, n-propyl, i-propyl, n-butyl, i-butyl, and t-butyl, cyclopropyl, cyclobutyl, cyclopentyl, cyclopentenyl, cyclohexyl, cyclohexenyl, phenyl, naphthyl, pyridyl, pyrimidinyl, pyrazinyl, pyridazinyl, pyrrolyl pyrazolyl, imidazolinyl, oxazolyl, isoxazolyl, thiazolyl, thienyl, furyl, benzofuryl, benzothienyl, benzoimidazolinyl, indolyl, indazolyl, quinolinyl, pyrrolidinyl, tetrahydrofuryl, tetrahydropyranyl, piperidinyl, piperazinyl, morpholinyl, homomorpholinyl, thiomorpholinyl, diazepanyl, and oxazepanyl, each of which except for hydrogen, is optionally substituted with one or more groups selected from halo, —OH, —O(C₁-C₄ alkyl), —CN, C₁-C₄ alkyl, —NH₂, —NH(C₁-C₄ alkyl), —N(C₁-C₄ alkyl)(C₁-C₄ alkyl), —C(O)NH₂, —C(O)NH(C₁-C₄ alkyl), —C(O)N(C₁-C₄ alkyl)(C₁-C₄ alkyl), —C(O)(C₁-C₄ alkyl), —NHC(O)(C₁-C₄ alkyl), —N(C₁-C₄ alkyl)C(O)(C₁-C₄ alkyl), —S(O)ₙNH₂, —S(O)ₙNH(C₁-C₄alkyl), —S(O)ₙN(C₁-C₄ alkyl)(C₁-C₄ alkyl), —S(O)ₙ(C₁-C₄ alkyl), —NHS(O)ₙ(C₁-C₄ alkyl), —N(C₁-C₄ alky)S(O)ₙ(C₁-C₄ alkyl), optionally substituted C₃-C₈ cycloalkyl, and optionally substituted 3-8 membered heterocycle, wherein C₁-C₄ alkyl is optionally substituted by halo, —OH, —OMe, or —CN.

In some embodiments, R⁵ and R⁶, R⁵ and R⁷, R⁵ and R⁸, R⁵ and R⁹, and R⁵ and R¹⁰ together with the atom(s) to which they are attached can form a ring, which is optionally substituted with one or more groups selected from halo, —OH, —O(C₁-C₄ alkyl), —CN, C₁-C₄ alkyl, —NH₂, —NH(C₁-C₄ alkyl), —N(C₁-C₄ alkyl)(C₁-C₄ alkyl), —C(O)NH₂, —C(O)NH(C₁-C₄ alkyl), —C(O)N(C₁-C₄ alkyl)(C₁-C₄ alkyl), —C(O)(C₁-C₄ alkyl), —NHC(O)(C₁-C₄ alkyl), —N(C₁-C₄ alkyl)C(O)(C₁-C₄ alkyl), —S(O)ₙNH₂, —S(O)ₙNH(C₁-C₄ alkyl), —S(O)ₙN(C₁-C₄ alkyl)(C₁-C₄ alkyl), —S(O)ₙ(C₁-C₄ alkyl), —NHS(O)ₙ(C₁-C₄ alkyl), —N(C₁-C₄ alky)S(O)ₙ(C₁-C₄ alkyl), optionally substituted C₃-C₈ cycloalkyl, and optionally substituted 3-8 membered heterocycle, wherein C₁-C₄ alkyl is optionally substituted by halo, —OH, —OMe, or —CN.

In some embodiments, n is 2.

In some embodiments, Lx is a bond.

In some embodiments, Lx is optionally substituted C₁-C₄ alkylene.

In some embodiments, the optionally substituted lower alkyl is chosen from —CF₃, —CF₂H, —CH₂NH₂, —CH₂CH₂NH₂, —CH₂OH, —CH₂CH₂OH, —CH₂OCH₃, —CH₂CH₂OCH₃.

Also provided is at least one compound chosen from compounds 1 to 323 and/or at least one pharmaceutically acceptable salt thereof.

The compounds described herein, and/or the pharmaceutically acceptable salts thereof, can be synthesized from commercially available starting materials by methods well known in the art, taken together with the disclosure in this patent application. The following schemes illustrate methods for preparation of most of the compounds disclosed herein.

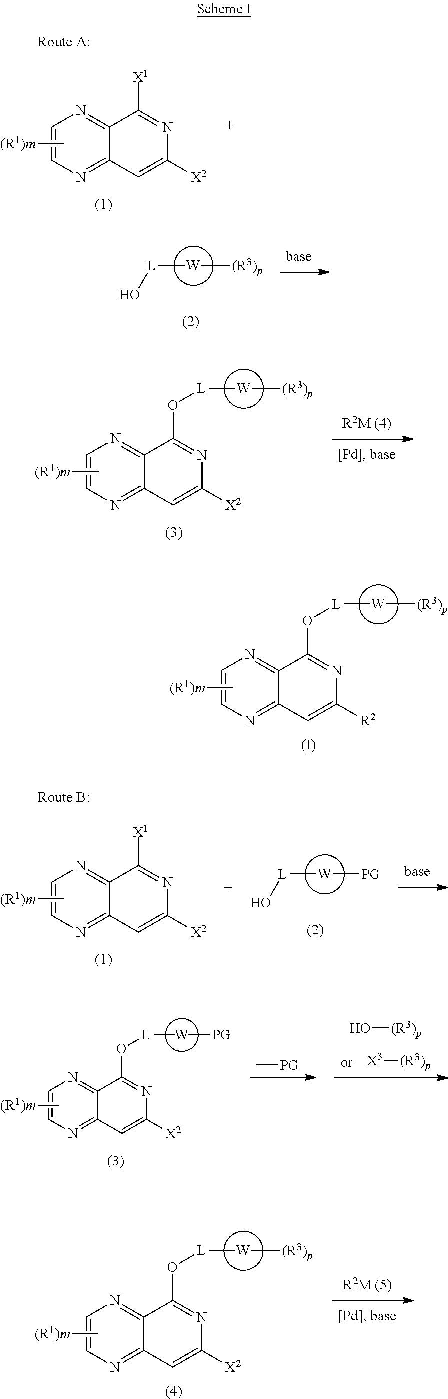

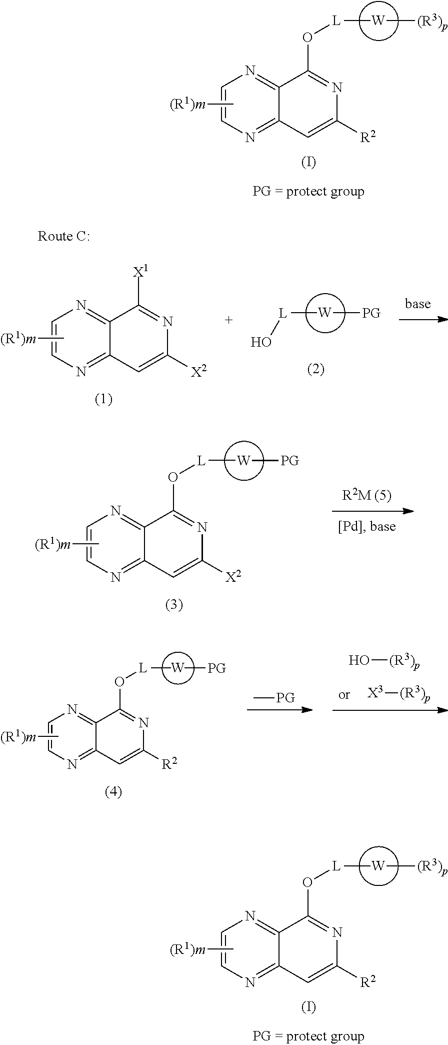

As shown in Scheme I, compound of formula (I) can be prepared by 3 routes.

Route A: compounds of formula (1), can react with compounds of formula (2), wherein m, R¹, L and W are as defined herein, X¹ and X² are halo chosen from Cl, Br or I, in the presence of a base, such as but not limited to K₂CO₃, Na₂CO₃, NaH, Et₃N or diisopropylethylamine (DIPEA), to give compounds of formula (3) that can react with compounds of formula (4), wherein R² is as defined herein, M is chosen from boronic acid/ester or a tin substituted with C₁-C₄ alkyl groups, under the catalysis of a palladium reagent, such as but not limited to PdCl₂, Pd(OAc)₂Pd₂(dba)₃ or Pd(PPh₃)₄, and a ligand, such as but not limited to Ph₃P, t-Bu₃P, 2,2′-bis(diphenylphosphino)-1,1′-binaphthalene (BINAP), 1,1′-bis(diphenylphosphino)ferrocene (dppf) or 1,3-bis(2,6-dipropylphenyl)-1H-imidazol-3-ium chloride, in the presence of a base, such as but not limited to K₂CO₃, Na₂CO₃, Cs₂CO₃, NaH, t-BuONa, t-BuOK, Et₃N, or diisopropylethylamine (DIPEA), to give the compounds of formula (I).
Route B: compounds of formula (1), can react with compounds of formula (2), wherein m, R¹, L and W are as defined herein, X¹ and X² are halo chosen from Cl, Br or I, in the presence of a base, such as but not limited to K₂CO₃, Na₂CO₃, NaH, Et₃N or diisopropylethylamine (DIPEA), to give compounds of formula (3) that can react with HO—(R³)ₚ or X³—(R³)ₚ after deprotection, wherein R³ and p are as defined herein, X³ is halo chosen from Cl, Br or I, to give compounds of formula (4) that can react with compounds of formula (5), wherein R² is as defined herein, M is chosen from boronic acid/ester or a tin substituted with C₁-C₄ alkyl groups, under the catalysis of a palladium reagent, such as but not limited to PdCl₂, Pd(OAc)₂Pd₂(dba)₃ or Pd(PPh₃)₄, and a ligand, such as but not limited to Ph₃P, t-Bu₃P, 2,2′-bis(diphenylphosphino)-1,1′-binaphthalene (BINAP), 1,1′-bis(diphenylphosphino)ferrocene (dppf) or 1,3-bis(2,6-dipropylphenyl)-1H-imidazol-3-ium chloride, in the presence of a base, such as but not limited to K₂CO₃, Na₂CO₃, Cs₂CO₃, NaH, t-BuONa, t-BuOK, Et₃N, or diisopropylethylamine (DIPEA), to give the compounds of formula (I).
Route C: in the presence of a base, such as but not limited to K₂CO₃, Na₂CO₃, NaH, Et₃N or diisopropylethylamine (DIPEA), compounds of formula (1) can react with compounds of formula (2), wherein m, R¹, L and W are as defined herein, X¹ and X² are halo chosen from Cl, Br or I, to give compounds of formula (3) that can react with compounds of formula (5) under the catalysis of a palladium reagent, such as but not limited to PdCl₂, Pd(OAc)₂, Pd₂(dba)₃ or Pd(PPh₃)₄, and a ligand, such as but not limited to Ph₃P, t-Bu₃P, 2,2′-bis(diphenylphosphino)-1,1′-binaphthalene (BINAP), 1,1′-bis(diphenylphosphino)ferrocene (dppf) or 1,3-bis(2,6-dipropylphenyl)-1H-imidazol-3-ium chloride, in the presence of a base, such as but not limited to K₂CO₃, Na₂CO₃, Cs₂CO₃, NaH, t-BuONa, t-BuOK, Et₃N, or diisopropylethylamine (DIPEA), to give the compounds of formula (4), which react with HO—(R³)ₚ or X³—(R³)ₚ after deprotection to give the compounds of formula (I), wherein R¹, R², R³, L, W, m, p are as defined herein, X¹, X², X³ are halo chosen from Cl, Br or I, M is chosen from boronic acid/ester or a tin substituted with C₁-C₄ alkyl groups.

The compounds thus obtained can be further modified at their peripheral positions to provide the desired compounds. Synthetic chemistry transformations are described, for example, in R. Larock, Comprehensive Organic Transformations, VCH Publishers (1989); T. W. Greene and P. G. M. Wuts, Protective Groups in Organic Synthesis, 3^{rd} Ed., John Wiley and Sons (1999); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995) and subsequent editions thereof.

Before use, the at least one compound and/or at least one pharmaceutically acceptable salt described herein, can be purified by column chromatography, high performance liquid chromatography, crystallization, or other suitable methods.

Also provided is a composition comprising at least one compound and/or at least one pharmaceutically acceptable salt described herein, and at least one pharmaceutically acceptable carrier.

A composition comprising at least one compound and/or at least one pharmaceutically acceptable salt described herein, can be administered in various known manners, such as orally, parenterally, by inhalation spray, or via an implanted reservoir. The term “parenteral” as used herein includes subcutaneous, intracutaneous, intravenous, intramuscular, intraarticular, intraarterial, intrasynovial, intrasternal, intrathecal, intralesional and intracranial injection or infusion techniques.

An oral composition can be any orally acceptable dosage form including, but not limited to, tablets, capsules, emulsions, and aqueous suspensions, dispersions and solutions. Commonly used carriers for tablets include lactose and corn starch. Lubricating agents, such as magnesium stearate, are also typically added to tablets. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added.

A sterile injectable composition (e.g., aqueous or oleaginous suspension) can be formulated according to techniques known in the art using suitable dispersing or wetting agents (such as, for example, Tween 80) and suspending agents. The sterile injectable Intermediate can also be a sterile injectable solution or suspension in a non-toxic parenterally acceptable diluent or solvent, for example, as a solution in 1,3-butanediol. Among the pharmaceutically acceptable vehicles and solvents that can be employed are mannitol, water, Ringer's solution and isotonic sodium chloride solution. In addition, sterile, fixed oils are conventionally employed as a solvent or suspending medium (e.g., synthetic mono- or di-glycerides). Fatty acids, such as oleic acid and its glyceride derivatives are useful in the Intermediate of injectables, as are natural pharmaceutically-acceptable oils, such as olive oil or castor oil, especially in their polyoxyethylated versions. These oil solutions or suspensions can also contain a long-chain alcohol diluent or dispersant, or carboxymethyl cellulose or similar dispersing agents.

An inhalation composition can be prepared according to techniques well known in the art of pharmaceutical formulation and can be prepared as solutions in saline, employing benzyl alcohol or other suitable preservatives, absorption promoters to enhance bioavailability, fluorocarbons, and/or other solubilizing or dispersing agents known in the art.

A topical composition can be formulated in form of oil, cream, lotion, ointment, and the like. Suitable carriers for the composition include vegetable or mineral oils, white petrolatum (white soft paraffin), branched chain fats or oils, animal fats and high molecular weight alcohols (greater than C12). In some embodiments, the pharmaceutically acceptable carrier is one in which the active ingredient is soluble. Emulsifiers, stabilizers, humectants and antioxidants may also be included as well as agents imparting color or fragrance, if desired. Additionally, transdermal penetration enhancers may be employed in those topical formulations. Examples of such enhancers can be found in U.S. Pat. Nos. 3,989,816 and 4,444,762.

Creams may be formulated from a mixture of mineral oil, self-emulsifying beeswax and water in which mixture the active ingredient, dissolved in a small amount of an oil, such as almond oil, is admixed. An example of such a cream is one which includes about 40 parts water, about 20 parts beeswax, about 40 parts mineral oil and about 1 part almond oil. Ointments may be formulated by mixing a solution of the active ingredient in a vegetable oil, such as almond oil, with warm soft paraffin and allowing the mixture to cool. An example of such an ointment is one which includes about 30% by weight almond oil and about 70% by weight white soft paraffin.

A pharmaceutically acceptable carrier refers to a carrier that is compatible with active ingredients of the composition (and in some embodiments, capable of stabilizing the active ingredients) and not deleterious to the subject to be treated. For example, solubilizing agents, such as cyclodextrins (which form specific, more soluble complexes with the at least one compound and/or at least one pharmaceutically acceptable salt described herein), can be utilized as pharmaceutical excipients for delivery of the active ingredients. Examples of other carriers include colloidal silicon dioxide, magnesium stearate, cellulose, sodium lauryl sulfate, and pigments such as D&C Yellow #10.

Suitable in vitro assays can be used to preliminarily evaluate the efficacy of the at least one compound and/or at least one pharmaceutically acceptable salt described herein, in inhibiting the activity of Syk kinase. The at least one compound and/or at least one pharmaceutically acceptable salt described herein, can further be examined for efficacy in treating inflammatory disease by in vivo assays. For example, the compounds described herein, and/or the pharmaceutically acceptable salts thereof, can be administered to an animal (e.g., a mouse model) having inflammatory disease and its therapeutic effects can be accessed. Based on the results, an appropriate dosage range and administration route for animals, such as humans, can also be determined.

Also provided is a method of inhibiting the activity of Syk kinase. The method comprises contacting the at least one kinase with an amount of at least one compound and/or at least one pharmaceutically acceptable salt described herein effective to inhibit the activity of the Syk kinase.

The at least one compound and/or at least one pharmaceutically acceptable salt described herein can be used to achieve a beneficial therapeutic or prophylactic effect, for example, in subjects with an inflammatory disease or inflammatory disorder. The term “inflammatory disease” or “inflammatory disorder” refers to pathological states resulting in inflammation, typically caused by neutrophil chemotaxis. Examples of such disorders include inflammatory skin diseases including psoriasis and atopic dermatitis; systemic scleroderma and sclerosis; responses associated with inflammatory bowel disease (IBD) (such as Crohn's disease and ulcerative colitis); ischemic reperfusion disorders including surgical tissue reperfusion injury, myocardial ischemic conditions such as myocardial infarction, cardiac arrest, reperfusion after cardiac surgery and constriction after percutaneous transluminal coronary angioplasty, stroke, and abdominal aortic aneurysms; cerebral edema secondary to stroke; cranial trauma, hypovolemic shock; asphyxia; adult respiratory distress syndrome; acute-lung injury; Behcet's Disease; dermatomyositis; polymyositis; multiple sclerosis (MS); dermatitis; meningitis; encephalitis; uveitis; osteoarthritis; lupus nephritis; autoimmune diseases such as rheumatoid arthritis (RA), Sjorgen's syndrome, vasculitis; diseases involving leukocyte diapedesis; central nervous system (CNS) inflammatory disorder, multiple organ injury syndrome secondary to septicaemia or trauma; alcoholic hepatitis; bacterial pneumonia; antigen-antibody complex mediated diseases including glomerulonephritis; sepsis; sarcoidosis; immunopathologic responses to tissue/organ transplantation; inflammations of the lung, including pleurisy, alveolitis, vasculitis, pneumonia, chronic bronchitis, bronchiectasis, diffuse panbronchiolitis, hypersensitivity pneumonitis, idiopathic pulmonary fibrosis (IPF), and cystic fibrosis; etc. The preferred indications include, without limitation, chronic inflammation, autoimmune diabetes, rheumatoid arthritis (RA), rheumatoid spondylitis, gouty arthritis and other arthritic conditions, multiple sclerosis (MS), asthma, systhemic lupus erythrematosus, adult respiratory distress syndrome, Behcet's disease, psoriasis, chronic pulmonary inflammatory disease, graft versus host reaction, Crohn's Disease, ulcerative colitis, inflammatory bowel disease (IBD), Alzheimer's disease, and pyresis, along with any disease or disorder that relates to inflammation and related disorders.

The at least one compound and/or at least one pharmaceutically acceptable salt described herein can be used to achieve a beneficial therapeutic or prophylactic effect, for example, in subjects with an autoimmune disease. The term “autoimmune disease” refers to a disease or disorder arising from and/or directed against an individual's own tissues or organs, or a co-segregate or manifestation thereof, or resulting condition therefrom. Examples of autoimmune diseases include, but are not limited to, lupus, myasthenia gravis, multiple sclerosis (MS), rheumatoid arthritis (RA), psoriasis, inflammatory bowel disease, asthma and idiopathic thrombocytopenic purpura, and myeloid proliferative disorder, such asmyelofibrosis, PV/ET (Post-Polycythemia/Essential Thrombocythemia Myelofibrosis).

In some embodiments, the at least one compound and/or at least one pharmaceutically acceptable salt described herein, is administered in conjunction with another therapeutic agent. In some embodiments, the other therapeutic agent is one that is normally administered to patients with the disease or condition being treated. For example, the other therapeutic agent may be an anti-inflammatory agent or an anti-neoplastic agent, depending on the disease or condition being treated. The at least one compound and/or at least one pharmaceutically acceptable salt described herein, may be administered with the other therapeutic agent in a single dosage form or as a separate dosage form. When administered as a separate dosage form, the other therapeutic agent may be administered prior to, at the same time as, or following administration of the at least one compound and/or at least one pharmaceutically acceptable salt described herein.

In some embodiments, the at least one compound and/or at least one pharmaceutically acceptable salt described herein, is administered in conjunction with an anti-inflammatory agent. Nonlimiting examples of anti-inflammatory agents include corticosteroids (e.g., fluticasone propionate, beclomethasone dipropionate, mometasone furoate, triamcinolone acetonide or budesonide), disease-modifying agents (e.g., antimalarials, methotrexate, sulfasalazine, mesalamine, azathioprine, 6-mercaptopurine, metronidazole, injectable and oral gold, or D-penicillamine), non-steroidal antiinflammatory drugs (e.g., acetominophen, aspirin, sodium salicylate, sodium cromoglycate, magnesium salicylate, choline magnesium salicylate, salicylsalicylic acid, ibuprofen, naproxen, diclofenac, diflunisal, etodolac, fenoprofen calcium, fluriprofen, piroxicam, indomethacin, ketoprofen, ketorolac tromethamine, meclofenamate, meclofenamate sodium, mefenamic acid, nabumetone, oxaprozin, phenyl butyl nitrone (PBN), sulindac, or tolmetin), COX-2 inhibitors, inhibitors of cytokine synthesis/release (e.g., anti-cytokine antibodies, anti-cytokine receptor antibodies, and the like).

### EXAMPLES

The examples below are intended to be purely exemplary and should not be considered to be limiting in any way. Efforts have been made to ensure accuracy with respect to numbers used (for example, amounts, temperature, etc.) but some experimental errors and deviations should be accounted for. Unless indicated otherwise, parts are parts by weight, temperature is in degrees of Centigrade, and pressure is at or near atmospheric. All MS data were checked by Agilent 6120 and/or Agilent 1100. All reagents, except intermediates, used in this invention are commercially available. All compound names except the reagents were generated by Chemdraw 12.0.

In the following examples, the abbreviations below are used:

Boc tert-butoxycarbonyl
Boc₂O di-t-butyl-dicarbonate

### CDI N,N′-Carbonyldiimidazole

DAST Diethylaminosulfur trifluoride
DCM dichloromethane

### DMF N,N-dimethylformamide

DMAP 4-dimethylaminopyridine

### DIPEA N,N-Diisopropylethylamine

EDCl 1-(3-Dimethylaminopropyl)-3-ethylcarbodiimide Hydrochloride
EtOAc/EA ethyl acetate
Et₃N triethylamine
HATU O-(7-azabenzotriazol-1-yl)-N,N,N′,N′-tetra-methyluronium hexafluorophosphate
HOAc acetic acid

### HOBt Hydroxybenzotriazole

mL milliliter(s)
mg milligram
min minute(s)
MeOH methanol
MsCl methanesulfonyl chloride
NaH Sodium hydride
PE petroleum ether

### Pd(dppf)Cl₂ 1,1′-Bis(diphenylphosphino)ferrocene-palladium(II)dichloride

Pd₂(dba)₃ tris(dibenzylideneacetone)dipalladium(0)
Pd(PPh₃)₄ tetrakis(triphenylphosphine)palladium(0)
PPh₃ triphenylphosphine
TBDMSCl tert-Butyldimethylsilyl chloride
TMSNCO trimethylsilyl isocyanate
THF tetrahydrofuran

### Intermediate 1

### 2-methyl-4-(4-(4,4,5,5-tetramethyl-1,3,2-dioxaborolan-2-yl)phenyl)morpholine

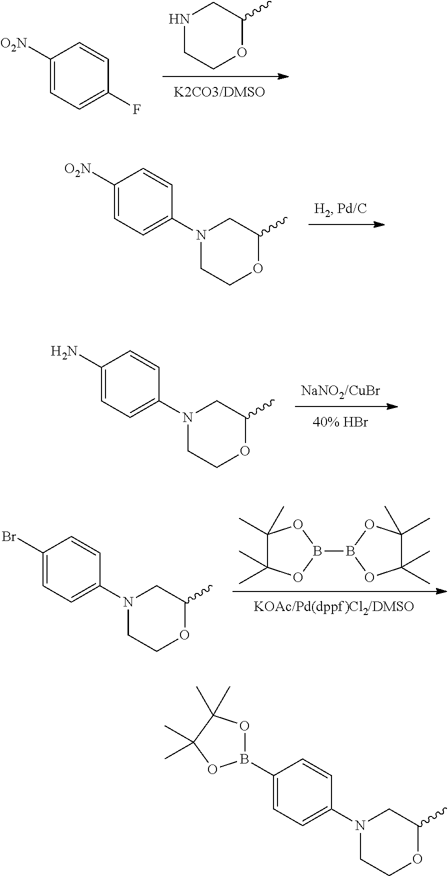

### (A) 4-(2-methylmorpholino)aniline

To a mixture of 1-fluoro-4-nitrobenzene (5.64 g, 40 mmol) and K₂CO₃ (11.1 g, 80 mmol) in DMSO (30 mL) was added 2-methylmorpholine (4.05 g, 40 mmol) then heated at 100° C. for 4 hours. This solution was poured into water (300 mL) and extracted with EA (3×100 mL). The combined organic phase was washed with brine and dried, filtered and Pd/C (1 g) was added to the filtrate. Charged with H₂, the solution was stirred at room temperature overnight. The catalyst was filtered and the filtrate was concentrated to give product as light red solid. MS (m/z): 223 (M+H)⁺.

### (B) 4-(4-bromophenyl)-2-methylmorpholine

To a solution of 4-(2-methylmorpholino)aniline (7.21 g, 37.5 mmol) in 100 mL 40% HBr solution was added a solution of NaNO₂ (2.59 g, 37.5 mmol) in 15 mL H₂O at −10˜0° C. The mixture was stirred for 30 minutes and added dropwise to a solution of CuBr (2.96 g, 20.6 mmol) in 30 mL 40% HBr solution. The resulting mixture was stirred and heated at 60° C. for 2 hours. Then the reaction solution was adjusted by 2N NaOH solution until pH>7, extracted with EA. The combined organic phase was washed with brine, dried and concentrated to give crude product as black oil. MS (m/z): 256 (M+H)⁺.

### (C) 2-methyl-4-(4-(4,4,5,5-tetramethyl-1,3,2-dioxaborolan-2-yl)phenyl)morpholine

A mixture of 4-(4-bromophenyl)-2-methylmorpholine (8 g, ˜31 mmol), 4,4,4′,4′,5,5,5′,5′-octamethyl-2,2′-bi(12-dioxaborolane) (10.3 g, 40.6 mmol), KOAc (4.6 g, 46.5 mmol) and PdCl₂(dppf) (2.26 g, 3.1 mmol) in DMSO (80 mL) was heated at 70° C. under N₂ for 4 hours. The reaction mixture was partitioned with EA and water. The combined organic phase was dried and concentrated, purification over silica gel chromatography, eluting with EA/PE=5:1, to give product as light yellow solid. MS (m/z): 304 (M+H)⁺.

### Intermediate 2

### (2S,6R)-2,6-dimethyl-4-(4-(4,4,5,5-tetramethyl-1,3,2-dioxaborolan-2-yl)phenyl)morpholine

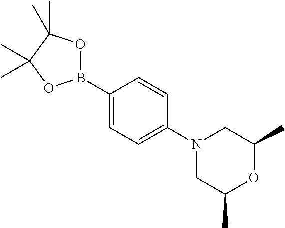

The title compound was prepared according to the procedures of Intermediate 1 using instead 1-fluoro-4-nitrobenzene and (2S,6R)-2,6-dimethylmorpholine. MS (m/z): 318 (M+H)⁺.

### Intermediate 3

### 4,4-difluoro-1-(4-(4,4,5,5-tetramethyl-1,3,2-dioxaborolan-2-yl)phenyl)piperidine

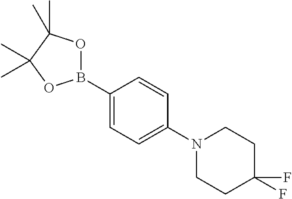 [SMILES: CC1(C)OB(c2ccc(N3CCC(F)(F)CC3)cc2)OC1(C)C]

The title compound was prepared according to the procedures of Intermediate 1 using instead 1-fluoro-4-nitrobenzene and 4,4-difluoropiperidine. MS (m/z): 324 (M+H)⁺.

### Intermediate 4

### 1-(4-(4,4,5,5-tetramethyl-1,3,2-dioxaborolan-2-yl)phenyl)piperidine

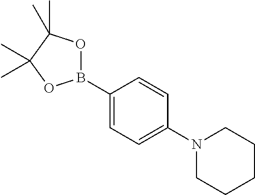 [SMILES: CC1(C)OB(c2ccc(N3CCCCC3)cc2)OC1(C)C]

The title compound was prepared according to the procedures of Intermediate 1 using instead 1-fluoro-4-nitrobenzene and piperidine. MS (m/z): 288 (M+H)⁺.

### Intermediate 5

### 2-(1-(4-(4,4,5,5-tetramethyl-1,3,2-dioxaborolan-2-yl)phenyl)piperidin-4-yl)propan-2-ol

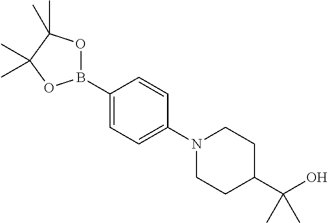 [SMILES: CC(C)(O)C1CCN(c2ccc(B3OC(C)(C)C(C)(C)O3)cc2)CC1]

The title compound was prepared according to the procedures of Intermediate 1 using instead 1-fluoro-4-nitrobenzene and 2-(piperidin-4-yl)propan-2-ol. MS (m/z): 346 (M+H)⁺.

### Intermediate 6

### 4-methoxy-1-(4-(4,4,5,5-tetramethyl-1,3,2-dioxaborolan-2-yl)phenyl)piperidine

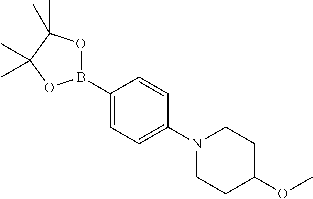

The title compound was prepared according to the procedures of Intermediate 1 using instead 1-fluoro-4-nitrobenzene and 4-methoxypiperidine. MS (m/z): 318 (M+H)⁺.

### Intermediate 7

### 1-(methylsulfonyl)-4-(4-(4,4,5,5-tetramethyl-1,3,2-dioxaborolan-2-yl)phenyl)piperazine

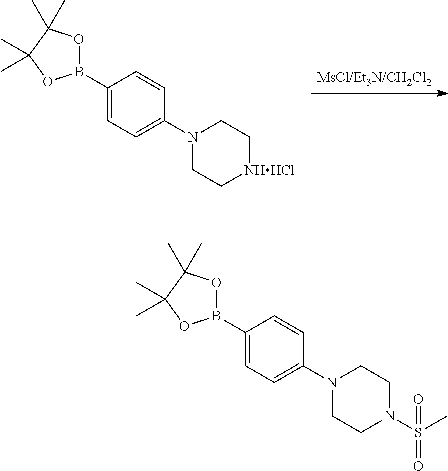

To a solution of 1-(4-(4,4,5,5-tetramethyl-1,3,2-dioxaborolan-2-yl)phenyl)piperazine hydrochloride (1.62 g, 5 mmol) in CH₂Cl₂ (60 mL) was added Et₃N (1.67 mL, 12 mmol) and MsCl (465 uL, 6 mmol) at 0° C. The reaction was stirred at 0° C. for 1 hour. Then the reaction was washed with aq.NaHCO₃ (15 mL), H₂O (15 mL) and brine (15 mL), dried over Na₂SO₄ and concentrated, purified by silica gel column chromatography (EA:PE=1:1) to give a yellow oil. MS (m/z): 367 (M+H)⁺.

### Intermediate 8

### 1-(ethylsulfonyl)-4-(4-(4,4,5,5-tetramethyl-1,3,2-dioxaborolan-2-yl)phenyl)piperazine

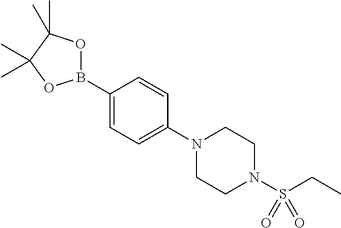 [SMILES: CCS(=O)(=O)N1CCN(c2ccc(B3OC(C)(C)C(C)(C)O3)cc2)CC1]

The title compound was prepared according to the procedures of Intermediate 7 using instead EtSO₂Cl. MS (m/z): 381 (M+H)⁺.

### Intermediate 9

### 4,4,5,5-tetramethyl-2-(4-(tetrahydro-2H-pyran-4-yl)phenyl)-1,3,2-dioxaborolane

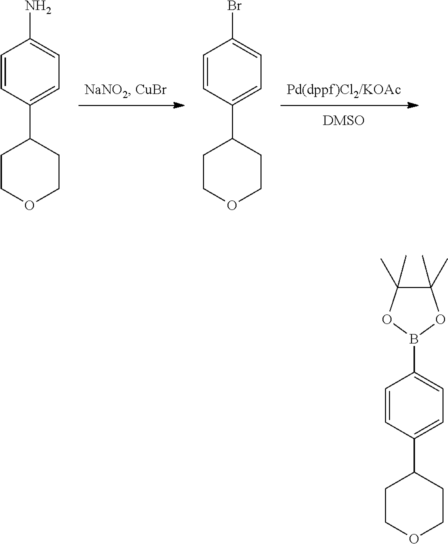

### (A) 4-(4-bromophenyl)-tetrahydro-2H-pyran

A solution of 4-(tetrahydro-2H-pyran-4-yl)benzenamine (1.79 g, 10.10 mmol) in 15 mL of HBr and 5 mL of water was stirred at 0° C. for 10 minutes, then 0.77 g of NaNO₂ was added to the mixture at −5° C.˜0° C. The mixture was stirred at −5° C. for 30 minutes. Then the solution of CuBr in 3 mL of HBr was added to the mixture, after that the mixture was heated at 100° C. for 2 hours. The mixture was cooled to room temperature, partitioned between 2N NaOH and EA, washed with water and aqueous NaCl, dried over Na₂SO₄. The volatiles were removed in vacuo, and the residue was purified by silica gel column chromatography with PE/EA (10:1˜4:1) to give 1.11 g of title compound.

### (B) 4,4,5,5-tetramethyl-2-(4-(tetrahydro-2H-pyran-4-yl)phenyl)-1,3,2-dioxaborolane

To a solution of 4-(4-bromophenyl)tetrahydro-2H-pyran (241 mg, 1 mmol) in dioxane (15 mL) was added KOAc (294 mg, 3 mmol), PdCl₂(dppf) (110 mg, 0.15 mmol) and 4,4,4′,4′,5,5,5′,5′-octamethyl-2,2′-bi(1,3,2-dioxaborolane) (381 mg, 1.5 mmol). The mixture was stirred at 80° C. overnight. The reaction was filtered and concentrated to give crude product, which was used for next step directly. MS (m/z): 289 (M+H)⁺

### Intermediate 10

### 5-(hydroxymethyl)piperidin-2-one

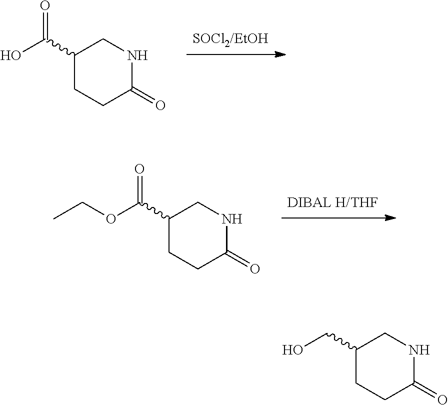

### (A) ethyl 6-oxopiperidine-3-carboxylate

SOCl₂ (2.93 g, 24.6 mmol) was dropped into a solution of 6-oxopiperidine-3-carboxylic acid (1.72 g, 12.3 mmol) in EtOH (50 mL) at 0° C. Then the reaction was stirred at room temperature for 24 hours. The reaction mixtures was concentrated and the residue was triturated with ether to give white solid. MS (m/z): 172 (M+H)⁺

### (B) 5-(hydroxymethyl)piperidin-2-one

To a solution of ethyl 6-oxopiperidine-3-carboxylate (171 mg, 1 mmol) in THF (5 mL) under N₂ at 70° C. was added 1.2N DIBAL H (2.5 mL, 3 mmol) dropwise. Then the mixture was stirred at 25° C. for 1 hour. The reaction was decomposed by dropwise addition of 120 uL MeOH in 1 mL of toluene, 1.2 mL of 30% K₂CO₃. The mixture was filtered and the granular precipitate was washed with 5 mL ethanol. Evaporation of the filtrate provided yellow oil. The oil was used for next step directly. MS (m/z): 130 (M+H)⁺.

### Intermediate 11

### 2-(4-(4-(4,4,5,5-tetramethyl-1,3,2-dioxaborolan-2-yl)phenyl)piperazin-1-yl)ethanol

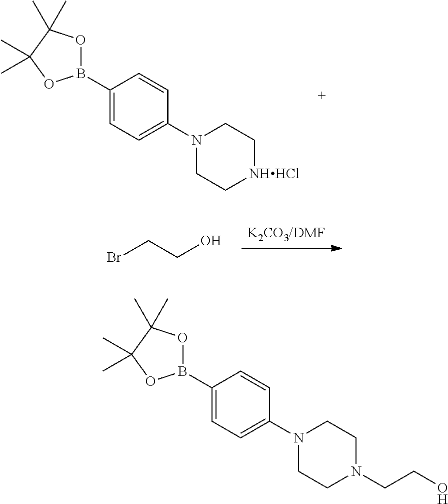

To a solution of 1-(4-(4,4,5,5-tetramethyl-1,3,2-dioxaborolan-2-yl)phenyl)piperazine hydrochloride (1.62 g, 5 mmol) in DMF (50 mL) was added K₂CO₃ (2.07 g, 15 mmol) and 2-bromoethanol (937.5 mg, 7.5 mmol). The mixture was stirred at 80° C. for 5 hours, then was poured into 30 mL water, extracted with EA (20 mL×3), the organic phase was washed with water and brine, concentrated to give brown solid. MS (m/z): 333 (M+H)⁺.

### Intermediate 12

### 1-(tetrahydro-2H-pyran-4-yl)-4-(4,4,5,5-tetramethyl-1,3,2-dioxaborolan-2-yl)-1H-pyrazol e

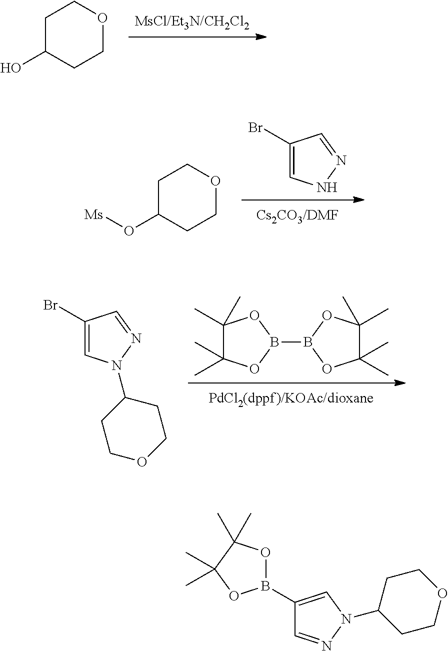

### (A) tetrahydro-2H-pyran-4-yl methanesulfonate

To a solution of tetrahydro-2H-pyran-4-ol (612 mg, 6 mmol) in DCM (5 mL) was added Et₃N (1002 uL, 7.2 mmol) and MsCl (510 uL, 6.6 mmol) at room temperature. The mixture was stirred at room temperature for 2 hours. After that the mixture was concentrated to give a white solid which was used for next step directly.

### (B) 4-bromo-1-(tetrahydro-2H-pyran-4-yl)-1H-pyrazole

To a solution of 4-bromo-1H-pyrazole (588 mg, 4 mmol) in DMF (15 mL) was added Cs₂CO₃ (1.95 g, 6 mmol) and tetrahydro-2H-pyran-4-yl methanesulfonate (6 mmol) at room temperature. The mixture was stirred at 120° C. for 18 hours. After that, the mixture was dissolved in 50 mL EA, washed with H₂O (25 mL) and brine (25 mL), dried over Na₂SO₄ and concentrated, purified by silica gel column chromatography (EA:PE=1:5) to give white solid. MS (m/z): 233 (M+H)⁺.

### (C) 1-(tetrahydro-2H-pyran-4-yl)-4-(4,4,5,5-tetramethyl-1,3,2-dioxaborolan-2-yl)-1H-pyrazole

To a solution of 4-bromo-1-(tetrahydro-2H-pyran-4-yl)-1H-pyrazole (745 mg, 3.21 mmol) in dioxane (15 mL) was added KOAc (944 mg, 9.63 mmol), PdCl₂(dppf) (352 mg, 0.48 mmol) and 4,4,4′,4′,5,5,5′,5′-octamethyl-2,2′-bi(1,3,2-dioxaborolane) (1.22 g, 4.82 mmol). The mixture was stirred at 80° C. for 24 hours, then was filtered and concentrated to give crude product, which was used for next step directly. MS (m/z): 279 (M+H)⁺.

### Intermediate 13

### 1-(4-(4-(4,4,5,5-tetramethyl-1,3,2-dioxaborolan-2-yl)phenyl)piperidin-1-yl)ethanone

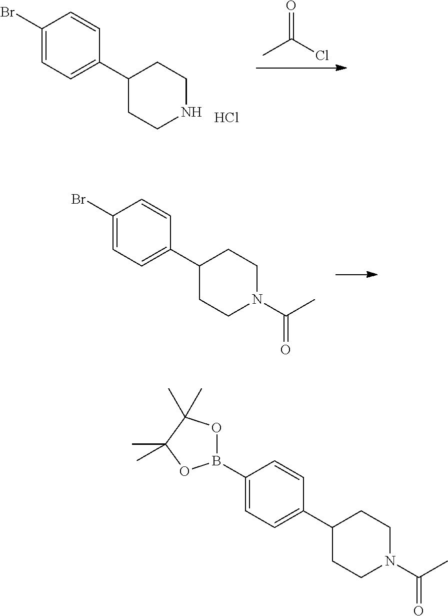

### (A) 1-(4-(4-bromophenyl)piperidin-1-yl)ethanone

The solution of 4-(4-bromophenyl)piperidine hydrochloride (500 mg, 1.81 mmol) in anhydrous THF was added TEA (366 mg, 3.62 mmol). The solution was cooled to 0° C. and added acetyl chloride (170 mg, 2.17 mmol) dropwise, stirred overnight at room temperature. The solvent was concentrated in vacuo, added water, extracted by EA. The organic phase was washed by 2N NaOH aqueous, brine, then dried over anhydrous Na₂SO₄, concentrated to give the title compound, which was used directly in the next step.

### (B) 1-(4-(4-(4,4,5,5-tetramethyl-1,3,2-dioxaborolan-2-yl)phenyl)piperidin-1-yl)ethanone

1-(4-(4-bromophenyl)piperidin-1-yl)ethanone (620 mg, 2.2 mmol), 4,4,5,5-tetramethyl-2-(4,4,5,5-tetramethyl-1,3,2-dioxaborolan-2-yl)-1,3,2-dioxaborolane (667 mg, 2.6 mmol)Cs₂CO₃ (1.43 g, 4.4 mmol) and Pd(dppf)Cl₂ (60 mg) was dissolved in dioxane in a flask. The mixture was charged with N₂, stirred at 50° C. for 5 hours. Then the solvent was removed in vacuo, the residue was purified by flash column chromatography (PE:EA=from 0:100 to 3:10) to give the title product. MS (m/z): 330 (M+H)⁺.

### Intermediate 14

### 1-(4-(4-(4,4,5,5-tetramethyl-1,3,2-dioxaborolan-2-yl)phenyl)piperazin-1-yl)ethanone

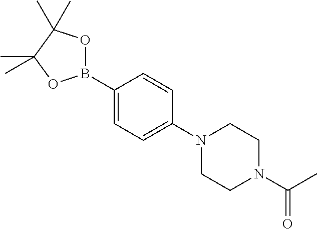 [SMILES: CC(=O)N1CCN(c2ccc(B3OC(C)(C)C(C)(C)O3)cc2)CC1]

The title compound was prepared according to the procedures of Intermediate 7 using instead MeCOCl. MS (m/z): 331 (M+H)⁺.

### Intermediate 15

### N,N-2-trimethyl-4-(4,4,5,5-tetramethyl-1,3,2-dioxaborolan-2-yl)aniline

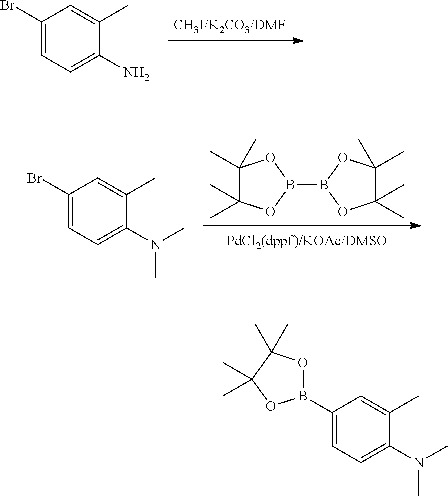

### (A) 4-bromo-N, N-2-trimethylaniline

To a solution of 4-bromo-2-methylaniline (558 mg, 3 mmol) in DMF (10 mL) was added K₂CO₃ (1242 mg, 9 mmol) and iodomethane (1278 mg, 9 mmol). The mixture was stirred at 100° C. for 24 hours. TLC and LC-MS showed the reaction had completed. The reaction solution was poured into 20 mL of H₂O, and extracted with EA, the organic phase was washed with water and brine, concentrated to give the products as light yellow oil. MS (m/z): 216 (M+2H)⁺

### (B) N, N-2-trimethyl-4-(4,4,5,5-tetramethyl-1,3,2-dioxaborolan-2-yl)aniline

To a solution of 4-bromo-N,N-2-trimethylaniline (571.7 mg, 2.67 mmol) in DMSO (20 mL) was added KOAc (787 mg, 8.01 mmol), PdCl₂(dppf)(293 mg, 0.4 mmol) and 4,4,4′,4′,5,5,5′,5′-octamethyl-2,2′-bi(1,3,2-dioxaborolane) (1.36 g, 5.34 mmol). The mixture was stirred at 80° C. for 6 hours under N₂. The reaction was added to 150 mL of water, extracted with EA, the organic phase was washed with brine, concentrated to give crude. The crude was purified by prep-TLC (EA:PE=1:5) to give white solid. MS (m/z): 262 (M+H)⁺.

### Intermediate 16

### 2-chloro-N,N-dimethyl-4-(4,4,5,5-tetramethyl-1,3,2-dioxaborolan-2-yl)aniline

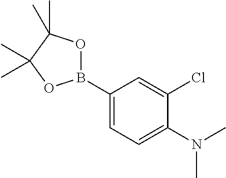

The title compound was prepared according to the procedures of Intermediate 15(A). MS (m/z): 282 (M+H)⁺.

### Intermediate 17

### 1-methyl-5-(4,4,5,5-tetramethyl-1,3,2-dioxaborolan-2-yl)-1H-indazole

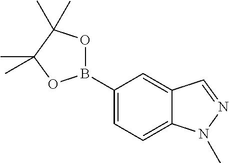 [SMILES: Cn1ncc2cc(B3OC(C)(C)C(C)(C)O3)ccc21]

The title compound was prepared according to the procedures of Intermediate 15(A). MS (m/z): 259 (M+H)⁺.

### Intermediate 18

### 1-methyl-6-(4,4,5,5-tetramethyl-1,3,2-dioxaborolan-2-yl)-1H-indole

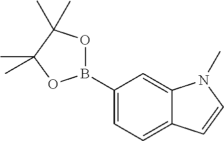 [SMILES: Cn1ccc2ccc(B3OC(C)(C)C(C)(C)O3)cc21]

The title compound was prepared according to the procedures of Intermediate 15(A). MS (m/z): 258 (M+H)⁺.

### Intermediate 19

### N-(2-methoxyethyl)-4-(4,4,5,5-tetramethyl-1,3,2-dioxaborolan-2-yl)aniline

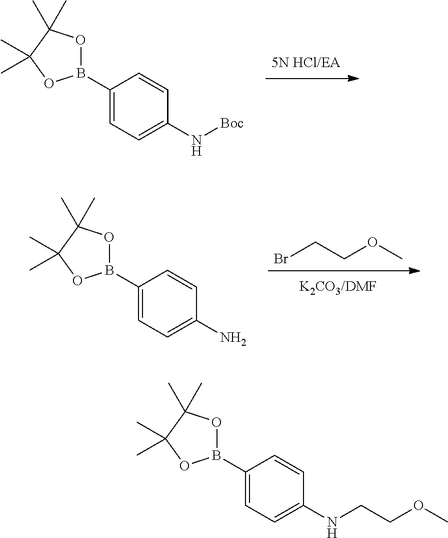

### (A) 4-(4,4,5,5-tetramethyl-1,3,2-dioxaborolan-2-yl)aniline

tert-butyl 4-(4,4,5,5-tetramethyl-1,3,2-dioxaborolan-2-yl)phenylcarbamate (300 mg, 0.94 mmol) was dissolved in a solution of HCl/EA and stirred for 4 hours at 20° C. The reaction was concentrated to give white solid, which was used for next step directly.

### (B) N-(2-methoxyethyl)-4-(4,4,5,5-tetramethyl-1,3,2-dioxaborolan-2-yl)aniline

To a solution of 4-(4,4,5,5-tetramethyl-1,3,2-dioxaborolan-2-yl)aniline (0.94 mmol) in DMF (10 mL) was added K₂CO₃ (270 mg, 1.5 mmol) and 1-bromo-2-methoxyethane (209 mg, 1.5 mmol), then the mixture was stirred at 100° C. for 24 hours. The solution was quenched with water and extracted with EA, the organic phase was washed with water and brine, concentrated and purified by prep-TLC (EA:PE=1:5) to give white solid. MS (m/z): 278 (M+H)⁺.

### Intermediate 20

### 2-(2-methoxy-4-(4,4,5,5-tetramethyl-1,3,2-dioxaborolan-2-yl)phenoxy)-N,N-dimethylethanamine

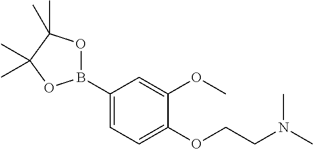

The title compound was prepared according to the procedures of Intermediate 19(B). MS (m/z): 322 (M+H)⁺.

### Intermediate 21

### N-methyl-2-(4-(4,4,5,5-tetramethyl-1,3,2-dioxaborolan-2-yl)phenoxy)acetamide

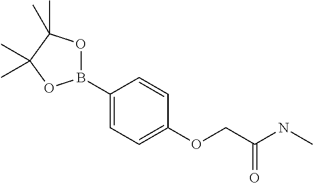

The title compound was prepared according to the procedures of Intermediate 19(B). MS (m/z): 292 (M+H)⁺.

### Intermediate 22

### N-methyl-4-(4,4,5,5-tetramethyl-1,3,2-dioxaborolan-2-yl)aniline

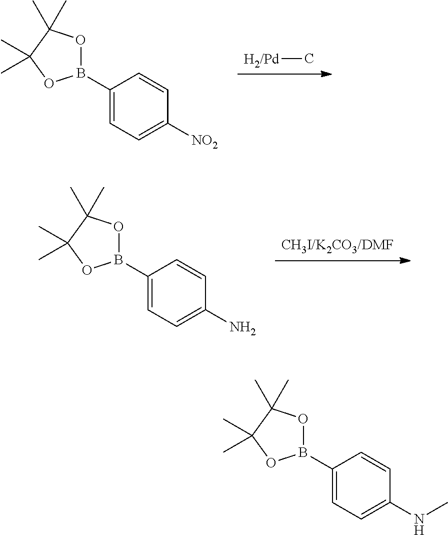

The title compound was prepared according to the procedures of Intermediate 1 (A) and 15 (A). MS (m/z): 234 (M+H)⁺.

### Intermediate 23

### 1-(4-(2-methyl-4-(4,4,5,5-tetramethyl-1,3,2-dioxaborolan-2-yl)phenyl)piperazin-1-yl)ethanone

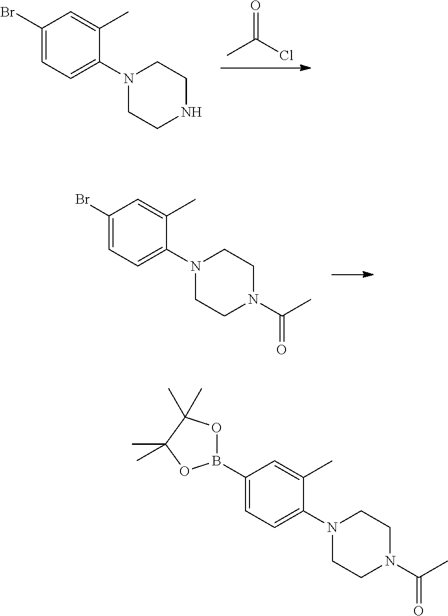

The title compound was prepared according to the procedures of Intermediate 13. MS (m/z): 345 (M+H)⁺.

### Intermediate 24

### 1-(2-methyl-4-(4,4,5,5-tetramethyl-1,3,2-dioxaborolan-2-yl)phenyl)-4-(methylsulfonyl)piperazine

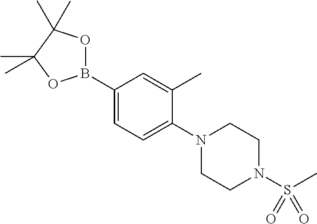

The title compound was prepared according to the procedures of Intermediate 13. MS (m/z): 381 (M+H)⁺.

### Intermediate 25

### 4-(4-(4,4,5,5-tetramethyl-1,3,2-dioxaborolan-2-yl)benzyl)morpholine

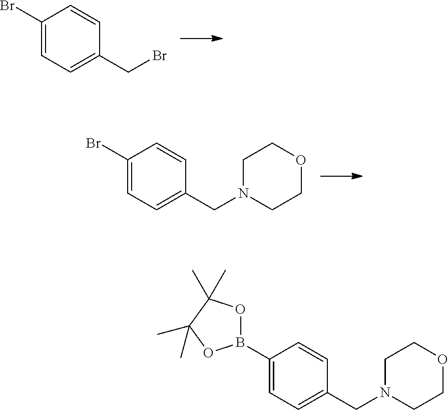 [SMILES: BrCc1ccc(Br)cc1 ; Brc1ccc(CN2CCOCC2)cc1 ; CC1(C)OB(c2ccc(CN3CCOCC3)cc2)OC1(C)C]

### (A) 4-(4-bromobenzyl)morpholine

1-bromo-4-(bromomethyl)benzene (2 g, 8 mmol) and morpholine (2.1 g, 24 mmol) was dissolved in anhydrous DMF, K₂CO₃ (5.53 g, 40 mmol) was added and the mixture was stirred overnight at 50° C. It was poured into water, extracted by EA/H₂O, the organic phase was washed by brine, dried over anhydrous Na₂SO₄, concentrated to give the title product as colorless oil (100% yield).

### (B) 4-(4-(4,4,5,5-tetramethyl-1,3,2-dioxaborolan-2-yl)benzyl)morpholine

The reactants 4-(4-bromobenzyl)morpholine (500 mg, 2 mmol), 4,4,5,5-tetramethyl-2-(4,4,5,5-tetramethyl-1,3,2-dioxaborolan-2-yl)-1,3,2-dioxaborolane (610 mg, 2.4 mmol), KOAc (294 mg, 3 mmol) and Pd(dppf)Cl₂ (50 mg), dioxane were mixed in a cube. The cube was sealed and reacted at 80° C. overnight under N₂ atmosphere. And then the mixture was purified by flash column chromatography (MeOH/H₂O) to give the title product as yellow solid (52% yield). MS (m/z): 304 (M+H)⁺.

### Intermediate 26

### N-methyl-N-(4-(4,4,5,5-tetramethyl-1,3,2-dioxaborolan-2-yl)benzyl)acetamide

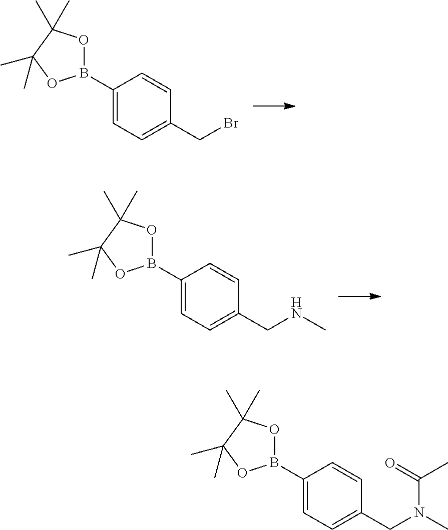

The reactant 2-(4-(bromomethyl)phenyl)-4,4,5,5-tetramethyl-1,3,2-dioxaborolane (400 mg, 1.34 mmol) was dissolved in the solution of MeNH₂ in MeOH (5 N, 5 mmol). The mixture was stirred at 40° C. for 4 hours, then the solvent was removed in vacuum, the residue was dissolved in DCM and cooled to 0° C. with ice bath. Then TEA (404 mg, 4 mmol) was added, and AcCl (160 mg, 2 mmol) was added dropwise. After that the ice bath was removed and the mixture was stirred at room temperature for 30 minutes, then it was partitioned with EA and H₂O. The organic phase was washed by brine, dried over anhydrous Na₂SO₄, concentrated to give the title product as white solid (77% yield). MS (m/z): 290 (M+H)⁺.

### Intermediate 27

### 2-(4-(4-(4,4,5,5-tetramethyl-1,3,2-dioxaborolan-2-yl)phenyl)piperidin-1-yl)ethanol

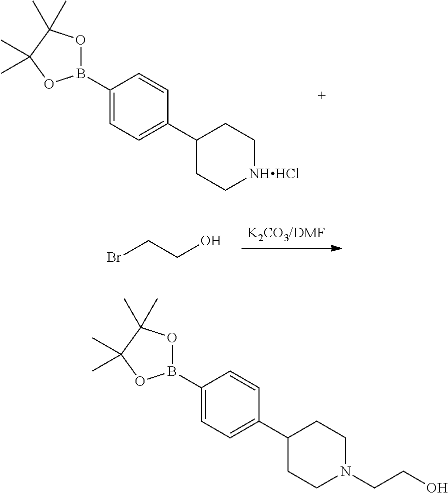

The title compound was prepared according to the procedures of Intermediate 11. MS (m/z): 332 (M+H)⁺.

### Intermediate 28

### N,N-dimethyl-2-(4-(4,4,5,5-tetramethyl-1,3,2-dioxaborolan-2-yl)phenyl)ethanamine

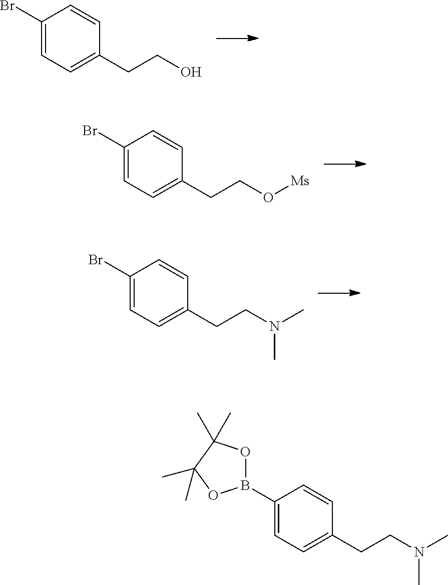

### (A) 4-bromophenethyl methanesulfonate

2-(4-bromophenyl)ethanol (2 g, 10 mmol) and TEA (1.515 g, 15 mmol) were dissolved in DCM and the mixture was cooled with ice-bath. Then MsCl (1.375 g, 12 mmol) was added slowly. After that the mixture was stirred for 2 hours, then poured into water, extracted DCM. The organic phase was concentrated to give the title product as colorless oil (97% yield).

### (B) 2-(4-bromophenyl)-N,N-dimethylethanamine

The reactant 4-bromophenethyl methanesulfonate (1 g, 3.58 mmol) and dimethylamine hydrochloride (880 mg, 10.74 mmol) were dissolved in DMF, K₂CO₃ (1.5 g, 10.74 mmol) was added and the mixture was stirred at 50° C. overnight. Then it was poured into water, extracted by EA. The organic phase was washed by brine, dried over anhydrous Na₂SO₄, concentrated to give the title product as brown solid (95% Yield).

### (C) N,N-dimethyl-2-(4-(4,4,5,5-tetramethyl-1,3,2-dioxaborolan-2-yl)phenyl) ethanamine

The reactant 2-(4-bromophenyl)-N,N-dimethylethanamine (500 mg, 2.2 mmol), 4,4,5,5-tetramethyl-2-(4,4,5,5-tetramethyl-1,3,2-dioxaborolan-2-yl)-1,3,2-dioxaborolane (660 mg, 2.6 mmol), KOAc (324 mg, 3.3 mmol) and Pd(dppf)Cl₂ (50 mg), dioxane were mixed in a cube. The cube was sealed and reacted at 80° C. overnight under N₂ atmosphere. After cooled it was purified by flash column chromatography (MeOH/H₂O) to give the title product as white solid (69% yield).

¹H NMR (400 MHz, CDCl₃) δ 7.75 (d, 2H), 7.22 (d, J=8.1 Hz, 2H), 3.16-3.12 (m, 4H), 2.74 (s, 6H), 1.33 (s, 12H)

### Intermediate 29

### 2-methyl-1-(4-(4,4,5,5-tetramethyl-1,3,2-dioxaborolan-2-yl)phenyl)propan-2-ol

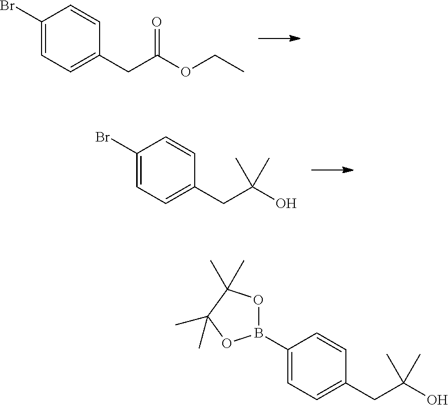 [SMILES: CCOC(=O)Cc1ccc(Br)cc1 ; CC(C)(O)Cc1ccc(Br)cc1 ; CC(C)(O)Cc1ccc(B2OC(C)(C)C(C)(C)O2)cc1]

### (A) 1-(4-bromophenyl)-2-methylpropan-2-ol

The mixture of ethyl 2-(4-bromophenyl)acetate (2.5 g, 10 mmol) in anhydrous THF was charged with N₂, cooled to 0° C. Then methylmagnesium bromide (2M, 6 mL, 12 mmol) was added dropwise, while the temperature was kept between 0˜5° C. After that the mixture was stirred at 0° C. for 2 hours. Then drops of water were added. After a while the mixture was poured into water, extracted by EA. The organic phase was washed by brine, dried over anhydrous Na₂SO₄, concentrated to give the title product as colorless oil (100% yield).

### (B) 2-methyl-1-(4-(4,4,5,5-tetramethyl-1,3,2-dioxaborolan-2-yl)phenyl) propan-2-ol

1-(4-bromophenyl)-2-methylpropan-2-ol (500 mg, 2.2 mmol), 4,4,5,5-tetramethyl-2-(4,4,5,5-tetramethyl-1,3,2-dioxaborolan-2-yl)-1,3,2-dioxaborolane (660 mg, 2.6 mmol), KOAc (324 mg, 3.3 mmol) and Pd(dppf)Cl₂, dioxane were mixed in a cube. The cube was sealed and reacted at 80° C. overnight under N₂ atmosphere. After cooling the mixture was partitioned with EA/H₂O, the organic phase was washed by brine, dried over anhydrous Na₂SO₄, concentrated to give title compound as black solid, which was used directly for the next step without further purification.

### Intermediate 30

### 1-(4-(4,4,5,5-tetramethyl-1,3,2-dioxaborolan-2-yl)phenyl)cyclopropanecarbonitrile

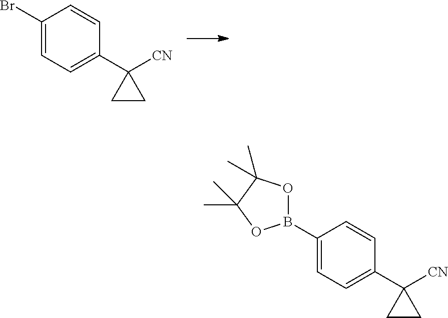 [SMILES: N#CC1(c2ccc(Br)cc2)CC1 ; CC1(C)OB(c2ccc(C3(C#N)CC3)cc2)OC1(C)C]

The title compound was prepared according to the procedures of Intermediate 15(B).

### Intermediate 31

### 1-(4-(4,4,5,5-tetramethyl-1,3,2-dioxaborolan-2-yl)phenyl)cyclobutanecarbonitrile

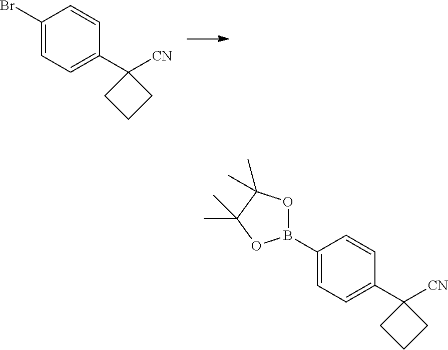 [SMILES: N#CC1(c2ccc(Br)cc2)CCC1 ; CC1(C)OB(c2ccc(C3(C#N)CCC3)cc2)OC1(C)C]

The title compound was prepared according to the procedures of Intermediate 15(B).

### Intermediate 32

### 2-methyl-2-(4-(4,4,5,5-tetramethyl-1,3,2-dioxaborolan-2-yl)phenyl)propan-1-ol

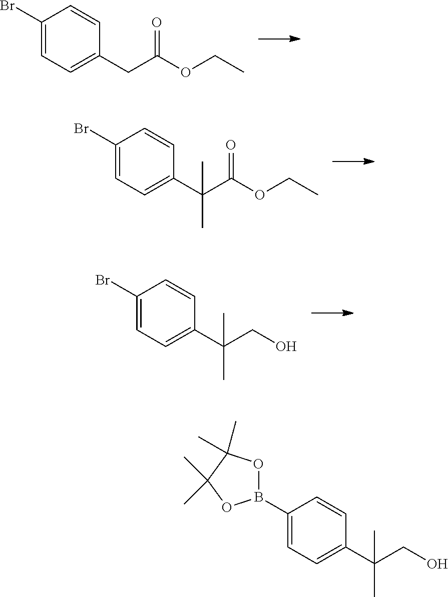

### (A) ethyl 2-(4-bromophenyl)-2-methylpropanoate

The mixture of ethyl 2-(4-bromophenyl)acetate (2.5 g, 10 mmol) in anhydrous THF was cooled to 0° C., then NaH (720 mg, 15 mmol) was added portion wise, while the temperature was kept between 0˜5° C. After that the mixture was stirred for 2 hours at room temperature, then it was cooled to 0° C. MeI (2.13 g, 15 mmol) was added and the mixture was stirred overnight at room temperature. Then drops of water were added. After a while the mixture was poured into water, extracted by EA, the organic phase was washed by brine, dried over anhydrous Na₂SO₄, concentrated to give the title product as colorless oil (98% Yield).

### (B) 2-(4-bromophenyl)-2-methylpropan-1-ol

Ethyl 2-(4-bromophenyl)-2-methylpropanoate (2.75 g, 10 mmol) in anhydrous THF was added dropwise to the mixture of LiAlH₄ (456 mg, 12 mmol) in anhydrous THF while the temperature was kept between 0-5° C. The mixture was stirred for 2 hours at 0° C. After that water (456 mg), 2N HCl (456 mg) and water (456 mg) were added sequentially. The mixture was filtrated to remove precipitation. The filtrate was concentrated to give the title product as colorless oil (89% Yield).

### (C) 2-methyl-2-(4-(4,4,5,5-tetramethyl-1,3,2-dioxaborolan-2-yl)phenyl) propan-1-ol

2-(4-bromophenyl)-2-methylpropan-1-ol (500 mg, 2 mmol), 4,4,5,5-tetramethyl-2-(4,4,5,5-tetramethyl-1,3,2-dioxaborolan-2-yl)-1,3,2-dioxaborolane (610 mg, 2.4 mmol), KOAc (300 mg, 3.0 mmol) and Pd(dppf)Cl₂ (50 mg), dioxane were mixed in a cube. The cube was sealed and reacted at 80° C. overnight under N₂ atmosphere. After cooling the mixture was partitioned with EA/H₂O, the organic phase was washed by brine, dried over anhydrous Na₂SO₄, concentrated to give the title product as black solid, which was used directly for the next step without further purification.

### Intermediate 33

### 1-(methylsulfonyl)-4-(4-(4,4,5,5-tetramethyl-1,3,2-dioxaborolan-2-yl)phenyl)piperidine

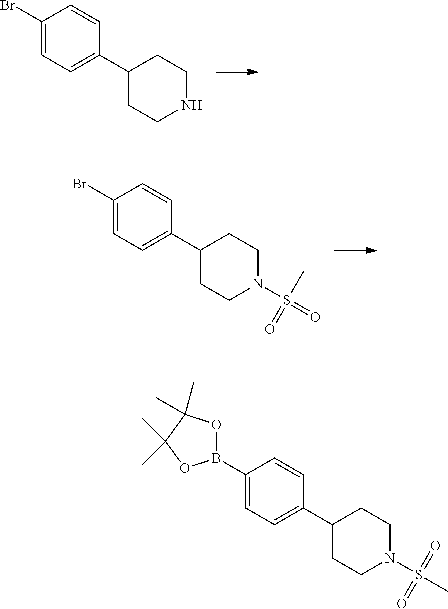

The title compound was prepared according to the procedures of Intermediate 13. MS (m/z): 366 (M+H)⁺.

### Intermediate 34 and 35

### (S)-tert-butyl 2-((R)-1-((7-chloropyrido[3,4-b]pyrazin-5-yl)oxy)ethyl)morpholine-4-carboxylate and (S)-tert-butyl 2-((S)-1-((7-chloropyrido[3,4-b]pyrazin-5-yl)oxy)ethyl)morpholine-4-carboxylate

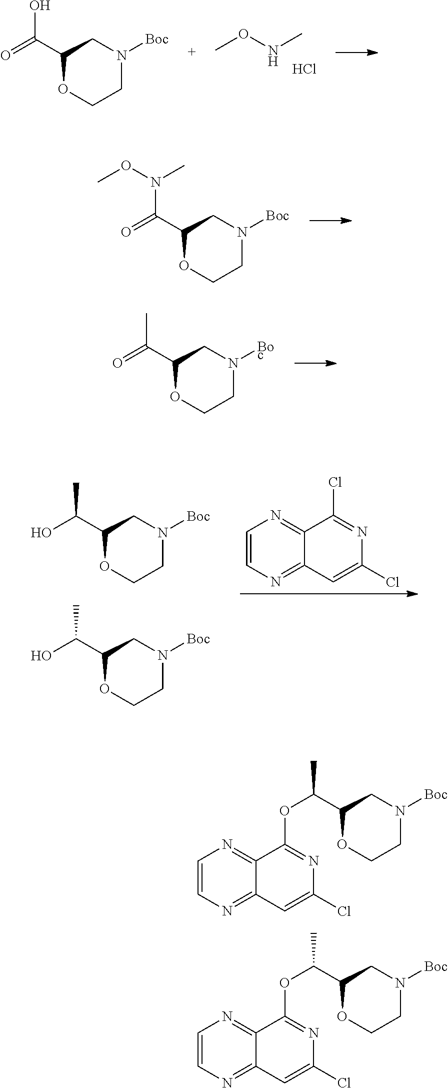

### (A) (S)-tert-butyl 2-(methoxy(methyl)carbamoyl)morpholine-4-carboxylate

A mixture of (S)-4-(tert-butoxycarbonyl)morpholine-2-carboxylic acid (6.93 g, 30 mmol), DIPEA (9.70 g, 75 mmol), and N,O-Dimethylhydroxylamine HCl (4.39 g, 45 mmol) in DCM (100 mL) was treated with HATU (22.8 g, 60 mmol) at room temperature. The reaction mixture was stirred for 16 hours and then poured into saturated aqueous sodium bicarbonate solution and extracted with CH₂Cl₂. The combined extracts were dried over MgSO₄, filtered, and concentrated to provide light yellow oil 14.95 g. MS (m/z): 175 (M+H-Boc)⁺

### (B) (S)-tert-butyl 2-acetylmorpholine-4-carboxylate

(S)-tert-butyl 2-(methoxy(methyl)carbamoyl)morpholine-4-carboxylate obtained above was dissolved in THF (60 mL) at room temperature under nitrogen, then the mixture was cooled to 0° C. Methylmagnesium bromide (3.0M solution in diethyl ether, 30 mL, 90 mmol) was added in portions. The reaction mixture was stirred at 0° C. for 1 hour, allowed to warm to room temperature and stirred for 16 hours. The mixture was again cooled to 0° C. and saturated aqueous ammonium chloride solution was slowly added. The mixture was extracted with EtOAc, and the organic phase was washed with brine, dried over MgSO₄, filtered and concentrated, purified by silica gel chromatography (petro ether:ethyl acetate=5:1) to provide 2.4 g colorless oil. MS (m/z): 130 (M+H-Boc)⁺.

¹H NMR (400 MHz, cdcl3) δ 4.20-4.08 (m, 1H), 3.98-3.93 (m, 1H), 3.89-3.78 (m, 2H), 3.59-3.52 (m, 1H), 2.99-2.91 (m, 1H), 2.84-2.76 (m, 1H), 2.22 (s, 3H), 1.46 (d, J=0.7, 9H).

### (C) (S)-tert-butyl 2-((R)-1-((7-chloropyrido[3,4-b]pyrazin-5-yl)oxy)ethyl)morpholine-4-carboxylate and (S)-tert-butyl 2-((S)-1-((7-chloropyrido[3,4-b]pyrazin-5-yl)oxy)ethyl)morpholine-4-carboxylate

To (S)-tert-butyl 2-acetylmorpholine-4-carboxylate (2.4 g, 10.5 mmol) in methanol (10 mL) at 0° C. was added sodium borohydride (0.59 g, 15.7 mmol). After 2 hours, the reaction was quenched with saturated ammonium chloride solution and extracted with dichloromethane. The organic phase was dried over magnesium sulfate, filtered, and concentrated. The residue was dissolved in N,N-dimethylformamide (50 mL) and the mixture was cooled to 5° C. under a nitrogen atmosphere. Sodium hydride (60% in mineral oil, 0.55 g, 13.6 mmol) was added portion-wise over 15 minutes and the mixture was stirred at 5° C. for 1 hour. 5,7-dichloropyrido[3,4-b]pyrazine (2.10 g, 10.5 mmol) was then added portion-wise and the mixture stirred at 5° C. for another 1 hour and quenched by addition of saturated aqueous ammonium chloride solution (50 mL). The solution was partitioned between ethyl acetate and water. The aqueous was re-extracted with ethyl acetate and the combined organic phases were washed with water, separated using a phase separation cartridge and concentrated to give brown oil. The crude residue was dissolved in DCM and purified by silica gel column chromatography eluting with 12-62% ethyl acetate in petroleum ether gradient. The appropriate fractions were combined and the solvent was evaporated to give 2 products:

p1; 967 mg, yield 23.3%, MS (m/z): 295 (M+H-Boc)⁺; ¹H NMR (400 MHz, cdcl3) δ 8.94 (d, J=1.8, 1H), 8.85 (d, J=1.8, 1H), 7.54 (s, 1H), 5.64-5.55 (m, 1H), 4.15-4.10 (m, 1H), 3.95-3.89 (m, 1H), 3.87-3.79 (m, 1H), 3.74-3.68 (m, 1H), 3.60-3.53 (m, 1H), 3.02-2.87 (m, 2H), 1.54 (d, J=6.4, 3H), 1.41 (s, 9H).

p2; 869 mg, yield 21%. MS (m/z): 295 (M+H-Boc)⁺; ¹H NMR (400 MHz, cdcl3) δ 8.93 (d, J=1.7, 1H), 8.86 (d, J=1.7, 1H), 7.52 (s, 1H), 5.73-5.63 (m, 1H), 4.06-3.97 (m, 1H), 3.95-3.89 (m, 1H), 3.86-3.79 (m, 1H), 3.78-3.71 (m, 1H), 3.59-3.51 (m, 1H), 3.02-2.94 (m, 1H), 2.92-2.83 (m, 1H), 1.50 (d, J=6.5, 3H), 1.45 (s, 9H).

### Intermediate 36

### 2-(1,1-dimethyl-1,3-dihydroisobenzofuran-5-yl)-4,4,5,5-tetramethyl-1,3,2-dioxaborolane

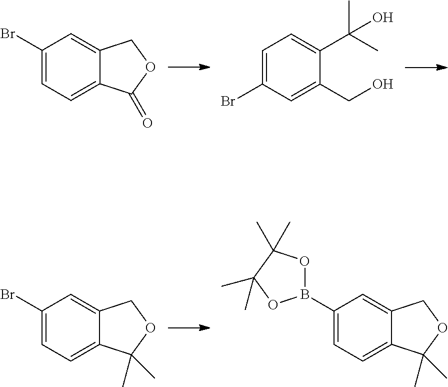

### (A) 2-(4-bromo-2-(hydroxymethyl)phenyl)propan-2-ol

A solution of 5-bromoisobenzofuran-1(3H)-one (4.26 g, 20 mmol) in dry tetrahydrofuran (100 mL) under argon was cooled in an ice bath. Methylmagnesium bromide (3M in diethylether, 20 mL, 60 mmol) was added drop wise and the resulting mixture was left to warm to room temperature overnight. The reaction mixture was cooled to 0° C. and saturated aqueous ammonium chloride was added. The mixture was extracted with ethyl acetate and the organic phase was dried over magnesium sulfate, filtered and concentrated. The crude product was filtered through a plug of silica gel with 50% ethyl acetate in heptane to give 2-(4-bromo-2-(hydroxymethyl)phenyl)propan-2-ol as white solid 1.76 g. Yield 36%.

¹H NMR (400 MHz, cdcl₃) δ 7.48 (d, J=2.2, 1H), 7.37 (dd, J=8.5, 2.2, 1H), 7.16 (d, J=8.5, 1H), 4.79 (s, 2H), 2.83 (s, 2H), 1.65 (s, 6H).

### (B) 5-bromo-1,1-dimethyl-1,3-dihydroisobenzofuran

Phosphoric acid (11.2 g, 115 mmol) was added to a suspension of 2-(4-bromo-2-(hydroxymethyl)phenyl)propan-2-ol (1.76 g, 7.2 mmol) in toluene (25 mL). The mixture was heated at 80° C. for 3 hours. The reaction was cooled to room temperature then to 0° C. The mixture was basified with 2M sodium hydroxide, then extracted with ethyl acetate (×2). The organic phase was dried over magnesium sulfate, filtered and concentrated to give 1.62 g 5-bromo-1,1-dimethyl-1,3-dihydroisobenzofuran as oil. Yield 99%.

¹H NMR (400 MHz, cdcl₃) δ 7.38 (d, J=8.0, 1H), 7.33 (s, 1H), 6.98 (d, J=8.0, 1H), 5.02 (s, 2H), 1.48 (s, 6H).

### (C) 2-(1,1-dimethyl-1,3-dihydroisobenzofuran-5-yl)-4,4,5,5-tetramethyl-1,3,2-dioxaborolane

A mixture of 5-bromo-1,1-dimethyl-1,3-dihydroisobenzofuran (1.62 g, 7.2 mmol), bis(pinacolato)diboron (2.69 g, 10.6 mmol), Pd(dppf)Cl₂ (205 mg, 0.28 mmol) and KOAc (2.09 g, 21.3 mmol) in anhydrous dioxane (80 mL) was heated at 100° C. for 4 hours. The reaction mixture was filtered and the solid was washed with CH₂Cl₂. The filtrate was concentrated in vacuo and purified by silica-gel chromatography eluting with Hexane-100% EtOAc (gradient) to afford crude product 1.9 g (yield 97%).

¹H NMR (400 MHz, cdcl₃) δ 7.73 (d, J=7.5, 1H), 7.66 (s, 1H), 7.13 (d, J=7.5, 1H), 5.06 (s, 2H), 1.49 (s, 6H), 1.34 (s, 12H).

### Intermediate 37

### 2-(1,1-dimethylisochroman-6-yl)-4,4,5,5-tetramethyl-1,3,2-dioxaborolane

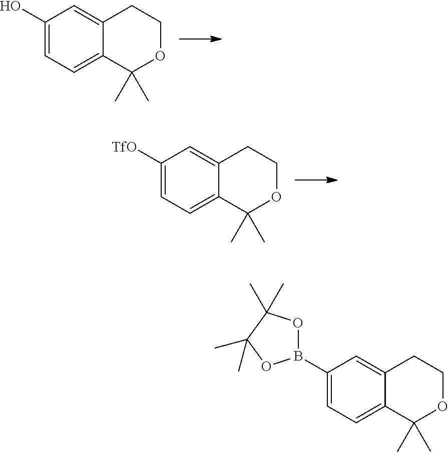

### (A) 1,1-dimethylisochroman-6-yl trifluoromethanesulfonate

To a stirred mixture of 1,1-dimethylisochroman-6-ol (1.78 g, 10 mmol) and triethylamine (3.03 g, 30 mmol) in dry dichloromethane (30 mL) under argon at 0° C. was added drop-wise trifluoromethanesulfonic anhydride (8.46 g, 30 mmol). The resulting mixture was allowed to warm slowly to 20° C. over 16 hours, then was poured into saturated aqueous sodium bicarbonate (50 mL) and extracted with dichloromethane (2×30 mL). The combined organic extracts were washed with brine (50 mL), dried over Na₂SO₄ and evaporated in vacuo. The residue was purified by silica gel chromatography eluting with ethyl acetate in hexane (10%-30%) to give 1,1-dimethylisochroman-6-yl trifluoromethanesulfonate as oil, which was used directly in the next step.

### (B) 2-(1,1-dimethylisochroman-6-yl)-4,4,5,5-tetramethyl-1,3,2-dioxaborolane

A mixture of 1,1-dimethylisochroman-6-yl trifluoromethanesulfonate, bis(pinacolato)diboron (3.81 g, 12 mmol), Pd(dppf)Cl₂ (292 mg, 0.4 mmol) and KOAc (2.94 g, 30 mmol) in anhydrous dioxane (80 mL) was heated at 100° C. for 4 hours. The reaction mixture was filtered and the solid was washed with CH₂Cl₂. The filtrate was concentrated in vacuo and purified by silica-gel chromatography eluting with Hexane-100% EtOAc (gradient) to afford oil 2.88 g (yield 100%).

¹H NMR (400 MHz, cdcl₃) δ 7.18-7.13 (m, 1H), 7.08-7.02 (m, 1H), 7.00-6.97 (m, 1H), 3.93 (t, J=5.6, 2H), 2.83 (t, J=5.4, 2H), 1.51 (s, 6H), 1.25 (s, 12H).

### Intermediate 38

### tert-butyl 3-(4-(4,4,5,5-tetramethyl-1,3,2-dioxaborolan-2-yl)phenyl)azetidine-1-carboxylate

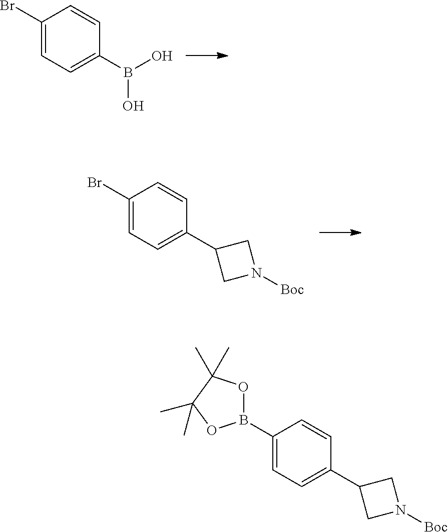 [SMILES: OB(O)c1ccc(Br)cc1 ; CC(C)(C)OC(=O)N1CC(c2ccc(Br)cc2)C1 ; CC(C)(C)OC(=O)N1CC(c2ccc(B3OC(C)(C)C(C)(C)O3)cc2)C1]

### (A) tert-butyl 3-(4-bromophenyl)azetidine-1-carboxylate

In the air, 4-bromophenylboronic acid (2.4 g, 12 mmol), NiI₂ (94 mg, 0.3 mmol), trans-2-aminocyclohexanol hydrochloride (45 mg, 0.3 mmol) and sodium hexamethyldisilazane (2.2 g, 12 mmol) were mixed in a microwave vial. The mixture was capped then placed under a nitrogen atmosphere. Isopropyl alcohol (10 mL) was added and the mixture was stirred under nitrogen for 5-10 minutes. 1-Boc-3-iodoazetidine (1.7 g, 6 mmol) was added in isopropyl alcohol (1 mL+1 mL rinse). The nitrogen atmosphere was removed and the mixture was heated to 80° C. under microwave irradiation. Heating was maintained at 80° C. for 30 minutes. After cooling the mixture was diluted with ethanol (10 mL) and filtered through a plug of celite. The filter cake was washed with ethanol (2×5 mL) and the filtrate was concentrated under vacuum to leave a crude oil. The oil was purified by preparative thin-layer chromatography using EtOAc/hexane (1:10) as eluent to give tert-butyl 3-(4-bromophenyl)azetidine-1-carboxylate 724 mg (yield 38%). MS (m/z): 212 (M+H-Boc)⁺

¹H NMR (400 MHz, CDCl₃) δ 7.47 (d, J=8.5, 2H), 7.18 (d, J=8.3, 2H), 4.34-4.29 (m, 2H), 3.94-3.90 (m, 2H), 3.72-3.63 (m, 1H), 1.46 (s, 9H).

### (B) tert-butyl 3-(4-(4,4,5,5-tetramethyl-1,3,2-dioxaborolan-2-yl)phenyl)azetidine-1-carboxylate

A mixture of tert-butyl 3-(4-bromophenyl)azetidine-1-carboxylate (0.72 g, 2.3 mmol), bis(pinacolato)diboron (0.88 g, 3.45 mmol), Pd(dppf)Cl₂ (67 mg, 0.09 mmol) and KOAc (0.68 g, 6.9 mmol) in anhydrous dioxane (30 mL) was heated at 100° C. for 4 hours. The reaction mixture was filtered and the solid was washed with CH₂Cl₂. The filtrate was concentrated in vacuo to afford crude tert-butyl 3-(4-(4,4,5,5-tetramethyl-1,3,2-dioxaborolan-2-yl)phenyl)azetidine-1-carboxylate 0.83 g (yield 100%), which was used directly in the next step. MS (m/z): 260 (M+H-Boc)⁺.

### Intermediate 39

### 1-(4-(4,4,5,5-tetramethyl-1,3,2-dioxaborolan-2-yl)phenyl)cyclobutanol

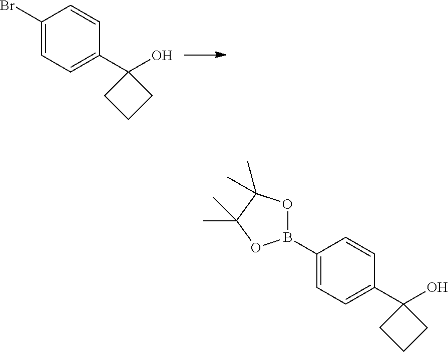 [SMILES: OC1(c2ccc(Br)cc2)CCC1 ; CC1(C)OB(c2ccc(C3(O)CCC3)cc2)OC1(C)C]

The title compound was prepared according to the procedures of Intermediate 15(B). ¹H NMR (400 MHz, CDCl₃) δ 7.83 (d, 2H), 7.51 (d, 2H), 2.61-2.53 (m, 2H), 2.42-2.34 (m, 2H), 2.20 (s, 1H), 2.06-2.01 (m, 1H), 1.76-1.64 (m, 1H), 1.35 (s, 12H).

### Intermediate 40

### 4-(4-(4,4,5,5-tetramethyl-1,3,2-dioxaborolan-2-yl)phenyl)piperidine hydrochloride

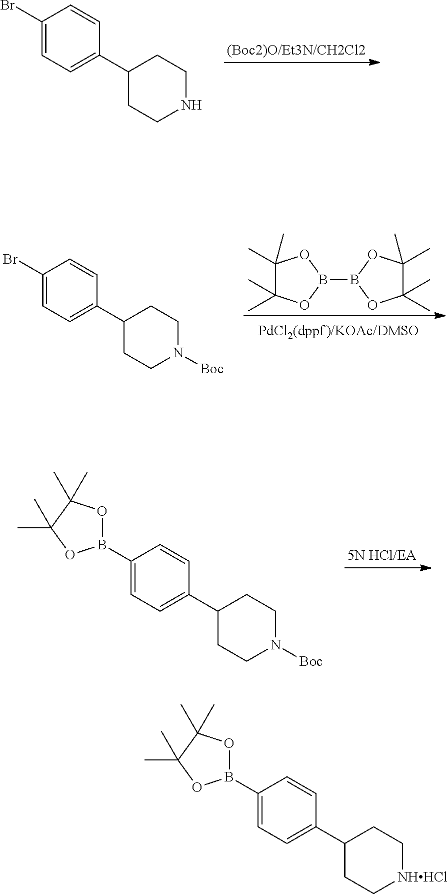

### (A) tert-butyl 4-(4-bromophenyl)piperidine-1-carboxylate

To a solution of 4-(4-bromophenyl)piperidine (2.4 g, 10 mmol) and Et₃N (1.4 mL, 10.5 mmol) in CH₂Cl₂ (30 mL) was added a solution of di-tert-butyl dicarbonate (2.29 g, 10.5 mmol) in CH₂Cl₂ (20 mL) dropwise at 0° C. The reaction mixture was stirred at room temperature for 4 hours. After that, the reaction was washed with NaHCO₃ (25 mL), H₂O (25 mL) and brine (25 mL), dried over Na₂SO₄ and concentrated to give crude oil. MS (m/z): 286 (M-t-butyl)⁺

### (B) tert-butyl 4-(4-(4,4,5,5-tetramethyl-1,3,2-dioxaborolan-2-yl)phenyl)piperidine-1-carboxylate

To a solution of tert-butyl 4-(4-bromophenyl)piperidine-1-carboxylate (10 mmol) in DMSO (1000 mL) was added KOAc (2.95 g, 30 mmol), PdCl₂(dppf) (1098 mg, 1.5 mmol) and 4,4,4′,4′,5,5,5′,5′-octamethyl-2,2′-bi(1,3,2-dioxaborolane) (5.08 g, 20 mmol). The mixture was stirred at 80° C. for 6 hours under N₂ atmosphere. The mixture was poured to 150 mL of water, extracted with EA. The organic phase was washed with brine, concentrated to give a crude. The crude was purified by column chromatography (CH₂Cl₂: MeOH=20:1) to give yellow oil. MS (m/z): 288 (M-C₅H₉O₂+H)⁺

### (C) 4-(4-(4,4,5,5-tetramethyl-1,3,2-dioxaborolan-2-yl)phenyl)piperidine hydrochloride

tert-butyl 4-(4-(4,4,5,5-tetramethyl-1,3,2-dioxaborolan-2-yl)phenyl)piperidine-1-carboxylate (10 mmol) was dissolved in 10 mL of EA and a solution of 5N HCl/EA (10 mL) was added into the solution. The reaction mixture was stirred for 8 hours at 20° C. Then the reaction mixture was concentrated to give crude product as white solid. MS (m/z): 288 (M+H)⁺

### Intermediate 41

### 1-methyl-5-(4,4,5,5-tetramethyl-1,3,2-dioxaborolan-2-yl)indolin-2-one

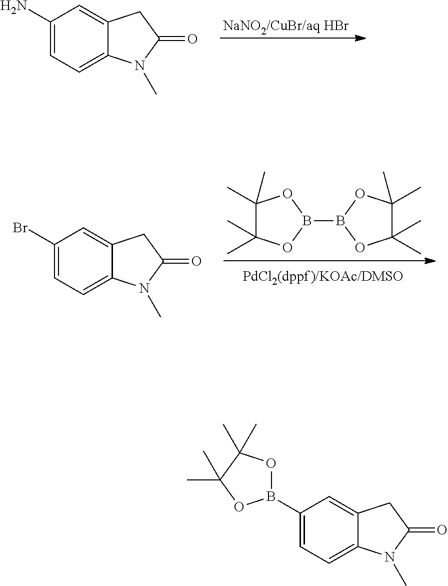

### (A) 5-bromo-1-methylindolin-2-one

To a solution of 5-amino-1-methylindolin-2-one (811 mg, 5 mmol) in 40 mL of 40% aqueous HBr was added a solution of NaNO₂ (380 mg, 55 mmol) in 3 mL of H₂O at 0° C. The mixture was stirred at 0° C. for 40 minutes. After that the mixture was slowly poured into a solution of CuBr (1.51 g, 10.5 mmol) in 10 mL aq. HBr at 0° C. The reaction mixture was heated to 60° C. and stirred for 2 hours. After cooling the mixture was basified with 2N aq. NaOH until pH=8˜9 and extracted with EA. The organic phase was washed with H₂O and brine, concentrated and purified by column chromatography (EA:PE=1:1) to give crude as solid. MS (m/z): 228 (M+2)⁺

### (B) 1-methyl-5-(4,4,5,5-tetramethyl-1,3,2-dioxaborolan-2-yl)indolin-2-one

To a solution of 5-bromo-1-methylindolin-2-one (140 mg, 0.62 mmol) in DMSO (10 mL) was added KOAc (183 mg, 1.86 mmol), PdCl₂(dppf) (68 mg, 0.093 mmol) and 4,4,4′,4′,5,5,5′,5′-octamethyl-2,2′-bi(1,3,2-dioxaborolane) (315 mg, 1.24 mmol). The mixture was stirred at 80° C. for 6 hours under N₂ atmosphere. The reaction was poured to 150 mL of water, extracted with EA. The organic phase was washed with brine, concentrated to give a crude. The crude was purified by column chromatography (EA:PE=1:3) to give yellow oil. MS (m/z): 274 (M+H)⁺

### Intermediate 42

### (6-(dimethylamino)-5-methylpyridin-3-yl)boronic acid

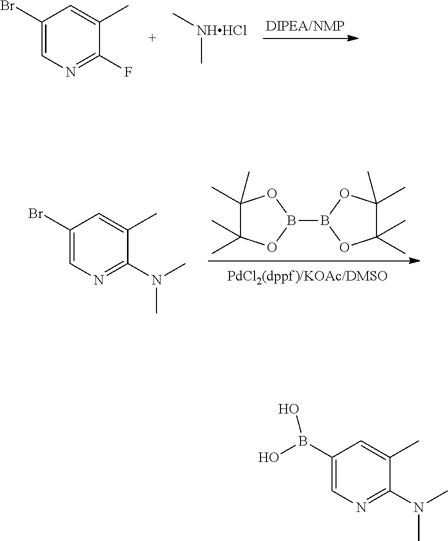

### (A) 5-bromo-N,N,3-trimethylpyridin-2-amine

To a solution of 5-bromo-2-fluoro-3-methylpyridine (475 mg, 2.5 mmol) in NMP (5 mL) in a tube was added dimethylamine hydrochloride (408 mg, 5 mmol) and N-ethyl-N-isopropylpropan-2-amine (1.68 mL, 10 mmol). The tube was sealed and heated in microwave at 180° C. for 1 hour. TLC and LC-Ms showed the reaction had completed and the desired compound was detected. The reaction mixture was poured into 30 mL of H₂O, and extracted with EA. The organic phase was washed with water and brine, dried and concentrated to give yellow oil. MS (m/z): 217 (M+2)⁺

### (B) 6-(dimethylamino)-5-methylpyridin-3-ylboronic acid

To a solution of 5-bromo-N,N,3-trimethylpyridin-2-amine (475 mg, 2.21 mmol) in DMSO (10 mL) was added KOAc (650 mg, 6.63 mmol), PdCl₂(dppf) (242 mg, 0.33 mmol) and 4,4,4′,4′,5,5,5′,5′-octamethyl-2,2′-bi(1,3,2-dioxaborolane) (1.12 g, 4.42 mmol). The mixture was stirred at 80° C. for 6 hours under N₂ atmosphere. The reaction mixture was poured to 150 mL of water, extracted with EA. The organic phase was washed with brine, concentrated to give crude. The crude was purified by column chromatography (EA:PE=1:1) to give yellow oil. MS (m/z): 181 (M+H)⁺

### Intermediate 43

### 1-(2-methyl-4-(4,4,5,5-tetramethyl-1,3,2-dioxaborolan-2-yl)phenyl)piperazine hydrochloride

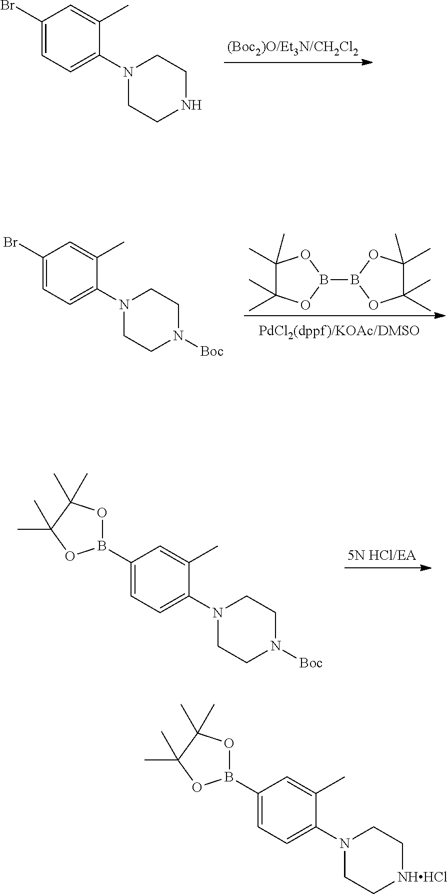

### (A) tert-butyl 4-(4-bromo-2-methylphenyl)piperazine-1-carboxylate

To a solution of 1-(4-bromo-2-methylphenyl)piperazine (2.55 g, 10 mmol) and Et₃N (1.4 mL, 10.5 mmol) in CH₂Cl₂ (30 mL) was added a solution of di-tert-butyl dicarbonate (2.29 g, 10.5 mmol) in CH₂Cl₂ (20 mL) dropwise at 0° C. The reaction mixture was stirred at room temperature for 4 hours. After that the reaction mixture was washed with aq. NaHCO₃ (25 mL), H₂O (25 mL) and brine (25 mL), dried over Na₂SO₄ and concentrated to give yellow oil. MS (m/z): 357 (M+H)⁺

### (B) tert-butyl 4-(2-methyl-4-(4,4,5,5-tetramethyl-1,3,2-dioxaborolan-2-yl)phenyl)piperazine-1-carboxylate

To a solution of tert-butyl 4-(4-bromo-2-methylphenyl)piperazine-1-carboxylate (10 mmol) in DMSO (100 mL) was added KOAc (2.95 g, 30 mmol), PdCl₂(dppf) (1098 mg, 1.5 mmol) and 4,4,4′,4′,5,5,5′,5′-octamethyl-2,2′-bi(1,3,2-dioxaborolane) (5.08 g, 20 mmol). The mixture was stirred at 80° C. for 6 hours under N₂ atmosphere. The reaction mixture was poured to 150 mL of water, extracted with EA. The organic phase was washed with brine, concentrated to give crude. The crude was purified by column chromatography (PE:EA=5:1) to give yellow oil. MS (m/z): 403 (M+H)⁺

### (C) 1-(2-methyl-4-(4,4,5,5-tetramethyl-1,3,2-dioxaborolan-2-yl)phenyl)piperazine hydrochloride

tert-butyl 4-(2-methyl-4-(4,4,5,5-tetramethyl-1,3,2-dioxaborolan-2-yl)phenyl)piperazine-1-carboxylate (10 mmol) was dissolved in 10 mL of EA and a solution of 5N HCl/EA (10 mL) was added into the solution. The reaction mixture was stirred for 8 hours at 20° C. The reaction mixture was concentrated to give white solid. MS (m/z): 303 (M+H)⁺

### Intermediate 44

### 1-methyl-6-(4,4,5,5-tetramethyl-1,3,2-dioxaborolan-2-yl)-1,2,3,4-tetrahydroquinoline

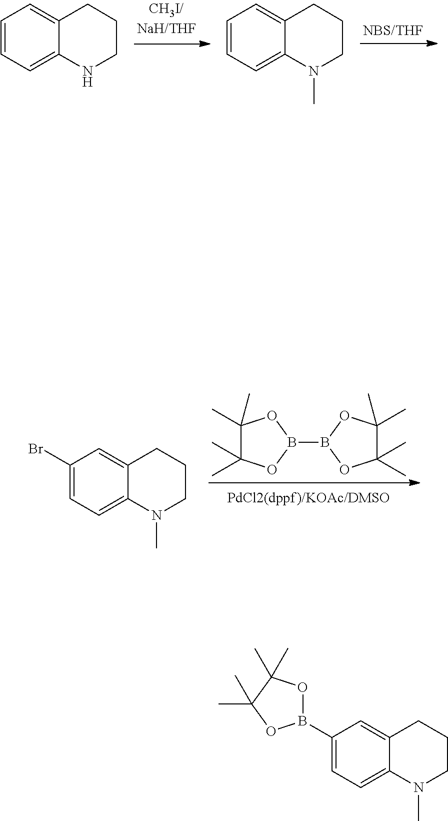

### (A) 1-methyl-1,2,3,4-tetrahydroquinoline

NaH (60%, 600 mg, 15 mmol) was added into a solution of 1,2,3,4-tetrahydroquinoline (1.33 g, 10 mmol) in THF (50 mL) at 0° C. and the mixture was stirred for 20 minutes. Then CH₃I (1.71 g, 15 mmol) was dropped into the reaction and the mixture was stirred for 16 hours at room temperature. The reaction solution was washed with saturated aq. NH₄Cl and extracted with EA. The organic phase was washed with water and brine, concentrated and purified by column chromatography (EA:PE=1:3) to give yellow oil. MS (m/z): 148 (M+H)⁺

### (B) 6-bromo-1-methyl-1,2,3,4-tetrahydroquinoline

NBS (1.06 g, 5.96 mmol) was added into a solution of 1-methyl-1,2,3,4-tetrahydroquinoline (877 mg. 5.96 mmol) in THF (20 mL) at −78° C. and the mixture was stirred for 3 hours at −78° C. and 16 hours at room temperature. The reaction mixture was washed with saturated aq. Na₂CO₃ and extracted with EA. The organic phase was washed with water and brine, concentrated to give yellow oil. MS (m/z): 403 (M+H)⁺

### (C) 1-methyl-6-(4,4,5,5-tetramethyl-1,3,2-dioxaborolan-2-yl)-1,2,3,4-tetrahydroquinoline

To a solution of 6-bromo-1-methyl-1,2,3,4-tetrahydroquinoline (1.35 g, 5.96 mmol) in DMSO (50 mL) was added KOAc (1.75 g, 17.88 mmol), PdCl₂(dppf) (651 mg, 0.85 mmol) and 4,4,4′,4′,5,5,5′,5′-octamethyl-2,2′-bi(1,3,2-dioxa borolane) (3.03 g, 11.92 mmol). The mixture was stirred at 80° C. for 6 hours under N₂ atmosphere. The reaction mixture was poured to 150 mL of water, extracted with EA. The organic phase was washed with brine, concentrated to give crude. The crude was purified by column chromatography (PE:EA=1:1) to give yellow oil. MS (m/z): 274 (M+H)⁺

### Intermediate 45

### 1-methyl-4-(2-methyl-4-(4,4,5,5-tetramethyl-1,3,2-dioxaborolan-2-yl)phenyl)piperazine

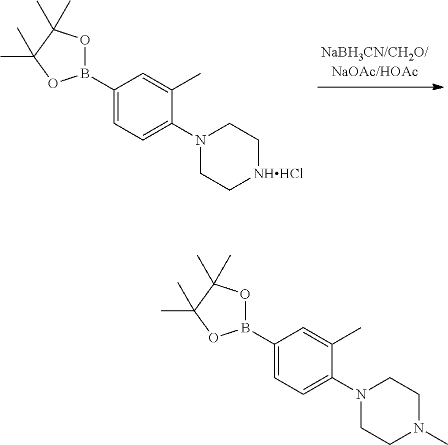

1-(2-methyl-4-(4,4,5,5-tetramethyl-1,3,2-dioxaborolan-2-yl)phenyl)piperazine hydrochloride (1.02 g, 3 mmol) was dissolved in 37% aqueous formaldehyde (30 mL) and acetic acid (1.8 g, 30 mmol). Sodium acetate (2.46 g, 30 mmol) was added and the mixture was cooled in ice/water bath. Sodium cyanoborohydride (377 mg, 6 mmol) was added and the mixture was stirred for 3 hours. Saturated aqueous NaHCO₃ was added until the mixture was basic. The mixture was extracted with DCM (×3) and the combined extract was dried over MgSO₄ and concentrated to give yellow solid. MS (m/z): 317 (M+H)⁺

### Intermediate 46

### 1-methyl-5-(4,4,5,5-tetramethyl-1,3,2-dioxaborolan-2-yl)indoline

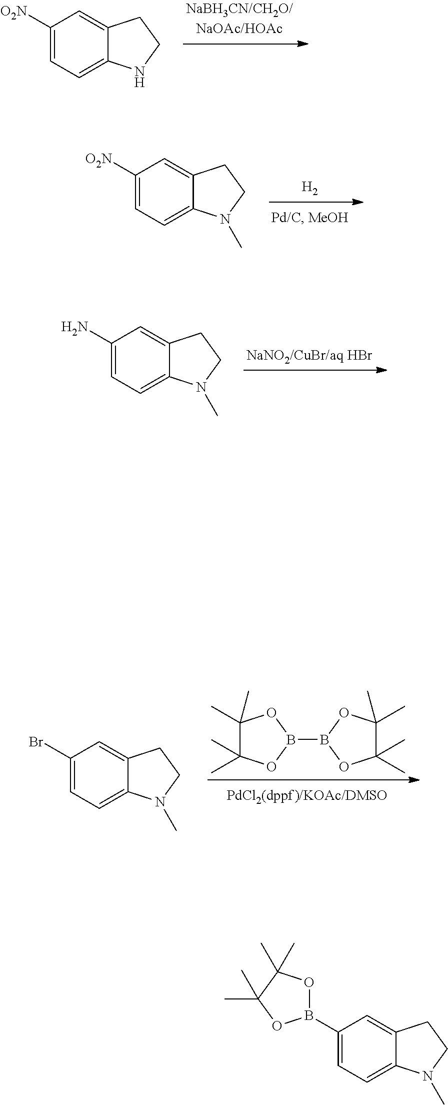

### (A) 1-methyl-5-nitroindoline

5-nitroindoline (1.64 g, 10 mmol) was dissolved in 37% aqueous formaldehyde (50 mL) and acetic acid (6.0 g, 100 mmol). Sodium acetate (8.2 g, 100 mmol) was added and the mixture was cooled in ice/water bath. Sodium cyanoborohydride (1.26 g, 20 mmol) was added and the mixture was stirred for 9 hours. Saturated aqueous NaHCO₃ was added until the mixture was basic. The mixture was extracted with DCM (×3) and the combined extracts were dried over MgSO₄ and concentrated to give yellow solid. MS (m/z): 179 (M+H)⁺

### (B) 1-methylindolin-5-amine

To a solution of 1-methyl-5-nitroindoline (10 mmol) in MeOH (30 mL) was added Pd/C (1 g), then the mixture was stirred for 4 hours at 20° C. under 1 atm H₂ atmosphere. The reaction mixture was filtered and the filtrate was concentrated, purified by column chromatography (EA:PE=1:1) to give gray solid. MS (m/z): 149 (M+H)⁺

### (C) 5-bromo-1-methylindoline

To a solution of 1-methylindolin-5-amine (960 mg, 6.48 mmol) in 10 mL of aq. HBr (40%) was added a solution of NaNO₂ (492 mg, 7.13 mmol) in 2 mL of H₂O at 0° C. The mixture was stirred at 0° C. for 40 minutes. The mixture was poured into a solution of CuBr (1.95 g, 13.6 mmol) in 10 mL aq. HBr at 0° C. Then the reaction mixture was heated to 60° C. and stirred for 2 hours. After cooling the mixture was basified with 2M aq. NaOH until pH=8˜9 and extracted with EA. The organic phase was washed with H₂O and brine, concentrated and purified by column chromatography (EA:PE=1:5) to give yellow solid. MS (m/z): 214 (M+2)⁺

### (D) 1-methyl-5-(4,4,5,5-tetramethyl-1,3,2-dioxaborolan-2-yl)indoline

To a solution of 5-bromo-1-methylindoline (47 mg, 0.22 mmol) in DMSO (5 mL) was added KOAc (65.3 mg, 0.66 mmol), PdCl₂(dppf) (24 mg, 0.35 mmol) and 4,4,4′,4′,5,5,5′,5′-octamethyl-2,2′-bi(1,3,2-dioxaborolane) (113 mg, 0.44 mmol). The mixture was stirred at 80° C. for 6 hours under N₂ atmosphere. The reaction mixture was poured to 150 mL of water, extracted with EA. The organic phase was washed with brine, concentrated to give crude. The crude was purified by column chromatography (EA:PE=1:20) to afford white solid. MS (m/z): 262 (M+H)⁺

### Intermediate 47

### (S)-4-(hydroxymethyl)-1-((S)-1-phenylethyl)pyrrolidin-2-one

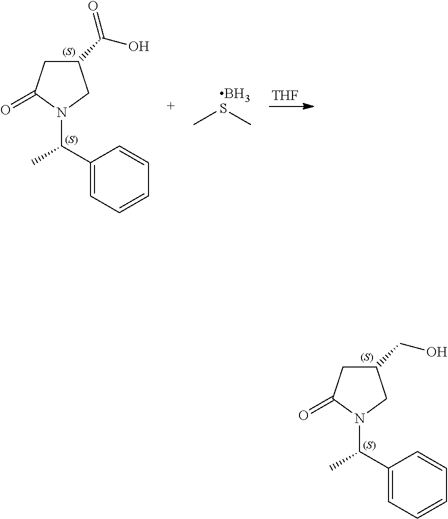

Borane dimethyl sulfide complex (2M in tetrahydrofuran 1.67 mL, 3.34 mmol) was dropped into a solution of (S)-5-oxo-1-((S)-1-phenylethyl)pyrrolidine-3-carboxylic acid (520 mg, 2.23 mol) in THF (10 mL) at 0° C. and the mixture was stirred for 3 hours at 25° C. The reaction was quenched with saturated aq. Na₂CO₃ and extracted with EA. The organic phase was washed with water and brine, concentrated to give yellow oil. MS (m/z): 220 (M+H)⁺

### Intermediate 48

### (R)-4-(hydroxymethyl)-1-((R)-1-phenylethyl)pyrrolidin-2-one

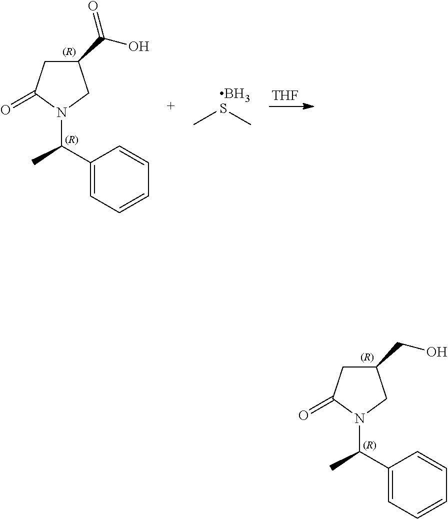

The title compound was prepared according to the procedures of Intermediate 47. MS (m/z): 220 (M+H)⁺.

### Intermediate 49

### tert-butyl (2-methyl-2-(4-(4,4,5,5-tetramethyl-1,3,2-dioxaborolan-2-yl)phenyl)propyl)carbamate

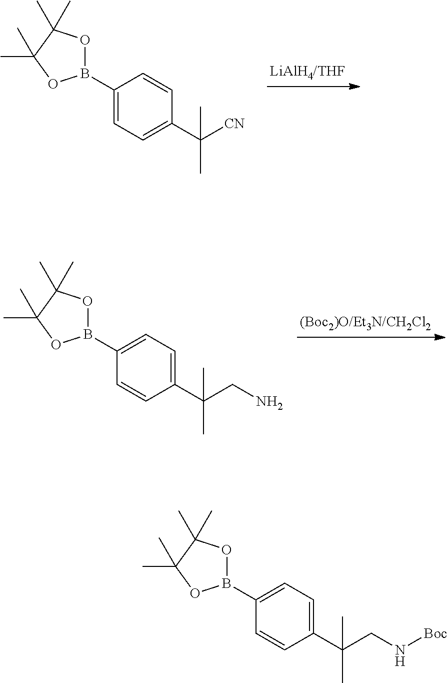 [SMILES: CC(C)(C#N)c1ccc(B2OC(C)(C)C(C)(C)O2)cc1 ; CC(C)(CN)c1ccc(B2OC(C)(C)C(C)(C)O2)cc1 ; CC(C)(C)OC(=O)NCC(C)(C)c1ccc(B2OC(C)(C)C(C)(C)O2)cc1]

### (A) 2-methyl-2-(4-(4,4,5,5-tetramethyl-1,3,2-dioxaborolan-2-yl)phenyl) propan-1-amine

To a mixture of LiAlH₄ (57 mg, 1.5 mmol) in dry THF (8 mL) was dropped into a solution of 2-methyl-2-(4-(4,4,5,5-tetramethyl-1,3,2-dioxaborolan-2-yl)phenyl)propanenitrile (271 mg, 1 mmol) in dry THF (2.0 mL) at 0° C. under N₂ atmosphere. After 30 minutes the cooling bath was removed and the mixture was stirred at room temperature for 3 hours. The mixture was again cooled to 0° C. and carefully quenched by the 2M aq. NaOH (0.5 mL). The resulting suspension was filtered and the filter cake was rinsed with THF. The filtrate was concentrated to give white oil. MS (m/z): 276 (M+H)⁺

### (B) tert-butyl 2-methyl-2-(4-(4,4,5,5-tetramethyl-1,3,2-dioxaborolan-2-yl)phenyl)propylcarbamate

To a solution of 2-methyl-2-(4-(4,4,5,5-tetramethyl-1,3,2-dioxaborolan-2-yl)phenyl)propan-1-amine (1 mmol) and Et₃N (153 uL, 1.1 mmol) in DCM (3 mL) was added a solution of di-tert-butyl dicarbonate (240 mg, 1.1 mmol) in DCM (2 mL) dropwise at 0° C. The reaction mixture was stirred at room temperature for 4 hours. After that the reaction mixture was washed with aq. NaHCO₃ (25 mL), H₂O (25 mL) and brine (25 mL), dried over Na₂SO₄ and concentrated to give yellow oil. MS (m/z): 376 (M+H)⁺

### Intermediate 50

### 3-(4-(4,4,5,5-tetramethyl-1,3,2-dioxaborolan-2-yl)phenyl)pentan-3-ol

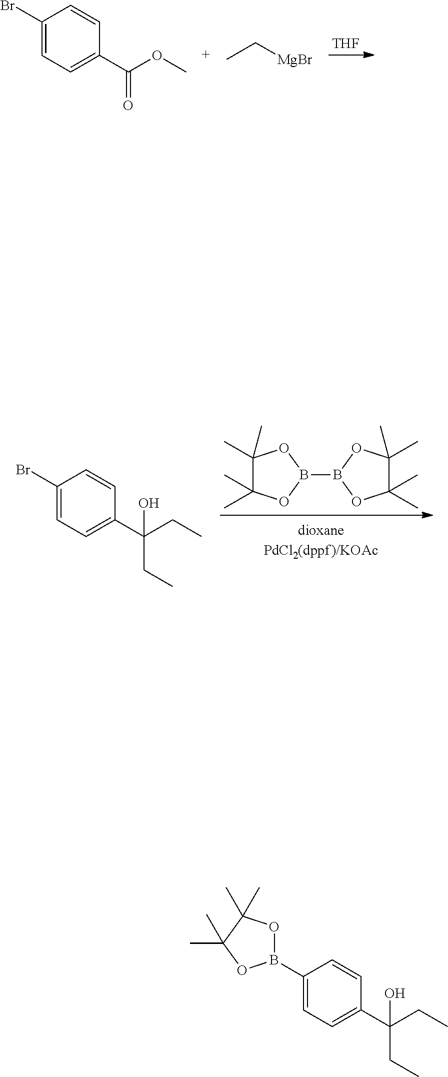

### (A) 3-(4-bromophenyl)pentan-3-ol

Ethylmagnesium bromide (3M in ether, 8 mL, 24 mmol) was dropped into a solution of methyl 4-bromobenzoate (2.15 g, 10 mmol) in THF (60 mL) at 0° C. and the mixture was stirred for 18 hours at 25° C. The reaction mixture was quenched with sat. aq. NH₄Cl and extracted with EA. The organic phase was washed with water and brine, dried and concentrated, purified by column chromatography (EA:PE=1:3) to give yellow oil. MS (m/z): 185 (M−2Ethyl)⁺

### (B) 3-(4-(4,4,5,5-tetramethyl-1,3,2-dioxaborolan-2-yl)phenyl)pentan-3-ol

To a solution of 3-(4-bromophenyl)pentan-3-ol (2.03 g, 8.35 mmol) in dioxane (85 mL) was added KOAc (2.47 g, 25.1 mmol), PdCl₂(dppf) (1.04 g, 1.25 mmol) and 4,4,4′,4′,5,5,5′,5′-octamethyl-2,2′-bi(1,3,2-dioxaborolane) (3.18 g, 12.5 mmol). The mixture was stirred at 100° C. for 3 hours under N₂ atmosphere. The reaction mixture was poured to 250 mL of water, extracted with EA. The organic phase was washed with brine, concentrated to give crude. The crude was purified by column chromatography (EA:PE=1:4) to give yellow solid. MS (m/z): 217 (M-C₄H₁₁O+H)⁺

### Intermediate 51

### N,N-dimethyl-1-(4-(4,4,5,5-tetramethyl-1,3,2-dioxaborolan-2-yl)phenyl)ethanamine

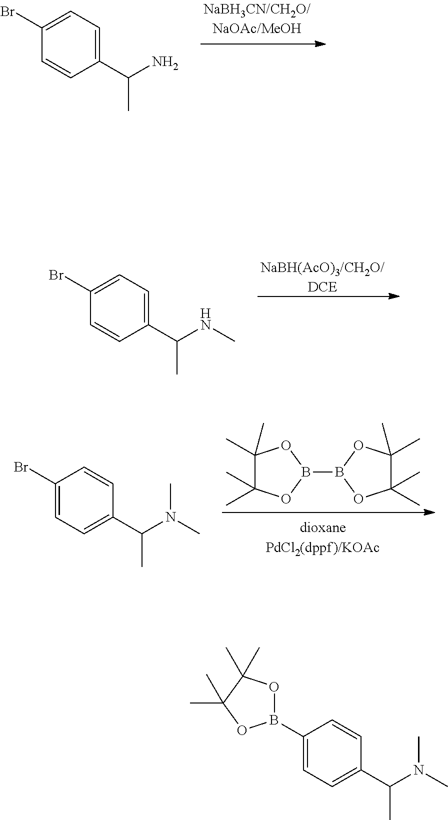

### (A) 1-(4-bromophenyl)-N-methylethanamine

1-(4-bromophenyl)ethanamine (1 g, 5 mmol) was dissolved in 37% aqueous formaldehyde (1.22 mL, 15 mmol) and MeOH (15 mL). Sodium acetate (1.64 g, 20 mmol) was added and the mixture was cooled in ice/water bath. Sodium cyanoborohydride (1.25 g, 20 mmol) was added and the mixture was stirred for 24 hours. Saturated aqueous sodium hydrogen carbonate was added until the mixture was basic. The mixture was extracted with DCM (×3) and the combined extracts were dried over MgSO₄ and concentrated to give yellow oil.

### (B) 1-(4-bromophenyl)-N,N-dimethylethanamine

1-(4-bromophenyl)-N-methylethanamine (5 mmol) in 37% aqueous formaldehyde (1.22 mL) and DCE (15 mL) was added NaBH(AcO)₃ (2.12 g, 20 mmol) at 0° C. and the mixture was stirred for 24 hours. Saturated aqueous sodium hydrogen carbonate was added until the mixture was basic. The mixture was extracted with DCM (×2) and the combined extracts were dried over MgSO₄ and concentrated, purified by thin-layer chromatography (DCM:MeOH=10:1) to give yellow solid. MS (m/z): 230 (M+2)⁺

### (C) N,N-dimethyl-1-(4-(4,4,5,5-tetramethyl-1,3,2-dioxaborolan-2-yl)phenyl)ethanamine

To a solution of 1-(4-bromophenyl)-N,N-dimethylethanamine (534 mg, 2.34 mmol) in dioxane (25 mL) was added KOAc (691 mg, 7.03 mmol), PdCl₂(dppf) (286 mg, 0.35 mmol) and 4,4,4′,4′,5,5,5′,5′-octamethyl-2,2′-bi(1,3,2-dioxaborolane) (891 mg, 3.51 mmol). The mixture was stirred at 100° C. for 3 hours under N₂ atmosphere. The reaction mixture was poured to 250 mL of water, extracted with EA. The organic phase was washed with brine, concentrated to give crude. The crude was purified by column chromatography (EA:PE=1:4) to give yellow solid. MS (m/z): 276 (M+H)⁺

### Intermediate 52

### 2-(4-(1-methoxyethyl)phenyl)-4,4,5,5-tetramethyl-1,3,2-dioxaborolane

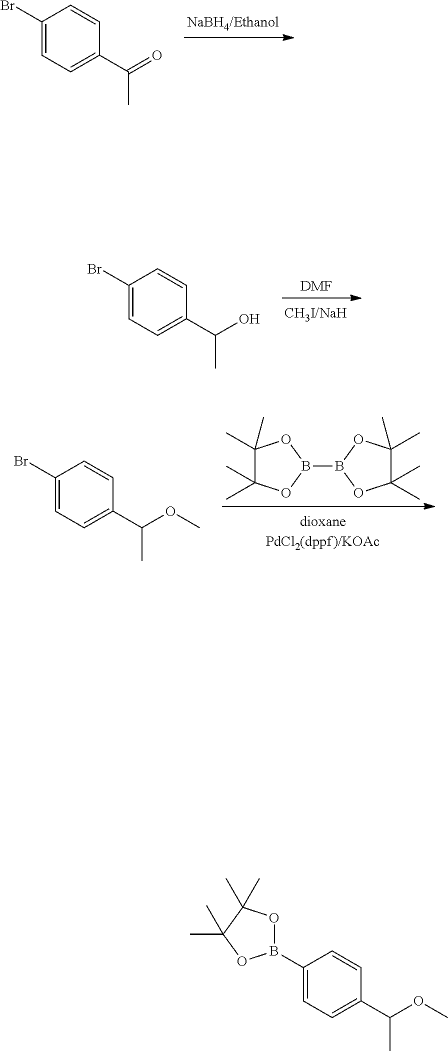

### (A) 1-(4-bromophenyl)ethanol

To a solution of 1-(4-bromophenyl)ethanone (1.99 g, 10 mmol) in EtOH (30 mL) at 0° C. was added NaBH₄ (1.14 g, 30 mmol) in portions, then the mixture was stirred for 20 minutes at 0° C. As TLC showed the reaction completed the mixture (cold) was poured into ice water, neutralized with 1N HCl solution until pH=6˜7, extracted with EA. The organic phase was washed with brine, dried, concentrated and purified by silica gel chromatography (eluting with PE/EA=5:1-->1:1) to give product as whit oil. MS (m/z): 284 (M-OH+H)⁺

### (B) 1-bromo-4-(1-methoxyethyl)benzene

To a solution of 1-(4-bromophenyl)ethanol (1.92 g, 9.95 mmol) in DMF (30 mL) was added NaH (60%, 597 mg, 14.93 mmol) at 0° C. and the mixture was stirred at 0° C. for 30 minutes. CH₃I (1.67 g, 11.94 mmol) was added into the reaction and the mixture was stirred at 20° C. for 24 hours. The reaction was quenched with sat. aq. NH₄Cl, extracted with EA (20 mL*3). The organic phase was washed with 30 mL of water and brine, concentrated and purified by column chromatography (EA:PE=1:4) to give brown solid.

### (C) 2-(4-(1-methoxyethyl)phenyl)-4,4,5,5-tetramethyl-1,3,2-dioxaborolane

To a solution of 1-bromo-4-(1-methoxyethyl)benzene (1.29 g, 6 mmol) in dioxane (15 mL) was added KOAc (1.77 g, 19 mmol), PdCl₂(dppf) (700 mg, 0.9 mmol) and 4,4,4′,4′,5,5,5′,5′-octamethyl-2,2′-bi(1,3,2-dioxaborolane) (2.29 g, 9 mmol). The mixture was stirred at 100° C. for 3 hours under N₂ atmosphere. The mixture was poured into 250 mL of water, extracted with EA. The organic phase was washed with brine, concentrated to give crude. The crude was purified by column chromatography (EA:PE=1:4) to give yellow solid. MS (m/z): 231 (M-MeO+H)⁺

### Intermediate 53

### tert-butyl (2-methyl-1-(4-(4,4,5,5-tetramethyl-1,3,2-dioxaborolan-2-yl)phenyl)propyl)carbamate

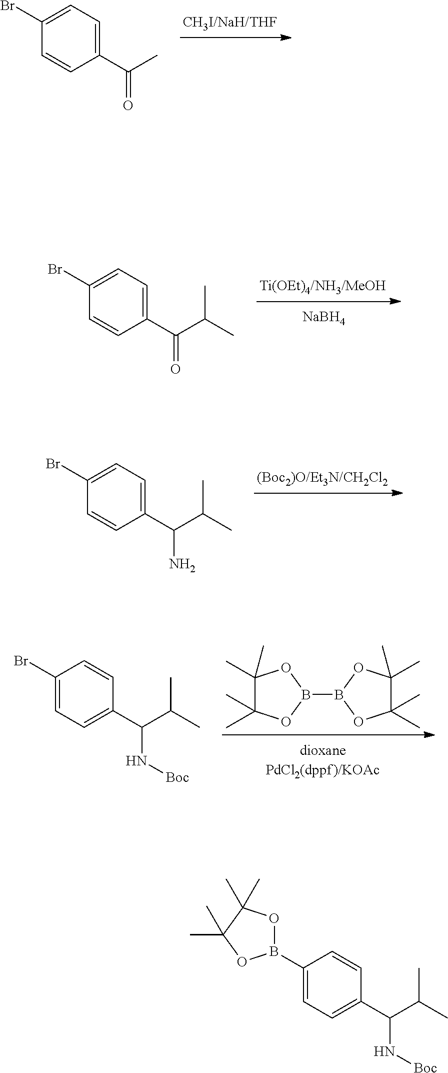

### (A) 1-(4-bromophenyl)-2-methyl propan-1-one

To a solution of 1-(4-bromophenyl)ethanone (1.99 g, 10 mmol) in THF (50 mL) was added NaH (60%, 880 mg, 22 mmol) at 0° C. and the mixture was stirred at 0° C. for 30 minutes. CH₃I (1.37 mL, 22 mmol) was added into the reaction and the mixture was stirred at 20° C. for 24 hours. The reaction was quenched with sat. aq. NH₄Cl, extracted with EA (20 mL*3). The organic phase was washed with 30 mL of water and brine, concentrated and purified by column chromatography (DCM:MeOH=50:1) to give brown solid. MS (m/z): 230 (M+2)⁺

### (B) 1-(4-bromophenyl)-2-methyl propan-1-amine

To a solution of 1-(4-bromophenyl)-2-methylpropan-1-one (1.83 g, 8.06 mmol) in MeOH (50 mL) was added NH₃/MeOH (7N, 11.5 mL, 80.6 mmol) and Ti(OEt)₄ (9.19 g, 40.3 mmol) at room temperature. The reaction mixture was stirred at room temperature for 18 hours. Then the reaction was cooled to 0° C. and NaBH₄ (1.06 g, 32.24 mmol) was added. The mixture was warmed to room temperature and stirred for 3 hours. The reaction mixture was poured into 2M aqueous NH₃ (900 mL), then filtered. The filtrate was extracted with EA (3×50 mL), and the combined extract was washed with water and brine, dried over Na₂SO₄, filtered and concentrated to give yellow oil. MS (m/z): 212 (M-NH₃+H)⁺

### (C) tert-butyl 1-(4-bromophenyl)-2-methyl propylcarbamate

To a solution of 1-(4-bromophenyl)-2-methylpropan-1-amine (1.07 g, 4.69 mmol) and Et₃N (718 uL, 5.16 mmol) in DCM (3 mL) was added a solution of di-tert-butyl dicarbonate (240 mg, 5.16 mmol) in DCM (2 mL) dropwise at 0° C. The reaction was stirred at room temperature for 4 hours. The reaction mixture was washed with aq. NaHCO₃ (25 mL), H₂O (25 mL) and brine (25 mL), dried over Na₂SO₄ and concentrated, purified by column chromatography (EA:PE=1:10) to give yellow oil. MS (m/z): 274 (M-t-butyl+2)⁺

### (D) tert-butyl 2-methyl-1-(4-(4,4,5,5-tetramethyl-1,3,2-dioxaborolan-2-yl)phenyl)propylcarbamate

To a solution of tert-butyl 1-(4-bromophenyl)-2-methylpropylcarbamate (1.17 g, 3.56 mmol) in dioxane (50 mL) was added KOAc (1.05 g, 10.69 mmol), PdCl₂(dppf) (446 mg, 0.54 mmol) and 4,4,4′,4′,5,5,5′,5′-octamethyl-2,2′-bi(1,3,2-dioxa borolane) (1.36 g, 5.35 mmol). The mixture was stirred at 100° C. for 3 hours under N₂ atmosphere. The reaction mixture was poured into 250 mL of water, extracted with EA. The organic phase was washed with brine, concentrated to give crude. The crude was purified by column chromatography (PE:EA=4:1) to give yellow oil. MS (m/z): 320 (M-t-butyl+H)⁺

### Intermediate 54

### (S)-6-(((7-chloropyrido[3,4-b]pyrazin-5-yl)oxy)methyl)-4-methylmorpholin-3-one

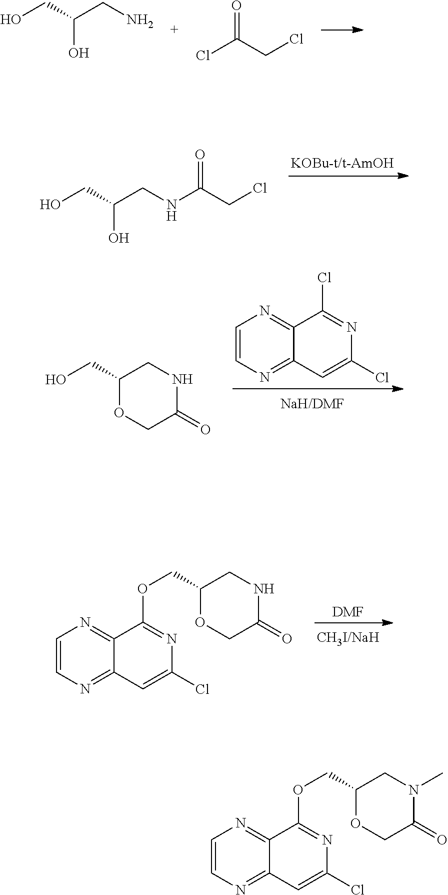

### (A) (S)-2-chloro-N-(2,3-dihydroxypropyl)acetamide

To a solution of(S)-3-aminopropane-1,2-diol (1.82 g, 20 mmol) and Et₃N (3.34 mL, 24 mmol) in DCM (40 mL) was dropped 2-chloroacetyl chloride (2.49 g, 22 mol) in DCM (10 mL) at 0° C. The reaction mixture was stirred at room temperature for 2 hours. The reaction solution was washed with sat. aq. NH₄Cl (5 mL), H₂O (5 mL) and brine (5 mL), dried over Na₂SO₄ and concentrated to give yellow solid. MS (m/z): 150 (M−H₂O+H)⁺

### (B) (S)-6-(hydroxymethyl)morpholin-3-one

To a stirred solution of potassium tert-butoxide (5.21 g, 36.7 mmol) in 60 mL tort-Butyl alcohol at room temperature was added(S)-2-chloro-N-(2,3-dihydroxypropyl)acetamide (2.46 g, 14.68 mmol) in 100 mL tert-Butyl alcohol slowly under nitrogen. After that the mixture was stirred for 1 hour, then MeOH (20 mL) and H₂O (1 mL) were added and the reaction mixture was stirred for an additional 20 minutes. The mixture was concentrated under vacuum and the residue was purified by flash column chromatography on silica gel with MeOH/EtOAc (20/80) to provide yellow oil. MS (m/z): 132 (M+H)⁺

### (C) (S)-6-((7-chloropyrido[4,3-b]pyrazin-5-yloxy)methyl)morpholin-3-one

To a solution of (S)-6-(hydroxymethyl)morpholin-3-one (1.86 g, 14.2 mmol) in DMF (60 mL) was added NaH (60%, 851 mg, 21.28 mmol) at 0° C. and the mixture was stirred at 0° C. for 15 minutes. After that 5,7-dichloropyrido[4,3-b]pyrazine (3.41 g, 17.02 mmol) was added and the mixture was stirred at 20° C. for 2 hours. The reaction was quenched with sat. aq. NH₄Cl, extracted with EA (20 mL*3), washed with 30 mL of water and brine, concentrated and purified by column chromatography (H₂O:MeOH=1:1) to give brown solid. MS (m/z): 295 (M+H)⁺

### (D) (S)-6-((7-chloropyrido[4,3-b]pyrazin-5-yloxy)methyl)-4-methylmorpholin-3-one

To a solution of (S)-6-((7-chloropyrido[4,3-b]pyrazin-5-yloxy)methyl)morpholin-3-one (1.6 g, 5.43 mmol) in DMF (50 mL) was added NaH (60%, 261 mg, 6.52 mmol) and CH₃I (406 uL, 6.52 mmol) at room temperature. The reaction was stirred at 20° C. for 1 hour. The reaction was quenched with sat. aq. NH₄Cl, extracted with EA (20 mL×3), washed with 30 mL of water and brine, concentrated and purified by thin-layer chromatography (DCM:MeOH=30:1) to give brown solid. MS (m/z): 309 (M+H)⁺

### Intermediate 55

### N-(2-(4-(4,4,5,5-tetramethyl-1,3,2-dioxaborolan-2-yl)phenyl)propan-2-yl)acetamide

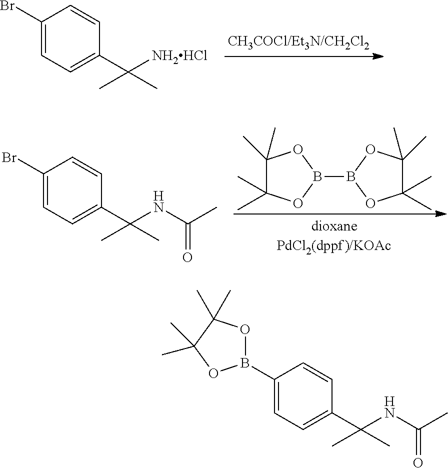

### (A) N-(2-(4-bromophenyl)propan-2-yl)acetamide

To a solution of 2-(4-bromophenyl)propan-2-amine hydrochloride (251 mg, 1 mmol) in DCM (10 mL) and Et₃N (350 uL, 2.5 mmol) was added acetyl chloride (86.4 mg, 1.1 mmol) at 0° C. The reaction mixture was stirred at room temperature for 4 hours. The reaction solution was washed with aq. NaHCO₃ (5 mL), H₂O (5 mL) and brine (5 mL), dried over Na₂SO₄ and concentrated to give white solid. MS (m/z): 256 (M+H)⁺

### (B) N-(2-(4-(4,4,5,5-tetramethyl-1,3,2-dioxaborolan-2-yl)phenyl)propan-2-yl)acetamide

To a solution of N-(2-(4-bromophenyl)propan-2-yl)acetamide (1 mmol) in dioxane (10 mL) was added KOAc (299 mg, 3 mmol), PdCl₂(dppf) (80 mg, 0.1 mmol) and 4,4,4′,4′,5,5,5′,5′-octamethyl-2,2′-bi(1,3,2-dioxa borolane) (381 mg, 1.5 mmol). The mixture was stirred at 100° C. for 3 hours under N₂ atmosphere. Then the reaction mixture was poured into 150 mL of water, extracted with EA. The organic phase was washed with brine, concentrated to give crude. The crude was purified by column chromatography (PE:EA=4:1) to give yellow solid. MS (m/z): 304 (M+H)⁺

### Intermediate 56

### 4-(4-(4,4,5,5-tetramethyl-1,3,2-dioxaborolan-2-yl)phenyl)tetrahydro-2H-pyran-4-ol

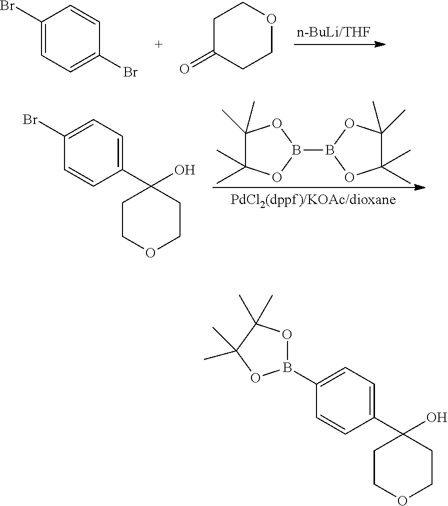

### (A) 4-(4-bromophenyl)tetrahydro-2H-pyran-4-ol

To a solution of 1,4-dibromobenzene (2.36 g, 10 mmol) in THF (50 mL) was slowly added a solution of 2.4N n-BuLi (4.2 mL, 10.5 mmol) at −78° C. and the mixture was stirred for 30 minutes. Dihydro-2H-pyran-4(3H)-one (1.05 g, 10 mmol) was added at the same temperature. Then the reaction mixture was warmed to room temperature slowly and stirred for 2 hours. After that the reaction was quenched with sat. aq. NH₄Cl, extracted with EA. The organic phase was washed with water and brine, concentrated to give yellow oil. MS (m/z): 241 (M−H₂O+H)⁺

### (B) 4-(4-(4,4,5,5-tetramethyl-1,3,2-dioxaborolan-2-yl)phenyl)tetrahydro-2H-pyran-4-ol

To a solution of 4-(4-bromophenyl)tetrahydro-2H-pyran-4-ol (10 mmol) in dioxane (70 mL) was added KOAc (2.95 g, 30 mmol), PdCl₂(dppf) (816 mg, 1 mmol) and 4,4,4′,4′,5,5,5′,5′-octamethyl-2,2′-bi(1,3,2-dioxa borolane) (3.81 g, 15 mmol). The mixture was stirred at 100° C. for 6 hours under N₂ atmosphere. The reaction mixture was poured into 150 mL of water, extracted with EA. The organic phase was washed with brine, concentrated to give crude. The crude was purified by column chromatography (PE:EA=5:1) to give white solid. MS (m/z): 287 (M−H₂O+H)⁺

### Intermediate 57

### 2-fluoro-N,N-dimethyl-4-(4,4,5,5-tetramethyl-1,3,2-dioxaborolan-2-yl)aniline

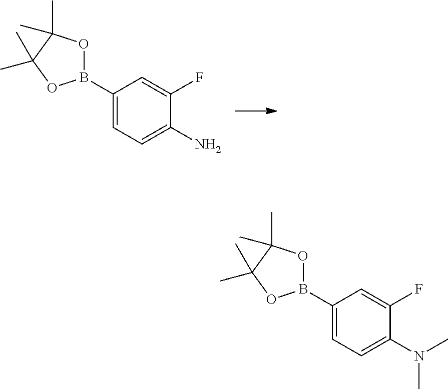

The title compound was prepared according to the procedures of Intermediate 15(A). MS (m/z): 266 (M+H)⁺.

### Intermediate 58

### 2-(4-isopropoxy-3-methoxyphenyl)-4,4,5,5-tetramethyl-1,3,2-dioxaborolane

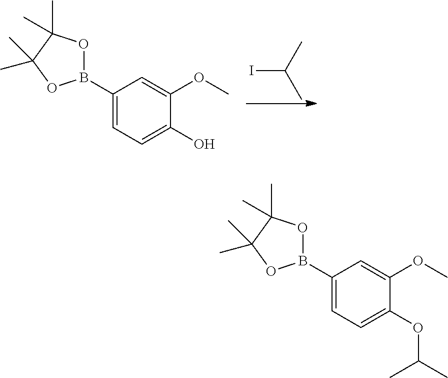

The title compound was prepared according to the procedures of Intermediate 15(A). MS (m/z): 293 (M+H)⁺.

### Intermediate 59

### 2-(3-isopropoxy-4-methoxyphenyl)-4,4,5,5-tetramethyl-1,3,2-dioxaborolane

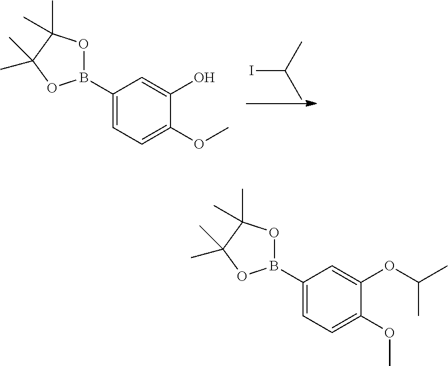

The title compound was prepared according to the procedures of Intermediate 15(A). MS (m/z): 293 (M+H)⁺.

### Intermediate 60

### N,N,2,6-tetramethyl-4-(4,4,5,5-tetramethyl-1,3,2-dioxaborolan-2-yl)aniline

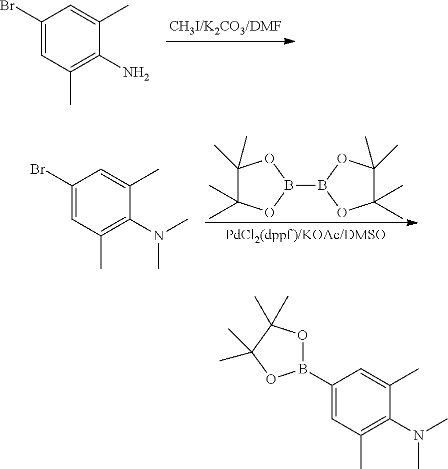

The title compound was prepared according to the procedures of Intermediate 15. MS (m/z): 276 (M+H)⁺.

### Intermediate 61

### N,N-dimethyl-1-(4-(4,4,5,5-tetramethyl-1,3,2-dioxaborolan-2-yl)phenyl)methanamine

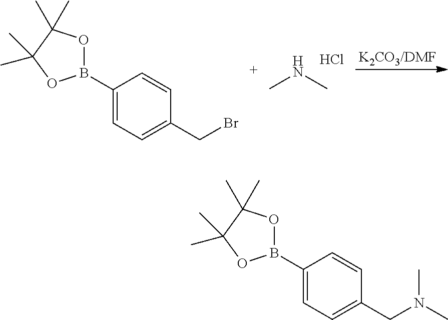

The title compound was prepared according to the procedures of Intermediate 11. MS (m/z): 262 (M+H)⁺.

### Intermediate 62

### 2-methyl-2-(4-(4,4,5,5-tetramethyl-1,3,2-dioxaborolan-2-yl)phenyl)propane-1,3-diol

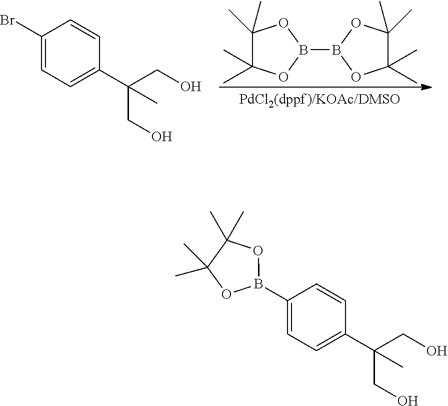

The title compound was prepared according to the procedures of Intermediate 15(B). MS (m/z): 275 (M−H₂O+H)⁺.

### Intermediate 63

### 2-methyl-2-(4-(4,4,5,5-tetramethyl-1,3,2-dioxaborolan-2-yl)phenyl)propanenitrile

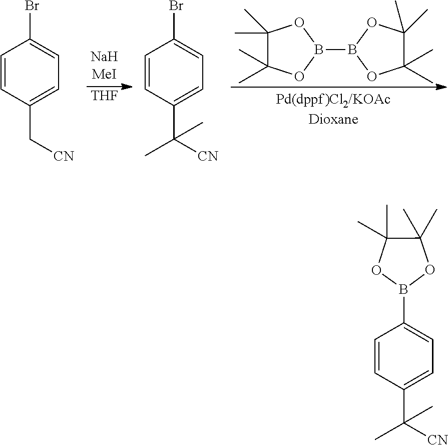

### (A) 2-(4-bromophenyl)-2-methylpropanenitrile

To a solution of 2-(4-bromophenyl)acetonitrile (3.05 g, 15.56 mmol) in dry THF (25 mL) at 0° C. was added NaH (1.37 g, 34.23 mmol). After stirring for 30 minutes at 0° C. MeI (6.63 g, 46.68 mmol) was added and the mixture was stirred at room temperature overnight. The mixture was quenched with saturated aqueous ammonium chloride (100 mL), extracted with EA (200 mL). The organic phase was dried over Na₂SO₄, concentrated in vacuo, and purified by flash column chromatography (PE:EA=1:0 to 4:1) to give 2.7 g of target compound. Yield: 77.4%.

### (B) 2-methyl-2-(4-(4,4,5,5-tetramethyl-1,3,2-dioxaborolan-2-yl)phenyl)propanenitrile

To a solution of 2-(4-bromophenyl)-2-methylpropanenitrile (500 mg, 2.23 mmol) and 4,4,4′,4′,5,5,5′,5′-octamethyl-2,2′-bi(1,3,2-dioxaborolane) (850 mg, 3.34 mmol) in dioxane (20 mL) was added Pd(dppf)Cl₂ (326 mg, 0.45 mmol) and KOAc (656 mg, 6.69 mmol). Under N₂ atmosphere the reaction mixture was stirred at 100° C. for 4 hours. The mixture was concentrated and the residue was purified by flash column chromatography (PE:EA=1:0 to 3:1) to give 432 mg of product as white solid. Yield: 71.4%. MS (m/z)=272 (M+H)⁺.

### Intermediate 64

### N,N-dimethyl-5-(4,4,5,5-tetramethyl-1,3,2-dioxaborolan-2-yl)pyridin-2-amine

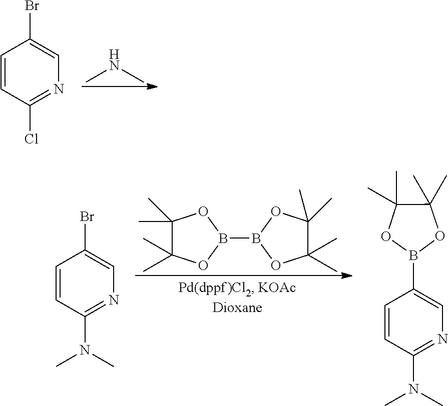

### (A) 5-bromo-N,N-dimethylpyridin-2-amine

The mixture of 5-bromo-2-chloropyridine (3.5 g, 18.19 mmol) in dimethylamine (10 mL) was stirred at 130° C. for 1 hour in a microwave reactor. The mixture was purified by flash column chromatography (MeOH:H₂O=0:1 to 10:1) to give 2.9 g crude product. MS (m/z)=202 (M+H)⁺203 (M+2)⁺.

### (B) N,N-dimethyl-5-(4,4,5,5-tetramethyl-1,3,2-dioxaborolan-2-yl)pyridin-2-amine

The title compound was prepared according to the procedures of Intermediate 63 (B). MS (m/z)=249 (M+H)⁺.

### Intermediate 65

### 2-(4-(4,4,5,5-tetramethyl-1,3,2-dioxaborolan-2-yl)phenyl)propan-2-ol

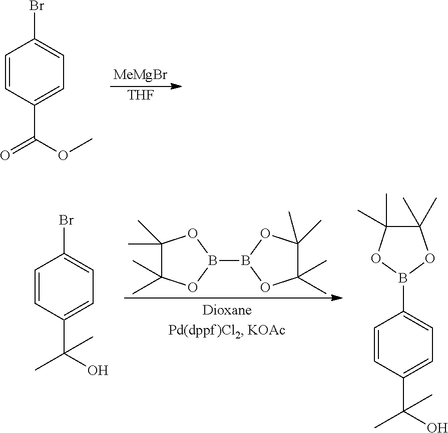

### (A) 2-(4-bromophenyl)propan-2-ol

To a solution of methyl 4-bromobenzoate (2.0 g, 9.30 mmol) in dry THF (60 mL) at 0° C. was added MeMgBr (9.3 mL, 27.90 mmol) under N₂ atmosphere. The mixture was stirred at room temperature for 2 hours. The mixture was quenched with saturated aqueous ammonium chloride (20 mL), and the reaction was partitioned between water (100 mL) and EA (200 mL). The organic phase was dried over Na₂SO₄, concentrated in vacuo, and the residue was purified by flash column chromatography (PE:EA=1:0 to 4:1) to give 1.6 g crude.

### (B) 2-(4-(4,4,5,5-tetramethyl-1,3,2-dioxaborolan-2-yl)phenyl)propan-2-ol

The title compound was prepared according to the procedures of Intermediate 63 (B). MS (m/z)=245 (M−18)⁺

### Intermediate 66

### 2-methyl-2-(4-(4,4,5,5-tetramethyl-1,3,2-dioxaborolan-2-yl)phenyl)propanamide

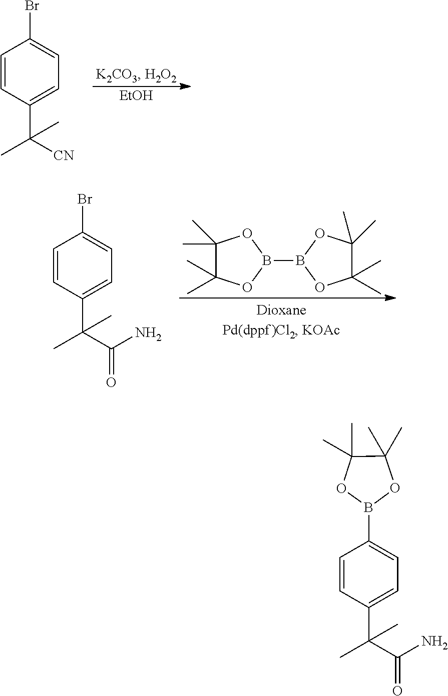

### (A) 2-(4-bromophenyl)-2-methylpropanamide

To a solution of 2-(4-bromophenyl)-2-methylpropanenitrile (672 mg, 3.0 mmol) in EtOH (10 mL) was added saturated aqueous potassium carbonate (7.0 mL) and 30% H₂O₂ (14 mL). The mixture was stirred at room temperature overnight. The mixture was partitioned between water (100 mL) and DCM (150 mL). The organic phase was dried over Na₂SO₄, concentrated to give 532 mg crude product as white solid. MS (m/z)=244 (M+H)⁺245 (M+2)⁺.

### (B) 2-methyl-2-(4-(4,4,5,5-tetramethyl-1,3,2-dioxaborolan-2-yl)phenyl)propanamide

The title compound was prepared according to the procedures of Intermediate 63 (B). MS (m/z)=290 (M+H)⁺.

### Intermediate 67

### tert-butyl (2-(4-(4,4,5,5-tetramethyl-1,3,2-dioxaborolan-2-yl)phenyl)propan-2-yl)carbamate

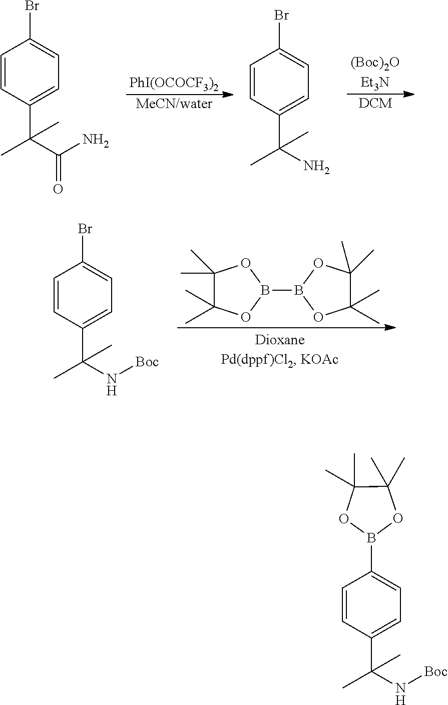

### (A) 2-(4-bromophenyl)propan-2-amine

To a solution of 2-(4-bromophenyl)-2-methylpropanamide (242 mg, 1 mmol) in MeCN/H₂O (4 mL/4 mL) was added PhI(OCOCF₃)₂ (430 mg, 1 mmol) in one portion. The mixture was stirred overnight at room temperature. The mixture was extracted with EA, the organic phase was dried over Na₂SO₄, concentrated, and the residue was purified by column chromatography (PE:EA=1:0 to 1:10) to give the target compound. MS (m/z)=197 (M−17)⁺, 198 (M−16)⁺

### (B) tert-butyl (2-(4-bromophenyl)propan-2-yl)carbamate

To a solution of 2-(4-bromophenyl)propan-2-amine (320 mg, 1.49 mmol) and Et₃N (302 mg, 2.98 mmol) in DCM (10 mL) was added (Boc)₂O (392 mg, 1.79 mmol) at 0° C. The mixture was stirred at room temperature overnight. The mixture was partitioned between water (300 mL) and DCM (150 mL). The organic phase was dried over Na₂SO₄, concentrated to give 436 mg crude product. MS (m/z)=197 (M−117)⁺, 200 (M−115)⁺.

### (C) tert-butyl (2-(4-(4,4,5,5-tetramethyl-1,3,2-dioxaborolan-2-yl)phenyl)propan-2-yl)carbamate

The title compound was prepared according to the procedures of Intermediate 63 (B). MS (m/z)=244 (M−118)⁺, 245 (M−117)⁺.

### Intermediate 68

### 2-(4-(2-methoxypropan-2-yl)phenyl)-4,4,5,5-tetramethyl-1,3,2-dioxaborolane

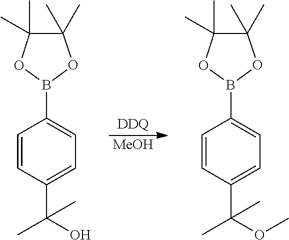

To a solution of 2-(4-(4,4,5,5-tetramethyl-1,3,2-dioxaborolan-2-yl)phenyl)propan-2-ol (514 mg, 2.0 mmol) in MeOH (10 mL) was added DDQ (908 mg, 4.0 mmol) at 0° C. The mixture was stirred at room temperature overnight. The mixture was concentrated, and the residue was purified by flash column chromatography (PE:EA=20:1 to 4:1) to give 200 mg product as white solid.

¹HNMR (400 MHz, CDCl₃) δ 7.79 (d, J=8.2 Hz, 2H), 7.40 (d, J=8.3 Hz, 2H), 3.06 (s, 3H), 1.51 (s, 6H), 1.33 (s, 12H).

### Intermediate 69

### 2,2-dimethyl-1-(4-(4,4,5,5-tetramethyl-1,3,2-dioxaborolan-2-yl)phenyl)propan-1-ol

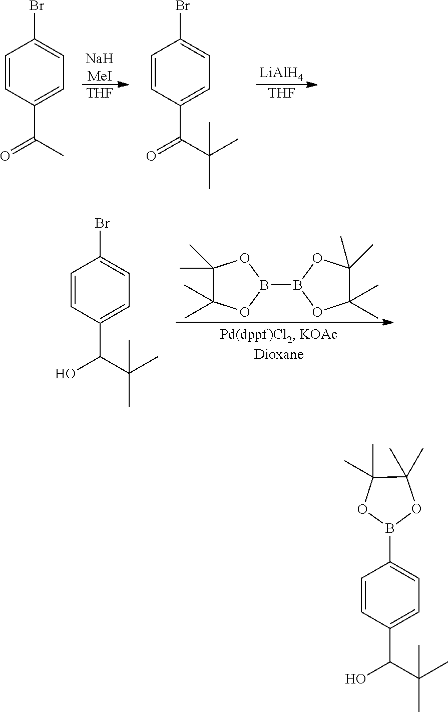

### (A) 1-(4-bromophenyl)-2,2-dimethylpropan-1-one

To a solution of 1-(4-bromophenyl)ethanone (4.0 g, 20.10 mmol) in dry THF (80 mL) at 0° C. was added NaH (3.2 g, 80.40 mmol) under N₂ atmosphere. After stirring for 30 minutes at 0° C. MeI (11.4 g, 80.40 mmol) was added and the mixture was stirred at room temperature overnight. The mixture was quenched with saturated aqueous ammonium chloride (100 mL), and extracted with EA (200 mL). The organic phase was dried over Na₂SO₄, concentrated in vacuo to give 4.5 g crude product

¹H NMR (400 MHz, CDCl₃) δ 7.55-7.58 (m, 2H), 7.51-7.53 (m, 2H), 1.32 (s, 9H).

### (B) 1-(4-bromophenyl)-2,2-dimethylpropan-1-ol

To a solution of 1-(4-bromophenyl)-2,2-dimethylpropan-1-one (4.5 g, 18.66 mmol) in dry THF (80 mL) at 0° C. was added LiAlH₄ (0.92 g, 24.12 mmol) under N₂ atmosphere. The mixture was stirred for 1 hour at 0° C. The mixture was quenched with water (100 mL), and extracted with EA (300 mL). The organic phase was dried over Na₂SO₄, concentrated to give 3.9 g product.

¹H NMR (400 MHz, CDCl₃) δ 7.42 (dd, J=8.4 Hz, 1.3 Hz, 2H), 7.16 (dd, J=8.2 Hz, 1.1 Hz, 2H), 4.33 (s, 1H), 0.89 (s, 9H).

### (C) 2,2-dimethyl-1-(4-(4,4,5,5-tetramethyl-1,3,2-dioxaborolan-2-yl)phenyl)propan-1-ol

The title compound was prepared according to the procedures of Intermediate 63 (B). MS (m/z)=289 (M+H)⁺.

¹H NMR (400 MHz, CDCl₃) δ 7.74 (d, J=7.9 Hz, 2H), 7.29 (d, J=8.0 Hz, 2H), 4.38 (s, 1H), 1.33 (s, 12H), 1.25-1.23 (m, 9H).

### Intermediate 70

### 2-methyl-1-(4-(4,4,5,5-tetramethyl-1,3,2-dioxaborolan-2-yl)phenyl)propan-1-ol

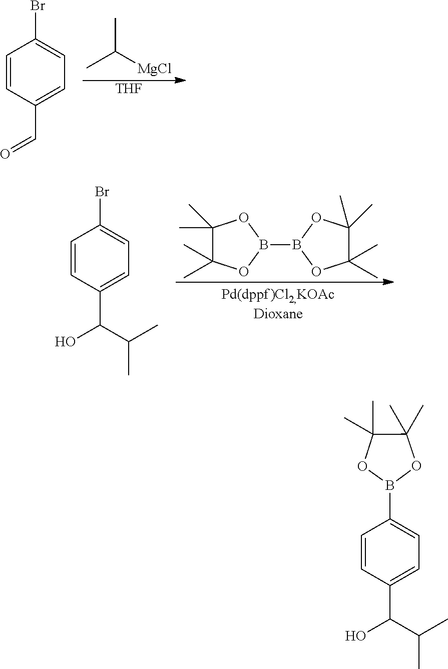

### (A) 1-(4-bromophenyl)-2-methylpropan-1-ol

To a solution of 4-bromobenzaldehyde (3.7 g, 20.0 mmol) in dry THF (80 mL) at 0° C. was added isopropylmagnesium chloride (12 mL, 24.0 mmol) under N₂ atmosphere. The mixture was stirred at 0° C. for 30 minutes. Then the mixture was stirred at room temperature for additional 30 minutes. The mixture was quenched with water (200 mL), extracted with EA (200 mL). The organic phase was dried over Na₂SO₄, concentrated in vacuo to give 4.6 g title compound.

¹H NMR (400 MHz, CDCl₃) δ 7.36-7.40 (m, 2H), 7.15-7.10 (m, 2H), 4.25 (d, J=6.6 Hz, 1H), 1.80-1.85 (m, 1H), 0.88 (d, J=6.7 Hz, 3H), 0.72 (d, J=6.8 Hz, 3H).

### (B) 2-methyl-1-(4-(4,4,5,5-tetramethyl-1,3,2-dioxaborolan-2-yl)phenyl)propan-1-ol

The title compound was prepared according to the procedures of Intermediate 63 (B).

¹H NMR (400 MHz, CDCl₃) δ 7.77 (d, J=8.1 Hz, 2H), 7.31 (d, J=8.2 Hz, 2H), 4.38 (d, J=6.6 Hz, 1H), 1.93-1.98 (m, 1H), 1.34 (s, 14H), 0.97 (d, J=6.7 Hz, 3H), 0.80 (d, J=6.8 Hz, 3H).

### Intermediate 71

### 2-(hydroxymethyl)-4-methylmorpholin-3-one

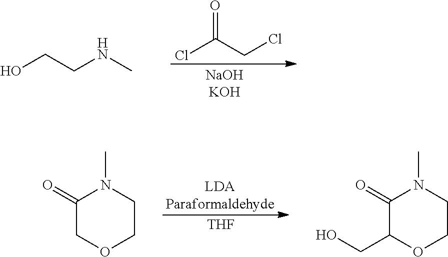

### (A) 4-methylmorpholin-3-one

A solution of 2-chloroacetyl chloride (7.62 ml, 0.1 mol) in DCM (150 mL) was added dropwise over 30 minutes to a suspension of 2-(methylamino)ethanol (8 mL, 0.1 mol) and NaOH (4.0 g, 0.1 mol) in DCM (100 mL) and water (100 mL) at 0° C., and the mixture was stirred at room temperature for 72 hours. Then the mixture was evaporated under reduced pressure. The residue was dissolved in EtOH (150 mL), and then KOH (5.6 g, 0.1 mol) was added. The mixture was stirred at 40° C. for 18 hours, and then filtered. The filtrate was concentrated under reduced pressure. The residue was purified by flash column chromatography (PE:EA=1:0 to 1:1 to 0:1) to give 5.78 g title compound. MS (m/z)=116 (M+H)⁺.

### (B) 2-(hydroxymethyl)-4-methylmorpholin-3-one

To a solution of DIPEA (1.21 g, 12.0 mmol) in dry THF (15 mL) at −78° C. was added n-BuLi (5 mL, 12.0 mmol) under N₂ atmosphere. The mixture was stirred at −78° C. for 15 minutes and added dropwise over 5 minutes into a suspension of 4-methylmorpholin-3-one (1.15 g, 10.0 mmol) in dry THF (5 mL). The mixture was stirred at −78° C. for 1 hour. Paraformaldehyde (0.36 g, 12 mmol) was added and the mixture was stirred at room temperature for 3 hours. The mixture was quenched with water (1 mL), and concentrated in vacuo, the residue was purified by flash column chromatography (DCM:MeOH=1:0 to 5:1) to give 438 mg product. MS (m/z)=146 (M+H)⁺.

¹H NMR (400 MHz, cdcl₃) δ 4.14-4.12 (m, 1H), 4.07-4.02 (m, 1H), 3.93-3.80 (m, 3H), 3.63-3.57 (m, 1H), 3.18-3.14 (m, 1H), 2.98 (s, 3H).

### Intermediate 72

### 2-(4-(1,1-difluoroethyl)phenyl)-4,4,5,5-tetramethyl-1,3,2-dioxaborolane

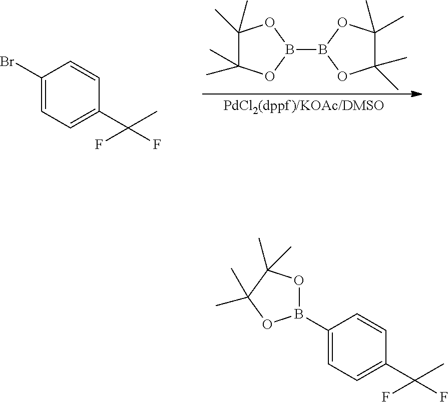

The title compound was prepared according to the procedures of Intermediate 15(B).

### Intermediate 73

### 2-(hydroxymethyl)morpholin-3-one

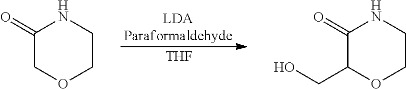 [SMILES: O=C1COCCN1 ; O=C1NCCOC1CO ; "THF"]

The title compound was prepared according to the procedures of Intermediate 71 (B).

### Intermediate 74

### 4,4,5,5-tetramethyl-2-(4-(3-methyloxetan-3-yl)phenyl)-1,3,2-dioxaborolane

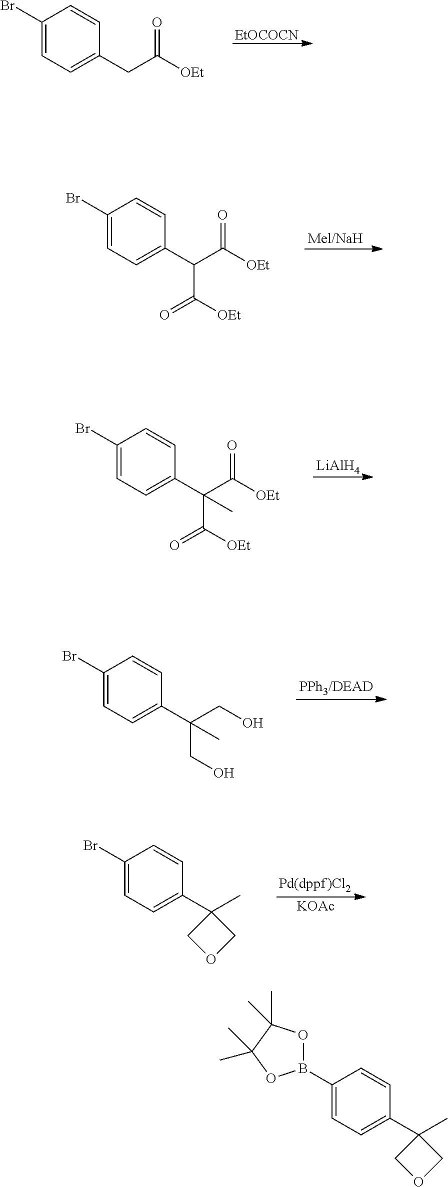

### (A) diethyl 2-(4-bromophenyl)malonate

To a solution of DIPEA (2.23 g, 22 mmol) in dry THF (40 mL) at −78° C. was added n-BuLi (9.12 mL, 22 mmol). After stirring for 30 minutes, ethyl carbonocyanidate (5.0 g, 21 mmol) was added and the mixture was stirred at room temperature for 48 hours. The mixture was quenched with water (15 mL) and partitioned between 1 N HCl (50 mL) and DCM (50 mL). The organic layer was dried over Na₂SO₄, concentrated in vacuo to give 7.1 g title compound.

¹H NMR (400 MHz, cdcl₃) δ 7.33-7.31 (m, 2H), 7.29-7.19 (m, 2H), 4.25-4.18 (m, 2H), 4.17-4.08 (m, 2H), 3.56 (s, 1H), 1.27-1.22 (m, 6H).

### (B) diethyl 2-(4-bromophenyl)-2-methylmalonate

To a solution of diethyl 2-(4-bromophenyl)malonate (7.1 g, 19.04 mmol) in dry THF (45 mL) at 0° C. was added NaH (1.0 g, 25.2 mmol). After stirring for 30 minutes at 0° C., MeI (5.96 g, 42 mmol) was added and the mixture was stirred at room temperature for 12 hours. The mixture was quenched with water (15 mL) and partitioned between 1 N HCl solution (50 mL) and DCM (50 mL). The organic layer was dried over Na₂SO₄, concentrated in vacuo to give 7.5 g title compound. MS (m/z)=272 (M+H)⁺.

### (C) 2-(4-bromophenyl)-2-methylpropane-1,3-diol

To a solution of diethyl 2-(4-bromophenyl)-2-methylmalonate (4.2 g, 12.76 mmol) in dry THF (60 mL) at 0° C. was added LiAlH₄ (1.06 g, 28.07 mmol). After stirring for 3 hours at 0° C., the mixture was quenched with water (10 mL) and partitioned between 1 N HCl (30 mL) and DCM (100 mL). The organic layer was dried over Na₂SO₄, concentrated in vacuo to give 3.1 g title compound.

¹H NMR (400 MHz, cdcl₃) δ 7.36-7.32 (m, 2H), 7.31-7.26 (m, 2H), 3.91 (d, J=11.0, 2H), 3.79 (d, J=11.0, 2H), 1.25 (d, J=0.5, 4H).

### (D) 3-(4-bromophenyl)-3-methyloxetane

2-(4-bromophenyl)-2-methylpropane-1,3-diol (3.1 g, 12.76 mmol), PPh₃ (6.69 g, 25.52 mmol) and DEAD (5.16 g, 25.52 mmol) were mixed in dry toluene (15 mL) in sealed tube and reacted in the microwave at 140° C. for 1.5 hours. The mixture was concentrated in vacuo, and the residue was purified by flash column chromatography (PE:EA=1:0 to 5:1) to give 245 mg title compound.

¹H NMR (400 MHz, cdcl₃) δ 7.33-7.31 (m, 2H), 7.15-7.13 (m, 2H), 4.91-4.90 (m, 2H), 4.63-4.61 (m, 2H), 1.70 (s, 3H).

### (E) 4,4,5,5-tetramethyl-2-(4-(3-methyloxetan-3-yl)phenyl)-1,3,2-dioxaborolane

The title compound was prepared according to the procedures of Intermediate 63 (B).

¹H NMR (400 MHz, cdcl₃) δ 7.32-7.30 (m, 2H), 7.15-7.13 (m, 2H), 4.91-4.90 (m, 2H), 4.62-4.61 (m, 2H), 1.70 (s, 3H), 1.25 (s, 12H).

### Intermediate 75

### tert-butyl 2-methyl-2-(4-(4,4,5,5-tetramethyl-1,3,2-dioxaborolan-2-yl)phenyl)propanoate

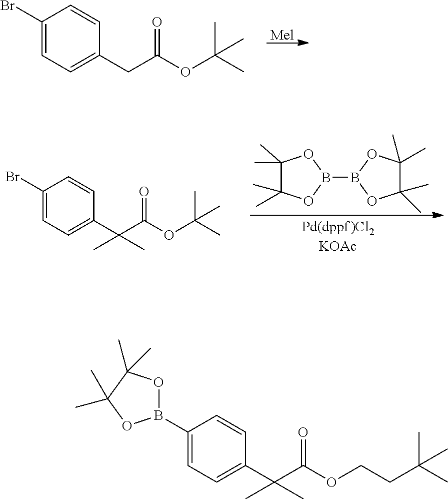

The title compound was prepared according to the procedures of Intermediate 63.

### Intermediate 76

### 1-(3-methyl-3-(4-(4,4,5,5-tetramethyl-1,3,2-dioxaborolan-2-yl)phenyl)azetidin-1-yl)ethanone

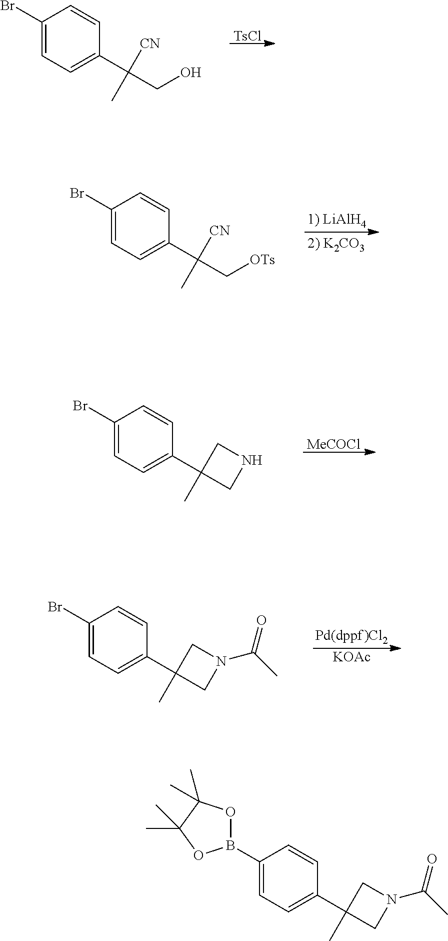

### (A) 2-(4-bromophenyl)-2-cyanopropyl 4-methylbenzenesulfonate

To a solution of 2-(4-bromophenyl)-3-hydroxy-2-methylpropanenitrile (1.5 g, 6.25 mmol) and Et₃N (1.26 g, 12.5 mmol) in DCM (25 mL) was added TsCl (1.79 g, 9.38 mmol) at 0° C. The mixture was stirred at room temperature overnight, then washed with 1 N HCl solution and water. The organic phase was dried over sodium sulphate, filtered and concentrated in vacuo to give 2.5 g title compound.

¹H NMR (400 MHz, cdcl₃) δ 7.66 (d, J=8.3, 2H), 7.48-7.43 (m, 2H), 7.30 (dd, J=8.0, 0.6, 2H), 7.25-7.19 (m, 3H), 4.13 (d, J=1.2, 2H), 2.44 (s, 3H), 1.72 (s, 3H).

### (B) 3-(4-bromophenyl)-3-methylazetidine

LiAlH₄ (0.28 g, 7.5 mmol) was added carefully to a solution of 2-(4-bromophenyl)-2-cyanopropyl 4-methylbenzenesulfonate (2.5 g, 6.25 mmol) in 20 mL of THF at 0° C. under nitrogen. The mixture was stirred at room temperature for 2 hours and then treated with an aqueous of sodium sulphate at room temperature for 30 minutes. Then the mixture was extracted with DCM, the organic phase was concentrated in vacuo. The residue and K₂CO₃ (1.73 g, 12.5 mmol) were mixed in EtOH (20 mL) and the mixture was stirred at 40° C. for 2 hours. Then it was filtered and concentrated in vacuo, and the residue was purified by column chromatography (MeOH/water=0:1˜10:1) to give 394 mg title compound. MS (m/z): 226 (M+H)⁺, 228 (M+2)⁺.

### (C) 1-(3-(4-bromophenyl)-3-methylazetidin-1-yl)ethanone

To a solution of 3-(4-bromophenyl)-3-methylazetidine (200 mg, 0.88 mmol) and Et₃N (178 mg, 1.76 mmol) in DCM (10 mL) was added acetyl chloride (104 mg, 1.33 mmol) at 0° C. After stirring at room temperature for 1 hour the mixture was concentrated to give crude product. MS (m/z): 269 (M+H)⁺, 270 (M+2)⁺.

### (D) 1-(3-methyl-3-(4-(4,4,5,5-tetramethyl-1,3,2-dioxaborolan-2-yl)phenyl)azetidin-1-yl)ethanone

The title compound was prepared according to the procedures of Intermediate 63 (B). MS (m/z): 316 (M+H)⁺.

### Intermediate 77

### 2-(4-fluoro-1,1-dimethylisochroman-6-yl)-4,4,5,5-tetramethyl-1,3,2-dioxaborolane

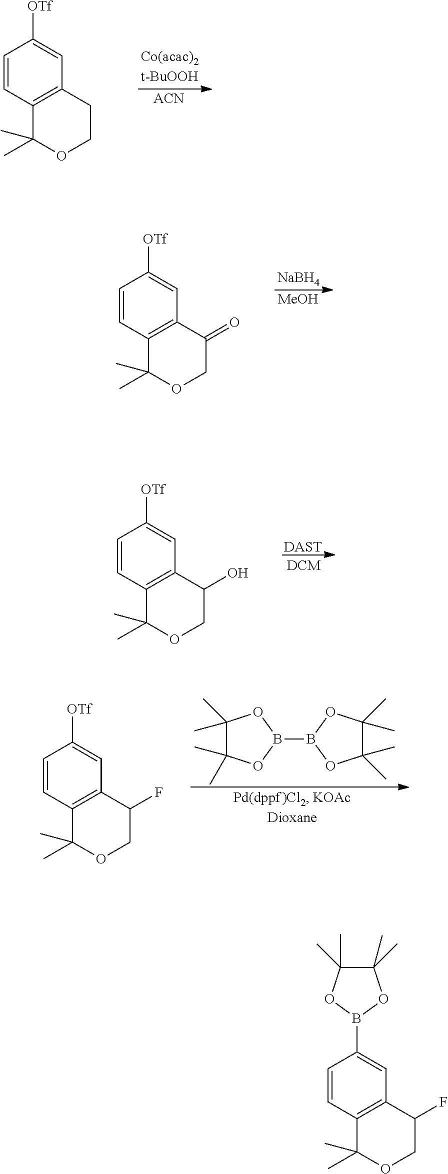

### (A) 1,1-dimethyl-4-oxoisochroman-6-yl trifluoromethanesulfonate

To a solution of 1,1-dimethylisochroman-6-yl trifluoromethanesulfonate (1.5 g, 4.83 mmol) and Co(acac)₂ (0.12 g, 0.48 mmol) in dry ACN (30 mL) was added t-BuOOH (2.17 g, 24.15 mmol) at 80° C. under nitrogen. The mixture was stirred at 80° C. for 4 hours. Then the mixture was concentrated in vacuo, and the residue was purified by column chromatography (PE/EA=1:0˜3:1) to give 0.24 g product.

¹H NMR (400 MHz, cdcl₃) δ 7.87 (d, J=2.7, 1 H), 7.46-7.43 (m, 1H), 7.37-7.33 (m, 1H), 4.45 (d, J=0.8, 2H), 1.63 (s, 6H).

### (B) 4-hydroxy-1,1-dimethylisochroman-6-yl trifluoromethanesulfonate

To a solution of 1,1-dimethyl-4-oxoisochroman-6-yl trifluoromethanesulfonate (240 mg, 0.74 mmol) in MeOH (10 mL) was added NaBH₄ (9 mg, 0.24 mmol) at 0° C. under nitrogen. The mixture was stirred at 0° C. for 1 hour, The mixture was quenched with 1 N HCl solution (15 mL) and DCM (50 mL). The organic layer was dried over Na₂SO₄, concentrated in vacuo to give 250 mg product.

¹H NMR (400 MHz, cdcl₃) δ 7.34 (s, 1H), 7.16 (d, J=1.5, 1H), 4.61-4.50 (m, 1H), 4.04-4.01 (m, 1H), 3.86-3.82 (m, 1H), 2.29 (s, 1H), 1.55 (s, 3H), 1.48 (s, 3H).

### (C) 4-fluoro-1,1-dimethylisochroman-6-yl trifluoromethanesulfonate

To a solution of 4-hydroxy-1,1-dimethylisochroman-6-yl trifluoromethanesulfonate (250 mg, 0.74 mmol) in dry DCM (10 mL) was added DAST (120 mg, 0.74 mmol) at 0° C. under nitrogen. The mixture was stirred at 0° C. for 1 hour. The mixture was quenched with 2 N NaHCO₃ solution (30 mL) and DCM (50 mL). The organic layer was dried over Na₂SO₄, concentrated in vacuo to give 252 mg title compound.

¹H NMR (400 MHz, cdcl₃) δ 7.33 (s, 1H), 7.24-7.23 (m, 1H), 5.43-5.27 (m, 1H), 4.13-4.06 (m, 1H), 4.07-4.02 (m, 1H), 1.58 (s, 3H), 1.48 (s, 3H).

### (D) 2-(4-fluoro-1,1-dimethylisochroman-6-yl)-4,4,5,5-tetramethyl-1,3,2-dioxaborolane

The title compound was prepared according to the procedures of Intermediate 63 (B).

¹H NMR (400 MHz, cdcl₃) δ 7.86 (s, 1H), 7.76 (d, J=7.8, 1 H), 7.14 (d, J=7.8, 1 H), 5.39-5.25 (m, 1H), 4.12-4.07 (m, 2H), 1.57 (s, 3H), 1.45 (s, 3H), 1.32 (s, 12H).

### Example 1

### Synthesis of Compounds 1-323

### Compound 1

### 4-(7-(4-morpholinophenyl)pyrido[4,3-b]pyrazin-5-yloxy)cyclohexanol

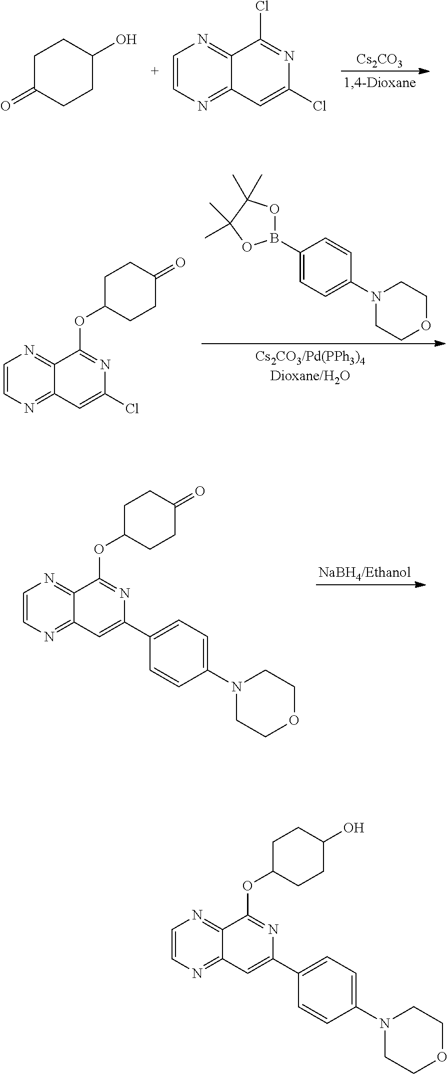

### (A) 4-(7-chloropyrido[4,3-b]pyrazin-5-yloxy)cyclohexanone

To a solution of 4-hydroxycyclohexanone (171 mg, 1.5 mmol) in dioxane was added Cs₂CO₃ (488 mg, 1.5 mmol) and 5,7-dichloropyrido[4,3-b]pyrazine (200 mg, 1.0 mmol) at room temperature. The mixture was stirred at 80° C. for 18 hours. After the 5,7-dichloropyrido[4,3-b]pyrazine was consumed, the reaction mixture was concentrated and the crude was used for next step directly.

### (B) 4-(7-(4-morpholinophenyl)pyrido[4,3-b]pyrazin-5-yloxy)cyclohexanone

To a solution of 4-(7-chloropyrido[4,3-b]pyrazin-5-yloxy)cyclohexanone from step (A) in dioxane/H₂O (15 mL/1.5 mL) was added Cs₂CO₃ (488.7 mg, 1.5 mmol), Pd(PPh₃)₄ (231 mg, 0.2 mmol) and 4-(4-(4,4,5,5-tetramethyl-1,3,2-dioxaborolan-2-yl)phenyl)morpholine (347 mg, 1.2 mmol). The mixture was stirred at 110° C. for 24 hours under N₂. The reaction mixture was filtered, concentrated and purified by silica gel column chromatography (EA:PE=2:1) to give yellow solid. MS (m/z): 405 (M+H)⁺

### (C) 4-(7-(4-morpholinophenyl)pyrido[4,3-b]pyrazin-5-yloxy)cyclohexanol

To a solution of 4-(7-(4-morpholinophenyl)pyrido[4,3-b]pyrazin-5-yloxy)cyclohexanone (70 mg, 0.17 mmol) in EtOH (5 mL) was added NaBH₄ (26 mg, 0.69 mmol) part wise at −30° C. Then the mixture was stirred for 20 minutes at −30° C. When TLC showed 4-(7-(4-morpholinophenyl)pyrido[4,3-b]pyrazin-5-yloxy)cyclohexanone had disappeared, the reaction solution (keep cold) was poured into ice water, neutralized with 1N HCl solution until pH=6˜7, then extracted with EA, washed with brine, dried, concentrated and purified by prep-TLC (DCM:MeOH=50:1) to give product as yellow solid. MS (m/z): 407 (M+H)⁺

### Compound 2

### 4-(4-(5-(2-(1H-pyrazol-4-yl)ethoxy)pyrido[3,4-b]pyrazin-7-yl)phenyl)morpholine

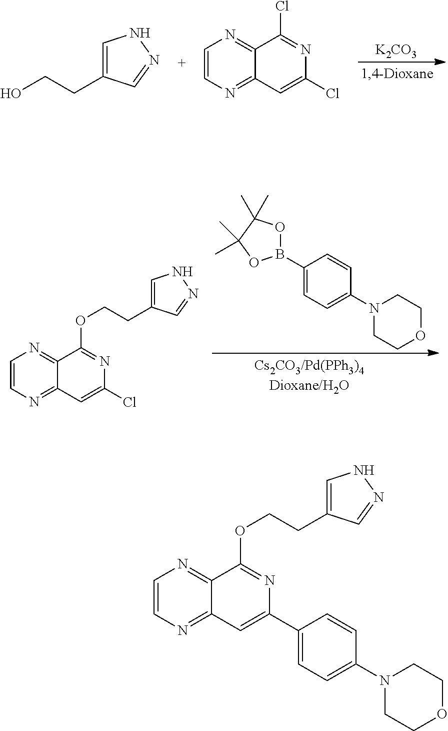

### (A) 5-(2-(1H-pyrazol-4-yl)ethoxy)-7-chloropyrido[3,4-b]pyrazine

The title compound was prepared according to the procedures of Compound 1(A) using instead 2-(1H-pyrazol-4-yl)ethanol. MS (m/z): 276 (M+H)⁺.

### (B) 4-(4-(5-(2-(1H-pyrazol-4-yl)ethoxy)pyrido[3,4-b]pyrazin-7-yl)phenyl)morpholine

The title compound was prepared according to the procedures of Compound 1(B). MS (m/z): 403 (M+H)⁺.

The following compounds were prepared according to the procedures of Compound 2 using the corresponding intermediates and reagents under appropriate conditions that will be recognized by one skilled in the art.

| Compound | Structure | MS (M + H)⁺ |
|---|---|---|
| 106 | 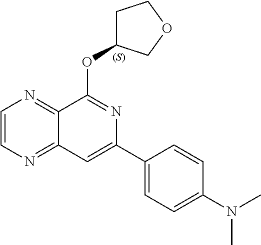 | 337 |
| 107 | 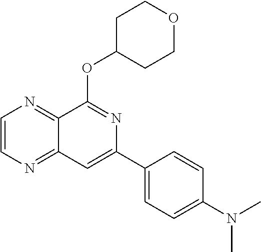 [SMILES: CN(C)c1ccc(-c2cc3nccnc3c(OC3CCOCC3)n2)cc1] | 351 |
| 123 | 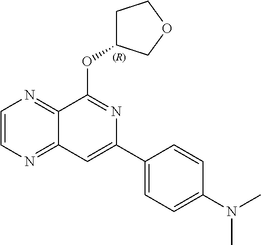 | 337 |
| 124 | 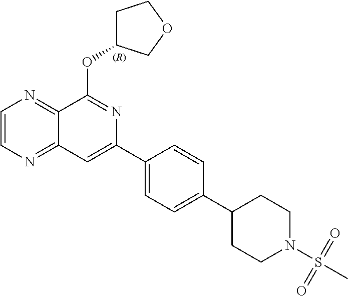 | 455 |
| 125 | 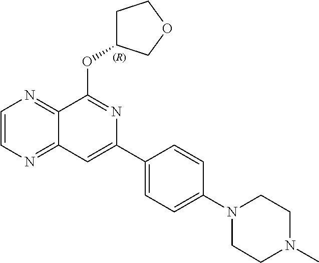 | 392 |
| 127 | 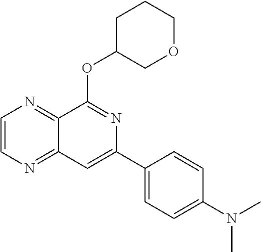 [SMILES: CN(C)c1ccc(-c2cc3nccnc3c(OC3CCCOC3)n2)cc1] | 351 |
| 133 | 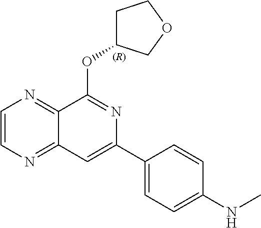 | 323 |
| 149 | 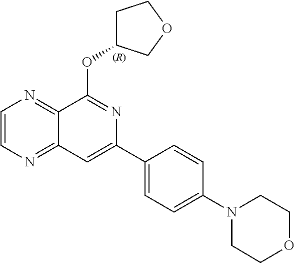 | 379 |
| 150 | 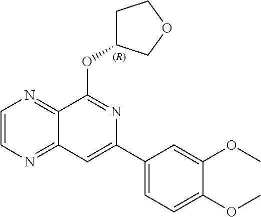 | 354 |
| 151 | 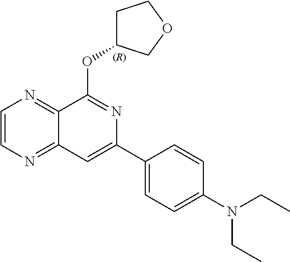 | 365 |
| 152 | 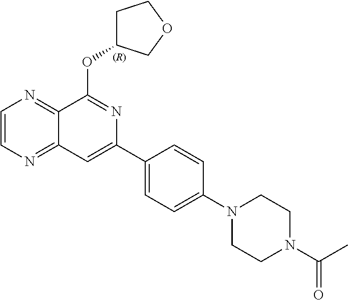 | 420 |
| 153 | 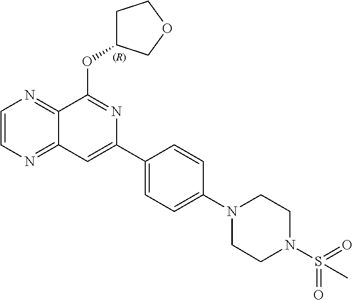 | 456 |
| 154 | 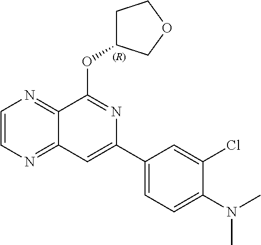 | 371 |
| 155 | 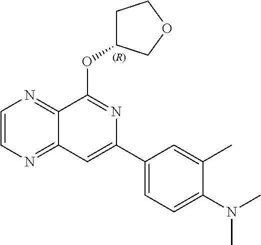 | 351 |
| 156 | 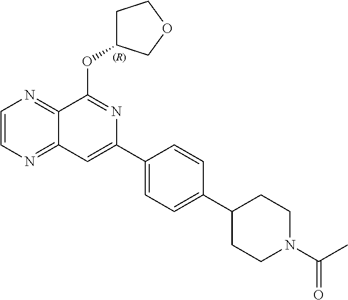 | 419 |
| 167 | 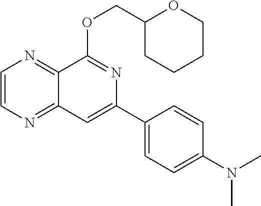 [SMILES: CN(C)c1ccc(-c2cc3nccnc3c(OCC3CCCCO3)n2)cc1] | 365 |
| 168 | 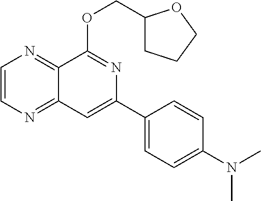 [SMILES: CN(C)c1ccc(-c2cc3nccnc3c(OCC3CCCO3)n2)cc1] | 351 |
| 169 | 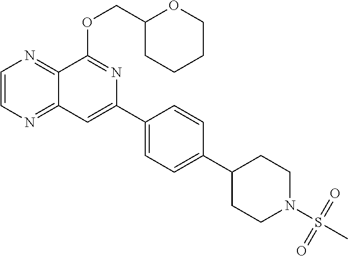 [SMILES: CS(=O)(=O)N1CCC(c2ccc(-c3cc4nccnc4c(OCC4CCCCO4)n3)cc2)CC1] | 483 |
| 170 | 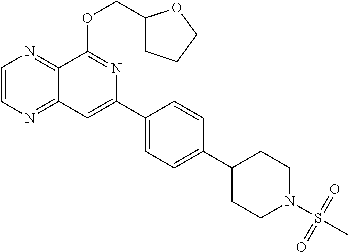 [SMILES: CS(=O)(=O)N1CCC(c2ccc(-c3cc4nccnc4c(OCC4CCCO4)n3)cc2)CC1] | 469 |
| 177 | 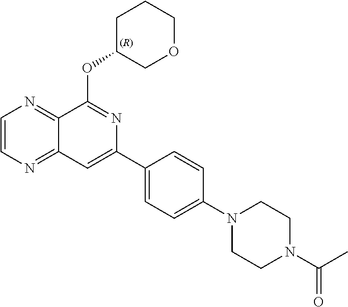 | 434 |
| 179 | 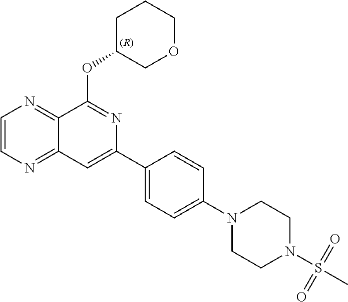 | 470 |
| 180 | 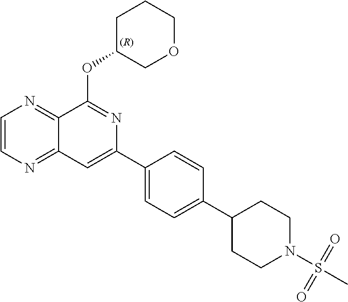 | 469 |
| 190 | 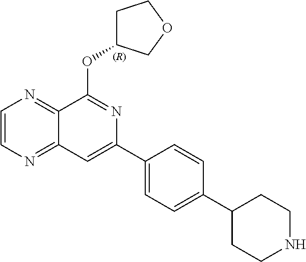 | 377 |
| 191 | 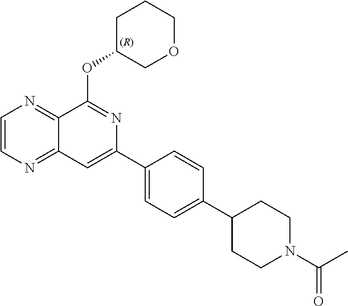 | 433 |
| 192 | 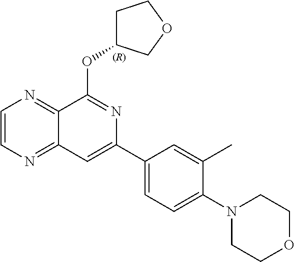 | 393 |
| 193 | 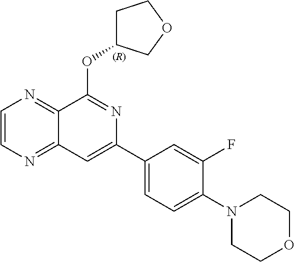 | 397 |
| 194 | 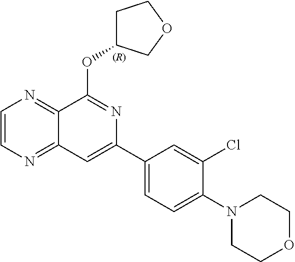 | 413 |
| 205 | 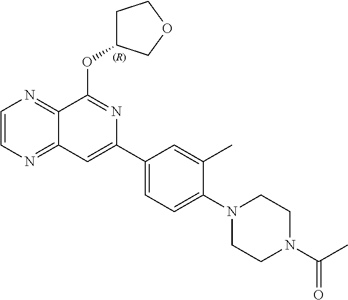 | 434 |
| 210 | 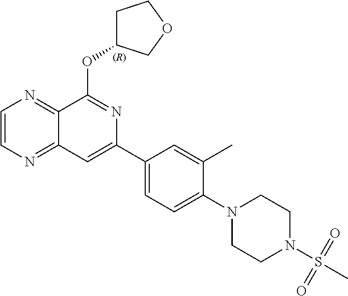 | 470 |
| 217 | 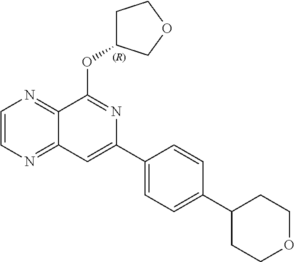 | 378 |
| 227 | 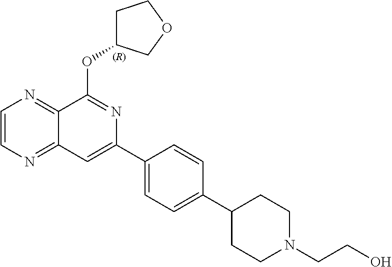 | 421 |
| 228 | 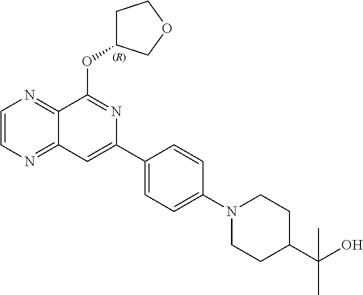 | 435 |
| 229 | 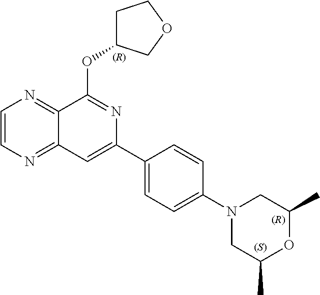 | 407 |

### Compound 3

### 4-(7-(4-morpholinophenyl)pyrido[4,3-b]pyrazin-5-yloxy)benzamide

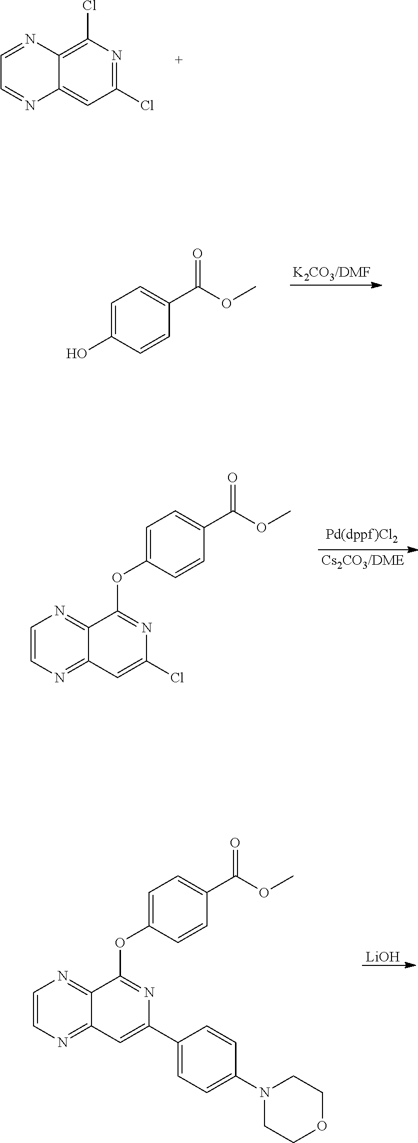 [SMILES: Clc1cc2nccnc2c(Cl)n1 ; COC(=O)c1ccc(O)cc1 ; COC(=O)c1ccc(Oc2nc(Cl)cc3nccnc23)cc1 ; COC(=O)c1ccc(Oc2nc(-c3ccc(N4CCOCC4)cc3)cc3nccnc23)cc1 ; "LiOH"]

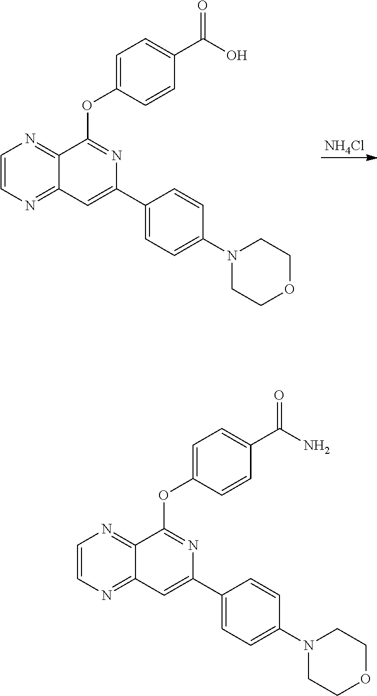 [SMILES: O=C(O)c1ccc(Oc2nc(-c3ccc(N4CCOCC4)cc3)cc3nccnc23)cc1 ; "NH4Cl" ; NC(=O)c1ccc(Oc2nc(-c3ccc(N4CCOCC4)cc3)cc3nccnc23)cc1]

### (A) methyl 4-(7-chloropyrido[4,3-b]pyrazin-5-yloxy)benzoate

The title compound was prepared according to the procedures of Compound 1(A) using instead methyl 4-hydroxybenzoate. MS (m/z): 316 (M+H)⁺.

### (B) methyl 4-(7-(4-morpholinophenyl)pyrido[4,3-b]pyrazin-5-yloxy)benzoate

A mixture of methyl 4-(7-chloropyrido[4,3-b]pyrazin-5-yloxy)benzoate (340 mg, 1.0 mmol), 4-(4-(4,4,5,5-tetramethyl-1,3,2-dioxaborolan-2-yl)phenyl)morpholine (347 mg, 1.2 mmol), Pd(dppf)Cl₂ (73 mg, 0.1 mmol) and Cs₂CO₃ (488 mg, 1.5 mmol) in dimethoxyethane/water (5 mL) was heated at 160° C. for 45 minutes in a microwave reactor. The mixture was cooled to room temperature, concentrated and purified by column chromatography (ethyl acetate in petro ether from 0% to 100%) then by C18 column to afford 96 mg title compound as yellow solid. MS (m/z): 443 (M+H)⁺.

### (C) 4-(7-(4-morpholinophenyl)pyrido[4,3-b]pyrazin-5-yloxy)benzoic acid

To a solution of methyl 4-(7-(4-morpholinophenyl)pyrido[4,3-b]pyrazin-5-yloxy)benzoate (96 mg, 0.22 mmol) in THF (10 mL) was added a solution of LiOH H₂O (28 mg, 0.66 mmol) in water (5 mL). The mixture was stirred at room temperature overnight. THF was removed in vacuo and the aqueous phase was acidified with 1N HCl to pH=4, the resulting acid was extracted with ethyl acetate and dried over anhydrous sodium sulfate. Solvent was removed in vacuo to afford 93 mg title compound as yellow solid.

### (D) 4-(7-(4-morpholinophenyl)pyrido[4,3-b]pyrazin-5-yloxy)benzamide

A mixture of 4-(7-(4-morpholinophenyl)pyrido[4,3-b]pyrazin-5-yloxy)benzoic acid (93 mg, 0.22 mmol), HATU (103 mg, 0.23 mmol), DIPEA (97 mg, 0.75 mmol) and NH₄Cl (24 mg, 0.45 mmol) in THF/dichloromethane (10 mL) was stirred at room temperature overnight. The mixture was purified by C18 column chromatography to give 30 mg title compound as yellow solid. MS (m/z): 428 (M+H)⁺.

### Compound 4

### 5-(((7-(4-morpholinophenyl)pyrido[3,4-b]pyrazin-5-yl)oxy)methyl)piperidin-2-one

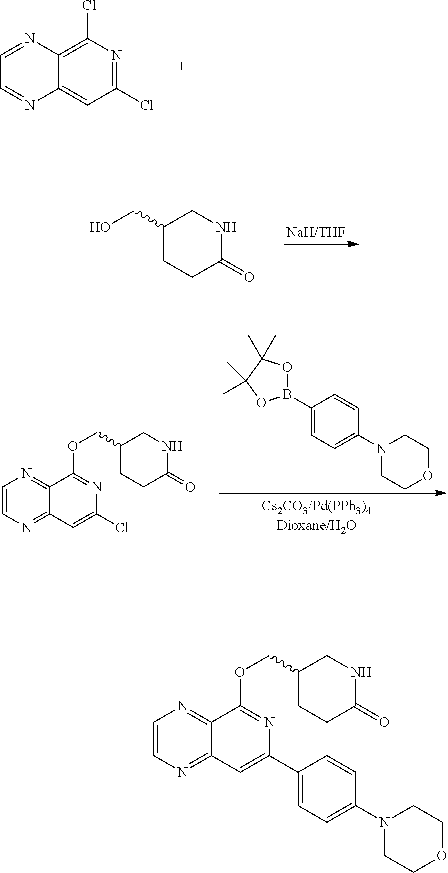 [SMILES: Clc1cc2nccnc2c(Cl)n1 ; O=C1CCC(CO)CN1 ; O=C1CCC(COc2nc(Cl)cc3nccnc23)CN1 ; CC1(C)OB(c2ccc(N3CCOCC3)cc2)OC1(C)C ; O=C1CCC(COc2nc(-c3ccc(N4CCOCC4)cc3)cc3nccnc23)CN1]

### (A) 5-(((7-chloropyrido[3,4-b]pyrazin-5-yl)oxy)methyl)piperidin-2-one

The title compound was prepared according to the procedures of Compound 1(A) using instead 5-(hydroxymethyl)piperidin-2-one. MS (m/z): 293 (M+H)⁺.

### (B) 5-(((7-(4-morpholinophenyl)pyrido[3,4-b]pyrazin-5-yl)oxy)methyl)piperidin-2-one

The title compound was prepared according to the procedures of Compound 1(B). MS (m/z): 420 (M+H)⁺.

The following compounds were prepared according to the procedures of Compound 4 using the corresponding intermediates and reagents under appropriate conditions that will be recognized by one skilled in the art.

| | | MS |
|---|---|---|
| Com- | | (M + |
| pound | Structure | H)⁺ |
| 5 | 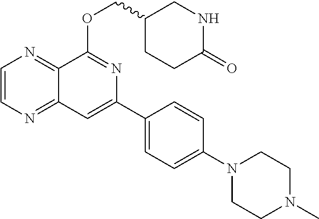 [SMILES: CN1CCN(c2ccc(-c3cc4nccnc4c(OCC4CCC(=O)NC4)n3)cc2)CC1] | 433 |
| 315 | 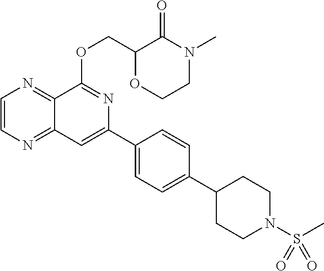 [SMILES: CN1CCOC(COc2nc(-c3ccc(C4CCN(S(C)(=O)=O)CC4)cc3)cc3nccnc23)C1=O] | 512 |
| 316 | 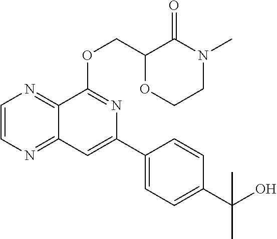 [SMILES: CN1CCOC(COc2nc(-c3ccc(C(C)(C)O)cc3)cc3nccnc23)C1=O] | 409 |
| 319 | 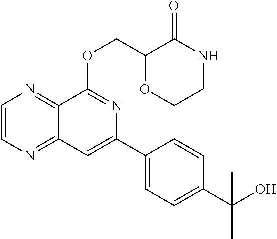 [SMILES: CC(C)(O)c1ccc(-c2cc3nccnc3c(OCC3OCCNC3=O)n2)cc1] | 395 |
| 320 | 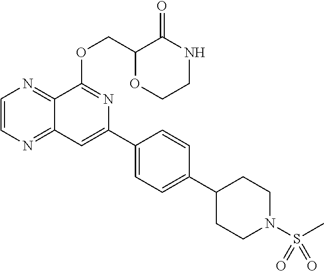 [SMILES: CS(=O)(=O)N1CCC(c2ccc(-c3cc4nccnc4c(OCC4OCCNC4=O)n3)cc2)CC1] | 498 |

### Compound 6

### (S)-2,2-difluoro-1-(2-((7-(4-(piperazin-1-yl)phenyl)pyrido[4,3-b]pyrazin-5-yloxy)methyl)morpholino)ethanone

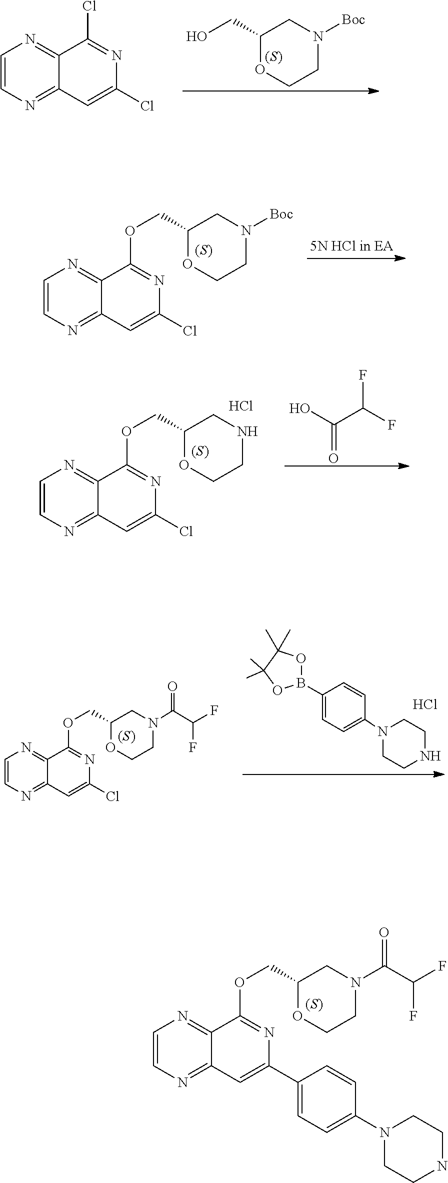

### (A) (S)-tert-butyl 2-((7-chloropyrido[4,3-b]pyrazin-5-yloxy)methyl)morpholine-4-carboxylate

To a mixture of 5,7-dichloropyrido[4,3-b]pyrazine (2.3 g, 11.51 mmol) and potassium carbonate (4.76 g, 34.52 mmol) in DMF (100 mL) was added (S)-tert-butyl 2-(hydroxymethyl)morpholine-4-carboxylate (5.0 g, 23.01 mmol), then the mixture was stirred at 40° C. for 72 hours. This solution was poured into water and extracted with EA. The combined organic phase was washed with brine, dried and purified by silica gel chromatography, eluting with MeOH/H₂O=1:10˜10:1, to give 1.83 g title compound.

### (B) (S)-1-(2-((7-chloropyrido[4,3-b]pyrazin-5-yloxy)methyl)morpholino)-2,2-difluoroethanone

To a solution of (S)-tert-butyl 2-((7-chloropyrido[4,3-b]pyrazin-5-yloxy)methyl)morpholine-4-carboxylate (1.26 g, 3.31 mmol) in EtOAc (20 mL) was added 5N HCl in EA (5 mL) dropwise, then stirred at room temperature for 2 hours. The reaction solution was concentrated to give (S)-2-((7-chloropyrido[4,3-b]pyrazin-5-yloxy)methyl)morpholine hydrochloride as brown solid, which was dissolved in DCM (60 mL). To the stirring solution was added EDCl (1.27 g, 6.62 mmol), HOBT (894 mg, 6.62 mmol), DIPEA (860 mg, 6.62 mmol) and 2,2-difluoroacetic acid (380 mg, 4.0 mmol). After stirring at room temperature overnight, the reaction solution was washed with brine, extracted with DCM, and purified over silica gel chromatography, eluting with DCM/MeOH=30:1, to give product as yellow solid. MS (m/z): 359 (M+H)⁺.

### (C)(S)-2,2-difluoro-1-(2-((7-(4-(piperazin-1-yl)phenyl)pyrido[4,3-b]pyrazin-5-yloxy)methyl)morpholino)ethanone

To a mixture of (S)-1-(2-((7-chloropyrido[4,3-b]pyrazin-5-yloxy)methyl)morpholino)-2,2-difluoroethanone (107 mg, 0.3 mmol), 1-(4-(4,4,5,5-tetramethyl-1,3,2-dioxaborolan-2-yl)phenyl)piperazine hydrochloride (109 mg, 0.36 mmol) and Cs₂CO₃ (293 mg, 0.9 mmol) in 15 mL dioxane/water (10:1) was added Pd(PPh₃)₄ (69 mg, 0.06 mmol). Then the mixture was heated at 100° C. under nitrogen atmosphere overnight. After cooling the reaction solution was extracted with EA (100 mL), washed with brine (50 mL). The organic phase was dried over anhydrous Na₂SO₄, concentrated and purified by prep-TLC (EA:MeOH=10:1) to give product as yellow solid. MS (m/z): 485 (M+H)⁺.

The following compounds were prepared according to the procedures of Compound 6 using the corresponding intermediates and reagents under appropriate conditions that will be recognized by one skilled in the art.

| Compound | Structure | MS (M + H)⁺ |
|---|---|---|
| 7 | 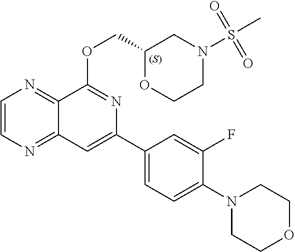 | 504 |
| 8 | 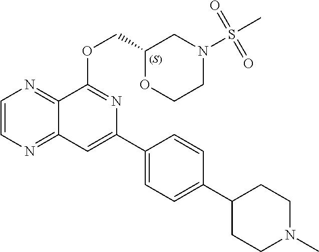 | 498 |
| 9 | 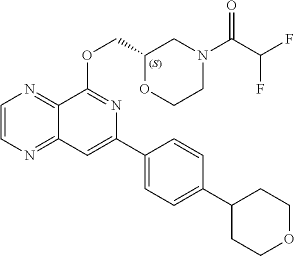 | 485 |
| 10 | 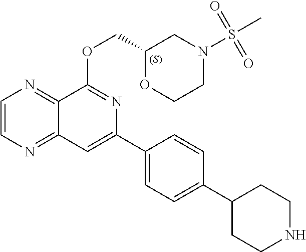 | 484 |
| 11 | 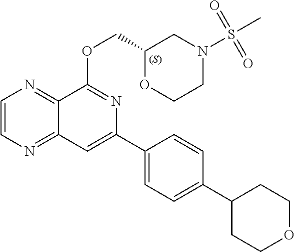 | 485 |
| 12 | 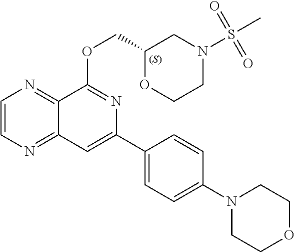 | 486 |
| 13 | 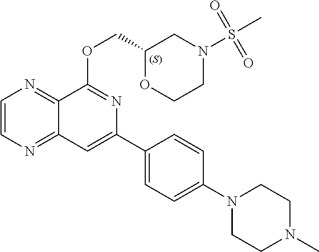 | 499 |
| 14 | 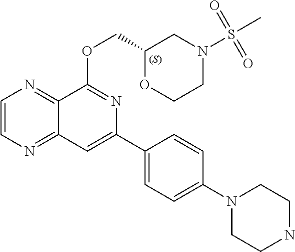 | 485 |
| 15 | 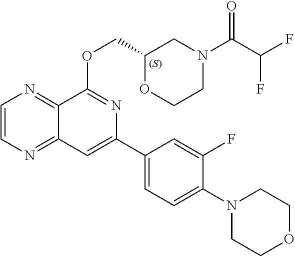 | 504 |
| 16 | 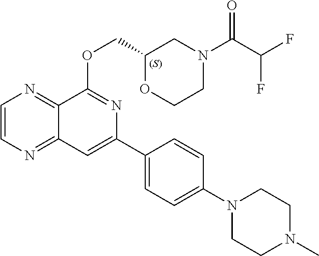 | 499 |
| 17 | 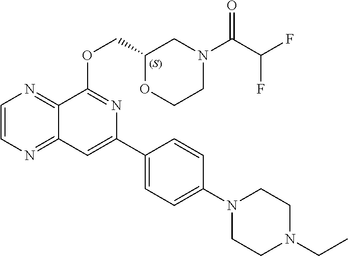 | 513 |
| 18 | 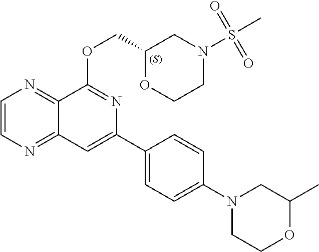 | 500 |
| 19 | 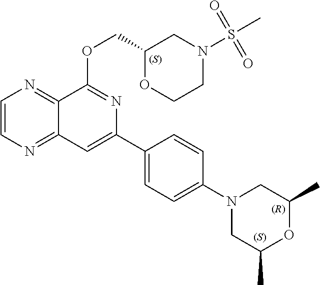 | 514 |
| 20 | 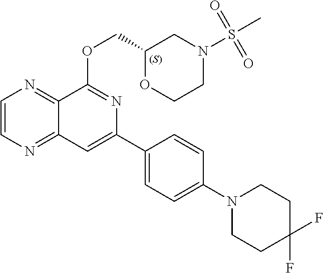 | 520 |
| 21 | 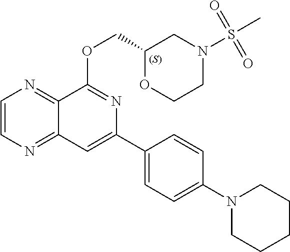 | 484 |
| 22 | 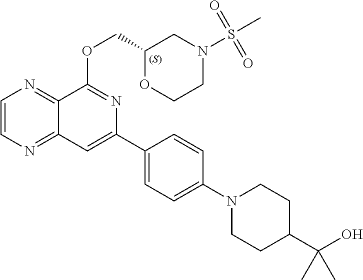 | 542 |
| 23 | 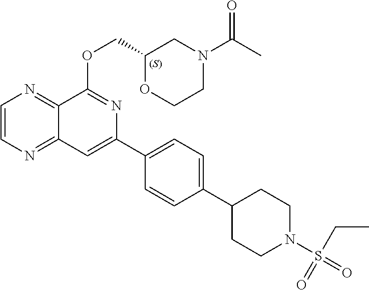 | 540 |
| 24 | 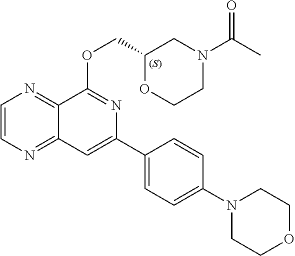 | 450 |
| 25 | 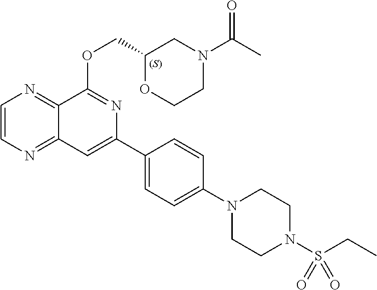 | 541 |
| 26 | 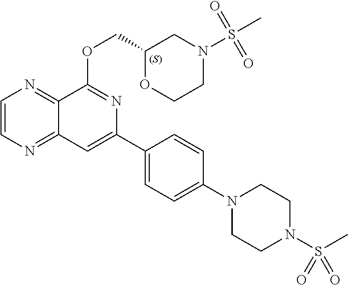 | 563 |
| 27 | 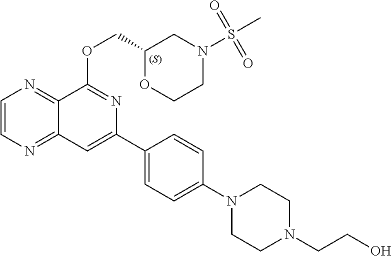 | 529 |
| 28 | 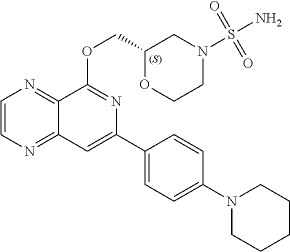 | 485 |
| 29 | 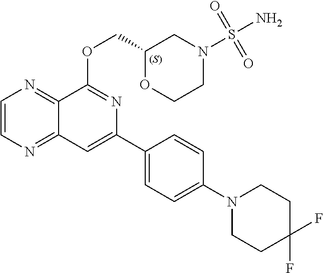 | 521 |
| 30 | 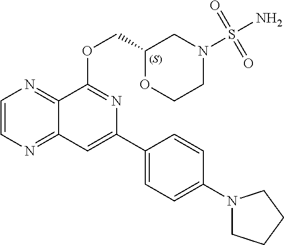 | 471 |
| 31 | 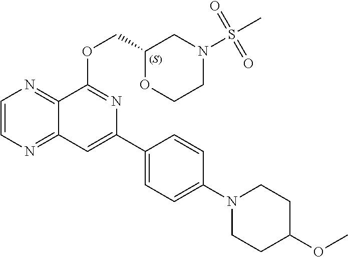 | 514 |
| 32 | 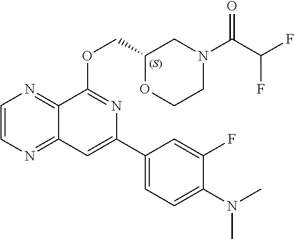 | 462 |
| 33 | 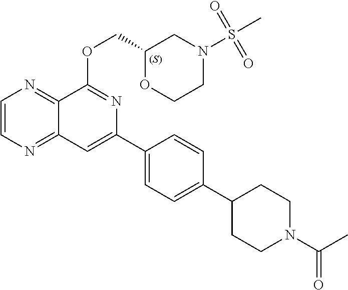 | 526 |
| 34 | 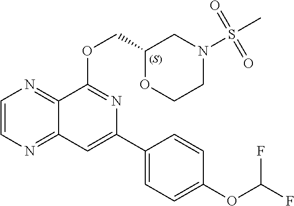 | 467 |
| 35 | 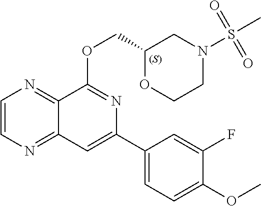 | 449 |
| 36 | 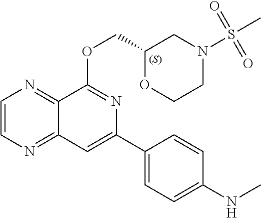 | 430 |
| 37 | 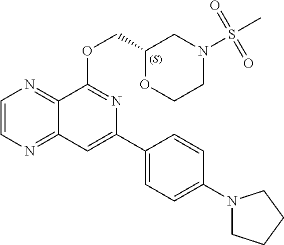 | 470 |
| 38 | 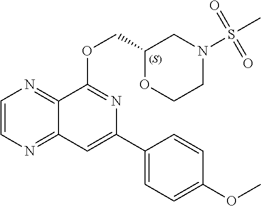 | 431 |
| 39 | 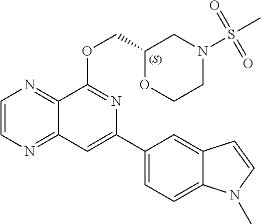 | 454 |
| 40 | 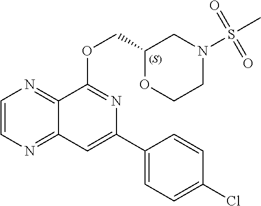 | 435 |
| 41 | 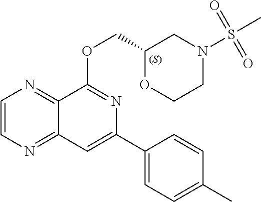 | 415 |
| 42 | 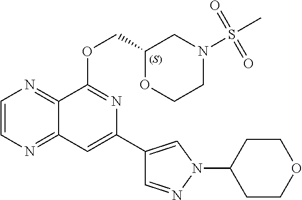 | 475 |
| 43 | 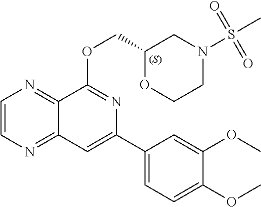 | 461 |
| 44 | 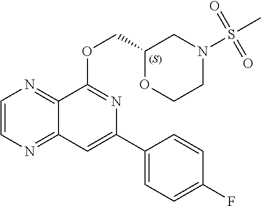 | 419 |
| 45 | 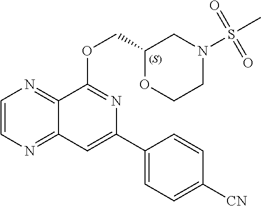 | 426 |
| 46 | 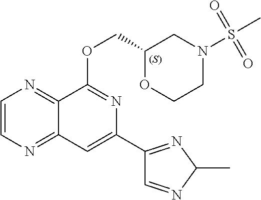 | 405 |
| 47 | 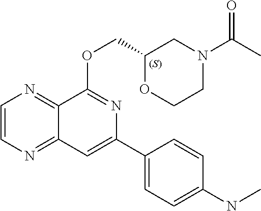 | 394 |
| 48 | 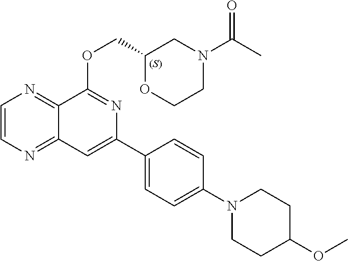 | 478 |
| 49 | 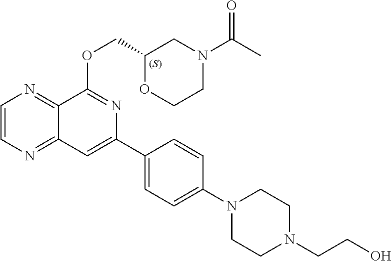 | 493 |
| 50 | 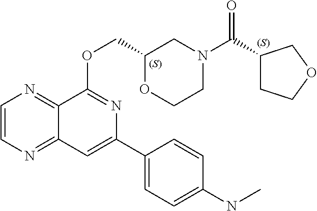 | 450 |
| 51 | 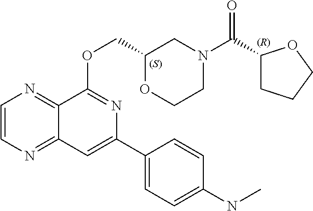 | 450 |
| 52 | 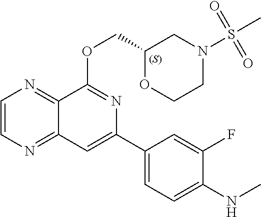 | 448 |
| 53 | 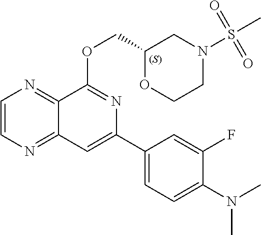 | 462 |
| 54 | 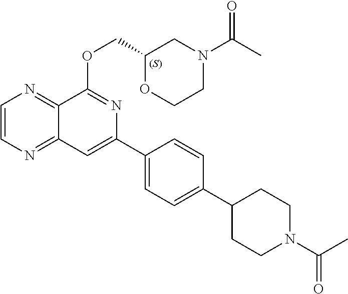 | 490 |
| 55 | 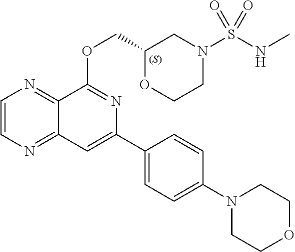 | 501 |
| 56 | 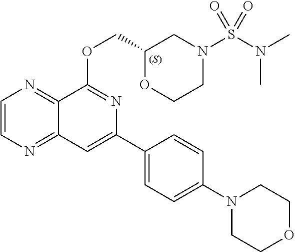 | 515 |
| 100 | 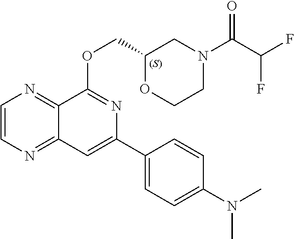 | 444 |
| 105 | 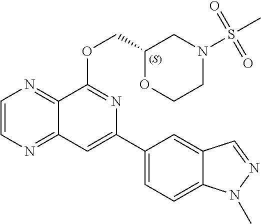 | 455 |
| 108 | 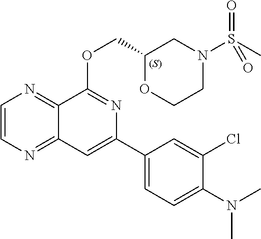 | 479 |
| 109 | 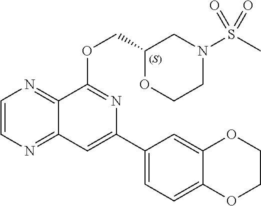 | 459 |
| 110 | 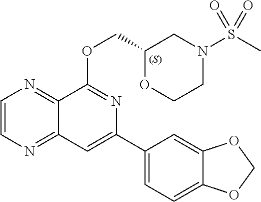 | 445 |
| 111 | 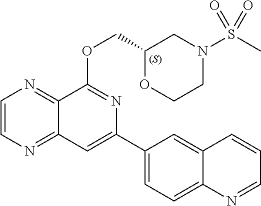 | 452 |
| 114 | 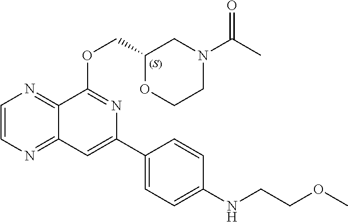 | 438 |
| 119 | 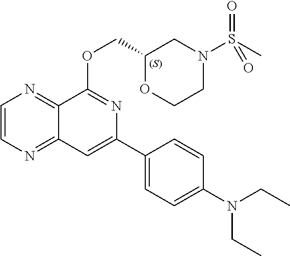 | 472 |
| 120 | 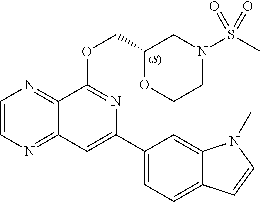 | 454 |
| 121 | 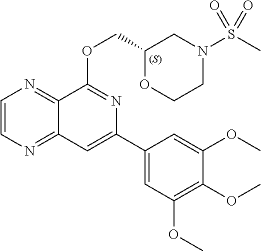 | 491 |
| 122 | 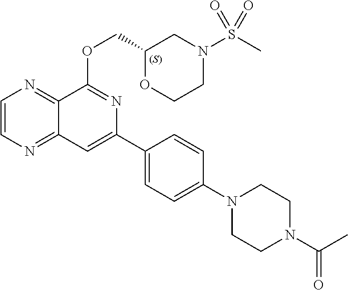 | 527 |
| 129 | 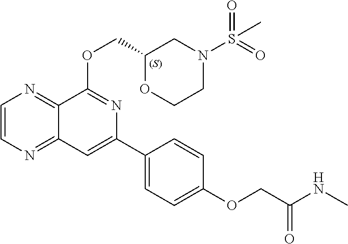 | 488 |
| 130 | 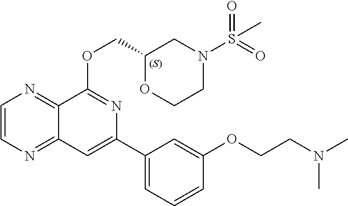 | 488 |
| 131 | 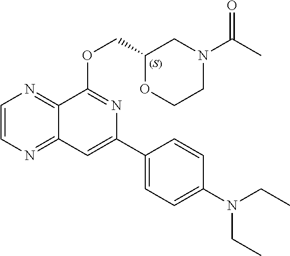 | 436 |
| 132 | 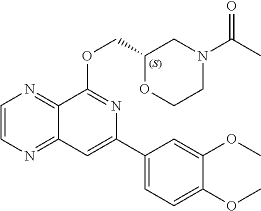 | 425 |
| 134 | 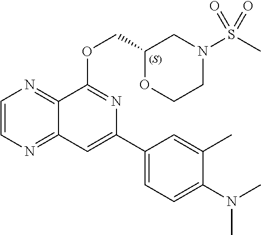 | 458 |
| 138 | 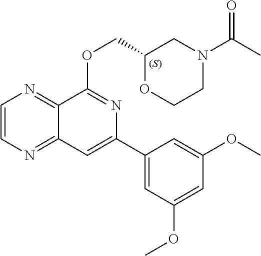 | 425 |
| 139 | 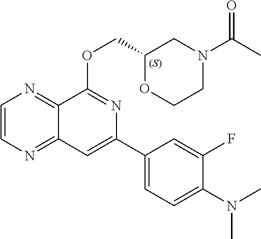 | 426 |
| 140 | 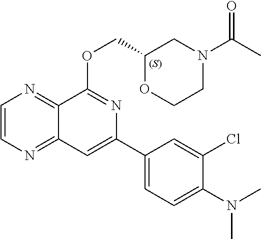 | 442 |
| 141 | 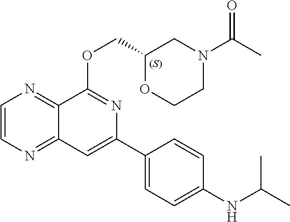 | 422 |
| 145 | 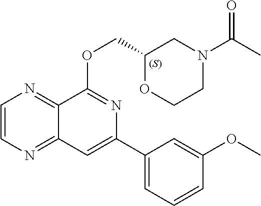 | 395 |
| 146 | 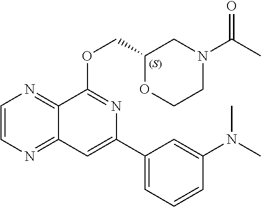 | 408 |
| 147 | 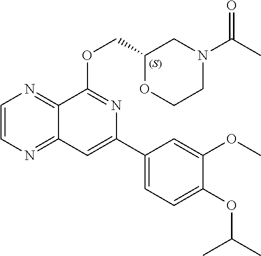 | 453 |
| 148 | 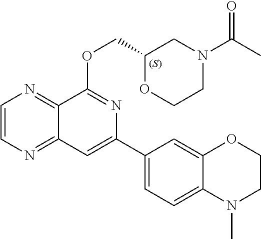 | 436 |
| 157 | 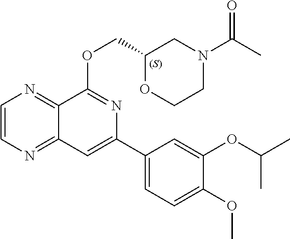 | 453 |
| 158 | 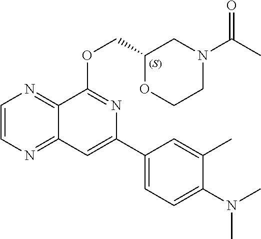 | 422 |
| 160 | 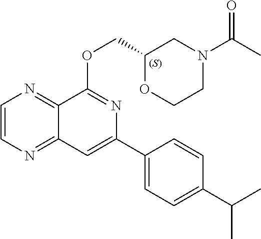 | 407 |
| 161 | 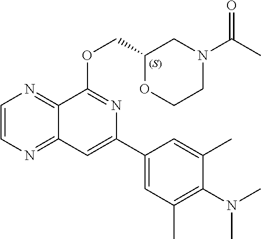 | 436 |
| 162 | 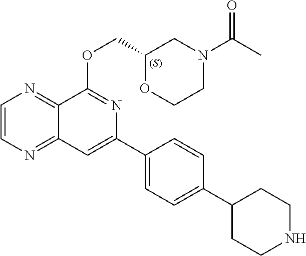 | 448 |
| 171 | 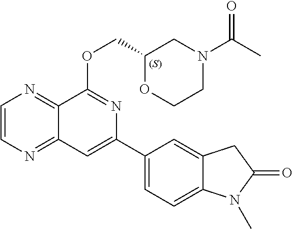 | 434 |
| 174 | 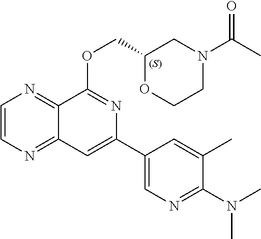 | 423 |
| 178 | 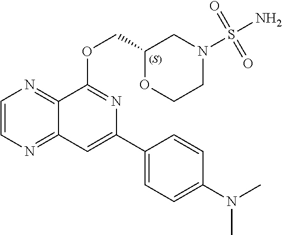 | 445 |
| 182 | 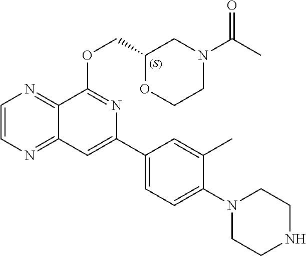 | 463 |
| 185 | 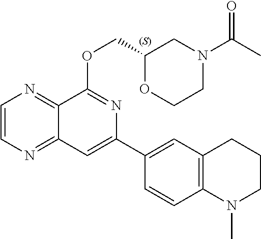 | 434 |
| 186 | 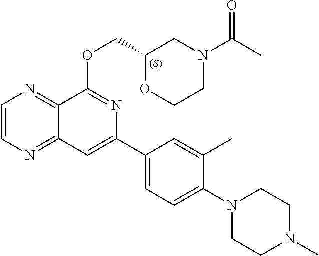 | 477 |
| 188 | 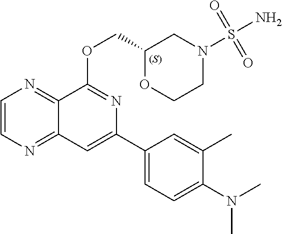 | 459 |
| 189 | 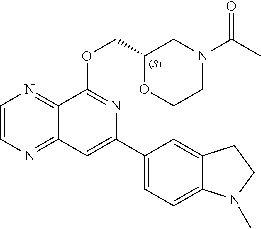 | 420 |
| 203 | 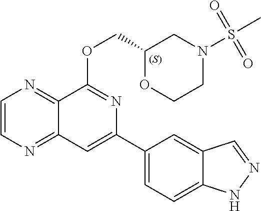 | 441 |
| 204 | 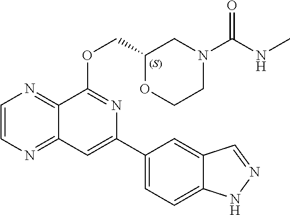 | 420 |
| 212 | 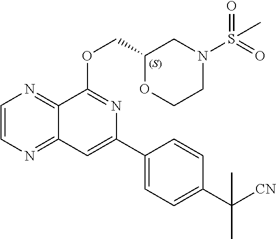 | 468 |
| 214 | 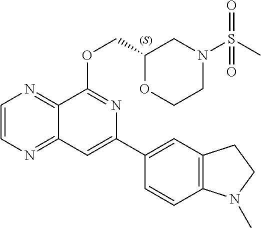 | 456 |
| 218 | 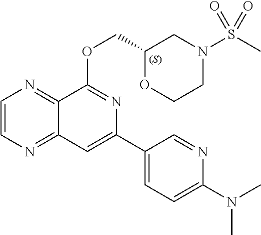 | 445 |
| 219 | 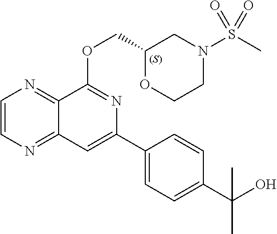 | 459 |
| 220 | 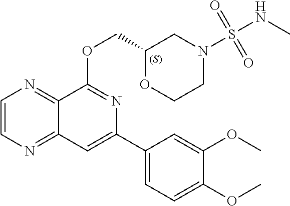 | 476 |
| 221 | 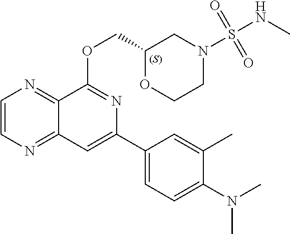 | 473 |
| 222 | 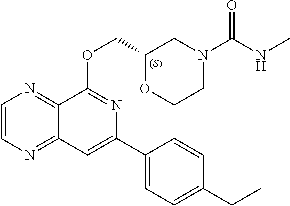 | 408 |
| 230 | 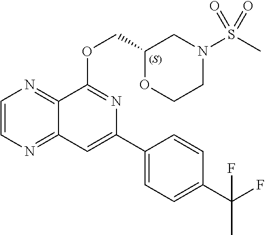 | 465 |
| 231 | 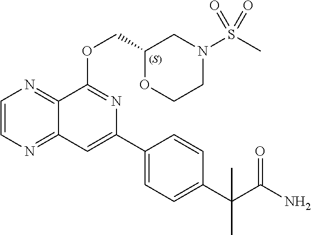 | 486 |
| 244 | 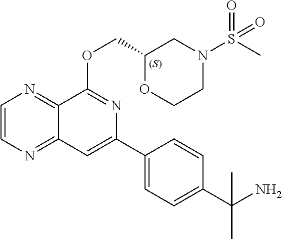 | 458 |
| 245 | 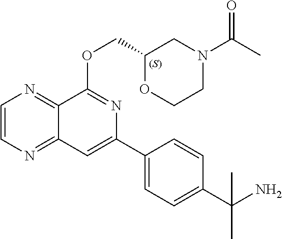 | 444 (M + Na) |
| 248 | 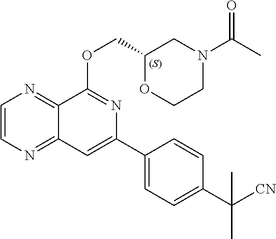 | 432 |
| 249 | 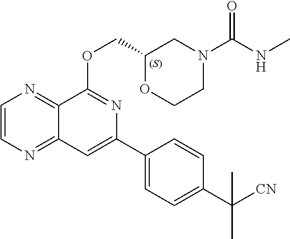 | 447 |
| 253 | 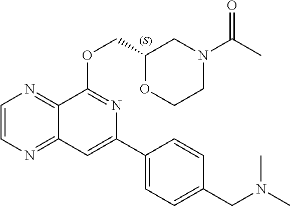 | 422 |
| 258 | 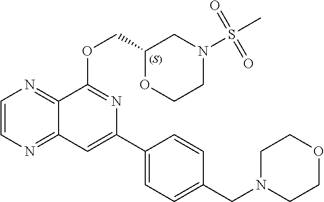 | 500 |
| 259 | 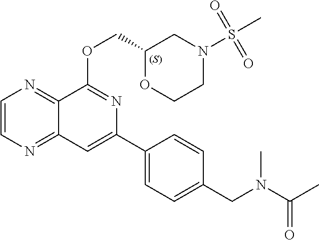 | 486 |
| 262 | 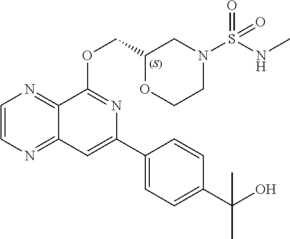 | 474 |
| 263 | 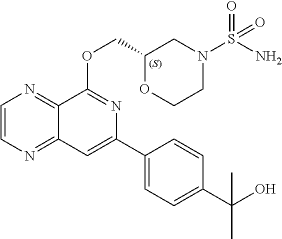 | 460 |
| 264 | 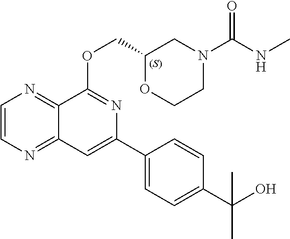 | 438 |
| 265 | 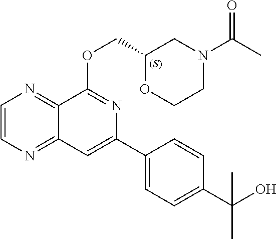 | 445 (M + Na) |
| 266 | 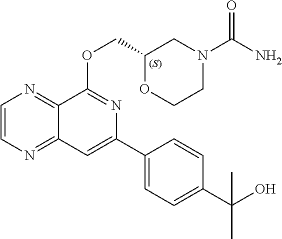 | 446 (M + Na) |
| 267 | 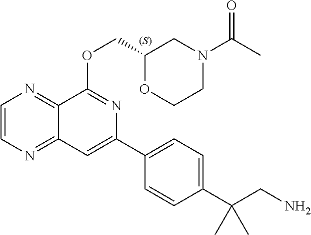 | 436 |
| 268 | 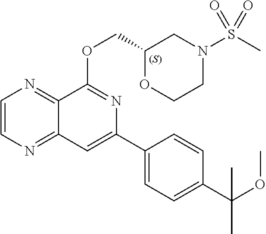 | 473 |
| 269 | 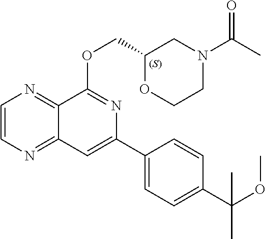 | 459 (M + Na) |
| 270 | 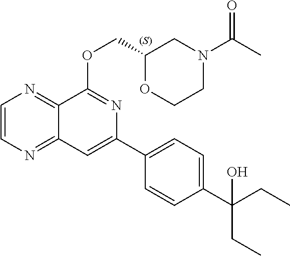 | 433 (M − 18 + H) |
| 271 | 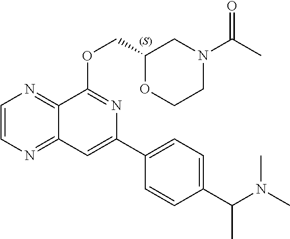 | 436 |
| 272 | 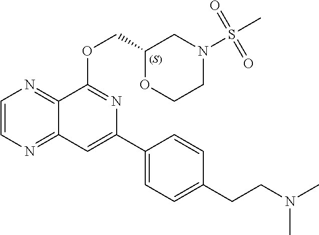 | 472 |
| 273 | 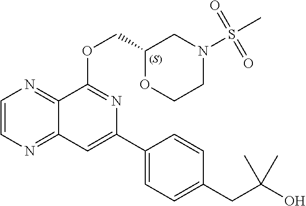 | 473 |
| 274 | 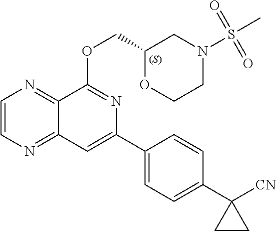 | 466 |
| 278 | 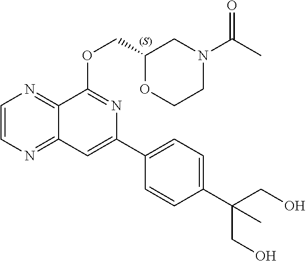 | 453 |
| 279 | 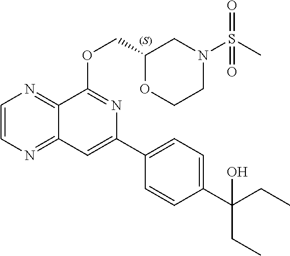 | 487 |
| 280 | 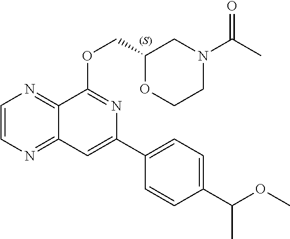 | 423 |
| 282 | 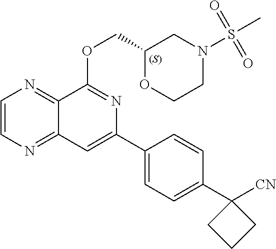 | 480 |
| 284 | 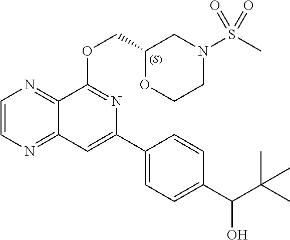 | 487 |
| 285 | 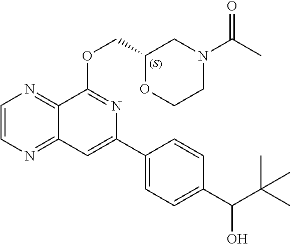 | 451 |
| 286 | 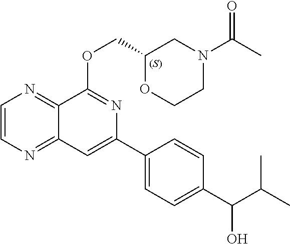 | 437 |
| 287 | 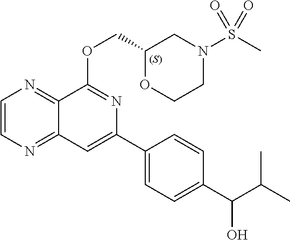 | 473 |
| 288 | 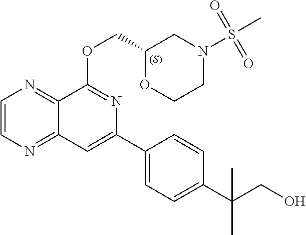 | 473 |
| 291 | 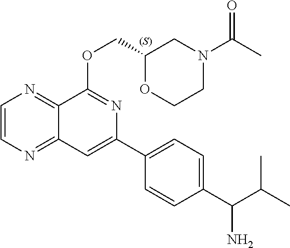 | 436 |
| 308 | 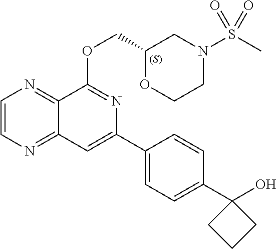 | 471 |
| 309 | 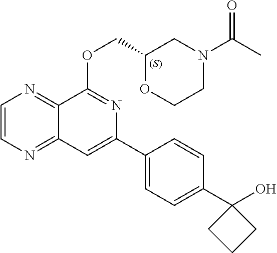 | 457 (M + Na) |
| 311 | 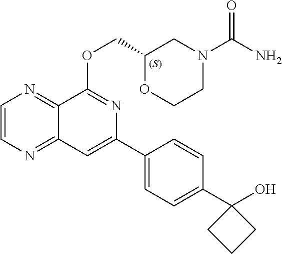 | 458 (M + Na) |
| 317 | 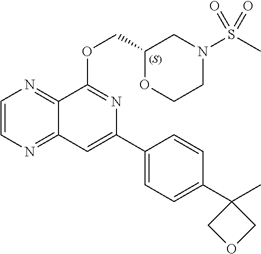 | 471 |
| 318 | 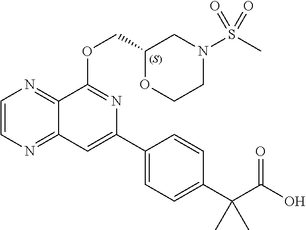 | 487 |
| 321 | 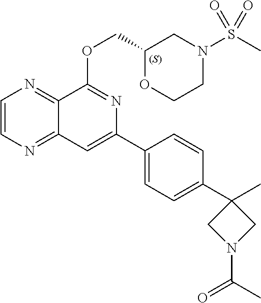 | 512 |
| 322 | 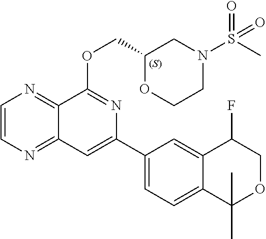 | 503 |
| 323 | 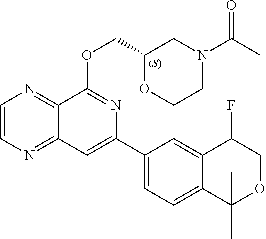 | 467 |

### Compound 57

### (S)-4-(methylsulfonyl)-2-((7-(4-(1-(methylsulfonyl)piperidin-4-yl)phenyl)pyrido[4,3-b]pyrazin-5-yloxy)methyl)morpholine

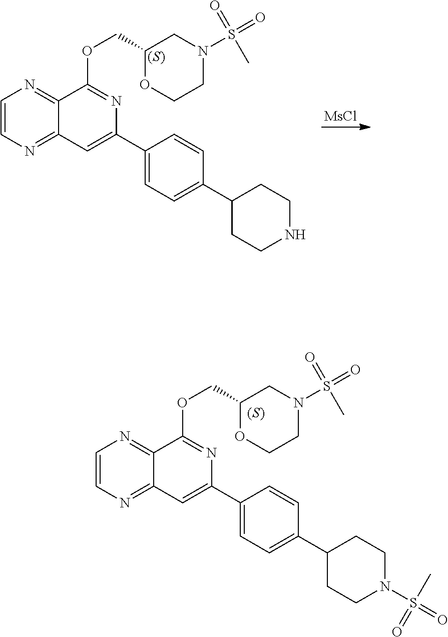

To a solution of (S)-4-(methylsulfonyl)-2-(((7-(4-(piperidin-4-yl)phenyl)pyrido[3,4-b]pyrazin-5-yl)oxy)methyl)morpholine (Compound 10) (121.0 mg, 0.25 mmol) and TEA (50 mg, 0.5 mmol) in DCM (3 mL) was added methanesulfonyl chloride (43 mg, 0.375 mmol) and the mixture was stirred at room temperature overnight. Then the reaction solution was concentrated and extracted with EA (100 mL), washed with brine (30 mL), dried over anhydrous Na₂SO₄ and purified by prep-TLC (DCM:MeOH=12:1) to give product as off-white solid. MS (m/z): 562 (M+H)⁺.

### Compound 58

### (S)-2-(4-(4-(5-((4-(methylsulfonyl)morpholin-2-yl)methoxy)pyrido[3,4-b]pyrazin-7-yl)phenyl)piperidin-1-yl)ethanol

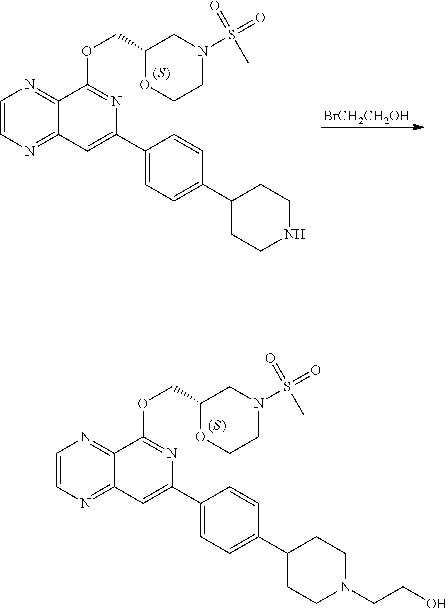

To a solution of (S)-4-(methylsulfonyl)-2-(((7-(4-(piperidin-4-yl)phenyl)pyrido[3,4-b]pyrazin-5-yl)oxy)methyl)morpholine (Compound 10) (75 mg, 0.155 mmol) and TEA (60 mg, 0.62 mmol) in DCM (3 mL) was added BrCH₂CH₂OH (58 mg, 0.465) dropwise. The mixture was stirred at room temperature for 4 days. Then it was concentrated and added EA, washed with brine, dried over Na₂SO₄ and purified by prep-TLC (DCM:MeOH=12:1) to give product as yellow solid. MS (m/z): 528 (M+H)⁺.

The following compound was prepared according to the procedures of Compound 58 using the corresponding intermediates and reagents under appropriate conditions that will be recognized by one skilled in the art.

| Com- | | MS |
|---|---|---|
| pound | Structure | (M + H)⁺ |
| 59 | 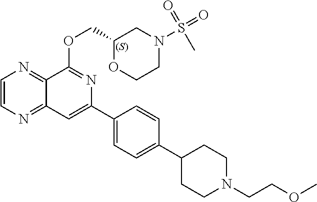 | 542 |
| 126 | 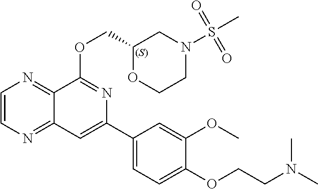 | 518 |

### Compound 60

### (S)-3-(dimethylamino)-1-(2-((7-(4-morpholinophenyl)pyrido[4,3-b]pyrazin-5-yloxy)methyl)morpholino)propan-1-one

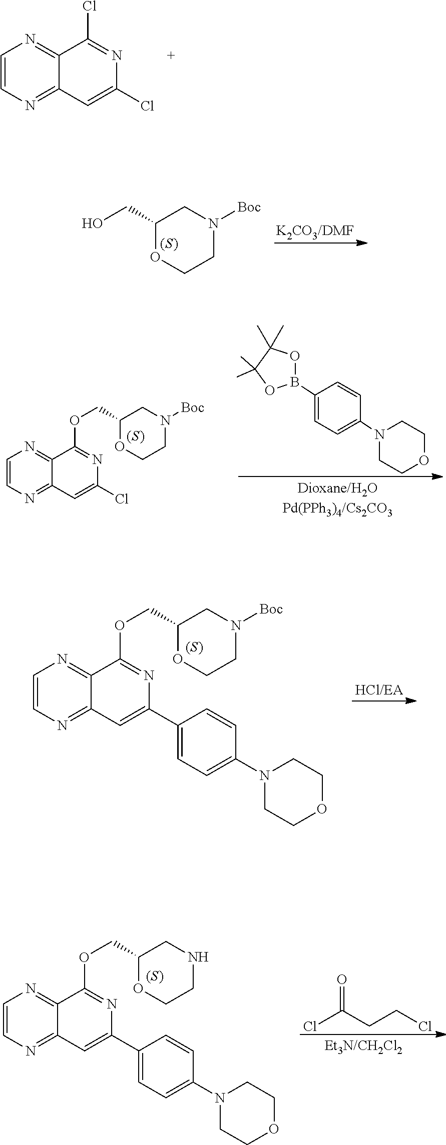

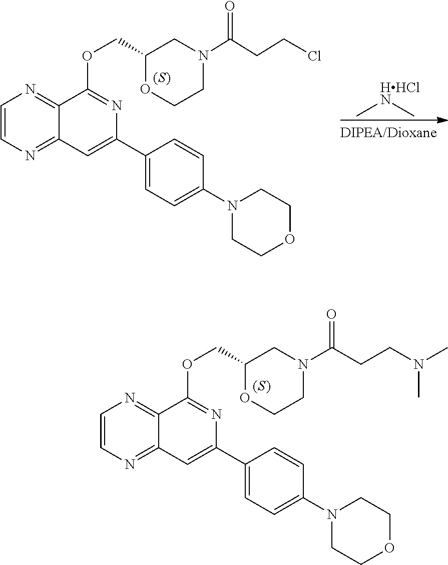

### (A) (S)-tert-butyl 2-((7-chloropyrido[4,3-b]pyrazin-5-yloxy)methyl)morpholine-4-carboxylate

To a solution of 5,7-dichloropyrido[4,3-b]pyrazine (11 g, 55 mmol) in DMF (200 mL) was added K₂CO₃ (13.8 g, 100 mmol) and (S)-tert-butyl 2-(hydroxymethyl)morpholine-4-carboxylate (10.86 g, 50 mmol). The mixture was stirred at 40° C. for 3 days. The reaction solution was poured into 600 mL water, extracted with EA (200 mL×3). The combined organic phase was washed with 300 mL water, brine, concentrated and purified by silica gel column chromatography (EA:PE=1:2) to give white solid. MS (m/z): 381 (M+H)⁺

### (B) (S)-tert-butyl 2-((7-(4-morpholinophenyl)pyrido[4,3-b]pyrazin-5-yloxy)methyl)morpholine-4-carboxylate

To a solution of (S)-tert-butyl 2-((7-chloropyrido[4,3-b]pyrazin-5-yloxy)methyl)morpholine-4-carboxylate (571 mg, 1.5 mmol) in dioxane/H₂O (5 mL/0.5 mL) was added Cs₂CO₃ (733 mg, 2.25 mmol), Pd(PPh₃)₄ (173 mg, 0.15 mmol) and 4-(4-(4,4,5,5-tetramethyl-1,3,2-dioxaborolan-2-yl)phenyl)morpholine (492 mg, 1.65 mmol). The mixture was stirred at 100° C. for 13 hours under N₂. The reaction solution was added into 100 mL water, extracted with EA. The organic phase was washed with brine, concentrated to give an crude product, which was purified by prep-TCL (DCM:MeOH=50:1) to give yellow solid. MS (m/z): 508 (M+H)⁺

### (C) (S)-2-((7-(4-morpholinophenyl)pyrido[4,3-b]pyrazin-5-yloxy)methyl)morpholine

(S)-tert-butyl 2-((7-(4-morpholinophenyl)pyrido[4,3-b]pyrazin-5-yloxy)methyl)morpholine-4-carboxylate (1.5 mmol) was dissolved in a solution of 5N HCl in EA (10 mL) and stirred for 4 hours at 20° C. The reaction solution was concentrated and washed with saturated NaHCO₃(aq.), water and brine, concentrated to give yellow solid. MS (m/z): 408 (M+H)⁺

### (D) (S)-3-chloro-1-(2-((7-(4-morpholinophenyl)pyrido[4,3-b]pyrazin-5-yloxy)methyl)morpholino)propan-1-one

To a solution of (S)-2-((7-(4-morpholinophenyl)pyrido[4,3-b]pyrazin-5-yloxy)methyl)morpholine (122 mg, 0.3 mmol) in CH₂Cl₂ (5 mL) was added Et₃N (63 uL, 0.45 mmol) and 3-chloropropanoyl chloride (57.2 mg, 0.45 mmol) at room temperature. The reaction solution was stirred at room temperature for 4 hours. After that, the reaction solution was washed with aqueous NaHCO₃ (5 mL), H₂O (5 mL) and brine (5 mL), dried over Na₂SO₄ and concentrated, purified by prep-TLC (CH₂Cl₂:MeOH=50:1) to give white solid. MS (m/z): 498 (M+H)⁺

### (E) (S)-3-(dimethylamino)-1-(2-((7-(4-morpholinophenyl)pyrido[4,3-b]pyrazin-5-yloxy)methyl)morpholino)propan-1-one

To a solution of (S)-3-chloro-1-(2-((7-(4-morpholinophenyl)pyrido[4,3-b]pyrazin-5-yloxy)methyl)morpholino)propan-1-one (111 mg, 0.22 mmol) in dioxane (5 mL) was added DIPEA (368 uL, 2.23 mmol) and dimethylamine hydrochloride (182 mg, 2.23 mmol) at room temperature. The reaction solution was sealed and heated in microwave reactor at 170° C. for 0.5 hour. After that, the reaction solution was concentrated and purified by prep-TLC(CH₂Cl₂:MeOH=40:1) to give yellow solid. MS (m/z): 507 (M+H)⁺

The following compounds were prepared according to the procedures of Compound 60 using the corresponding intermediates and reagents under appropriate conditions that will be recognized by one skilled in the art.

| Compound | Structure | MS (M + H)⁺ |
|---|---|---|
| 61 | 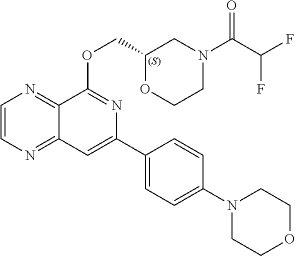 | 486 |
| 62 | 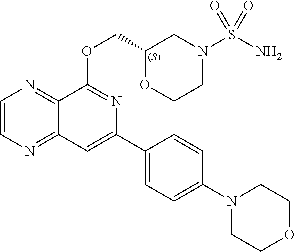 | 487 |
| 63 | 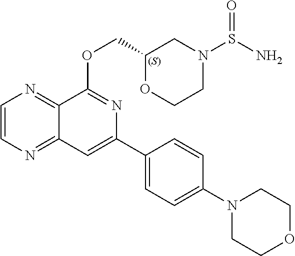 | 451 |
| 64 | 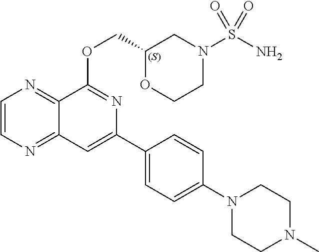 | 500 |
| 65 | 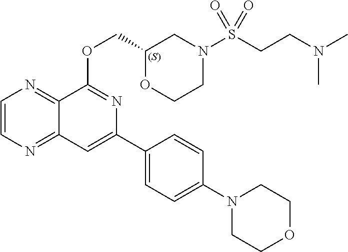 | 543 |
| 66 | 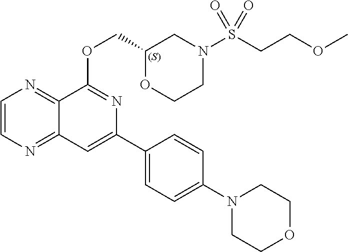 | 530 |
| 67 | 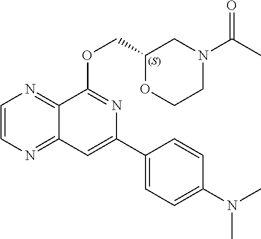 | 408 |
| 68 | 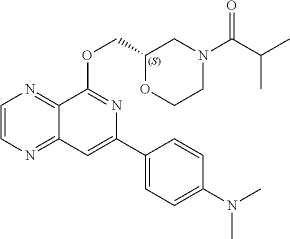 | 436 |
| 69 | 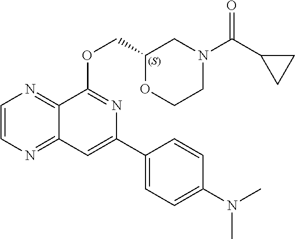 | 434 |
| 70 | 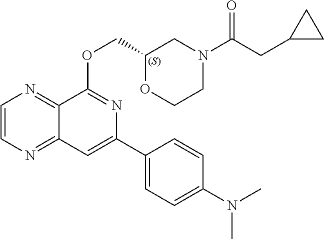 | 448 |
| 71 | 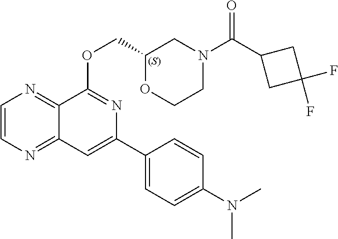 | 484 |
| 72 | 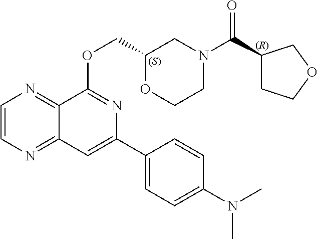 | 464 |
| 73 | 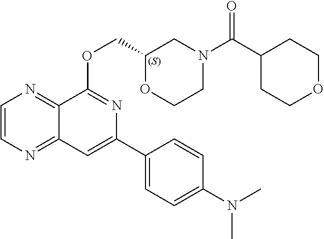 | 478 |
| 74 | 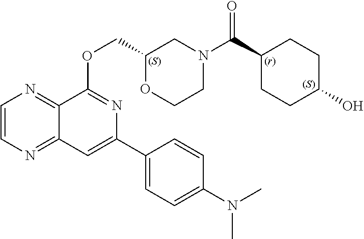 | 492 |
| 75 | 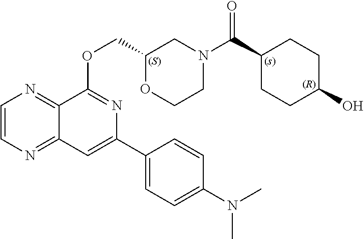 | 492 |
| 76 | 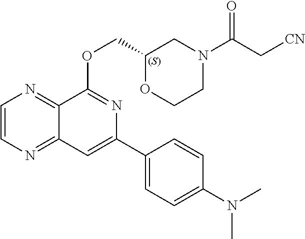 | 433 |
| 77 | 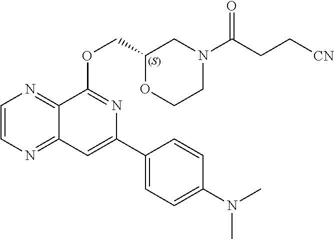 | 447 |
| 78 | 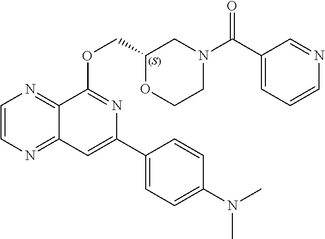 | 471 |
| 79 | 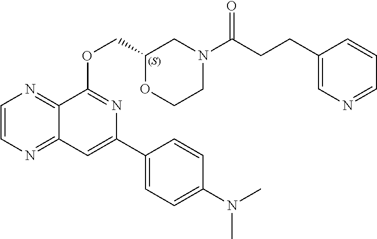 | 499 |
| 80 | 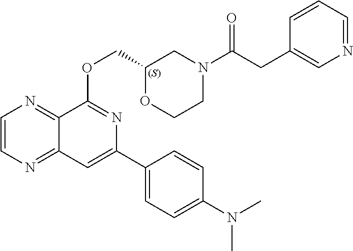 | 485 |
| 81 | 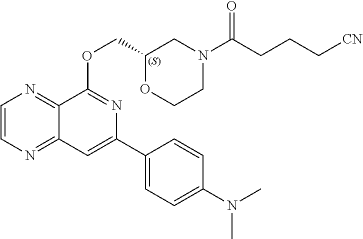 | 461 |
| 82 | 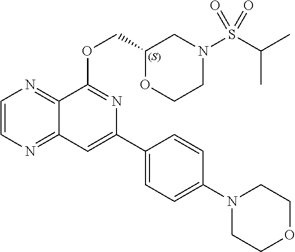 | 514 |
| 83 | 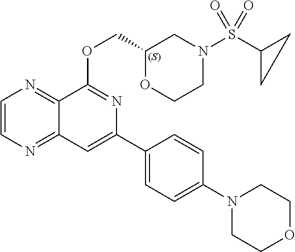 | 512 |
| 84 | 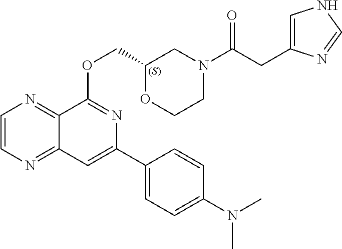 | 474 |
| 85 | 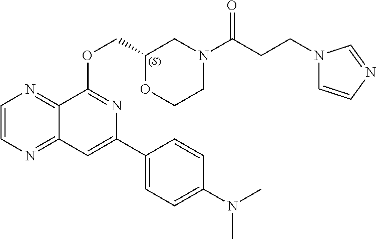 | 488 |
| 86 | 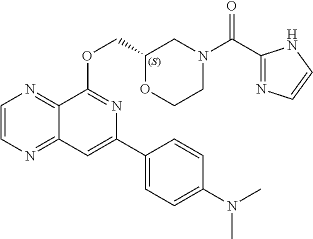 | 460 |
| 87 | 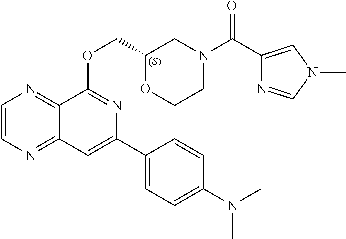 | 474 |
| 88 | 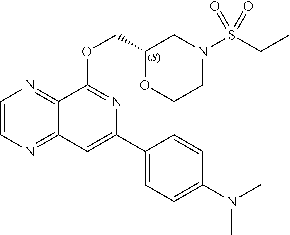 | 458 |
| 89 | 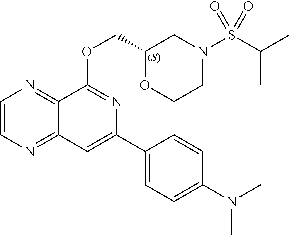 | 472 |
| 90 | 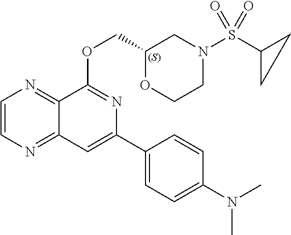 | 470 |
| 91 | 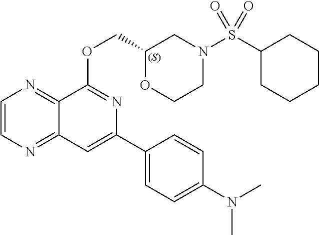 | 512 |
| 92 | 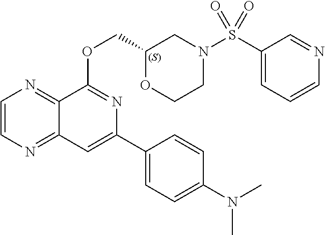 | 507 |
| 93 | 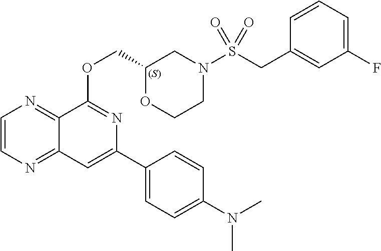 | 538 |
| 94 | 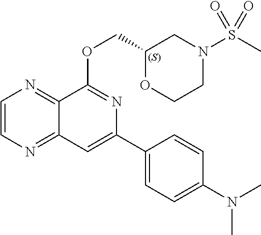 | 444 |
| 95 | 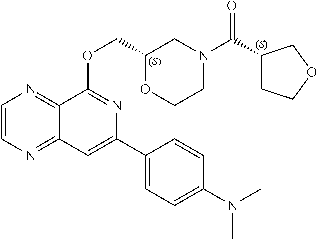 | 464 |
| 96 | 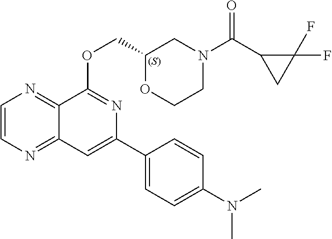 | 470 |
| 97 | 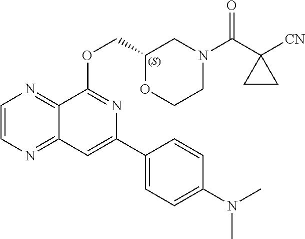 | 459 |
| 98 | 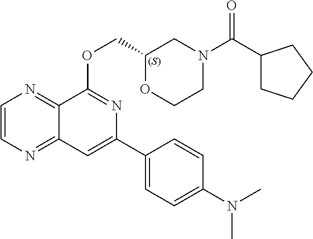 | 462 |
| 99 | 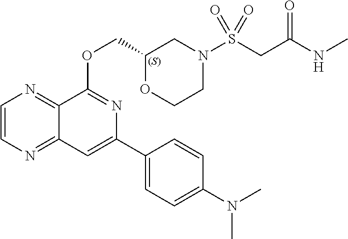 | 501 |
| 112 | 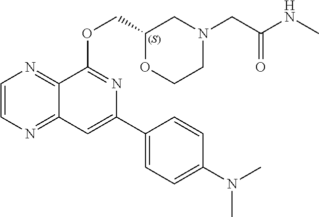 | 437 |
| 113 | 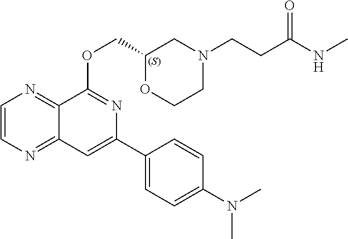 | 451 |
| 128 | 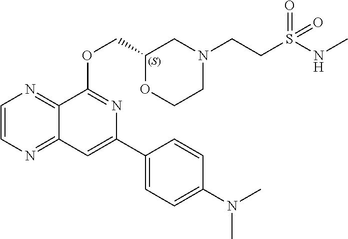 | 487 |
| 136 | 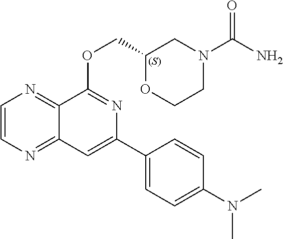 | 409 |
| 137 | 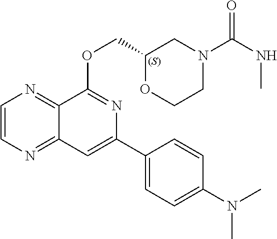 | 423 |
| 143 | 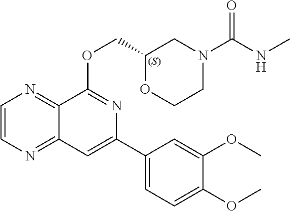 | 440 |
| 159 | 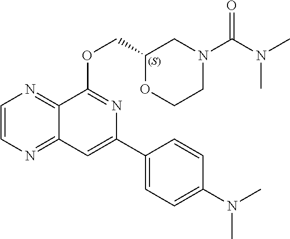 | 437 |
| 164 | 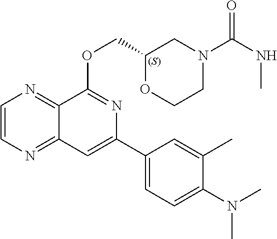 | 437 |
| 166 | 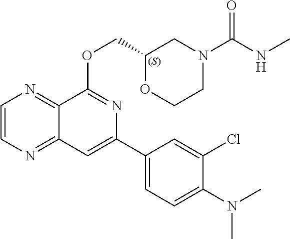 | 457 |
| 172 | 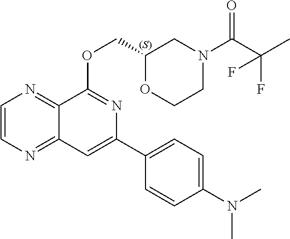 | 458 |
| 173 | 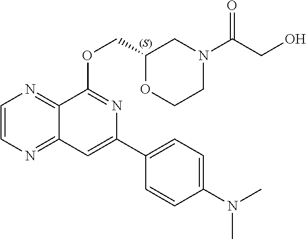 | 424 |
| 176 | 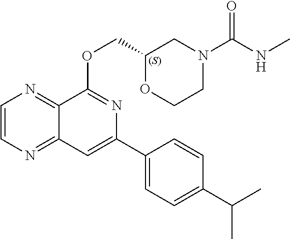 | 422 |
| 181 | 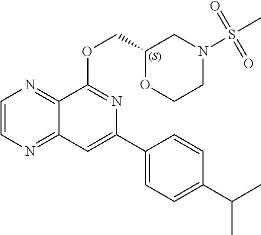 | 443 |
| 184 | 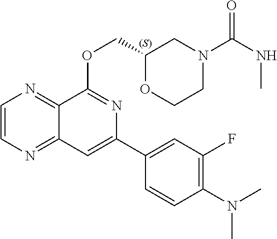 | 441 |
| 187 | 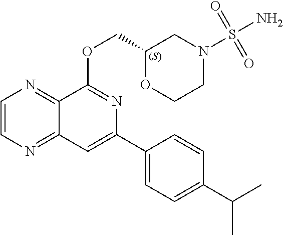 | 444 |
| 195 | 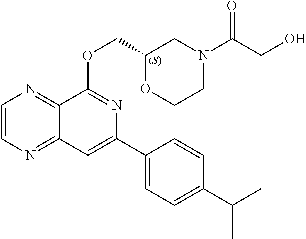 | 423 |
| 196 | 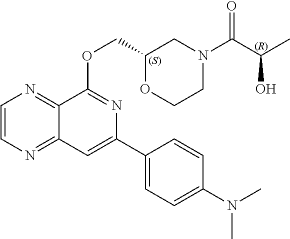 | 438 |
| 197 | 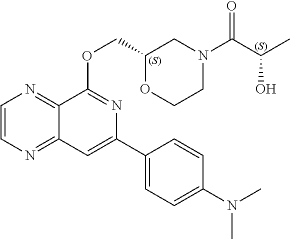 | 438 |
| 198 | 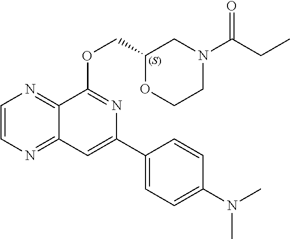 | 422 |
| 200 | 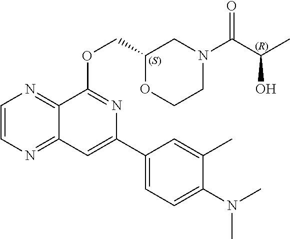 | 452 |
| 201 | 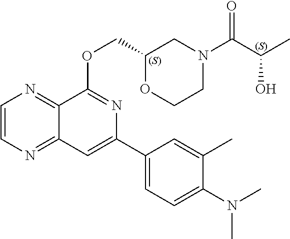 | 452 |
| 202 | 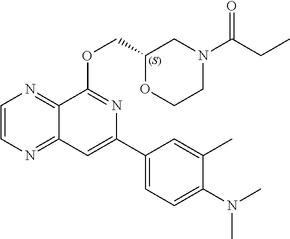 | 436 |
| 207 | 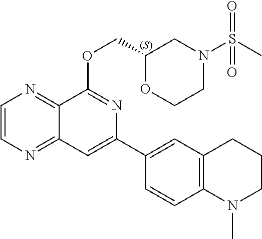 | 470 |
| 208 | 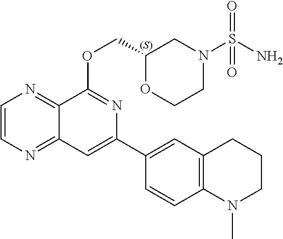 | 471 |
| 211 | 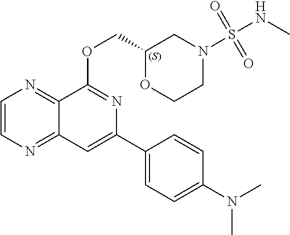 | 459 |
| 215 | 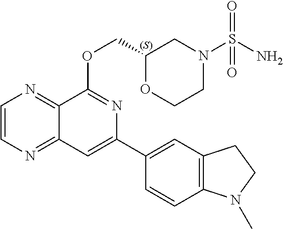 | 457 |
| 233 | 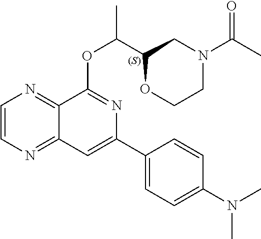 | 422 |
| 235 | 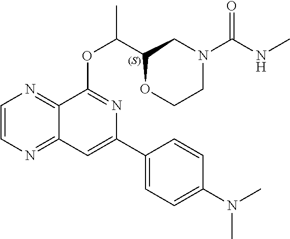 | 437 |
| 236 | 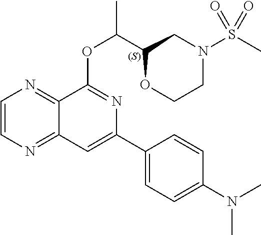 | 458 |
| 237 | 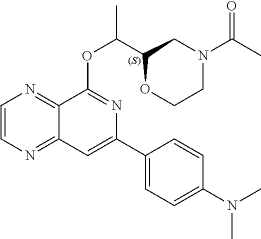 | 422 |
| 239 | 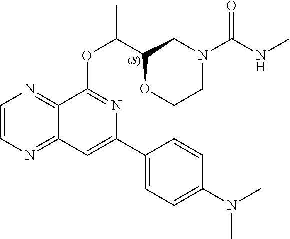 | 437 |
| 240 | 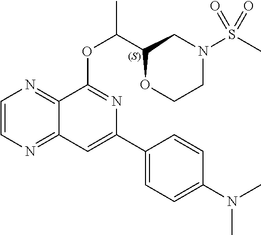 | 458 |
| 246 | 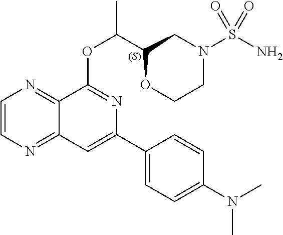 | 459 |
| 247 | 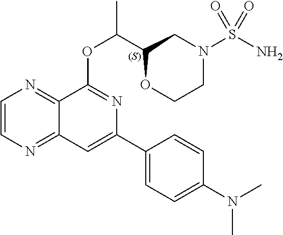 | 459 |
| 250 | 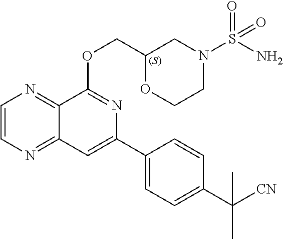 | 469 |
| 251 | 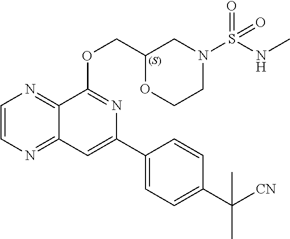 | 483 |
| 254 | 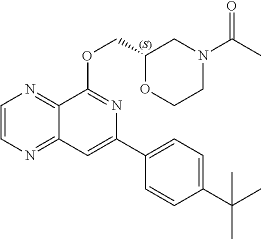 | 421 |
| 256 | 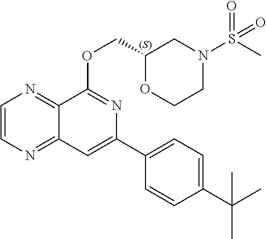 | 457 |
| 257 | 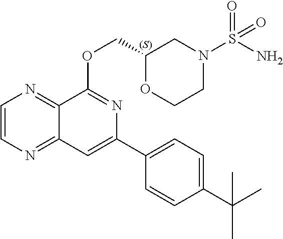 | 458 |
| 260 | 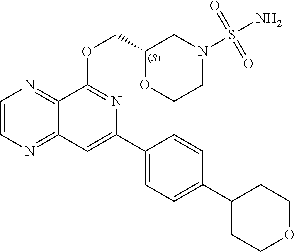 | 486 |
| 276 | 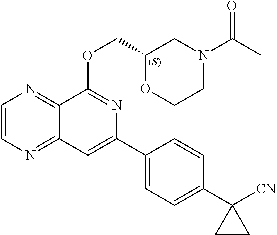 | 430 |
| 283 | 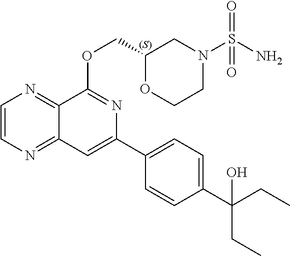 | 488 |
| 289 | 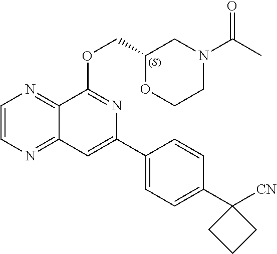 | 444 |
| 297 | 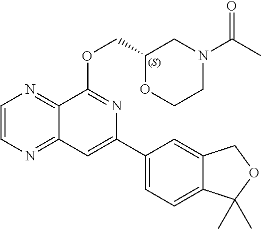 | 435 |
| 299 | 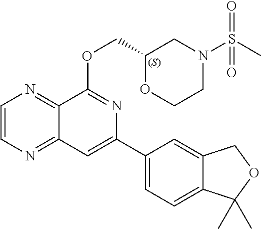 | 471 |
| 300 | 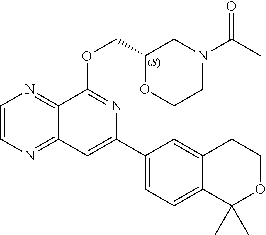 | 449 |
| 302 | 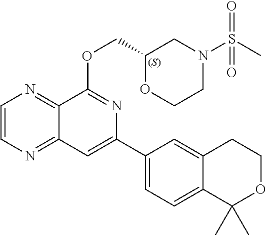 | 485 |
| 312 | 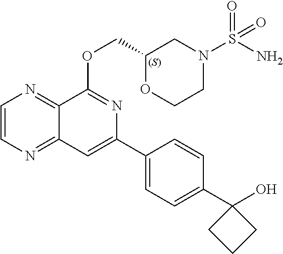 | 472 |
| 313 | 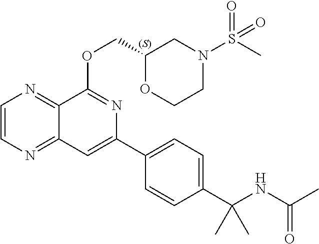 | 500 |
| 314 | 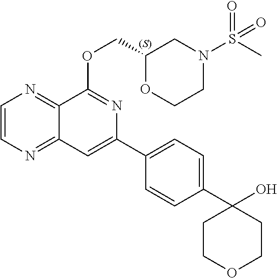 | 501 |

### Compound 101

### (S)—N,N-dimethyl-4-(5-((4-(2-(2-methyl-1H-imidazol-1-yl)ethylsulfonyl)morpholin-2-yl)methoxy)pyrido[4,3-b]pyrazin-7-yl)aniline

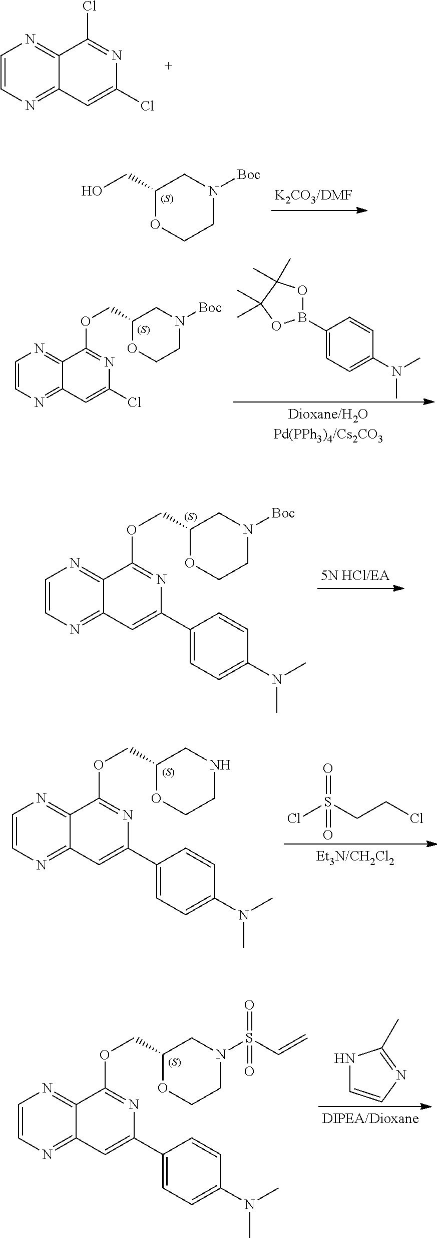

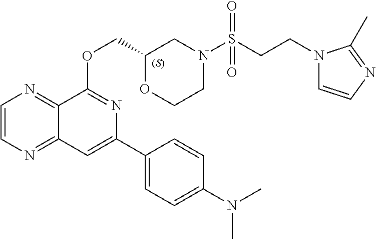

### (A) (S)-tert-butyl 2-((7-chloropyrido[4,3-b]pyrazin-5-yloxy)methyl)morpholine-4-carboxylate

The title compound was prepared according to the procedures of Compound 60(A). MS (m/z): 381 (M+H)⁺

### (B) (S)-tert-butyl 2-((7-(4-(dimethylamino)phenyl)pyrido[4,3-b]pyrazin-5-yloxy)methyl)morpholine-4-carboxylate

The title compound was prepared according to the procedures of Compound 60(B). MS (m/z): 466 (M+H)⁺

### (C) (S)—N,N-dimethyl-4-(5-(morpholin-2-ylmethoxy)pyrido[4,3-b]pyrazin-7-yl)aniline

The title compound was prepared according to the procedures of Compound 60(C). MS (m/z): 366 (M+H)⁺

### (D) (S)—N,N-dimethyl-4-(5-((4-(vinylsulfonyl)morpholin-2-yl)methoxy)pyrido[4,3-b]pyrazin-7-yl)aniline

To a solution of (S)—N,N-dimethyl-4-(5-(morpholin-2-ylmethoxy)pyrido[4,3-b]pyrazin-7-yl)aniline (292.8 mg, 0.8 mmol) in CH₂Cl₂ (5 mL) was added Et₃N (278 uL, 2 mmol) and 2-chloroethanesulfonyl chloride (152.4 mg, 1.2 mmol) at room temperature. The reaction solution was stirred at room temperature for 4 hours. After that, the reaction solution was washed with aqueous NaHCO₃ (5 mL), H₂O (5 mL) and brine (5 mL), dried over Na₂SO₄ and concentrated, purified by prep-TLC (CH₂Cl₂:MeOH=70:1) to give white solid. MS (m/z): 456 (M+H)⁺

### (E) (S)—N,N-dimethyl-4-(5-((4-(2-(2-methyl-1H-imidazol-1-yl)ethylsulfonyl)morpholin-2-yl)methoxy)pyrido[4,3-b]pyrazin-7-yl)aniline

To a solution of (S)—N,N-dimethyl-4-(5-((4-(vinylsulfonyl)morpholin-2-yl)methoxy)pyrido[4,3-b]pyrazin-7-yl)aniline (60 mg, 0.13 mmol) in dioxane (5 mL) was added DIPEA (165 uL, 1 mmol) and 2-methyl-1H-imidazole (82.1 mg, 1 mmol) at room temperature. The reaction solution was sealed in a tube and heated in microwave reactor at 170° C. for 1 hour. After that, the reaction solution was concentrated and purified by prep-TLC(CH₂Cl₂:MeOH=40:1) to give yellow solid. MS (m/z): 538 (M+H)⁺

The following compounds were prepared according to the procedures of Compound 101 using the corresponding intermediates and reagents under appropriate conditions that will be recognized by one skilled in the art.

| Compound | Structure | MS (M + H)⁺ |
|---|---|---|
| 102 | 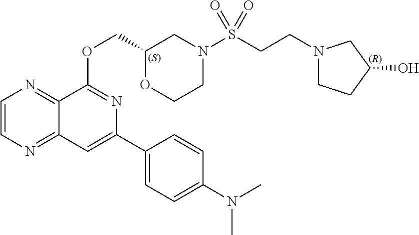 | 543 |
| 103 | 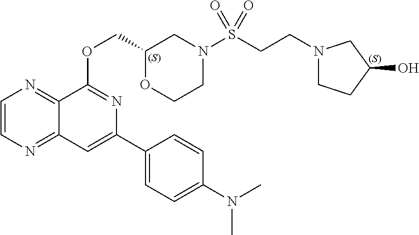 | 543 |
| 104 | 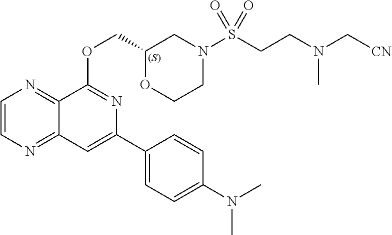 | 526 |

### Compound 115

### (S)—N-(2-(2-(((7-(4-(dimethylamino)phenyl)pyrido[3,4-b]pyrazin-5-yl)oxy)methyl)morpholino)ethyl)acetamide

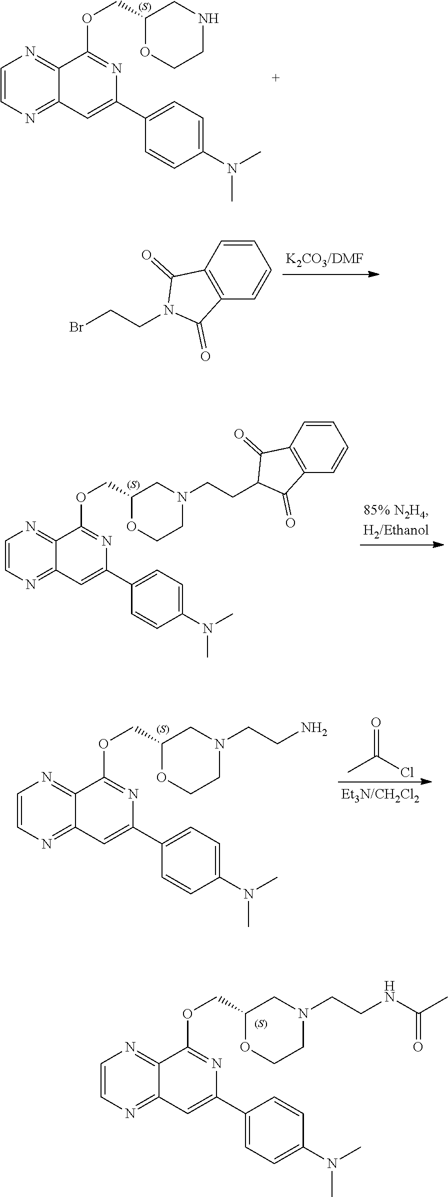

### (A) (S)-2-(2-(2-((7-(4-(dimethylamino)phenyl)pyrido[4,3-b]pyrazin-5-yloxy)methyl)morpholino)ethyl)isoindoline-1,3-dione

To a solution of Compound 101 (C) (732 mg, 2 mmol) in DMF (5 mL) was added K₂CO₃ (552 mg, 4 mmol) and 2-(2-bromoethyl)isoindoline-1,3-dione (1016 mg, 4 mmol) at room temperature. The reaction was stirred at 100° C. for 24 hours. After that, the reaction solution was extracted with EA, washed with water (5 mL) and brine (5 mL), dried over dry Na₂SO₄ and concentrated, purified by prep-TLC(CH₂Cl₂:MeOH=45:1) to give solid. MS (m/z): 539 (M+H)⁺

### (B) (S)-4-(5-((4-(2-aminoethyl)morpholin-2-yl)methoxy)pyrido[4,3-b]pyrazin-7-yl)-N,N-dimethylaniline

To a solution of (S)-2-(2-(2-((7-(4-(dimethylamino)phenyl)pyrido[4,3-b]pyrazin-5-yloxy)methyl)morpholino)ethyl)isoindoline-1,3-dione (279 mg, 0.52 mmol) in ethanol (5 mL) was added 85% N₂H₄.H₂O (52 mg, 1.04 mmol) at room temperature. The mixture was refluxed for 4 hours. After that, the mixture was adjusted to PH˜7 with 2N HCl solution, concentrated, purified by prep-TLC(CH₂Cl₂:MeOH=15:1) to give yellow solid. MS (m/z): 409 (M+H)⁺

### (C) (S)—N-(2-(2-((7-(4-(dimethylamino)phenyl)pyrido[4,3-b]pyrazin-5-yloxy)methyl)morpholino)ethyl)acetamide

To a solution of (S)-4-(5-((4-(2-aminoethyl)morpholin-2-yl)methoxy)pyrido[4,3-b]pyrazin-7-yl)-N,N-dimethylaniline (27 mg, 0.066 mmol) in CH₂Cl₂ (5 mL) was added Et₃N (14 uL, 0.099 mmol) and acetyl chloride (7.8 mg, 0.099 mmol) at room temperature. The reaction mixture was stirred at room temperature for 4 hours. After that, the reaction mixture was washed with NaHCO₃ (5 mL), H₂O (5 mL) and brine (5 mL), dried over Na₂SO₄ and concentrated, purified by prep-TLC(CH₂Cl₂:MeOH=45:1) to give yellow solid. MS (m/z): 451 (M+H)⁺

The following compounds were prepared according to the procedures of Compound 115 using the corresponding intermediates and reagents under appropriate conditions that will be recognized by one skilled in the art.

| Compound | Structure | MS (M + H)⁺ |
|---|---|---|
| 116 | 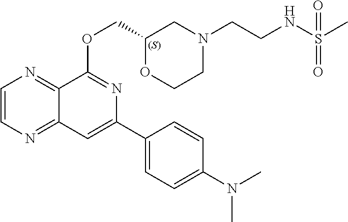 | 487 |
| 117 | 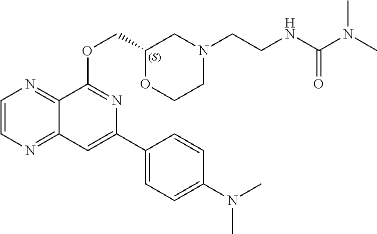 | 480 |
| 118 | 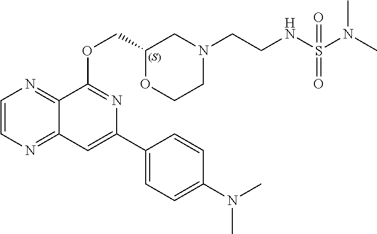 | 517 |

### Compound 135

### ((S)-2-(((7-(4-(methylamino)phenyl)pyrido[3,4-b]pyrazin-5-yl)oxy)methyl)morpholino)((S)-1-methylpyrrolidin-3-yl)methanone

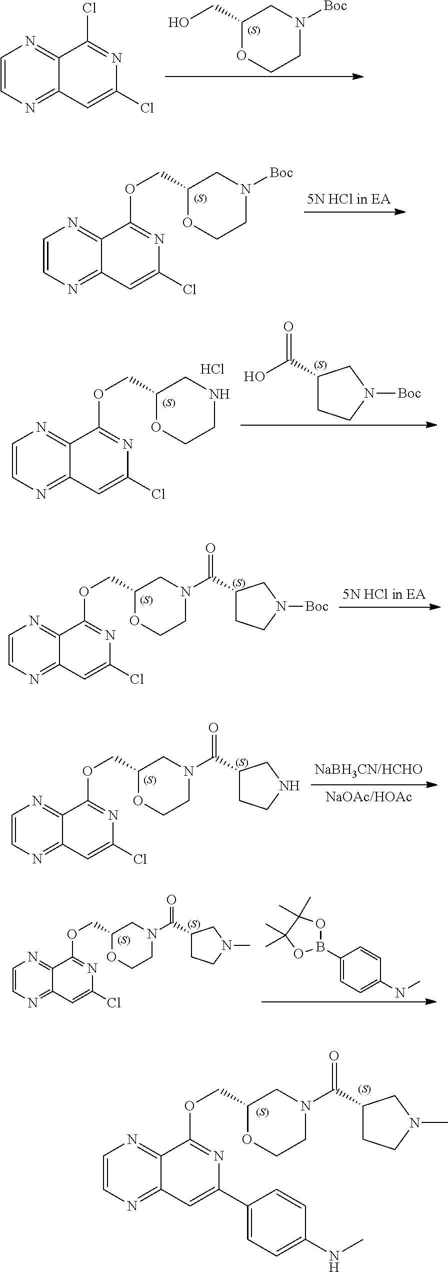

### (A) (S)-tert-butyl 2-(((7-chloropyrido[3,4-b]pyrazin-5-yl)oxy)methyl)morpholine-4-carboxylate

The title compound was prepared according to the procedures of Compound 6 (A).

### (B) (S)-2-(((7-chloropyrido[3,4-b]pyrazin-5-yl)oxy)methyl)morpholine

The title compound was prepared according to the procedures of Compound 6 (B). MS (m/z): 281 (M+H)⁺

### (C) (S)-tert-butyl 3-((S)-2-(((7-chloropyrido[3,4-b]pyrazin-5-yl)oxy)methyl)morpholine-4-carbonyl)pyrrolidine-1-carboxylate

The title compound was prepared according to the procedures of Compound 6 (B).

### (D) ((S)-2-(((7-chloropyrido[3,4-b]pyrazin-5-yl)oxy)methyl)morpholino)((S)-pyrrolidin-3-yl)methanone

The title compound was prepared according to the procedures of Compound 6 (B). MS (m/z): 378 (M+H)⁺

### (E) US)-2-(((7-chloropyrido[3,4-b]pyrazin-5-yl)oxy)methyl)morpholino)((S)-1-methylpyrrolidin-3-yl)methanone

((S)-2-(((7-chloropyrido[3,4-b]pyrazin-5-yl)oxy)methyl)morpholino)((S)-pyrrolidin-3-yl)methanone (0.43 mmol) was dissolved in 37% aqueous formaldehyde (10 mL) and acetic acid (258 mg, 4.3 mmol). NaOAc (352.6 mg, 4.3 mmol) was added and the mixture was cooled with ice-water bath. NaBH₃CN (27 mg, 0.43 mmol) was added to the mixture and the reaction solution was stirred for 3 hours. Saturated aq. NaHCO₃ was added until pH>7. The mixture was extracted with DCM twice. Organic phases were combined and dried over dry MgSO₄, concentrated, purified by prep-TLC (DCM:MeOH=10:1) to give yellow solid. MS (m/z): 392 (M+H)⁺

### (F) ((S)-2-(((7-(4-(methylamino)phenyl)pyrido[3,4-b]pyrazin-5-yl)oxy)methyl)morpholino)((S)-1-methylpyrrolidin-3-yl)methanone

The title compound was prepared according to the procedures of Compound 6 (C). MS (m/z): 463 (M+H)⁺

### Compound 142

### (S)-2-(((7-(3,4-dimethoxyphenyl)pyrido[3,4-b]pyrazin-5-yl)oxy)methyl)morpholine-4-carboxamide

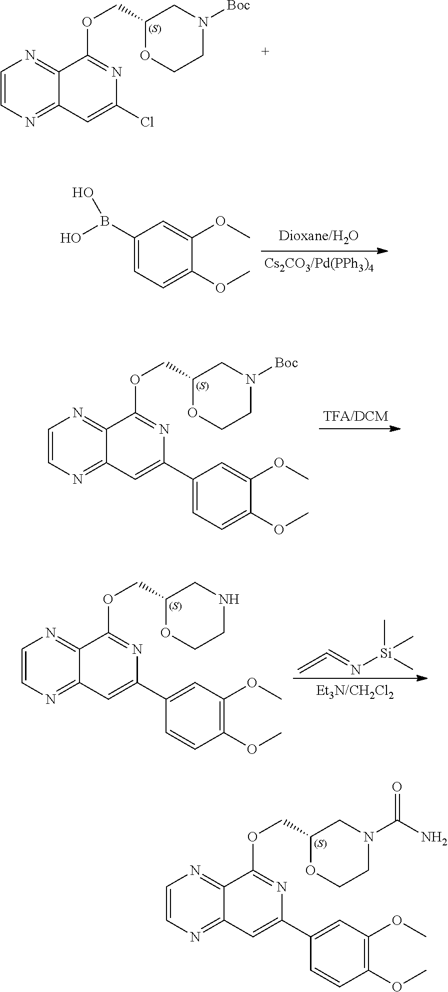

### (A) (S)-tert-butyl 2-((7-(3,4-dimethoxyphenyl)pyrido[4,3-b]pyrazin-5-yloxy)methyl)morpholine-4-carboxylate

To a solution of (S)-tert-butyl 2-((7-chloropyrido[4,3-b]pyrazin-5-yloxy)methyl)morpholine-4-carboxylate (190.4 mg, 0.5 mmol) in dioxane/H₂O (5 mL/0.5 mL) was added Cs₂CO₃ (244.4 mg, 0.75 mmol), Pd(PPh₃)₄ (58 mg, 0.05 mmol) and 3,4-dimethoxyphenylboronic acid (100 mg, 0.55 mmol). The mixture was sealed in a tube and heated in microwave reactor at 160° C. for 1 hour under N₂. The reaction mixture was filtered, the filtrate was concentrated and purified by column chromatography (DCM:MeOH=70:1) to give title compound. MS (m/z): 483 (M+H)⁺

### (B)(S)-2-((7-(3,4-dimethoxyphenyl)pyrido[4,3-b]pyrazin-5-yloxy)methyl)morpholine

(S)-tert-butyl 2-((7-(3,4-dimethoxyphenyl)pyrido[4,3-b]pyrazin-5-yloxy)methyl)morpholine-4-carboxylate (160 mg, 0.33 mmol) was dissolved in a solution of TFA/CH₂Cl₂ (8 mL/8 mL) and the mixture was stirred for 4 hours at 20° C. The reaction mixture was concentrated and the residue was dissolved in 50 mL n-BuOH. The organic phase was washed with sat. aq. NaHCO₃, water and brine, dried and concentrated to give title compound. MS (m/z): 383 (M+H)⁺

### (C) (S)-2-((7-(3,4-dimethoxyphenyl)pyrido[4,3-b]pyrazin-5-yloxy)methyl)morpholine-4-carboxamide

To a solution of (S)-2-((7-(3,4-dimethoxyphenyl)pyrido[4,3-b]pyrazin-5-yloxy)methyl)morpholine (25 mg, 0.065 mmol) in CH₂Cl₂ (5 mL) was added Et₃N (18 uL, 0.13 mmol) and isocyanatotrimethylsilane (15 mg, 0.13 mmol) at room temperature. The reaction mixture was stirred at room temperature for 20 hours. After that the reaction solution was washed with aq. NaHCO₃ (5 mL), H₂O (5 mL) and brine (5 mL), dried over Na₂SO₄ and concentrated, purified on thin-layer chromatography (CH₂Cl₂:MeOH=30:1) to give title compound. MS (m/z): 426 (M+H)⁺

The following compounds were prepared according to the procedures of Compound 142 using the corresponding intermediates and reagents under appropriate conditions that will be recognized by one skilled in the art.

| | | MS |
|---|---|---|
| Compound | Structure | (M + H)⁺ |
| 163 | 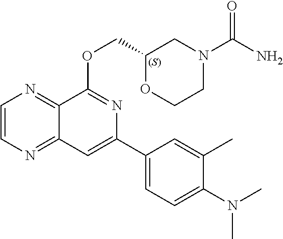 | 423 |
| 165 | 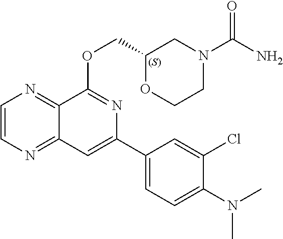 | 443 |
| 175 | 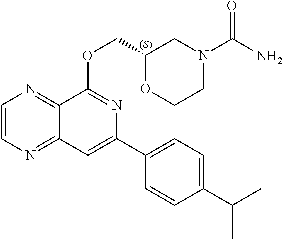 | 408 |
| 183 | 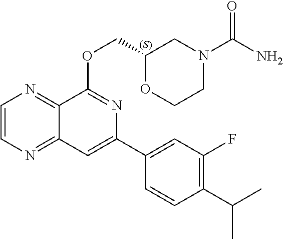 | 427 |
| 209 | 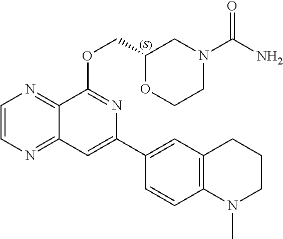 | 435 |
| 216 | 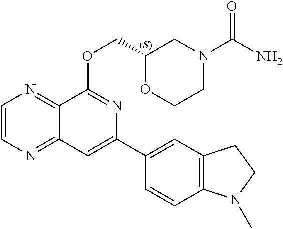 | 421 |
| 234 | 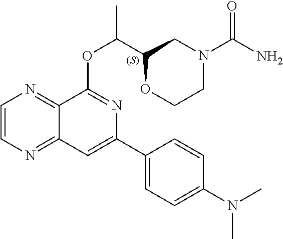 | 423 |
| 238 | 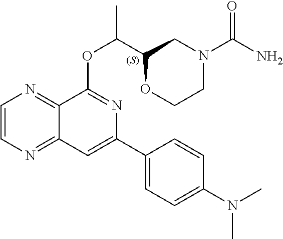 | 423 |
| 252 | 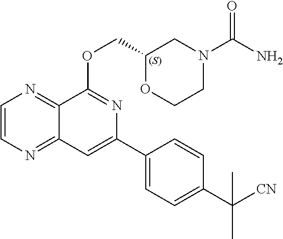 | 433 |
| 255 | 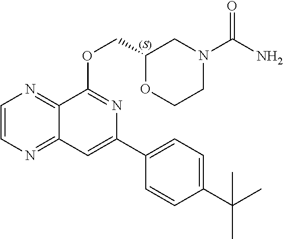 | 422 |
| 275 | 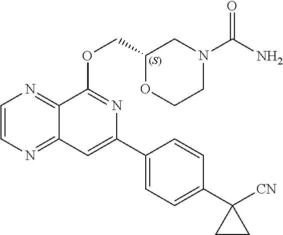 | 431 |
| 281 | 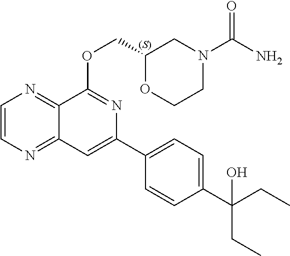 | 452 |
| 290 | 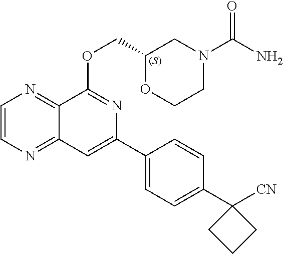 | 445 |
| 298 | 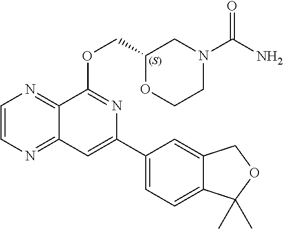 | 436 |
| 301 | 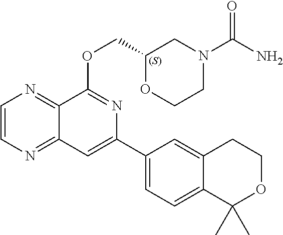 | 450 |

### Compound 144

### (S)-1-(2-(((7-(4-(isopropyl(methyl)amino)phenyl)pyrido[3,4-b]pyrazin-5-yl)oxy)methyl)morpholino)ethanone

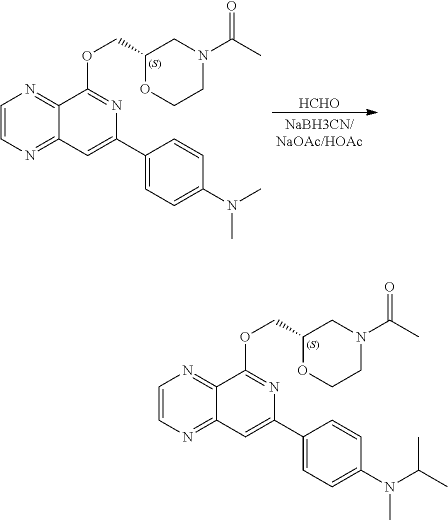

Compound 141 (21 mg, 0.05 mmol) was dissolved in 37% aqueous formaldehyde (2 mL) and acetic acid (30 mg, 0.5 mmol). Sodium acetate (41 mg, 0.5 mmol) was added and the mixture was cooled in ice/water bath. Sodium cyanoborohydride (6.3 mg, 0.1 mmol) was added and the mixture was allowed to stir for 3 hours. Saturated aqueous sodium hydrogen carbonate was added until the mixture was basic. The mixture was extracted with DCM (×3) and the combined extract was dried (MgSO₄) and concentrated, purified by thin-layer chromatography (CH₂Cl₂:MeOH=40:1) to give title compound. MS (m/z): 436 (M+H)⁺

### Compound 199

### (S)-azetidin-1-yl(2-(((7-(4-(dimethylamino)phenyl)pyrido[3,4-b]pyrazin-5-yl)oxy)methyl)morpholino)methanone

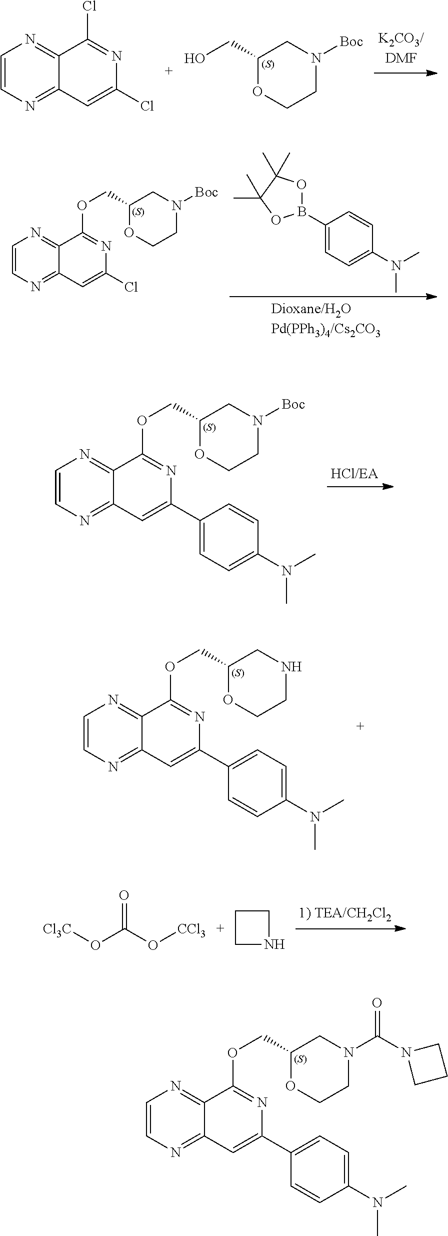

### (A) (S)—N,N-dimethyl-4-(5-(morpholin-2-ylmethoxy)pyrido[3,4-b]pyrazin-7-yl)aniline

The title compound was prepared according to the procedures of Compound 60(A) (C).

### (B)(S)-azetidin-1-yl(2-(((7-(4-(dimethylamino)phenyl)pyrido[3,4-b]pyrazin-5-yl)oxy)methyl)morpholino)methanone

To a solution of bis(trichloromethyl) carbonate (71.2 mg, 0.24 mmol) in CH₂Cl₂ (5 mL) was dropped a solution of (S)—N,N-dimethyl-4-(5-(morpholin-2-ylmethoxy)pyrido[4,3-b]pyrazin-7-yl)aniline (73 mg, 0.2 mmol) and TEA (84 uL, 0.6 mmol) in CH₂Cl₂ (5 mL) at 0° C. The mixture was stirred at 0° C. for 0.5 hours. TLC showed the compound (A) had disappeared, and then azetidine was added and the mixture was stirred at 20° C. for 18 hours. The reaction mixture was washed with sat. aq. NaHCO₃ (5 mL), H₂O (5 mL) and brine (5 mL), dried over Na₂SO₄ and concentrated, purified by thin-layer chromatography (CH₂Cl₂:MeOH=50:1) to give title compound. MS (m/z): 449 (M+H)⁺

### Compound 206

### (S)-4-(((7-(4-(dimethylamino)phenyl)pyrido[3,4-b]pyrazin-5-yl)oxy)methyl)pyrrolidin-2-one

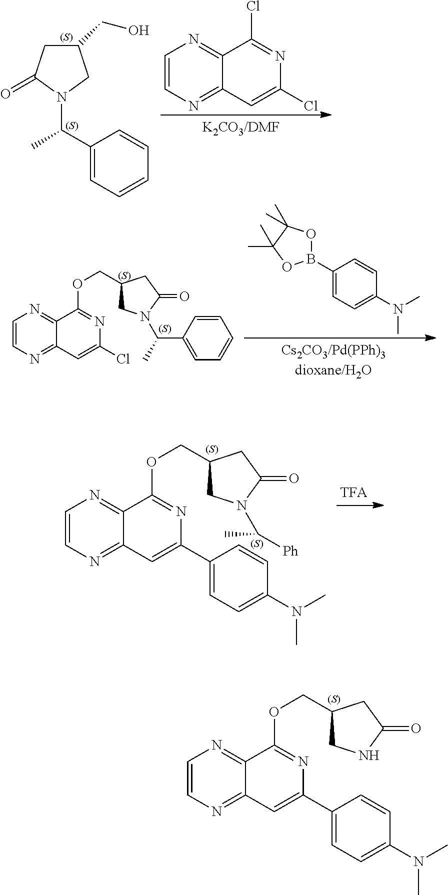

### (A) (S)-4-(((7-(4-(dimethylamino)phenyl)pyrido[3,4-b]pyrazin-5-yl)oxy)methyl)-1-((S)-1-phenylethyl)pyrrolidin-2-one

The title compound was prepared according to the procedures of Compound 2. MS (m/z): 468 (M+H)⁺

### (B) (S)-4-(((7-(4-(dimethylamino)phenyl)pyrido[3,4-b]pyrazin-5-yl)oxy)methyl)pyrrolidin-2-one

(S)-4-(((7-(4-(dimethylamino)phenyl)pyrido[3,4-b]pyrazin-5-yl)oxy)methyl)-1-((S)-1-phenylethyl)pyrrolidin-2-one (46.8 mg, 0.1 mmol) was dissolved in TFA (2 mL) in tube. The tube was sealed and heated in a microwave reactor at 150° C. for 75 minutes. After cooling the reaction mixture was concentrated and the residue was dissolved in DCM (10 mL). The organic phase was washed with sat. aq. NaHCO₃, water, and brine, dried and concentrated to give crude product, which was purified by thin-layer chromatography (DCM:MeOH=40:1) to give title compound. MS (m/z): 364 (M+H)⁺

The following compounds were prepared according to the procedures of Compound 206 using the corresponding intermediates and reagents under appropriate conditions that will be recognized by one skilled in the art.

| Com- | | MS |
|---|---|---|
| pound | Structure | (M + H)⁺ |
| 223 | 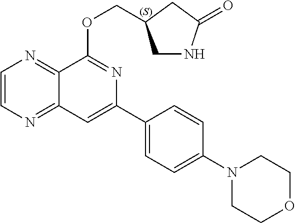 | 406 |
| 224 | 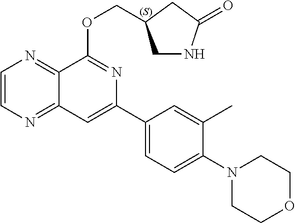 | 420 |
| 225 | 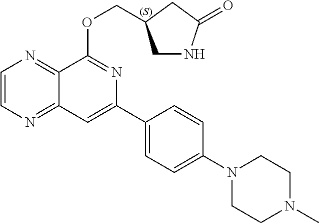 | 419 |
| 226 | 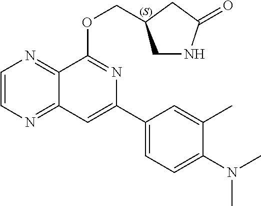 | 378 |
| 232 | 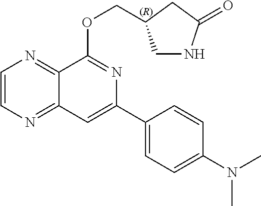 | 364 |
| 243 | 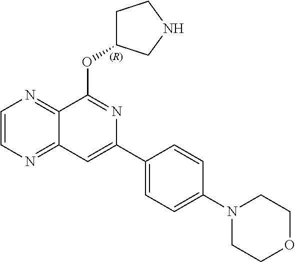 | 378 |

### Compound 213

### 1-(4-(5-(((S)-4-(methylsulfonyl)morpholin-2-yl)methoxy)pyrido[3,4-b]pyrazin-7-yl)phenyl)ethanol

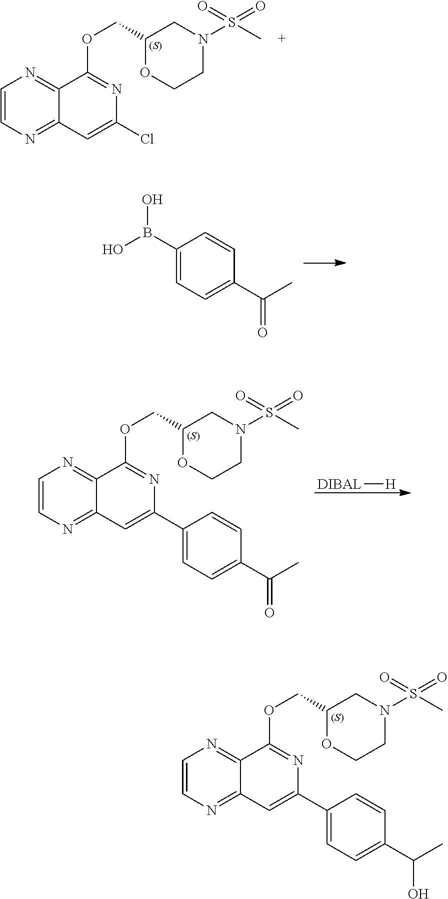

### (A) (S)-1-(4-(5-((4-(methylsulfonyl)morpholin-2-yl)methoxy)pyrido[3,4-b]pyrazin-7-yl)phenyl)ethanone

The title compound was prepared according to the procedures of Compound 6. MS (m/z): 443 (M+H)⁺

### (B) 1-(4-(5-(((S)-4-(methylsulfonyl)morpholin-2-yl)methoxy)pyrido[3,4-b]pyrazin-7-yl)phenyl)ethanol

To a solution of (S)-1-(4-(5-((4-(methylsulfonyl)morpholin-2-yl)methoxy)pyrido[3,4-b]pyrazin-7-yl)phenyl)ethanone (45 mg, 0.10 mmol) in DCM (5 mL) was added DIBAL-H (0.11 mL, 0.11 mmol) at −78° C. under N₂ atmosphere. The mixture was stirred for 30 minutes at −78° C. The mixture was quenched with saturated aqueous solution of ammonium chloride (1 mL), and the reaction solution was partitioned between water (10 mL) and DCM (20 mL). The organic phase was dried over Na₂SO₄, concentrated in vacuo, and the residue purified by flash column chromatography (MeOH:H₂O=0:1 to 10:1) to give 25 mg of title compound as white solid. MS (m/z)=445 [M+H]⁺;

### Compound 241

### (S)-2-(((7-(4-(tetrahydro-2H-pyran-4-yl)phenyl)pyrido[3,4-b]pyrazin-5-yl)oxy)methyl)morpholine-4-carboxamide

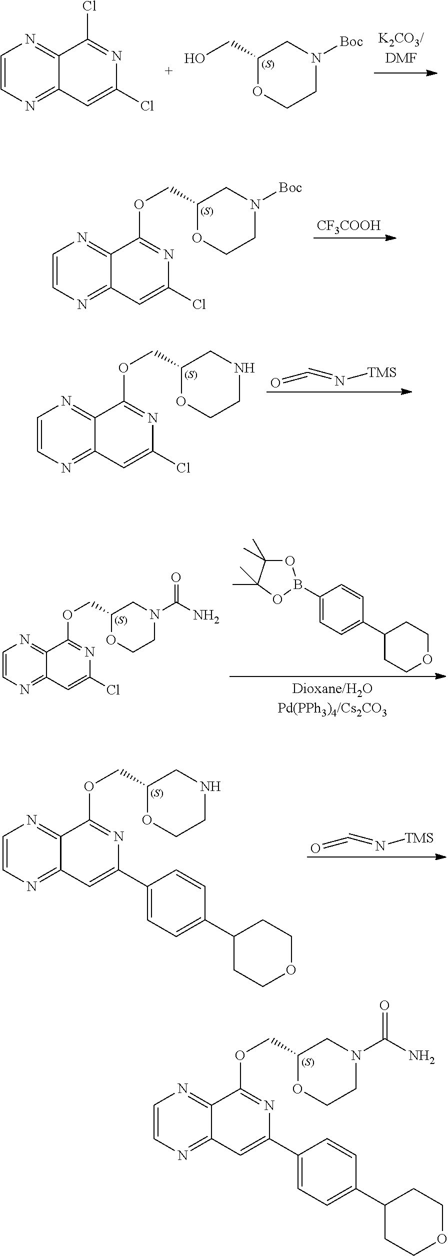

### (A) (S)-tert-butyl 2-(((7-chloropyrido[3,4-b]pyrazin-5-yl)oxy)methyl)morpholine-4-carboxylate

The title compound was prepared according to the procedures of Compound 60 (A).

### (B) (S)-2-(((7-chloropyrido[3,4-b]pyrazin-5-yl)oxy)methyl)morpholine

The title compound was prepared according to the procedures of Compound 142 (B). MS (m/z): 281 (M+H)⁺

### (C) (S)-2-(((7-chloropyrido[3,4-b]pyrazin-S-yl)oxy)methyl)morpholine-4-carboxamide

The title compound was prepared according to the procedures of Compound 142 (C). MS (m/z): 324 (M+H)⁺

### (D) (S)-2-(((7-(4-(tetrahydro-2H-pyran-4-yl)phenyl)pyrido[3,4-b]pyrazin-5-yl)oxy)methyl)morpholine

The title compound was prepared according to the procedures of Compound 142 (A).

### (E) (S)-2-(((7-(4-(tetrahydro-2H-pyran-4-yl)phenyl)pyrido[3,4-b]pyrazin-5-yl)oxy)methyl)morpholine-4-carboxamide

The title compound was prepared according to the procedures of Compound 142 (C). MS (m/z): 450 (M+H)⁺

The following compounds were prepared according to the procedures of Compound 241 using the corresponding intermediates and reagents under appropriate conditions that will be recognized by one skilled in the art.

| Compound | Structure | MS (M + H)⁺ |
|---|---|---|
| 242 | 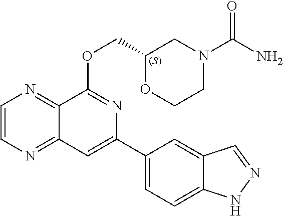 | 406 |

### Compound 261

### (S)-4-(((7-(4-(1-(methylsulfonyl)piperidin-4-yl)phenyl)pyrido[3,4-b]pyrazin-5-yl)oxy)methyl)pyrrolidin-2-one

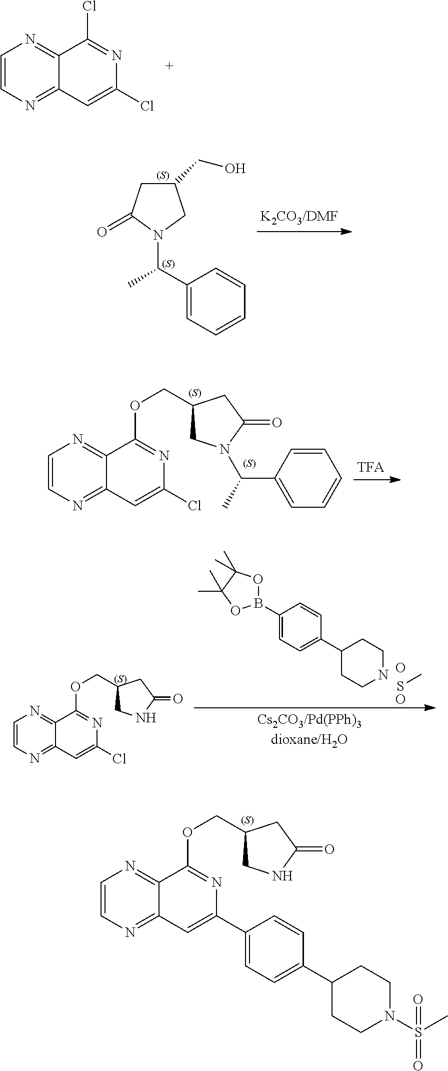

### (A) (S)-4-(((7-chloropyrido[3,4-b]pyrazin-5-yl)oxy)methyl)-1-((S)-1-phenylethyl)pyrrolidin-2-one

The title compound was prepared according to the procedures of Compound 2 (A). MS (m/z): 383 (M+H)⁺

### (B) (S)-4-(((7-chloropyrido[3,4-b]pyrazin-5-yl)oxy)methyl)pyrrolidin-2-one

The title compound was prepared according to the procedures of Compound 206 (C). MS (m/z): 279 (M+H)⁺

### (C) (S)-4-(((7-(4-(1-(methylsulfonyl)piperidin-4-yl)phenyl)pyrido[3,4-b]pyrazin-5-yl)oxy)methyl)pyrrolidin-2-one

The title compound was prepared according to the procedures of Compound 2 (B). MS (m/z): 482 (M+H)⁺

### Compound 277

### (S)-4-(methylsulfonyl)-2-(((7-(4-(prop-1-en-2-yl)phenyl)pyrido[3,4-b]pyrazin-5-yl)oxy)methyl)morpholine

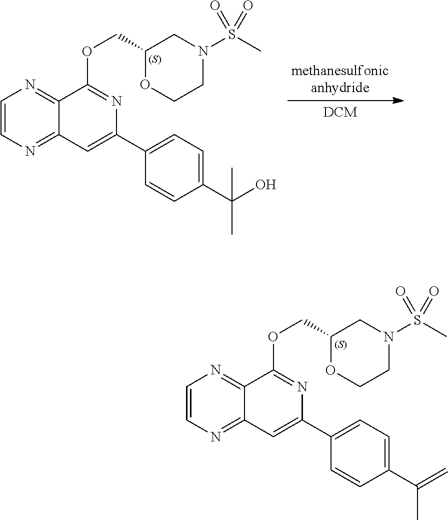

To a solution of compound 219 (25 mg, 0.05 mmol) in DCM (10 mL) was added Et₃N (22 mg, 0.22 mmol) and methanesulfonic anhydride (20 mg, 0.11 mmol) at 0° C. The mixture was stirred at room temperature for 1 hour. The mixture was concentrated in vacuo and the residue was purified by flash column chromatography (MeOH:H₂O=0:1 to 10:1) to give 15 mg of product as yellow solid. MS (m/z)=441 (M+H)⁺

### Compound 292

### (S)-2-(((7-(3,4-dimethoxyphenyl)pyrido[3,4-b]pyrazin-5-yl)oxy)methyl)morpholine-4-sulfonamide

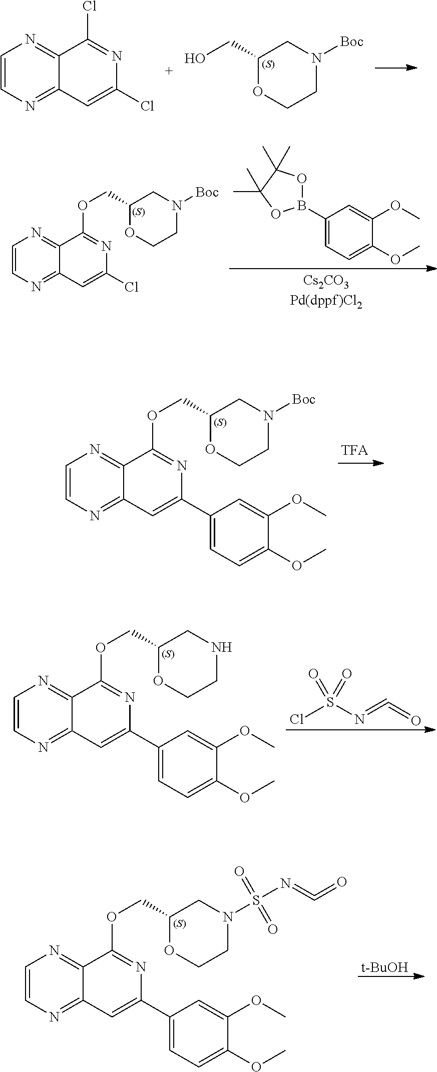

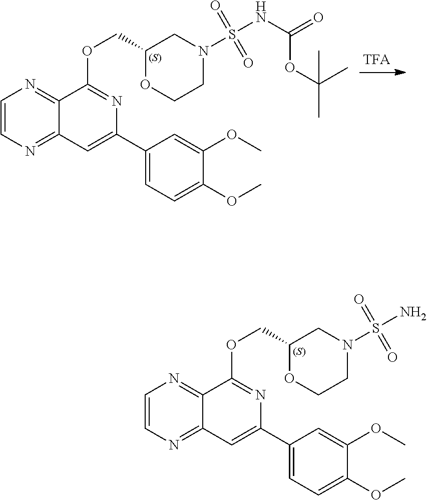

### (A) (S)-tert-butyl 2-(((7-chloropyrido[3,4-b]pyrazin-5-yl)oxy)methyl)morpholine-4-carboxylate

The title compound was prepared according to the procedures of Compound 60 (A).

### (B) (S)-tert-butyl 2-(((7-(3,4-dimethoxyphenyl)pyrido[3,4-b]pyrazin-5-yl)oxy)methyl)morpholine-4-carboxylate

The title compound was prepared according to the procedures of Compound 60 (B) using different catalyst.

### (C) (S)-2-(((7-(3,4-dimethoxyphenyl)pyrido[3,4-b]pyrazin-5-yl)oxy)methyl)morpholine

The title compound was prepared according to the procedures of Compound 60 (C) using different acid. MS (m/z)=383 (M+H)⁺

### (D) (S)-tert-butyl (2-(((7-(3,4-dimethoxyphenyl)pyrido[3,4-b]pyrazin-5-yl)oxy)methyl)morpholino)sulfonylcarbamate

(S)-2-(((7-(3,4-dimethoxyphenyl)pyrido[3,4-b]pyrazin-5-yl)oxy)methyl)morpholine (300 mg, 0.78 mmol) was dissolved in DCM (3 mL). TEA (315 mg, 3.12 mmol) was added, and then sulfuryl chloride isocyanate (220 mg, 1.56 mmol) was added slowly. The mixture was stirred for 3 hours at room temperature. Thent-BuOH (2 mL) was added and the mixture was stirred overnight at room temperature. The mixture was concentrated in vacuum and the residue was used directly in the next step.

### (E) (S)-2-(((7-(3,4-dimethoxyphenyl)pyrido[3,4-b]pyrazin-5-yl)oxy)methyl)morpholine-4-sulfonamide

(S)-tert-butyl (2-(((7-(3,4-dimethoxyphenyl)pyrido[3,4-b]pyrazin-5-yl)oxy)methyl)morpholino)sulfonylcarbamate (437 mg, 0.78 mmol) in DCM (2 mL) was added CF₃COOH (2 mL) and the mixture was stirred for 2 hours at room temperature. The mixture was concentrated in vacuum and the residue was purified by flash column chromatography (DCM/MeOH=100/0 to 100/10) to give the title product. MS (m/z)=462 (M+H)⁺

The following compounds were prepared according to the procedures of Compound 292 using the corresponding intermediates and reagents under appropriate conditions that will be recognized by one skilled in the art.

| Compound | Structure | MS (M + H)⁺ |
|---|---|---|
| 293 | 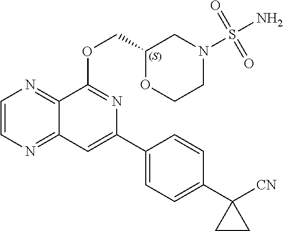 | 467 |
| 294 | 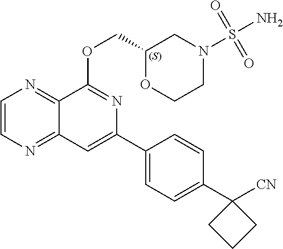 | 481 |

### Compound 295

### (S)-1-(2-(((7-(4-(1-acetylazetidin-3-yl)phenyl)pyrido[3,4-b]pyrazin-5-yl)oxy)methyl)morpholino)ethanone

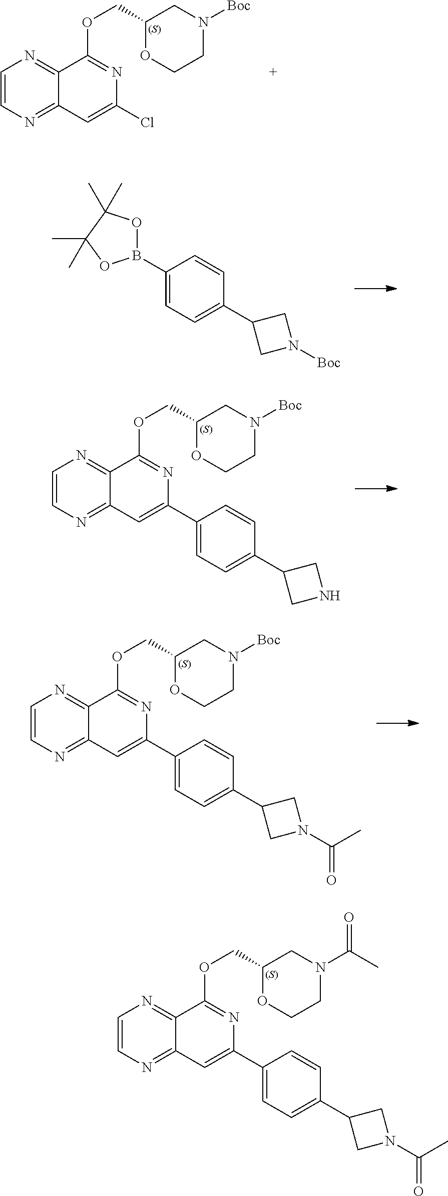

### (A) (S)-tert-butyl 2-(((7-(4-(azetidin-3-yl)phenyl)pyrido[3,4-b]pyrazin-5-yl)oxy)methyl)morpholine-4-carboxylate

tert-butyl 3-(4-(4,4,5,5-tetramethyl-1,3,2-dioxaborolan-2-yl)phenyl)azetidine-1-carboxylate (0.83 g, 2.3 mmol) was dissolved in 3N HCl in acetate (15 mL), and the mixture was stirred at room temperature for 3 hours until TLC indicated Boc group was removed. The volatile materials were removed in vacuo. To the residue was added (S)-tert-butyl 2-(((7-chloropyrido[3,4-b]pyrazin-5-yl)oxy)methyl)morpholine-4-carboxylate (0.95 g, 2.5 mmol), Pd(dppf)Cl₂ (169 mg, 0.23 mmol), Cs₂CO₃ (2.25 g, 6.9 mmol) and dioxane/H₂O (30 mL/3 mL). The reaction mixture was heated at 90° C. overnight. The mixture was cooled to room temperature, concentrated and purified by silica-gel column chromatography eluting with EtOAc/methanol (gradient) to afford title compound 1.03 g. MS (m/z): 478 (M+H)⁺.

### (B) (S)-tert-butyl 2-(((7-(4-(1-acetylazetidin-3-yl)phenyl)pyrido[3,4-b]pyrazin-5-yl)oxy)methyl)morpholine-4-carboxylate

To the solution of (S)-tert-butyl 2-(((7-(4-(azetidin-3-yl)phenyl)pyrido[3,4-b]pyrazin-5-yl)oxy)methyl)morpholine-4-carboxylate (382 mg, 0.80 mmol) in dichloromethane (15 mL) was added triethylamine (242 mg, 2.40 mmol) and acetyl chloride (94 mg, 1.20 mmol). The resulting mixture was stirred at room temperature for 16 hours. The reaction mixture was diluted with dichloromethane (20 mL) and washed with saturated aqueous sodium bicarbonate solution. The layers were separated, and the aqueous layer was extracted further with dichloromethane (15 mL). The combined organic layers were washed with brine, dried (Na₂SO₄), and concentrated in vacuo to afford product 416 mg. MS (m/z): 420 (M+H-Boc)⁺.

### (C) (S)-1-(2-(((7-(4-(1-acetylazetidin-3-yl)phenyl)pyrido[3,4-b]pyrazin-5-yl)oxy)methyl)morpholino)ethanone

(S)-tert-butyl 2-(((7-(4-(1-acetylazetidin-3-yl)phenyl)pyrido[3,4-b]pyrazin-5-yl)oxy)methyl)morpholine-4-carboxylate (208 mg, 0.40 mmol) was dissolved in 3N HCl in acetate (15 mL), and the mixture was stirred at room temperature for 1 hour until TLC indicated Boc group was removed. The volatile materials were removed in vacuo and the residue was dissolved in dichloromethane (15 mL). To the resulted solution was added triethylamine (120 mg, 1.20 mmol) and acetyl chloride (47 mg, 0.60 mmol). The resulting mixture was stirred at room temperature for 16 hours. The reaction mixture was concentrated under reduced pressure and purified using C₁₈ column chromatography to give title compound as pale yellow solid. MS (m/z): 462 (M+H)⁺.

The following compounds were prepared according to the procedures of Compound 295 using the corresponding intermediates and reagents under appropriate conditions that will be recognized by one skilled in the art.

| Com- | | MS |
|---|---|---|
| pound | Structure | (M + H)⁺ |
| 296 | 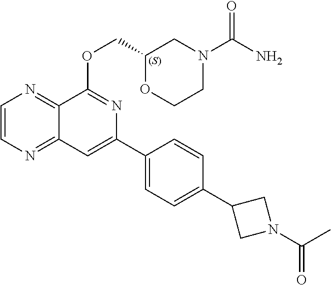 | 463 |

### Compound 303

### (S)-4-methyl-6-(((7-(4-morpholinophenyl)pyrido[3,4-b]pyrazin-5-yl)oxy)methyl)morpholin-3-one

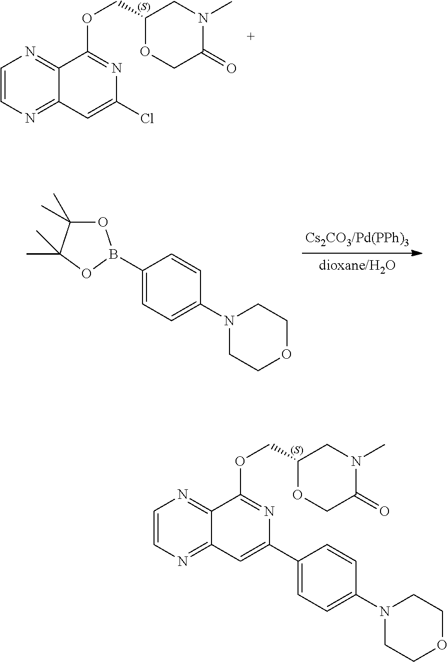

The title compound was prepared according to the procedures of Compound 60 (B). MS (m/z): 436 (M+H)⁺

The following compounds were prepared according to the procedures of Compound 303 using the corresponding intermediates and reagents under appropriate conditions that will be recognized by one skilled in the art.

| Com- | | MS |
|---|---|---|
| pound | Structure | (M + H)⁺ |
| 304 | 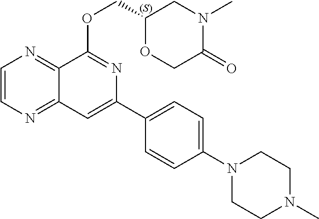 | 449 |
| 305 | 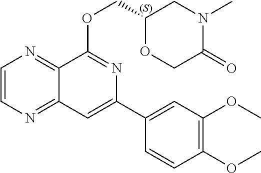 | 411 |
| 306 | 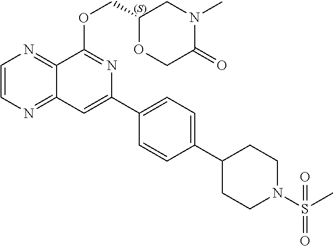 | 512 |
| 307 | 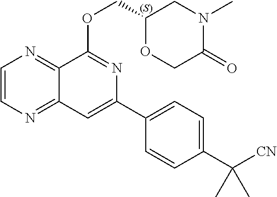 | 418 |
| 310 | 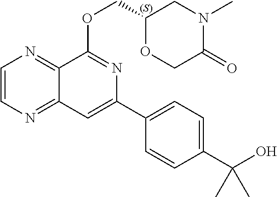 | 431 (M + Na) |

¹H-NMR data of some compounds are provided:

| Compound | 1H-NMR |
|---|---|
| 1 | 1H NMR (400 MHz, cdcl3) δ 8.89 (s, 1H), 8.78 (s, 1H), 8.11~8.09 (d, J = 8.0 Hz, 2H), 7.81 (s, |
| | 1H), 7.04~7.02 (d, J = 8.4 Hz, 2H), 5.58~5.51 (m, 1H), 3.91~3.81 (m, 5H), 3.30~3.28 (t, |
| | J = 4.8 Hz, 4H), 2.42~2.37 (m, 2H), 2.17~2.13 (d, J = 17.1 Hz, 2H), 1.89~1.79 (m, 2H), 1.65~1.55 |
| | (m, 2H). |
| 2 | 1H NMR (400 MHz, cdcl3) δ 8.97~8.94 (dd, J = 1.6 Hz, 12.0 Hz, 2H), 8.63 (s, 1H), 8.19~8.17 (m, |
| | 3H), 7.90 (s, 1H), 7.04~7.02 (d, J = 9.2 Hz, 2H), 3.93~3.89 (m, 6H), 3.31~3.28 (t, J = 4.8 Hz, |
| | 4H), 2.92~2.89 (t, J = 6.0 Hz, 2H). |
| 3 | 1H NMR (400 MHz, dmso) δ 9.13 (d, J = 1.2, 1H), 8.96 (d, J = 1.1, 1H), 8.09 (s, 1H), 8.03 (d, J = |
| | 8.4, 3H), 7.87 (d, J = 8.7, 2H), 7.45 (d, J = 8.3, 2H), 7.39 (s, 1H), 6.98 (d, J = 8.8, 2H), 3.75- |
| | 3.69 (m, 4H), 3.21-3.17 (m, 4H). |
| 4 | 1H NMR (400 MHz, cdcl3) δ 8.93 (s, 1H), 8.79 (s, 1H), 8.11~8.08 (d, J = 8.8 Hz, 2H), 7.86 (s, |
| | 1H), 7.04~7.02 (d, J = 8.8 Hz, 2H), 5.75 (s, 1H), 4.80~4.67 (m, 2H), 3.91~3.89 (t, J = 4.4 Hz, |
| | 4H), 3.67~3.65 (d, J = 10.4 Hz, 1H), 3.37~3.28 (m, 5H), 2.72~2.64 (m, 1H), 2.60~2.42 (m, 2H), |
| | 2.19~2.13 (m, 1H), 1.86~1.76 (m, 1H). |
| 5 | 1H NMR (400 MHz, cdcl3) δ 8.91~8.90 (d, J = 1.6 Hz, 1H), 8.77~8.76 (d, J = 2.0 Hz, 1H), |
| | 8.08~8.06 (d, J = 8.8 Hz, 2H), 7.83 (s, 1H), 7.04~7.02 (d, J = 8.8 Hz, 2H), 5.97 (s, 1H), |
| | 4.78~4.65 (m, 2H), 3.68~3.62 (m, 1H), 3.36~3.33 (t, J = 6.4 Hz, 4H), 2.71~2.58 (m, 5H), |
| | 2.57~2.41 (m, 3H), 2.36 (s, 3H), 2.17~2.13 (m, 1H), 1.84~1.74 (m, 1H). |
| 6 | 1H NMR (400 MHz, cdcl3) δ 8.99 (s, 1H), 8.85 (s, 1H), 8.16 (d, J = 8.7, 2H), 7.93 (d, J = 3.7, |
| | 1H), 7.10 (d, J = 6.4, 2H), 6.19 (t, J = 53.8, 1H), 5.00-4.81 (m, 2H), 4.54 (dd, J = 107.0, 13.3, |
| | 1H), 4.36-4.00 (m, 3H), 3.83-3.70 (m, 1H), 3.55-3.42 (m, 1H), 3.40-3.32 (m, 4H), 3.19- |
| | 3.13 (m, 4H), 3.13-3.01 (m, 1H). |
| 7 | 1H NMR (400 MHz, cdcl3) δ 8.95 (s, 1H), 8.83 (s, 1H), 7.93-7.83 (m, 3H), 7.04 (t, J = 8.5, 1H), |
| | 4.81 (ddd, J = 53.4, 11.5, 5.4, 2H), 4.32-4.19 (m, 1H), 4.12-4.06 (m, 2H), 3.96-3.86 (m, |
| | 5H), 3.81 (td, J = 11.3, 2.2, 1H), 3.62 (d, J = 11.7, 1H), 3.25-3.15 (m, 4H), 2.98-2.91 (m, 3H), |
| | 2.91-2.84 (m, 1H), 2.82 (s, 3H). |
| 8 | 1H NMR (400 MHz, cdcl3) δ 8.96 (s, 1H), 8.84 (s, 1H), 8.10 (d, J = 8.1, 2H), 7.95 (s, 1H), 7.39 |
| | (d, J = 8.1, 2H), 4.82 (ddd, J = 50.5, 11.5, 5.4, 2H), 4.32-4.18 (m, 1H), 4.09 (d, J = 11.7, 1H), |
| | 3.90 (d, J = 11.5, 1H), 3.80 (td, J = 11.5, 2.4, 1H), 3.61 (d, J = 11.4, 1H), 3.16 (d, J = 11.4, 2H), |
| | 2.94 (td, J = 11.8, 3.5, 1H), 2.90-2.84 (m, 1H), 2.81 (s, 3H), 2.69-2.56 (m, 1H), 2.45 (s, 3H), |
| | 2.27 (t, J = 11.0, 2H), 2.06-1.88 (m, 5H). |
| 9 | 1H NMR (400 MHz, cdcl3) δ 8.95 (s, 1H), 8.84 (s, 1H), 8.12~8.10 (d, J = 7.2 Hz, 2H), 7.95~7.94 |
| | (d, J = 3.6 Hz, 1H), 7.39~7.37 (d, J = 7.2 Hz, 2H), 6.25~5.98 (t, J = 53.6 Hz, 1H), 4.88~4.78 (m, |
| | 2H), 4.61~4.31 (dd, J = 13.2 Hz, 107.2 Hz, 1H), 4.25~3.96 (m, 5H), 3.73~3.65 (m, 1H), |
| | 3.60~3.54 (t, J = 11.2 Hz, 2H), 3.46~3.36 (m, 1H), 3.09~2.98 (m, 1H), 2.89~2.81 (m, 1H), |
| | 1.94~1.77 (m, 4H). |
| 10 | 1H NMR (400 MHz, cdcl3) δ 8.99 (d, J = 1.8, 1H), 8.87 (d, J = 1.8, 1H), 8.14 (d, J = 8.3, 2H), |
| | 7.98 (s, 1H), 7.42 (d, J = 8.3, 2H), 4.86 (ddd, J = 48.8, 11.5, 5.4, 2H), 4.34-4.24 (m, 1H), 4.13 |
| | (dd, J = 11.7, 1.8, 1H), 3.94 (d, J = 11.5, 1H), 3.84 (td, J = 11.4, 2.6, 1H), 3.64 (d, J = 10.7, 1H), |
| | 3.29 (d, J = 12.0, 2H), 3.01-2.94 (m, 1H), 2.94-2.86 (m, 2H), 2.85-2.80 (m, 4H), 2.79-2.71 |
| | (m, 1H), 1.93 (d, J = 12.2, 2H), 1.79-1.71 (m, 2H). |
| 11 | 1H NMR (400 MHz, cdcl3) δ 9.03 (d, J = 1.7, 1H), 8.91 (d, J = 1.8, 1H), 8.18 (d, J = 8.3, 2H), |
| | 8.02 (s, 1H), 7.45 (d, J = 8.3, 2H), 4.90 (ddd, J = 49.6, 11.6, 5.5, 2H), 4.37-4.29 (m, 1H), 4.24- |
| | 4.12 (m, 3H), 3.97 (d, J = 11.5, 1H), 3.87 (td, J = 11.5, 2.5, 1H), 3.71-3.59 (m, 3H), 3.01 (td, |
| | J = 11.6, 3.3, 1H), 2.97-2.90 (m, 2H), 2.88 (s, 3H), 2.02-1.85 (m, 4H). |
| 12 | 1H NMR (400 MHz, dmso) δ 9.03 (s, 1H), 8.84 (s, 1H), 8.12 (d, J = 8.5 Hz, 2H), 7.93 (s, 1H), |
| | 7.03 (d, J = 8.7 Hz, 2H), 4.71-4.55 (m, 2H), 3.98 (d, J = 12.4 Hz, 2H), 3.74 (d, J = 17.5 Hz, |
| | 4H), 3.62 (dd, J = 24.1, 11.4 Hz, 3H), 3.19 (s, 4H), 2.90 (s, 3H), 2.87-2.76 (m, 2H). |
| 13 | 1H NMR (400 MHz, dmso) δ 9.19 (d, J = 1.9 Hz, 1H), 9.01 (d, J = 1.9 Hz, 1H), 8.26 (d, J = 9.0 |
| | Hz, 2H), 8.08 (s, 1H), 7.18 (d, J = 9.0 Hz, 2H), 4.81 (qd, J = 11.5, 5.3 Hz, 2H), 4.21-4.11 (m, |
| | 2H), 3.84-3.72 (m, 2H), 3.56 (d, J = 4.8 Hz, 1H), 3.41-3.36 (m, 4H), 3.07 (s, 3H), 3.03-2.95 |
| | (m, 2H), 2.58 (d, J = 5.0 Hz, 4H), 2.36 (s, 3H). |
| 14 | 1H NMR (400 MHz, dmso) δ 9.51-9.47 (m, 1H), 9.32-9.29 (m, 1H), 8.56 (d, J = 8.8 Hz, 2H), |
| | 8.37 (s, 1H), 7.47 (d, J = 8.9 Hz, 2H), 5.17-5.06 (m, 2H), 4.45 (d, J = 10.9 Hz, 2H), 4.15-4.03 |
| | (m, 2H), 3.85 (s, 1H), 3.64-3.58 (m, 4H), 3.38 (s, 3H), 3.32 (dd, J = 7.3, 4.2 Hz, 1H), 3.28 (d, |
| | J = 5.2 Hz, 5H). |
| 15 | 1 H NMR (400 MHz, cdcl 3) δ 9.12 (s, 1H), 9.01 (s, 1H), 8.05 (d, J = 6.4, 3H), 7.21 (t, J = 8.2, |
| | 1H), 6.29 (t, J = 53.7, 1H), 4.99 (d, J = 14.6, 2H), 4.81-4.12 |
| | (m, 5H), 4.08 (s, 4H), 3.88 (s, 1H), 3.58 (dd, J = 26.5, 14.1, 1H), 3.37 (s, 4H), 3.29-3.12 (m, |
| | 1H). |
| 16 | 1H NMR (400 MHz, cdcl3) δ 8.91 (d, J = 1.7, 1H), 8.78 (d, J = 1.2, 1H), 8.08 (d, J = 8.7, 2H), |
| | 7.85 (d, J = 3.6, 1H), 7.03 (dd, J = 8.7, 1.8, 2H), 6.12 (t, J = 53.6, 1H), 4.93-4.72 (m, 2H), 4.46 |
| | (dd, J = 106.6, 13.0, 1H), 4.27-3.92 (m, 3H), 3.74-3.62 (m, 1H), 3.50-3.38 (m, 1H), 3.37- |
| | 3.31 (m, 4H), 3.04 (dt, J = 23.5, 11.7, 1H), 2.65-2.54 (m, 4H), 2.38 (s, 3H). |
| 17 | 1H NMR (400 MHz, cdcl3) δ 8.91 (dd, J = 3.5, 1.8, 1H), 8.78 (t, J = 1.5, 1H), 8.08 (dd, J = 8.9, |
| | 1.9, 2H), 7.85 (d, J = 3.8, 1H), 7.03 (dd, J = 9.0, 2.8, 2H), 6.11 (t, J = 53.4, 1H), 4.96-4.72 (m, |
| | 2H), 4.46 (dd, J = 106.3, 13.1, 1H), 4.27-3.92 (m, 3H), 3.77-3.59 (m, 1H), 3.50-3.38 (m, |
| | 1H), 3.38-3.31 (m, 4H), 3.13-2.93 (m, 1H), 2.70-2.58 (m, 4H), 2.50 (q, J = 7.2, 2H), 1.15 (t, |
| | J = 7.2, 3H). |
| 18 | 1H NMR (400 MHz, cdcl3) δ 8.92~8.91 (d, J = 2.0 Hz, 1H), 8.79~8.78 (d, J = 2.0 Hz, 1H), |
| | 8.10~8.08 (d, J = 8.8 Hz, 2H), 7.86 (s, 1H), 7.03~7.00 (d, J = 8.8 Hz, 2H), 4.90~4.74 (m, 2H), |
| | 4.30~4.23 (m, 1H), 4.12~4.03 (m, 2H), 3.92~3.89 (d, J = 11.6 Hz, 1H), 3.85~3.76 (m, 3H), |
| | 3.62~3.54 (m, 3H), 2.97~2.83 (m, 3H), 2.81 (s, 3H), 2.62~2.57 (t, J = 11.6 Hz, 1H), 1.29~1.28 |
| | (d, J = 6.0 Hz, 3H). |
| 19 | 1H NMR (400 MHz, cdcl3) δ 8.92~8.91 (d, J = 2.0 Hz, 1H), 8.79~8.78 (d, J = 2.0 Hz, 1H), |
| | 8.09~8.07 (d, J = 8.8 Hz, 2H), 7.86 (s, 1H), 7.02~7.00 (d, J = 8.8 Hz, 2H), 4.90~4.74 (m, 2H), |
| | 4.29~4.23 (m, 1H), 4.11~4.08 (d, J = 11.6 Hz, 1H), 3.92~3.89 (d, J = 11.9 Hz, 1H), 3.85~3.77 |
| | (m, 3H), 3.62~3.59 (d, J = 12.0 Hz, 3H), 2.97~2.81 (m, 5H), 2.55~2.49 (t, J = 11.6 Hz, 2H), |
| | 1.30~1.29 (d, J = 6.0 Hz, 6H). |
| 20 | 1H NMR (400 MHz, cdcl3) δ 8.93~8.92 (d, J = 2.0 Hz, 1H), 8.80~8.79 (d, J = 2.0 Hz, 1H), |
| | 8.10~8.07 (d, J = 9.2 Hz, 2H), 7.86 (s, 1H), 7.06~7.04 (d, J = 9.2 Hz, 2H), 4.90~4.73 (m, 2H), |
| | 4.29~4.23 (m, 1H), 4.11~4.08 (d, J = 11.6 Hz, 1H), 3.92~3.89 (d, J = 11.2 Hz, 1H), 3.83~3.77 |
| | (m, 1H), 3.62~3.59 (d, J = 10.4 Hz, 1H), 3.52~3.48 (m, 3H), 2.97~2.82 (m, 6H), 2.17~2.07 (m, |
| | 4H). |
| 21 | 1H NMR (400 MHz, cdcl3) δ 8.91~8.90 (d, J = 1.6 Hz, 1H), 8.77~8.76 (d, J = 2.0 Hz, 1H), |
| | 8.07~8.05 (d, J = 8.8 Hz, 2H), 7.84 (s, 1H), 7.03~7.01 (d, J = 9.2 Hz, 2H), 4.90~4.74 (m, 2H), |
| | 4.29~4.23 (m, 1H), 4.11~4.08 (d, J = 10.0 Hz, 1H), 3.91~3.88 (d, J = 12.0 Hz, 1H), 3.83~3.77 |
| | (m, 1H), 3.62~3.59 (d, J = 12.0 Hz, 1H), 3.33~3.30 (t, J = 5.2 Hz, 4H), 2.97~2.90 (m, 1H), 2.89~ |
| | 2.83 (t, J = 10.4 Hz, 1H), 2.81 (s, 3H), 1.76~1.69 (m, 4H), 1.66~1.62 (m, 2H). |
| 22 | 1H NMR (400 MHz, cdcl3) δ 8.91~8.90 (d, J = 2.0 Hz, 1H), 8.77~8.76 (d, J = 2.0 Hz, 1H), |
| | 8.08~8.06 (d, J = 8.8 Hz, 2H), 7.84 (s, 1H), 7.05~7.03 (d, J = 9.2 Hz, 2H), 4.90~4.74 (m, 2H), |
| | 4.30~4.23 (m, 1H), 4.11~4.08 (d, J = 13.6 Hz, 1H), 3.96~3.88 (m, 3H), 3.83~3.77 (m, 1H), |
| | 3.62~3.59 (d, J = 12.4 Hz, 1H), 2.97~2.75 (m, 7H), 1.91~1.89 (d, J = 9.2 Hz, 2H), 1.24~1.23 (m, |
| | 10H). |
| 23 | 1H NMR (400 MHz, cdcl3) δ 8.97 (dd, J = 9.2, 1.8, 1H), 8.85 (t, J = 1.9, 1H), 8.12 (d, J = 8.2, |
| | 2H), 7.96 (d, J = 10.6, 1H), 7.37 (d, J = 8.1, 2H), 4.92-4.73 (m, 2H), 4.56 (dd, J = 108.6, 12.4, |
| | 1H), 4.15-4.06 (m, 1H), 4.06-3.57 (m, 4H), 3.42-3.25 (m, 1H), 3.02 (q, J = 7.4, 2H), 2.98- |
| | 2.78 (m, 3H), 2.77-2.67 (m, 1H), 2.12 (d, J = 7.5, 3H), 1.99 (d, J = 11.2, 2H), 1.87 (qd, J = |
| | 12.7, 4.0, 2H), 1.42 (t, J = 7.4, 3H). |
| 24 | 1H NMR (400 MHz, cdcl3) δ 8.92 (dd, J = 9.1, 1.8, 1H), 8.84-8.75 (m, 1H), 8.11 (d, J = 8.7, |
| | 2H), 7.86 (d, J = 10.1, 1H), 7.02 (dd, J = 9.0, 2.9, 2H), 4.92-4.72 (m, 2H), 4.55 (dd, J = 107.7, |
| | 13.4, 1H), 4.14-4.06 (m, 1H), 4.06-3.58 (m, 1H), 3.41-3.25 (m, 5H), 2.96-2.76 (m, 1H), |
| | 2.11 (d, J = 6.7, 3H). |
| 25 | 1H NMR (400 MHz, cdcl3) δ 8.93 (dd, J = 9.3, 1.8, 1H), 8.81 (t, J = 1.5, 1H), 8.11 (d, J = 8.8, |
| | 2H), 7.87 (d, J = 10.5, 1H), 7.04 (dd, J = 9.0, 2.6, 2H), 4.92-4.72 (m, 2H), 4.56 (dd, J = 110.1, |
| | 13.3, 1H), 4.10 (dt, J = 15.8, 8.0, 1H), 4.06 (s, 1H), 3.99-3.59 (m, 2H), 3.55-3.45 (m, 4H), |
| | 3.43-3.36 (m, 5H), 3.30 (dd, J = 13.1, 10.5, 1H), 3.02 (q, J = 7.4, 2H), 2.96-2.76 (m, 1H), |
| | 2.12 (d, J = 6.9, 3H), 1.42 (t, J = 7.4, 3H). |
| 26 | 1H NMR (400 MHz, cdcl3) δ 8.94~8.93 (d, J = 1.6 Hz, 1H), 8.81~8.80 (d, J = 2.0 Hz, 1H), |
| | 8.11~8.09 (d, J = 8.8 Hz, 2H), 7.87 (s, 1H), 7.06~7.04 (d, J = 8.8 Hz, 2H), 4.90~4.73 (m, 2H), |
| | 4.29~4.23 (m, 1H), 4.12~4.08 (m, 1H), 3.92~3.88 (m, 1H), 3.83~3.77 (m, 1H), 3.62~3.60 (d, J = |
| | 9.2 Hz, 1H), 3.43 (s, 8H), 2.97~2.91 (m, 1H), 2.89~2.85 (m, 4H), 2.84 (s, 3H). |
| 27 | 1H NMR (400 MHz, cdcl3) δ 8.92~8.91 (d, J = 1.6 Hz, 1H), 8.78~8.77 (d, J = 2.0 Hz, 1H), |
| | 8.09~8.07 (d, J = 8.8 Hz, 2H), 7.85 (s, 1H), 7.04~7.02 (d, J = 9.2 Hz, 2H), 4.90~4.73 (m, 2H), |
| | 4.28~4.23 (m, 1H), 4.11~4.08 (d, J = 13.2 Hz, 1H), 3.91~3.88 (d, J = 11.6 Hz, 1H), 3.83~3.77 |
| | (m, 1H), 3.69~3.67 (t, J = 4.8 Hz, 2H), 3.62~3.59 (d, J = 11.2 Hz, 1H), 3.36~3.33 (t, J = 4.8 Hz, |
| | 4H), 2.97~2.83 (m, 2H), 2.81 (s, 3H), 2.72~2.70 (t, J = 5.2 Hz, 4H), 2.65~2.62 (t, J = 5.2 Hz, 2H). |
| 28 | 1H NMR (400 MHz, cdcl3) δ 8.75 (s, 1H), 8.68 (s, 1H), 8.03~8.01 (d, J = 8.4 Hz, 2H), 7.73 (s, |
| | 1H), 7.02~7.00 (d, J = 8.4 Hz, 2H), 4.98~4.94 (dd, J = 4.8 Hz, 11.2 Hz, 1H), 4.66~4.62 (dd, J = |
| | 4.8 Hz, 11.2 Hz, 1H), 4.24~4.18 (m, 1H), 4.10~4.08 (d, J = 9.6 Hz, 1H), 3.84~3.77 (m, 2H), |
| | 3.54~3.51 (d, J = 12.0 Hz, 1H), 3.33~3.30 (t, J = 4.4 Hz, 4H), 2.97~2.91 (m, 1H), 2.89~2.83 (t, |
| | J = 10.4 Hz, 1H), 1.76~1.70 (m, 4H), 1.66~1.62 (m, 2H). |
| 29 | 1H NMR (400 MHz, cdcl3) δ 8.81~8.80 (d, J = 1.6 Hz, 1H), 8.73~8.72 (d, J = 1.6 Hz, 1H), |
| | 8.07~8.05 (d, J = 9.2 Hz, 2H), 7.78 (s, 1H), 7.06~7.03 (d, J = 9.2 Hz, 2H), 4.98~4.93 (dd, J = |
| | 5.2 Hz, 12.0 Hz, 1H), 4.69~4.65 (dd, J = 4.8 Hz, 11.6 Hz, 1H), 4.25~4.20 (m, 1H), 4.11~4.08 (d, |
| | J = 12.0 Hz, 1H), 3.85~3.77 (m, 2H), 3.54~3.49 (m, 5H), 2.99~2.85 (m, 2H), 2.17~2.08 (m, 4H). |
| 30 | 1H NMR (400 MHz, cdcl3) δ 8.72 (s, 1H), 8.64 (s, 1H), 8.05~8.03 (d, J = 8.4 Hz, 2H), 7.70 (s, |
| | 1H), 6.68~6.66 (d, J = 8.0 Hz, 2H), 5.03~4.99 (dd, J = 4.8 Hz, 11.6 Hz, 1H), 4.91 (s, 2H), |
| | 4.66~4.62 (dd, J = 5.2 Hz, 11.6 Hz, 1H), 4.25~4.20 (m, 1H), 4.12~4.09 (d, J = 12.4 Hz, 1H), |
| | 3.84~3.77 (m, 2H), 3.55~3.52 (d, J = 10.4 Hz, 1H), 3.41~3.38 (m, 4H), 2.99~2.84 (m, 2H), |
| | 2.08~2.05 (m, 4H). |
| 31 | 1H NMR (400 MHz, cdcl3) δ 8.91~8.90 (d, J = 1.6 Hz, 1H), 8.77~8.76 (d, J = 1.6 Hz, 1H), |
| | 8.08~8.05 (d, J = 8.8 Hz, 2H), 7.84 (s, 1H), 7.04~7.02 (d, J = 8.8 Hz, 2H), 4.90~4.74 (m, 2H), |
| | 4.29~4.23 (m, 1H), 4.11~4.07 (m, 1H), 3.92~3.88 (m, 1H), 3.83~3.77 (m, 1H), 3.69~3.59 (m, |
| | 3H), 3.46~3.40 (m, 4H), 3.13~3.06 (m, 2H), 2.97~2.84 (m, 2H), 2.81 (s, 3H), 2.07~2.00 (m, 2H), |
| | 1.78~1.69 (m, 2H). |
| 32 | 1H NMR (400 MHz, cdcl3) δ 8.94~8.93 (dd, J = 1.6 Hz, 3.6 Hz, 1H), 8.80 (s, 1H), 7.86~7.83 (m, |
| | 3H), 6.98~6.93 (m, 1H), 6.76~5.98 (m, 1H), 4.88~4.76 (m, 2H), 4.61~4.32 (dd, J = 13.2 Hz, 103.6 |
| | Hz, 1H), 4.24~3.96 (m, 3H), 3.74~3.66 (m, 1H), 3.47~3.37 (m, 1H), 3.10~3.02 (m, 1H), 2.97 (s, |
| | 6H). |
| 33 | 1H NMR (400 MHz, cdcl3) δ 8.97 (d, J = 1.5, 1H), 8.85 (d, J = 1.4, 1H), 8.11 (d, J = 8.2, 2H), |
| | 7.96 (s, 1H), 7.36 (d, J = 8.3, 2H), 4.75-4.86 (m, 3H), 4.31-4.20 (m, 1H), 4.14-4.05 (m, 1H), |
| | 3.98 (d, J = 13.3, 1H), 3.91 (d, J = 11.5, 1H), 3.81 (td, J = 11.4, 2.5, 1H), 3.61 (d, J = 11.9, 1H), |
| | 3.21 (t, J = 12.0, 1H), 2.96 (dd, J = 11.5, 3.3, 1H), 2.89 (q, J = 4.7, 1H), 2.86-2.83 (m, 1H), |
| | 2.82 (s, 3H), 2.67 (td, J = 12.7, 1.9, 1H), 2.16 (s, 3H), 1.96 (t, J = 13.9, 2H), 1.76-1.67 (m, 2H). |
| 34 | 1H NMR (400 MHz, cdcl3) δ 8.98~8.97 (d, J = 1.6 Hz, 1H), 8.87~8.86 (d, J = 2.0 Hz, 1H), |
| | 8.17~8.15 (d, J = 8.8 Hz, 2H), 7.94 (s, 1H), 7.28~7.25 (d, J = 8.8 Hz, 2H), 6.79~6.42 (t, J = 74.0 |
| | Hz, 1H), 4.90~4.73 (m, 2H), 4.28~4.22 (m, 1H), 4.11~4.09 (d, J = 11.6 Hz, 1H), 3.92~3.89 (d, |
| | J = 11.2 Hz, 1H), 3.84~3.78 (m, 1H), 3.63~3.60 (d, J = 11.6 Hz, 1H), 2.97~2.85 (m, 2H), 2.82 (s, |
| | 3H). |
| 35 | 1H NMR (400 MHz, cdcl3) δ 8.96~8.95 (d, J = 2.0 Hz, 1H), 8.84~8.83 (d, J = 2.0 Hz, 1H), |
| | 7.95~7.88 (m, 3H), 7.11~7.07 (t, J = 8.4 Hz, 1H), 4.90~4.72 (m, 2H), 4.28~4.22 (m, 1H), |
| | 4.12~4.09 (dd, J = 1.6 Hz, 11.6 Hz, 1H), 3.98 (s, 3H), 3.92~3.88 (m, 1H), 3.85~3.78 (m, 1H), |
| | 3.63~3.60 (d, J = 12.0 Hz, 1H), 2.97~2.85 (m, 2H), 2.82 (s, 3H). |
| 36 | 1H NMR (400 MHz, cdcl3) δ 8.90~8.89 (d, J = 2.0 Hz, 1H), 8.75~8.74 (d, J = 2.0 Hz, 1H), |
| | 8.05~8.02 (d, J = 8.8 Hz, 2H), 7.81 (s, 1H), 6.73~6.70 (d, J = 8.8 Hz, 2H), 4.89~4.73 (m, 2H), |
| | 4.29~4.22 (m, 1H), 4.11~4.08 (dd, J = 2.0 Hz, 11.6 Hz, 1H), 3.91~3.88 (d, J = 11.6 Hz, 1H), |
| | 3.83~3.77 (m, 1H), 3.62~3.59 (d, J = 10.8 Hz, 1H), 2.97~2.84 (m, 5H), 2.81 (s, 3H). |
| 37 | 1H NMR (400 MHz, cdcl3) δ 8.88~8.87 (d, J = 1.6 Hz, 1H), 8.73~8.72 (d, J = 1.6 Hz, 1H), |
| | 8.08~8.06 (d, J = 8.8 Hz, 2H), 7.79 (s, 1H), 6.68~8.65 (d, J = 8.8 Hz, 2H), 4.90~4.74 (m, 2H), |
| | 4.29~4.23 (m, 1H), 4.11~4.08 (d, J = 9.6 Hz, 1H), 3.91~3.88 (d, J = 11.6 Hz, 1H), 3.83~3.77 (m, |
| | 1H), 3.62~3.59 (d, J = 11.2 Hz, 1H), 3.40~3.37 (t, J = 6.4 Hz, 4H), 2.97~2.84 (m, 2H), 2.81 (s, |
| | 3H), 2.07~2.04 (t, J = 6.4 Hz, 4H). |
| 38 | 1H NMR (400 MHz, cdcl3) δ 8.94~8.93 (d, J = 1.6 Hz, 1H), 8.81~8.80 (d, J = 1.6 Hz, 1H), |
| | 8.13~8.10 (d, J = 8.8 Hz, 2H), 7.88 (s, 1H), 7.05~7.03 (d, J = 8.8 Hz, 2H), 4.90~4.74 (m, 2H), |
| | 4.28~4.22 (m, 1H), 4.1~4.08 (dd, J = 2.0 Hz, 12.0 Hz, 1H), 3.91~3.88 (m, 4H), 3.84~3.77 (m, |
| | 1H), 3.62~3.59 (d, J = 10.8 Hz, 1H), 2.97~2.85 (m, 2H), 2.82 (s, 3H). |
| 39 | 1H NMR (400 MHz, dmso) δ 9.06 (d, J = 1.7, 1H), 8.88 (d, J = 1.6, 1H), 8.49 (s, 1H), 8.12- |
| | 8.00 (m, 2H), 7.54 (d, J = 8.6, 1H), 7.37 (d, J = 2.9, 1H), 6.55 (d, J = 2.9, 1H), 4.72 (d, J = 3.2, |
| | 2H), 4.06 (s, 1H), 4.01 (d, J = 13.1, 1H), 3.82 (s, 3H), 3.70 (d, J = 11.4, 1H), 3.63 (t, J = 10.2, |
| | 1H), 2.92 (s, 3H), 2.87 (dd, J = 13.4, 8.4, 3H). |
| 40 | 1H NMR (400 MHz, dmso) δ 9.10 (s, 1H), 8.94 (s, 1H), 8.27 (d, J = 8.3, 2H), 8.10 (d, J = 12.1, |
| | 1H), 7.55 (d, J = 8.2, 2H), 4.73-4.61 (m, 2H), 4.01 (d, J = 6.1, 1H), 3.69-3.53 (m, 2H), 2.91 (s, |
| | 5H), 2.85 (dd, J = 19.0, 7.5, 2H). |
| 41 | 1H NMR (400 MHz, dmso) δ 9.18 (d, J = 1.8, 1H), 9.01 (d, J = 1.8, 1H), 8.24 (d, J = 8.2, 2H), |
| | 8.14 (s, 1H), 7.41 (d, J = 8.1, 2H), 4.82-4.71 (m, 2H), 4.17-4.07 (m, 2H), 3.72 (ddd, J = 14.2, |
| | 12.7, 7.1, 2H), 3.01 (s, 3H), 3.00-2.89 (m, 2H), 2.45 (s, 3H). |
| 42 | 1H NMR (400 MHz, dmso) δ 9.01 (d, J = 1.8, 1H), 8.83 (d, J = 1.8, 1H), 8.51 (s, 1H), 8.17 (s, |
| | 1H), 7.77 (s, 1H), 4.64 (d, J = 5.2, 2H), 4.44 (t, J = 10.6, 1H), 4.09 (t, J = 20.7, 4H), 3.69-3.57 |
| | (m, 3H), 3.51 (dd, J = 13.9, 5.9, 2H), 2.91 (s, 3H), 2.89-2.77 (m, 2H), 1.98 (d, J = 14.7, 4H). |
| 43 | 1H NMR (400 MHz, dmso) δ 9.07 (d, J = 1.9, 1H), 8.90 (d, J = 1.9, 1H), 8.07 (s, 1H), 7.85 (dd, |
| | J = 8.4, 2.1, 1H), 7.80 (d, J = 2.1, 1H), 7.08 (d, J = 8.5, 1H), 4.74-4.64 (m, 2H), 4.13-4.02 (m, |
| | 1H), 4.02-3.96 (m, 1H), 3.89 (d, J = 2.7, 3H), 3.80 (d, J = 14.2, 3H), 3.69-3.57 (m, 2H), 2.92 |
| | (s, 3H), 2.89-2.82 (m, 2H). |
| 44 | 1H NMR (400 MHz, dmso) δ 9.13 (d, J = 1.9, 1H), 8.98 (d, J = 1.8, 1H), 8.35 (dd, J = 8.9, 5.5, |
| | 2H), 8.14 (s, 1H), 7.37 (t, J = 8.9, 2H), 4.78-4.65 Mol. (m, 2H), 4.12-3.98 (m, 2H), 3.74- |
| | 3.59 (m, 2H), 3.41 (d, J = 11.8, 1H), 2.95 (s, 3H), 2.93-2.83 (m, 2H). |
| 45 | 1H NMR (400 MHz, dmso) δ 9.18 (d, J = 1.8, 1H), 9.03 (d, J = 1.8, 1H), 8.52-8.46 (m, 2H), |
| | 8.32 (s, 1H), 8.00 (d, J = 8.4, 2H), 4.81-4.66 (m, 2H), 4.05 (ddd, J = 18.4, 8.7, 2.5, 2H), 3.72- |
| | 3.61 (m, 2H), 3.41 (d, J = 11.1, 1H), 2.94 (d, J = 4.6, 5H), 2.94-2.85 (m, 2H). |
| 46 | 1H NMR (400 MHz, dmso) δ 9.04 (d, J = 1.8, 1H), 8.86 (d, J = 1.9, 1H), 8.42 (s, 1H), 8.17 (s, |
| | 1H), 7.77 (s, 1H), 4.70-4.61 (m, 2H), 4.06-3.99 (m, 2H), 3.92 (s, 3H), 3.72-3.61 (m, 2H), |
| | 3.41 (d, J = 11.7, 1H), 2.94 (s, 3H), 2.92-2.80 (m, 2H). |
| 47 | 1H NMR (400 MHz, cdcl3) δ 8.90~8.88 (dd, J = 1.6 Hz, 9.2 Hz, 1H), 8.75 (s, 1H), 8.05~8.03 (d, |
| | J = 8.4 Hz, 2H), 7.81~7.78 (d, J = 10.4 Hz, 1H), 6.72~6.69 (dd, J = 2.8 Hz, 8.4 Hz, 2H), 4.88~4.72 |
| | (m, 2H), 4.70~4.40 (dd, J = 12.8 Hz, 106.4 Hz, 1H), 4.13~3.93 (m, 3H), 3.70~3.58 (m, 1H), |
| | 3.40~3.25 (m, 1H), 2.93~2.77 (m, 4H), 2.11~2.10 (d, J = 6.4 Hz, 3H). |
| 48 | 1H NMR (400 MHz, cdcl3) δ 8.92~8.90 (dd, J = 2.0 Hz, 9.2 Hz, 1H), 8.77 (s, 1H), 8.08~8.06 (d, |
| | J = 8.8 Hz, 2H), 7.85~7.83 (d, J = 9.6 Hz, 1H), 7.04~7.01 (dd, J = 3.2 Hz, 8.8 Hz, 2H), |
| | 4.89~4.73 (m, 2H), 4.70~4.40 (dd, J = 14.4 Hz, 106.4 Hz, 1H), 4.11~3.93 (m, 2H), 3.68~3.62 (t, |
| | J = 11.2 Hz, 4H), 3.43~3.26 (m, 5H), 3.12~3.07 (m, 2H), 2.94~2.77 (m, 1H), 2.12~2.10 (d, J = |
| | 7.2 Hz, 3H), 2.06~2.02 (m, 2H), 1.78~1.69 (m, 2H). |
| 49 | 1H NMR (400 MHz, cdcl3) δ 8.93~8.90 (d, J = 9.2 Hz, 1H), 8.78 (s, 1H), 8.11~8.08 (d, J = 8.8 |
| | Hz, 2H), 7.87~7.84 (d, J = 10.0 Hz, 1H), 7.04~7.01 (dd, J = 2.8 Hz, 9.2 Hz, 2H), 4.90~4.73 (m, |
| | 2H), 4.70~4.40 (dd, J = 14.4 Hz, 107.2 Hz, 1H), 4.12~3.93 (m, 3H), 3.70~3.62 (m, 4H), |
| | 3.40~3.26 (m, 5H), 2.95~2.77 (m, 1H), 2.72~2.70 (t, J = 4.4 Hz, 4H), 2.65~2.62 (t, J = 5.6 Hz, |
| | 2H), 2.12~2.10 (d, J = 7.2 Hz, 3H). |
| 50 | 1H NMR (400 MHz, cdcl3) δ 8.91~8.88 (d, J = 11.6 Hz, 1H), 8.75 (s, 1H), 8.05~8.03 (d, J = 8.4 |
| | Hz, 2H), 7.81~7.79 (d, J = 9.6 Hz, 1H), 6.72~6.70 (d, J = 8.8 Hz, 2H), 4.89~4.43 (m, 3H), 4.11~ |
| | 4.00 (m, 3H), 3.96~3.71 (m, 4H), 3.65~3.58 (m, 1H), 3.40~3.19 (m, 2H), 2.96~2.83 (m, 4H), |
| | 2.25~1.96 (m, 2H). |
| 51 | 1H NMR (400 MHz, cdcl3) δ 8.90~8.88 (m, 1H), 8.75~8.73 (m, 1H), 8.10~8.03 (dd, J = 8.8 Hz, |
| | 19.2 Hz, 2H), 7.81~7.79 (d, J = 8.0 Hz, 1H), 6.73~6.70 (m, 2H), 4.85~4.33 (m, 4H), 4.21~4.02 |
| | (m, 2H), 3.97~3.77 (m, 2H), 3.73~3.52 (m, 2H), 3.34~3.06 (m, 1H), 3.01~2.64 (m, 5H), |
| | 2.39~2.21 (m, 1H), 2.06~1.82 (m, 2H). |
| 52 | 1H NMR (400 MHz, cdcl3) δ 8.92~8.91 (d, J = 2.0 Hz, 1H), 8.78~8.77 (d, J = 2.0 Hz, 1H), |
| | 7.88~7.82 (m, 2H), 7.80 (s, 1H), 6.80~6.76 (t, J = 8.4 Hz, 1H), 4.89~4.72 (m, 2H), 4.28~4.22 (m, |
| | 2H), 4.12~4.08 (m, 1H), 3.91~3.88 (d, J = 11.6 Hz, 1H), 3.84~3.78 (m, 1H), 3.63~3.60 (d, J = |
| | 10.4 Hz, 1H), 2.98~2.97 (d, J = 4.8 Hz, 3H), 2.94~2.85 (m, 2H), 2.82 (s, 3H). |
| 53 | 1H NMR (400 MHz, cdcl3) δ 8.94~8.93 (d, J = 1.6 Hz, 1H), 8.81~8.80 (d, J = 2.0 Hz, 1H), |
| | 7.87~7.82 (m, 3H), 6.98~6.94 (t, J = 8.8 Hz, 1H), 4.90~4.72 (m, 2H), 4.28~4.22 (m, 1H), |
| | 4.12~4.09 (dd, J = 1.6 Hz, 11.6 Hz, 1H), 3.91~3.88 (d, J = 11.2 Hz, 1H), 3.85~3.78 (m, 1H), |
| | 3.63~3.60 (d, J = 11.6 Hz, 1H), 2.97 (s, 6H), 2.94~2.85 (m, 2H), 2.82 (s, 3H). |
| 54 | 1H NMR (400 MHz, cdcl3) δ 8.98~8.95 (m, 1H), 8.85~8.82 (d, J = 10.4 Hz, 1H), 8.17~8.10 (dd, |
| | J = 8.0 Hz, 17.6 Hz, 2H), 7.96~7.93 (m, 1H), 7.36~7.34 (d, J = 8.4 Hz, 2H), 4.89~4.73 (m, 2H), |
| | 4.70~4.40 (dd, J = 11.2 Hz, 107.6 Hz, 1H), 4.09~3.93 (m, 3H), 3.70~3.50 (m, 3H), 3.40~3.12 |
| | (m, 2H), 2.93~2.77 (m, 2H), 2.69~2.63 (t, J = 10.8 Hz, 1H), 2.15~2.10 (m, 6H), 1.99~1.92 (t, J = |
| | 12.8 Hz, 2H), 1.74~1.64 (dd, J = 12.8 Hz, 25.6 Hz, 2H). |
| 55 | 1H NMR (400 MHz, cdcl3) δ 8.92~8.91 (d, J = 1.6 Hz, 1H), 8.78~8.78 (d, J = 2.0 Hz, 1H), |
| | 8.11~8.09 (d, J = 8.8 Hz, 2H), 7.86 (s, 1H), 7.03~7.01 (d, J = 8.8 Hz, 2H), 4.88~4.72 (m, 2H), |
| | 4.25~4.19 (m, 1H), 4.07~4.05 (d, J = 10.4 Hz, 1H), 3.913.89 (t, J = 4.8 Hz, 4H), 3.81~3.73 (m, |
| | 2H), 3.51~3.48 (d, J = 12.8 Hz, 1H), 3.30~3.27 (t, J = 4.8 Hz, 4H), 3.08~3.02 (m, 1H), 2.98~2.93 |
| | (t, J = 11.6 Hz, 1H), 2.75 (s, 3H). |
| 56 | 1H NMR (400 MHz, cdcl3) δ 8.92~8.91 (d, J = 1.6 Hz, 1H), 8.79~8.78 (d, J = 2.0 Hz, 1H), |
| | 8.11~8.09 (d, J = 8.8 Hz, 2H), 7.86 (s, 1H), 7.03~7.01 (d, J = 9.2 Hz, 2H), 4.87~4.72 (m, 2H), |
| | 4.24~4.18 (m, 1H), 4.06~4.02 (m, 1H), 3.91~3.89 (t, J = 4.8 Hz, 4H), 3.78~3.71 (m, 2H), |
| | 3.48~3.45 (d, J = 12.0 Hz, 1H), 3.30~3.27 (t, J = 4.8 Hz, 4H), 3.11~3.04 (m, 1H), 3.02~2.96 (dd, |
| | J = 10.4 Hz, 12.0 Hz, 1H), 2.84 (s, 6H). |
| 57 | 1H NMR (400 MHz, cdcl3) δ 8.97 (d, J = 1.9, 1H), 8.86 (d, J = 1.8, 1H), 8.12 (d, J = 8.3, 2H), |
| | 7.96 (s, 1H), 7.37 (d, J = 8.3, 2H), 4.83 (ddd, J = 49.3, 11.6, 5.4, 2H), 4.26 (dtd, J = 10.3, 5.3, |
| | 2.7, 1H), 4.14-4.06 (m, 1H), 3.99 (d, J = 11.9, 2H), 3.94-3.87 (m, 1H), 3.81 (td, J = 11.4, 2.7, |
| | 1H), 3.61 (dd, J = 10.7, 1.4, 1H), 2.94 (td, J = 11.5, 3.3, 1H), 2.90-2.77 (m, 1H), 2.76-2.66 |
| | (m, 1H), 2.03 (d, J = 13.0, 2H), 1.91 (ddd, J = 16.2, 12.7, 4.1, 2H). |
| 58 | 1H NMR (400 MHz, cdcl3) δ 8.97 (d, J = 1.9, 1H), 8.85 (d, J = 1.9, 1H), 8.11 (d, J = 8.4, 2H), |
| | 7.96 (s, 1H), 7.40 (d, J = 8.3, 2H), 4.83 (ddd, J = 50.5, 11.6, 5.4, 2H), 4.27 (dtd, J = 10.5, 5.4, |
| | 2.7, 1H), 4.10 (ddd, J = 11.7, 3.1, 1.3, 1H), 3.94-3.87 (m, 1H), 3.81 (td, J = 11.5, 2.7, 1H), 3.72 |
| | (t, J = 5.2, 2H), 3.61 (d, J = 10.7, 1H), 3.18 (d, J = 11.7, 2H), 2.94 (td, J = 11.5, 3.3, 1H), 2.87 |
| | (dd, J = 11.5, 10.3, 1H), 2.82 (s, 3H), 2.72-2.61 (m, 3H), 2.33 (t, J = 13.4, 2H), 1.99-1.91 (m, |
| | 5H). |
| 59 | 1H NMR (400 MHz, cdcl3) δ 8.96 (d, J = 1.8, 1H), 8.84 (d, J = 1.8, 1H), 8.10 (d, J = 8.3, 2H), |
| | 7.95 (s, 1H), 7.39 (d, J = 8.3, 2H), 4.82 (ddd, J = 50.7, 11.6, 5.4, 2H), 4.35-4.19 (m, 1H), 4.10 |
| | (ddd, J = 11.7, 3.0, 1.2, 1H), 3.95-3.85 (m, 1H), 3.81 (td, J = 11.4, 2.6, 1H), 3.72-3.51 (m, |
| | 3H), 3.39 (s, 3H), 3.35-3.07 (m, 2H), 2.94 (td, J = 11.5, 3.3, 1H), 2.90-2.84 (m, 1H), 2.82 (s, |
| | 3H), 2.78-2.49 (m, 3H), 2.37-2.07 (m, 2H), 2.07-1.76 (m, 4H). |
| 60 | 1H NMR (400 MHz, cdcl3) δ 8.93~8.91 (dd, J = 1.6 Hz, 6.8 Hz, 1H), 8.81~8.78 (dd, J = 1.6 Hz, |
| | 6.4 Hz, 1H), 8.12~8.09 (t, J = 8.8 Hz, 2H), 7.86~7.85 (d, J = 6.0 Hz, 1H), 7.05~7.01 (t, J = 8.8 |
| | Hz, 2H), 4.87~4.70 (m, 2H), 4.65~4.62 (d, J = 13.6 Hz, 1H), 4.40~4.36 (d, J = 13.2 Hz, 1H), |
| | 4.18~4.04 (m, 2H), 3.91~3.88 (t, J = 4.8 Hz, 4H), 3.78~3.61 (m, 2H), 3.40~3.27 (m, 6H), |
| | 3.23~2.81 (m, 4H), 2.76 (s, 6H). |
| 61 | 1H NMR (400 MHz, cdcl3) δ 8.93~9.92 (dd, J = 1.6 Hz, 3.6 Hz, 1H), 8.79 (s, 1H), 8.11~8.09 (dd, |
| | J = 2.0 Hz, 9.2 Hz, 2H), 7.87~7.86 (d, J = 4.0 Hz, 1H), 7.04~7.01 (dd, J = 2.8 Hz, 8.8 Hz, 2H), |
| | 6.25~5.98 (t, J = 53.6 Hz, 1H), 4.89~4.77 (m, 2H), 4.62~4.31 (dd, J = 12.8 Hz, 108.0 Hz, 1H), |
| | 4.25~3.96 (m, 3H), 3.91~3.89 (t, J = 4.4 Hz, 4H), 3.73~3.65 (m, 1H), 3.47~3.36 (m, 1H), |
| | 3.30~3.28 (t, J = 4.8 Hz, 4H), 3.10~2.98 (m, 1H). |
| 62 | 1H NMR (400 MHz, cdcl3) δ 8.84 (s, 1H), 8.75 (s, 1H), 8.09~8.07 (d, J = 8.0 Hz, 2H), 7.81 (s, |
| | 1H), 7.03~7.01 (d, J = 8.0 Hz, 2H), 4.96~4.93 (M, 1H), 4.72~4.68 (m, 1H), 4.28~4.23 (m, 1H), |
| | 4.12~4.09 (d, J = 12.0 Hz, 1H), 3.90~3.79 (m, 6H), 3.55~3.49 (t, J = 12.8 Hz, 1H), 3.30 (s, 4H), |
| | 3.00~2.84 (m, 2H). |
| 63 | 1H NMR (400 MHz, cdcl3) δ 8.93~8.92 (d, J = 1.6 Hz, 1H), 8.77~8.76 (d, J = 1.6 Hz, 1H), |
| | 8.12~8.09 (d, J = 9.2 Hz, 2H), 7.86 (s, 1H), 7.03~7.00 (d, J = 9.2 Hz, 2H), 4.89~4.84 (m, 1H), |
| | 4.74~4.70 (m, 1H), 4.59 (s, 2H), 4.17~4.11 (m, 1H), 4.05~4.00 (m, 2H), 3.91~3.89 (t, J = |
| | 4.8 Hz, 4H), 3.80~3.76 (d, J = 13.2 Hz, 1H), 3.73~3.66 (m, 1H), 3.30~3.27 (t, J = 4.8 Hz, 4H), |
| | 3.16~3.02 (m, 2H). |
| 64 | 1H NMR (400 MHz, cdcl3) δ 8.78 (s, 1H), 8.71 (s, 1H), 8.04~8.02 (d, J = 6.4 Hz, 2H), 7.75 (s, |
| | 1H), 7.02~7.00 (d, J = 8.0 Hz, 2H), 4.96~4.91 (m, 1H), 4.68~4.62 (m, 1H), 4.24~4.18 (m, 1H), |
| | 4.09~4.06 (d, J = 14.8 Hz, 1H), 3.83~3.77 (t, J = 12.8 Hz, 2H), 3.54~3.50 (m, 1H), 3.34 (s, 4H), |
| | 2.95~2.82 (m, 2H), 2.59 (s, 4H), 2.37 (s, 3H). |
| 65 | 1H NMR (400 MHz, cdcl3) δ 8.93~8.92 (d, J = 1.6 Hz, 1H), 8.79~8.78 (d, J = 1.6 Hz, 1H), |
| | 8.11~8.09 (d, J = 8.8 Hz, 2H), 7.86 (s, 1H), 7.03~7.01 (d, J = 9.2 Hz, 2H), 4.89~4.73 (m, 2H), |
| | 4.25~4.20 (m, 1H), 4.08~4.05 (d, J = 12.8 Hz, 1H), 3.91~3.89 (t, J = 4.8 Hz, 4H), 3.80~3.73 (m, |
| | 1H), 3.61~3.58 (d, J = 12.0 Hz, 1H), 3.30~3.27 (t, J = 5.2 Hz, 4H), 3.12~2.97 (m, 4H), 2.78~2.74 |
| | (m, 2H), 2.25 (s, 6H). |
| 66 | 1H NMR (400 MHz, cdcl3) δ 8.92~8.91 (d, J = 1.6 Hz, 1H), 8.79~8.78 (d, J = 1.6 Hz, 1H), |
| | 8.11~8.09 (d, J = 9.2 Hz, 2H), 7.86 (s, 1H), 7.03~7.01 (d, J = 9.2 Hz, 2H), 4.884.79 (m, 2H), |
| | 4.254.20 (m, 1H), 4.08~4.04 (m, 1H), 3.91~3.89 (t, J = 4.8 Hz, 4H), 3.86~3.85 (t, J = 2.0 Hz, 1H), |
| | 3.80~3.73 (m, 3H), 3.59~3.55 (m, 1H), 3.36 (s, 3H), 3.303.27 (t, J = 5.2 Hz, 4H), 3.24~3.21 (t, |
| | J = 6.0 Hz, 2H), 3.12~3.05 (m, 1H), 3.04~2.98 (dd, J = 10.0 Hz, 11.6 Hz, 1H). |
| 67 | 1H NMR (400 MHz, cdcl3) δ 8.90~8.80 (d, J = 8.0 Hz, 1H), 8.74 (s, 1H), 8.09~8.07 (d, J = 8.4 |
| | Hz, 2H), 7.82~7.80 (d, J = 9.6 Hz, 1H), 6.82~6.81 (d, J = 5.2 Hz, 2H), 4.89~4.66 (m, 3H), |
| | 4.43~3.93 (m, 3H), 3.68~3.59 (dd, J = 11.6 Hz, 24.0 Hz, 1H), 3.40~3.26 (m, 1H), 3.06 (s, 6H), |
| | 2.93~2.78 (m, 1H), 2.12~2.10 (d, J = 7.2 Hz, 3H)./1 H NMR (400 MHz, cdcl 3) δ 8.90~8.88 |
| | (dd, J = 0.8 Hz, 8.8 Hz, 1H), 8.74 (s, 1H), 8.09~8.07 (d, J = 8.8 Hz, 2H), 7.82~7.80 (d, J = 9.2 |
| | Hz, 1H), 6.83~6.80 (dd, J = 3.6 Hz, 8.8 Hz, 2H), 4.89~4.73 (m, 2H), 4.70~4.40 (m, 1H), |
| | 4.13~4.06 (m, 2H), 4.04~4.01 (dd, J = 3.2 Hz, 11.2 Hz, 1H), 3.97~3.59 (m, 2H), 3.40~3.23 (m, |
| | 1H), 3.06 (s, 6H), 2.94~2.78 (m, 1H), 2.12~2.10 (d, J = 7.2 Hz, 3H). |
| 68 | 1H NMR (400 MHz, cdcl3) δ 8.90~8.88 (d, J = 7.6 Hz, 1H), 8.74 (s, 1H), 8.09~8.07 (d, J = 8.8 |
| | Hz, 2H), 7.82~7.80 (d, J = 6.4 Hz, 1H), 6.83~8.81 (d, J = 8.8 Hz, 2H), 4.89~4.44 (m, 3H), |
| | 4.09~3.74 (m, 3H), 3.68~3.58 (dd, J = 12.0 Hz, 24.4 Hz, 1H), 3.38~3.23 (m, 1H), 3.06 (s, 6H), |
| | 2.92~2.75 (m, 2H), 1.16~1.07 (m, 6H). |
| 69 | 1H NMR (400 MHz, cdcl3) δ 8.89 (s, 1H), 8.74~8.73 (d, J = 1.6 Hz, 1H), 8.09~8.07 (d, J = 8.8 |
| | Hz, 2H), 7.81 (s, 1H), 6.82~6.80 (d, J = 8.8 Hz, 2H), 4.89~4.65 (m, 3H), 4.42~4.33 (t, J = |
| | 16.8 Hz, 1H), 4.14~4.01 (m, 2H), 3.73~3.60 (m, 1H), 3.45~3.32 (m, 1H), 3.06 (s, 6H), 3.00~2.83 |
| | (m, 1H), 1.75~1.69 (m, 1H), 0.98 (s, 2H), 0.78~0.72 (d, J = 23.2 Hz, 2H). |
| 70 | 1H NMR (400 MHz, cdcl3) δ 8.90~8.88 (d, J = 8.8 Hz, 1H), 8.74 (s, 1H), 8.09~8.07 (d, J = 8.4 |
| | Hz, 2H), 7.82~7.80 (d, J = 7.6 Hz, 1H), 6.84~6.80 (m, 2H), 4.90~4.43 (m, 3H), 4.10~3.60 (m, |
| | 4H), 3.37~3.22 (m, 1H), 3.06 (s, 6H), 2.95~2.83 (m, 1H), 2.33~2.21 (m, 2H), 1.07~0.98 (m, 1H), |
| | 0.58~0.49 (dd, J = 7.6 Hz, 31.2 Hz, 2H), 0.18~0.12 (d, J = 23.6 Hz, 2H). |
| 71 | 1H NMR (400 MHz, cdcl3) δ 8.8.91~8.88 (d, J = 10.4 Hz, 1H), 8.75 (s, 1H), 8.09~8.06 (dd, |
| | J = 2.8 Hz, 8.8 Hz, 2H), 7.84~7.80 (dd, J = 4.8 Hz, 9.2 Hz, 1H), 6.83~6.81 (dd, J = 2.4 Hz,, 8.8 Hz, |
| | 2H), 4.90~4.37 (m, 3H), 4.11~4.02 (m, 2H), 3.92~3.52 (m, 2H), 3.37~3.18 (m, 1H), 3.07~3.06 |
| | (d, J = 4.8 Hz, 7H), 3.01~2.86 (m, 3H), 2.80~2.65 (m, 2H). |
| 72 | 1H NMR (400 MHz, cdcl3) δ 8.90~8.88 (d, J = 9.2 Hz, 1H), 8.74 (s, 1H), 8.09~8.07 (d, J = 8.8 |
| | Hz, 2H), 7.82~7.80 (d, J = 9.2 Hz, 1H), 6.83~6.81 (d, J = 7.2 Hz, 2H), 4.92~4.41 (m, 3H), |
| | 4.09~3.73 (m, 7H), 3.67~3.58 (dd, J = 12.8 Hz, 24.8 Hz, 1H), 3.40~3.19 (m, 2H), 3.06 (s, 6H), |
| | 2.98~2.80 (m, 1H), 2.28~1.96 (m, 2H). |
| 73 | 1H NMR (400 MHz, cdcl3) δ 8.90~8.88 (d, J = 10.4 Hz, 1H), 8.74 (s, 1H), 8.09~8.07 (d, J = 9.2 |
| | Hz, 2H), 7.82~7.80 (d, J = 9.2 Hz, 1H), 6.83~6.80 (d, J = 8.4 Hz, 2H), 4.92~4.40 (m, 3H), |
| | 4.08~3.71 (m, 5H), 3.65~3.60 (t, J = 11.2 Hz, 1H), 3.44~3.27 (m, 3H), 3.06 (s, 6H), 2.95~2.69 |
| | (m, 2H), 1.96~1.83 (m, 2H), 1.66~1.52 (m, 2H). |
| 74 | 1H NMR (400 MHz, cdcl3) δ 8.90~8.87 (d, J = 12.4 Hz, 1H), 8.75 (s, 1H), 8.09~8.07 (d, J = 8.8 |
| | Hz, 2H), 7.83~7.80 (d, J = 12.8 Hz, 1H), 6.84~6.81 (d, J = 8.8 Hz, 2H), 4.95~4.40 (m, 3H), |
| | 4.07~3.71 (m, 3H), 3.66~3.57 (dd, J = 12.4 Hz, 26.4 Hz, 2H), 3.38~3.22 (m, 1H), 3.06 (s, 6H), |
| | 2.93~2.78 (m, 1H), 2.44~2.36 (m, 1H), 2.08~1.93 (m, 2H), 1.81~1.73 (m, 1H), 1.64~1.51 (m, |
| | 2H), 1.34~1.07 (m, 3H). |
| 75 | 1H NMR (400 MHz, cdcl3) δ 8.89~8.87 (d, J = 7.6 Hz, 1H), 8.73 (s, 1H), 8.09~8.07 (d, J = 8.8 |
| | Hz, 2H), 7.81~7.80 (d, J = 5.6 Hz, 1H), 6.82~6.80 (d, J = 8.0 Hz, 2H), 4.93~4.41 (m, 3H), |
| | 4.06~3.70 (m, 4H), 3.66~3.59 (m, 1H), 3.47 (s, 1H), 3.39~3.22 (m, 1H), 3.06 (s, 6H), 2.94~2.79 |
| | (m, 1H), 2.52~2.45 (m, 1H), 2.00~1.74 (m, 4H), 1.61~1.41 (m, 4H). |
| 76 | 1H NMR (400 MHz, cdcl3) δ 8.91~8.88 (d, J = 9.2 Hz, 1H), 8.74~8.73 (d, J = 1.6 Hz, 1H), |
| | 8.08~8.06 (dd, J = 2.4 Hz, 8.8 Hz, 2H), 7.83~7.81 (d, J = 6.4 Hz, 1H), 6.83~6.81 (d, J = 8.8 Hz, |
| | 2H), 4.93~4.74 (m, 2H), 4.66~4.33 (dd, J = 14.0 Hz, 119.6 Hz, 1H), 4.10~3.88 (m, 3H), |
| | 3.75~3.37 (m, 4H), 3.07 (s, 6H), 3.03~2.89 (m, 1H). |
| 77 | 1H NMR (400 MHz, cdcl3) δ 8.92~8.88 (dd, J = 2.0 Hz, 12.0 Hz, 1H), 8.76~8.74 (dd, J = 1.6 Hz, |
| | 7.2 Hz, 1H), 8.09~8.07 (d, J = 8.8 Hz, 2H), 7.83~7.81 (d, J = 11.6 Hz, 1H), 6.84~6.81 (d, J = 9.2 |
| | Hz, 2H), 4.91~4.74 (m, 2H), 4.68~4.35 (dd, J = 12.4 Hz, 116.4 Hz, 1H), 4.11~4.02 (m, 2H), |
| | 3.96~3.57 (m, 2H), 3.41~3.23 (m, 1H), 3.07~3.06 (d, J = 2.4 Hz, 6H), 3.02~2.85 (m, 1H), |
| | 2.78~2.65 (m, 4H). |
| 78 | 1H NMR (400 MHz, cdcl3) δ 8.88~8.87 (d, J = 1.6 Hz, 1H), 8.72~8.63 (m, 3H), 8.06 (s, 2H), |
| | 7.81 (s, 1H), 7.73 (s, 1H), 7.23 (s, 1H), 6.83~6.81 (d, J = 8.8 Hz, 2H), 4.84~4.50 (m, 3H), |
| | 4.17~3.98 (m, 2H), 3.88~3.58 (m, 2H), 3.43~3.29 (m, 1H), 3.21~3.06 (m, 7H). |
| 79 | 1H NMR (400 MHz, cdcl3) δ 8.89~8.88 (d, J = 3.2 Hz, 1H), 8.73 (s, 1H), 8.49~8.41 (m, 2H), |
| | 8.09~8.06 (dd, J = 4.4 Hz, 8.4 Hz, 2H), 7.81~7.80 (d, J = 4.0 Hz, 1H), 7.56~7.48 (dd, J = 7.2 Hz, |
| | 26.0 Hz, 1H), 7.21~7.13 (m, 1H), 6.84~6.79 (t, J = 10.0 Hz, 2H), 4.88~4.39 (m, 3H), 4.07~3.49 |
| | (m, 4H), 3.29~3.20 (dd, J = 14.8 Hz, 25.6 Hz, 1H), 3.05 (s, 6H), 3.02~2.79 (m, 3H), 2.72~2.57 |
| | (m, 2H). |
| 80 | 1H NMR (400 MHz, cdcl3) δ 8.92~8.87 (dd, J = 1.6 Hz, 16.8 Hz, 1H), 8.76~8.73 (dd, J = 1.6 Hz, |
| | 12.8 Hz, 1H), 8.52~8.37 (m, 2H), 8.08~8.06 (d, J = 8.8 Hz, 2H), 7.83~7.80 (d, J = 12.4 Hz, 1H), |
| | 7.64~7.54 (dd, J = 7.6 Hz, 30.8 Hz, 1H), 7.25~7.09 (s, 1H), 6.83~6.79 (dd, J = 4.4 Hz, 8.0 Hz, |
| | 2H), 4.90~4.41 (m, 3H), 4.11~3.90 (m, 3H), 3.73~3.69 (d, J = 16.0 Hz, 2H), 3.65~3.48 (m, 1H), |
| | 3.39~3.24 (m, 1H), 3.05~3.04 (d, J = 4.0 Hz, 6H), 2.99~2.83 (m, 1H). |
| 81 | 1H NMR (400 MHz, cdcl3) δ 8.90~8.88 (d, J = 8.4 Hz, 1H), 8.75 (s, 1H), 8.09~8.07 (d, J = 8.8 |
| | Hz, 2H), 7.83~7.80 (d, J = 8.8 Hz, 1H), 6.84~6.81 (d, J = 8.4 Hz, 2H), 4.90~4.75 (m, 2H), |
| | 4.69~4.37 (dd, J = 13.2 Hz, 114.4 Hz, 1H), 4.10~3.59 (m, 4H), 3.38~3.24 (m, 1H), 3.06 (s, 6H), |
| | 2.96~2.81 (m, 1H), 2.53~2.43 (m, 4H), 2.04~1.95 (m, 2H). |
| 82 | 1H NMR (400 MHz, cdcl3) δ 8.92 (s, 1H), 8.79 (s, 1H), 8.10 (d, J = 8.7, 2H), 7.86 (s, 1H), 7.02 |
| | (d, J = 8.7, 2H), 4.79 (ddd, J = 16.5, 11.7, 5.3, 2H), 4.27-4.12 (m, 1H), 4.04 (d, J = 10.4, 1H), |
| | 3.96-3.81 (m, 5H), 3.74 (t, J = 11.4, 1H), 3.63 (d, J = 12.8, 1H), 3.33-3.05 (m, 4H), 1.36 (d, |
| | J = 6.8, 6H). |
| 83 | 1H NMR (400 MHz, cdcl3) δ 8.93 (s, 1H), 8.79 (s, 1H), 8.10 (d, J = 8.4, 2H), 7.87 (s, 1H), 7.02 |
| | (d, J = 8.2, 2H), 4.82 (ddd, J = 15.9, 11.5, 5.7, 2H), 4.31-4.17 (m, 1H), 4.08 (d, J = 11.3, 1H), |
| | 3.90 (s, 5H), 3.79 (t, J = 10.7, 1H), 3.60 (d, J = 10.8, 1H), 3.29 (s, 4H), 3.15-2.91 (m, 2H), 2.34- |
| | 2.22 (m, 1H), 1.25 (s, 4H). |
| 84 | 1H NMR (400 MHz, cdcl3) δ 8.82~8.77 (d, J = 21.2 Hz, 1H), 8.67~8.61 (d, J = 22.8 Hz, 1H), |
| | 8.03~7.97 (t, J = 10.4 Hz, 2H), 7.74~7.68 (d, J = 22.4 Hz, 1H), 7.51~7.41 (d, J = 37.2 Hz, 1H), |
| | 6.86 (s, 1H), 6.76~6.72 (t, J = 8.4 Hz, 2H), 4.75~4.56 (m, 2H), 4.38~4.18 (dd, J = 12.4 Hz, 66.8 |
| | Hz, 1H), 3.94~3.64 (m, 4H), 3.51~3.40 (m, 2H), 3.30~3.19 (m, 1H), 3.01~3.00 (d, J = 6.0 Hz, |
| | 6H), 2.89~2.78 (m, 1H). |
| 85 | 1H NMR (400 MHz, cdcl3) δ 8.90~8.89 (d, J = 6.8 Hz, 1H), 8.74~8.73 (d, J = 3.6 Hz, 1H), |
| | 8.09~8.06 (dd, J = 4.0 Hz, 8.4 Hz, 2H), 7.82~7.81 (d, J = 6.8 Hz, 1H), 7.52~7.49 (d, J = 13.2 Hz, |
| | 1H), 7.03~6.88 (m, 2H), 6.84~6.80 (t, J = 6.0 Hz, 2H), 4.87~4.33 (m, 4H), 4.30~4.26 (t, J = 6.4 |
| | Hz, 1H), 4.08~3.94 (m, 2H), 3.91~3.48 (m, 2H), 3.30~3.18 (m, 1H), 3.06 (s, 6H), 2.97~2.67 (m, |
| | 3H). |
| 86 | 1H NMR (400 MHz, cdcl3) δ 8.88 (s, 1H), 8.75 (s, 1H), 8.11~8.08 (d, J = 8.8 Hz, 2H), 7.81 (s, |
| | 1H), 7.18~7.10 (m, 2H), 6.83~6.79 (t, J = 9.2 Hz, 2H), 6.23~5.94 (dd, J = 14.0 Hz, 102.4 Hz, |
| | 1H), 4.92~4.50 (m, 3H), 4.27~4.08 (m, 2H), 3.81~3.72 (dd, J = 12.4 Hz, 24.0 Hz, 1H), 3.59~3.49 |
| | (dd, J = 15.2 Hz, 27.6 Hz, 1H), 3.22~3.05 (m, 8H). |
| 87 | 1H NMR (400 MHz, cdcl3) δ 8.89~8.88 (d, J = 2.0 Hz, 1H), 8.75~8.74 (d, J = 1.6 Hz, 1H), |
| | 8.09~8.07 (d, J = 8.8 Hz, 2H), 7.81 (s, 1H), 7.68 (s, 1H), 7.64 (s, 1H), 6.83~6.81 (d, J = 9.2 Hz, |
| | 2H), 4.88~4.77 (m, 2H), 4.60~4.42 (m, 1H), 4.19~4.13 (m, 1H), 4.07~4.04 (d, J = 10.8 Hz, 1H), |
| | 3.87 (s, 3H), 3.73~3.67 (t, J = 11.2 Hz, 1H), 3.38~3.18 (m, 2H), 3.06~2.96 (m, 7H). |
| 88 | 1H NMR (400 MHz, cdcl3) δ 8.89~8.88 (d, J = 1.6 Hz, 1H), 8.75~8.74 (d, J = 1.6 Hz, 1H), |
| | 8.09~8.06 (d, J = 8.8 Hz, 2H), 7.81 (s, 1H), 6.83~6.81 (d, J = 9.2 Hz, 2H), 4.88~4.73 (m, 2H), |
| | 4.25~4.19 (m, 1H), 4.09~4.05 (dd, J = 2.0 Hz, 11.6 Hz, 1H), 3.91~3.88 (d, J = 12.0 Hz, 1H), |
| | 3.80~3.74 (m, 1H), 3.62~3.59 (d, J = 12.0 Hz, 1H), 3.10~3.03 (m, 7H), 3.02~2.95 (m, 3H), |
| | 1.40~1.36 (t, J = 7.6 Hz, 3H). |
| 89 | 1H NMR (400 MHz, cdcl3) δ 8.89~8.88 (d, J = 1.6 Hz, 1H), 8.75~8.74 (d, J = 2.0 Hz, 1H), |
| | 8.09~8.06 (d, J = 8.8 Hz, 2H), 7.81 (s, 1H), 6.83~6.81 (d, J = 8.8 Hz, 2H), 4.86~4.72 (m, 2H), |
| | 4.23~4.17 (m, 1H), 4.05~4.02 (dd, J = 2.0 Hz, 12.0 Hz, 1H), 3.92~3.89 (d, J = 12.4 Hz, 1H), |
| | 3.78~3.71 (m, 1H), 3.64~3.61 (d, J = 12.8 Hz, 1H), 3.24~3.07 (m, 3H), 3.06 (s, 6H), 1.36~1.34 |
| | (dd, J = 1.6 Hz, 7.2 Hz, 6H). |
| 90 | 1H NMR (400 MHz, cdcl3) δ 8.89~8.88 (d, J = 2.0 Hz, 1H), 8.75~8.74 (d, J = 2.0 Hz, 1H), |
| | 8.09~8.07 (d, J = 8.8 Hz, 2H), 7.81 (s, 1H), 6.83~6.81 (d, J = 8.8 Hz, 2H), 4.90~4.73 (m, 2H), |
| | 4.28~4.22 (m, 1H), 4.09~4.06 (dd, J = 1.2 Hz, 11.2 Hz, 1H), 3.90~3.87 (d, J = 11.6 Hz, 1H), |
| | 3.82~3.76 (m, 1H), 3.62~3.59 (d, J = 12.0 Hz, 1H), 3.12~2.98 (m, 8H), 2.31~2.24 (m, 1H), |
| | 1.19~1.15 (m, 2H), 1.00~0.95 (m, 2H). |
| 91 | 1H NMR (400 MHz, cdcl3) δ 8.89~8.88 (d, J = 2.0 Hz, 1H), 8.75~8.74 (d, J = 1.6 Hz, 1H), |
| | 8.09~8.06 (d, J = 9.2 Hz, 2H), 7.81 (s, 1H), 6.83~6.81 (d, J = 9.2 Hz, 2H), 4.85~4.72 (m, 2H), |
| | 4.22~4.16 (m, 1H), 4.04~4.01 (d, J = 10.4 Hz, 1H), 3.90~3.87 (d, J = 12.0 Hz, 1H), 3.77~3.70 |
| | (m, 1H), 3.63~3.60 (d, J = 12.4 Hz, 1H), 3.28~3.06 (m, 8H), 2.96~2.88 (m, 1H), 2.13~2.10 (d, |
| | J = 10.8 Hz, 2H), 1.87~1.83 (d, J = 13.2 Hz, 2H), 1.56~1.45 (m, 2H), 1.29~1.15 (m, 4H). |
| 92 | 1H NMR (400 MHz, cdcl3) δ 8.99~8.98 (d, J = 2.0 Hz, 1H), 8.90~8.89 (d, J = 1.6 Hz, 1H), |
| | 8.83~8.82 (dd, J = 1.2 Hz, 4.8 Hz, 1H), 8.75~8.74 (d, J = 1.6 Hz, 1H), 8.07~8.05 (d, J = 8.8 Hz, |
| | 2H), 8.01~7.98 (m, 1H), 7.82 (s, 1H), 7.45~7.41 (dd, J = 4.8 Hz, 8.0 Hz, 1H), 6.83~6.81 (d, J = |
| | 8.8 Hz, 2H), 4.84~4.66 (m, 2H), 4.30~4.24 (m, 1H), 4.06~4.02 (dd, J = 1.6 Hz, 11.6 Hz, 1H), |
| | 3.93~3.90 (d, J = 11.6 Hz, 1H), 3.84~3.78 (m, 1H), 3.65~3.62 (d, J = 12.0 Hz, 1H), 3.07 (s, 6H), |
| | 2.60~2.54 (m, 1H), 2.50~2.44 (t, J = 12.0 Hz, 1H). |
| 93 | 1H NMR (400 MHz, cdcl3) δ 8.91~8.90 (d, J = 1.6 Hz, 1H), 8.76~8.75 (d, J = 1.6 Hz, 1H), |
| | 8.08~8.06 (d, J = 8.8 Hz, 2H), 7.82 (s, 1H), 7.327.27 (dd, J = 7.2 Hz, 13.6 Hz, 1H), 7.16~7.11 (t, |
| | J = 7.6 Hz, 2H), 7.03~6.98 (t, J = 8.4 Hz, 1H), 6.84~6.82 (d, J = 8.8 Hz, 2H), 4.85~4.68 (m, 2H), |
| | 4.19 (s, 2H), 4.15~4.09 (m, 1H), 3.97~3.94 (d, J = 10.0 Hz, 1H), 3.83~3.80 (d, J = 12.0 Hz, 1H), |
| | 3.67~3.60 (m, 1H), 3.47~3.44 (d, J = 12.4 Hz, 1H), 3.06 (s, 6H), 2.92~2.82 (m, 2H). |
| 94 | 1H NMR (400 MHz, cdcl3) δ 8.90~8.89 (d, J = 1.6 Hz, 1H), 8.75~8.74 (d, J = 1.6 Hz, 1H), |
| | 8.09~8.06 (d, J = 9.2 Hz, 2H), 7.82 (s, 1H), 6.83~6.81 (d, J = 9.2 Hz, 2H), 4.90~4.74 (m, 2H), |
| | 4.29~4.23 (m, 1H), 4.11~4.08 (dd, J = 2.8 Hz, 11.2 Hz, 1H), 3.91~3.88 (d, J = 11.6 Hz, 1H), |
| | 3.84~3.77 (m, 1H), 3.62~3.59 (d, J = 11.6 Hz, 1H), 3.06 (s, 6H), 2.97~2.84 (m, 2H), 2.81 (s, |
| | 3H). |
| 95 | 1H NMR (400 MHz, cdcl3) δ 8.91~8.88 (dd, J = 2.0 Hz, 11.6 Hz, 1H), 8.75~8.74 (d, J = 1.6 Hz, |
| | 1H), 8.09~8.07 (d, J = 8.8 Hz, 2H), 7.83~7.80 (d, J = 9.6 Hz, 1H), 6.83~6.80 (dd, J = 4.0 Hz, 8.8 |
| | Hz, 2H), 4.90~4.74 (m, 2H), 4.71~4.43 (dd, J = 13.2 Hz, 100.4 Hz, 1H), 4.11~3.71 (s, 7H), |
| | 3.65~3.58 (m, 1H), 3.41~3.17 (m, 2H), 3.06 (s, 6H), 2.97~2.83 (m, 1H), 2.27~1.98 (m, 2H). |
| 96 | 1H NMR (400 MHz, cdcl3) δ 8.90~8.88 (m, 1H), 8.74~8.73 (d, J = 1.6 Hz, 1H), 8.09~8.07 (d, J = |
| | 7.6 Hz, 2H), 7.82~7.80 (d, J = 8.0 Hz, 1H), 6.83~6.81 (d, J = 8.4 Hz, 2H), 4.99~4.76 (m, 2H), |
| | 4.72~4.34 (m, 1H), 4.20~3.82 (s, 3H), 3.74~3.60 (m, 1H), 3.51~3.32 (m, 1H), 3.06 (s, 6H), |
| | 3.01~2.89 (m, 1H), 2.63~2.44 (m, 1H), 2.20~2.07 (m, 1H), 1.73~1.65 (m, 1H). |
| 97 | 1H NMR (400 MHz, cdcl3) δ 8.89~8.88 (d, J = 2.0 Hz, 1H), 8.75~8.74 (d, J = 2.0 Hz, 1H), |
| | 8.10~8.08 (d, J = 8.8 Hz, 2H), 7.81 (s, 1H), 6.84~6.81 (d, J = 8.8 Hz, 2H), 4.94~4.78 (m, 2H), |
| | 4.55~4.52 (d, J = 13.6 Hz, 1H), 4.28~4.24 (d, J = 13.6 Hz, 1H), 4.17~4.06 (m, 2H), 3.79~3.65 |
| | (m, 1H), 3.59~3.39 (m, 1H), 3.06~2.95 (m, 7H), 1.58~1.44 (m, 4H). |
| 98 | 1H NMR (400 MHz, cdcl3) δ 8.90~8.87 (d, J = 9.2 Hz, 1H), 8.74 (s, 1H), 8.09~8.07 (d, J = 8.8 |
| | Hz, 2H), 7.82~7.80 (d, J = 9.6 Hz, 1H), 6.83~6.80 (dd, J = 4.0 Hz, 8.8 Hz, 2H), 4.90~4.75 (m, |
| | 2H), 4.71~4.43 (dd, J = 12.4 Hz, 99.2 Hz, 1H), 4.14~3.77 (m, 3H), 3.66~3.58 (dd, J = 12.4 Hz, |
| | 21.6 Hz, 1H), 3.36~3.20 (m, 1H), 3.06 (s, 6H), 2.93~2.80 (m, 2H), 1.84~1.67 (m, 6H), 1.59~1.48 |
| | (m, 2H). |
| 99 | 1H NMR (400 MHz, cdcl3) δ 8.89~8.88 (d, J = 2.0 Hz, 1H), 8.74~8.73 (d, J = 2.0 Hz, 1H), |
| | 8.08~8.06 (d, J = 9.2 Hz, 2H), 7.81 (s, 1H), 6.83~6.81 (d, J = 8.8 Hz, 2H), 6.41 (s, 1H), |
| | 4.89~4.70 (m, 2H), 4.24~4.18 (m, 1H), 4.08~4.04 (dd, J = 2.0 Hz, 11.6 Hz, 1H), 3.93~3.90 (d, |
| | J = 12.0 Hz, 1H), 3.85 (s, 2H), 3.79~3.73 (m, 1H), 3.62~3.59 (d, J = 12.0 Hz, 1H), 3.18~3.12 (m, |
| | 1H), 3.10~3.04 (s, 7H), 2.84~2.83 (d, J = 4.8 Hz, 3H). |
| 100 | 1H NMR (400 MHz, cdcl3) δ 8.90~8.89 (dd, J = 2.0 Hz, 3.6 Hz, 1H), 8.75~8.74 (t, J = 1.2 Hz, |
| | 1H), 8.09~8.06 (dd, J = 2.4 Hz, 9.2 Hz, 2H), 7.82~7.81 (d, J = 3.6 Hz, 1H), 6.84~6.81 (dd, J = |
| | 3.2 Hz, 9.2 Hz Hz, 2H), 6.25~5.98 (m, 1H), 4.88~4.76 (m, 2H), 4.61~4.32 (dd, J = 13.2 Hz, 104.4 |
| | Hz, 1H), 4.24~3.95 (m, 3H), 3.73~3.65 (m, 1H), 3.47~3.35 (m, 1H), 3.10~2.97 (m, 7H). |
| 101 | 1H NMR (400 MHz, cdcl3) δ 8.90~8.89 (d, J = 2.0 Hz, 1H), 8.74~8.73 (d, J = 2.0 Hz, 1H), |
| | 8.08~8.06 (d, J = 8.8 Hz, 2H), 7.82 (s, 1H), 6.94~6.91 (d, J = 8.8 Hz, 1H), 6.82~6.80 (m, 3H), |
| | 4.87~4.73 (m, 2H), 4.33~4.29 (t, J = 7.6 Hz, 2H), 4.20~4.14 (m, 1H), 4.08~4.05 (d, J = 12.0 Hz, |
| | 1H), 3.89~3.86 (d, J = 11.6 Hz, 1H), 3.76~2.69 (m, 1H), 3.58~3.55 (d, J = 12.4 Hz, 1H), |
| | 3.27~3.22 (t, J = 7.6 Hz, 2H), 3.06 (s, 6H), 3.00~2.93 (m, 2H), 2.43~2.39 (d, J = 15.6 Hz, 3H). |
| 102 | 1H NMR (400 MHz, cdcl3) δ 8.90~8.89 (d, J = 2.0 Hz, 1H), 8.73~8.72 (d, J = 2.0 Hz, 1H), |
| | 8.09~8.07 (d, J = 9.2 Hz, 2H), 7.81 (s, 1H), 6.83~6.81 (d, J = 8.8 Hz, 2H), 4.87~4.76 (m, 2H), |
| | 4.38~4.35 (m, 1H), 4.21~4.16 (m, 1H), 4.09~4.06 (d, J = 11.6 Hz, 1H), 3.87~3.84 (d, J = 12.0 |
| | Hz, 1H), 3.80~3.74 (m, 1H), 3.60~3.57 (d, J = 12.4 Hz, 1H), 3.18~3.11 (m, 4H), 3.06 (s, 6H), |
| | 2.98~2.90 (m, 3H), 2.76~2.74 (d, J = 9.6 Hz, 1H), 2.48~2.44 (dd, J = 5.2 Hz, 9.6 Hz, 1H), |
| | 2.30~2.13 (m, 2H), 1.82~1.74 (m, 1H). |
| 103 | 1H NMR (400 MHz, cdcl3) δ 8.90~8.89 (d, J = 1.6 Hz, 1H), 8.73~8.72 (d, J = 1.6 Hz, 1H), |
| | 8.09~8.06 (d, J = 9.2 Hz, 2H), 7.81 (s, 1H), 6.83~6.81 (d, J = 8.8 Hz, 2H), 4.87~4.75 (m, 2H), |
| | 4.38~4.34 (m, 1H), 4.23~4.17 (m, 1H), 4.09~4.06 (d, J = 10.0 Hz, 1H), 3.88~3.85 (d, J = 11.6 |
| | Hz, 1H), 3.80~3.74 (m, 1H), 3.61~3.58 (d, J = 11.6 Hz, 1H), 3.16~3.09 (m, 4H), 3.06 (s, 6H), |
| | 2.94~2.88 (m, 3H), 2.76~2.74 (d, J = 9.6 Hz, 1H), 2.49~2.45 (dd, J = 5.2 Hz, 10.0 Hz, 1H), |
| | 2.40~2.39 (d, J = 6.0 Hz, 1H), 2.30~2.24 (dd, J = 8.4 Hz, 15.2 Hz, 1H), 2.19~2.11 (m, 1H), |
| | 1.78~1.72 (m, 1H). |
| 104 | 1H NMR (400 MHz, cdcl3) δ 8.90~8.89 (d, J = 1.6 Hz, 1H), 8.75~8.74 (d, J = 2.0 Hz, 1H), |
| | 8.09~8.06 (d, J = 8.8 Hz, 2H), 7.82 (s, 1H), 6.83~6.81 (d, J = 8.8 Hz, 2H), 4.88~4.75 (m, 2H), |
| | 4.24~4.18 (m, 1H), 4.09~4.06 (d, J = 11.6 Hz, 1H), 3.91~3.88 (d, J = 12.0 Hz, 1H), 3.80~3.74 |
| | (m, 1H), 3.62~3.59 (d, J = 11.2 Hz, 1H), 3.56 (s, 2H), 3.11~3.06 (m, 8H), 3.04~2.94 (m, 4H), |
| | 2.40 (s, 3H). |
| 105 | 1H NMR (400 MHz, dmso) δ 9.12 (d, J = 1.9, 1H), 8.95 (d, J = 1.8, 1H), 8.73 (s, 1H), 8.34 (dd, |
| | J = 8.9, 1.6, 1H), 8.19 (s, 1H), 8.16 (s, 1H), 7.77 (d, J = 8.9, 1H), 4.76 (d, J = 4.7, 2H), 4.10 (s, |
| | 4H), 4.06-4.01 (m, 1H), 3.74 (d, J = 11.6, 1H), 3.66 (dd, J = 11.5, 8.9, 1H), 3.45-3.40 (m, |
| | 1H), 2.96 (s, 3H), 2.94-2.87 (m, 2H). |
| 106 | 1H NMR (400 MHz, dmso) δ 9.02 (d, J = 1.9, 1H), 8.82 (d, J = 1.8, 1H), 8.12 (d, J = 9.0, 2H), |
| | 7.87 (s, 1H), 6.83 (d, J = 9.0, 2H), 5.89 (td, J = 4.8, 2.4, 1H), 4.14 (dd, J = 10.4, 4.9, 1H), 4.00- |
| | 3.91 (m, 2H), 3.85 (td, J = 8.2, 4.9, 1H), 3.01 (s, 6H), 2.43 (dt, J = 14.7, 8.0, 1H), 2.26-2.18 (m, |
| | 1H). |
| 107 | 1H NMR (400 MHz, dmso) δ 9.02 (d, J = 1.8, 1H), 8.82 (d, J = 1.8, 1H), 8.10 (d, J = 8.9, 2H), |
| | 7.85 (s, 1H), 6.83 (d, J = 8.9, 2H), 5.68-5.55 (m, 1H), 3.97 (dt, J = 11.1, 4.1, 2H), 3.67-3.58 |
| | (m, 2H), 3.06-2.94 (m, 6H), 2.21 (d, J = 9.6, 2H), 1.88-1.78 (m, 2H). |
| 108 | 1H NMR (400 MHz, cdcl3) δ 8.96~8.95 (d, J = 2.0 Hz, 1H), 8.83~8.82 (d, J = 1.6 Hz, 1H), |
| | 8.17~8.16 (d, J = 2.0 Hz, 1H), 7.99~7.96 (dd, J = 2.0 Hz, 8.4 Hz, 1H), 7.88 (s, 1H), 7.17~7.15 (d, |
| | J = 8.4 Hz, 1H), 4.89~4.73 (m, 2H), 4.28~4.23 (m, 1H), 4.12~4.09 (dd, J = 1.6 Hz, 11.6 Hz, 1H), |
| | 3.91~3.88 (d, J = 11.6 Hz, 1H), 3.85~2.78 (m, 1H), 3.63~3.60 (d, J = 10.4 Hz, 1H), 2.97~2.92 |
| | (m, 8H), 2.82 (s, 3H). |
| 109 | 1H NMR (400 MHz, dmso) δ 9.09 (s, 1H), 8.93 (s, 1H), 8.00 (s, 1H), 7.78 (s, 2H), 6.99 (d, J = |
| | 9.0, 1H), 4.69 (dd, J = 20.0, 13.4, 2H), 4.32 (s, 4H), 4.03 (d, J = 9.1, 2H), 3.66 (dd, J = 21.0, |
| | 12.3, 2H), 3.46-3.36 (m, 2H), 3.02-2.82 (m, 5H). |
| 110 | 1H NMR (400 MHz, dmso) δ 9.14 (d, J = 1.6, 1H), 8.98 (d, J = 1.7, 1H), 8.08 (s, 1H), 7.90 (dd, |
| | J = 10.8, 2.6, 2H), 7.11 (d, J = 8.1, 1H), 6.18 (s, 2H), 4.82-4.68 (m, 2H), 4.08 (d, J = 10.0, 2H), |
| | 3.78-3.66 (m, 2H), 3.47 (d, J = 11.7, 1H), 3.00 (s, 3H), 2.94 (dd, J = 18.9, 7.8, 2H). |
| 111 | 1H NMR (400 MHz, dmso) δ 9.17 (d, J = 1.8, 1H), 9.01 (d, J = 1.8, 1H), 8.98-8.91 (m, 2H), |
| | 8.67 (dd, J = 8.9, 2.0, 1H), 8.53 (d, J = 7.6, 1H), 8.34 (s, 1H), 8.16 (t, J = 7.3, 1H), 7.66-7.58 |
| | (m, 1H), 4.81 (d, J = 5.2, 2H), 4.16-4.09 (m, 1H), 4.04 (d, J = 11.9, 1H), 3.76 (d, J = 11.4, 1H), |
| | 3.67 (td, J = 11.5, 2.6, 1H), 3.43 (d, J = 12.2, 1H), 2.96 (s, 3H), 2.95-2.88 (m, 2H). |
| 112 | 1H NMR (400 MHz, cdcl3) δ 8.88~8.87 (d, J = 2.0 Hz, 1H), 8.73~8.72 (d, J = 2.9 Hz, 1H), |
| | 8.09~8.07 (d, J = 8.8 Hz, 2H), 7.80 (s, 1H), 7.08~7.07 (d, J = 4.0 Hz, 1H), 6.81~6.79 (d, J = 9.2 |
| | Hz, 2H), 4.82~4.68 (m, 2H), 4.26~4.20 (m, 1H), 4.00~3.96 (m, 1H), 3.78~3.72 (m, 1H), 3.05 (s, |
| | 8H), 3.03~2.99 (m, 1H), 2.82~2.81 (d, J = 4.8 Hz, 3H), 2.70~2.67 (dd, J = 1.6 Hz, 11.2 Hz, 1H), |
| | 2.47~2.41 (m, 1H), 2.38~2.33 (t, J = 11.2 Hz, 1H). |
| 113 | 1H NMR (400 MHz, cdcl3) δ 8.89~8.88 (d, J = 2.0 Hz, 1H), 8.74~8.73 (d, J = 2.0 Hz, 1H), |
| | 8.09~8.07 (d, J = 8.8 Hz, 2H), 7.80~7.78 (m, 2H), 6.82~6.79 (d, J = 8.8 Hz, 2H), 4.85~4.68 (m, |
| | 2H), 4.26~4.21 (m, 1H), 4.02~4.00 (d, J = 11.2 Hz, 1H), 3.77~3.71 (m, 1H), 3.12~3.10 (d, J = |
| | 11.2 Hz, 1H), 3.05 (s, 6H), 2.81~2.78 (d, J = 11.6 Hz, 1H), 2.76~2.75 (d, J = 4.8 Hz, 3H), |
| | 2.72~2.61 (m, 2H), 2.43~2.40 (t, J = 6.4 Hz, 2H), 2.30~2.24 (m, 1H), 2.20~2.15 (t, J = 10.8 Hz, |
| | 1H). |
| 114 | 1H NMR (400 MHz, cdcl3) δ 8.90~8.87 (dd, J = 1.6 Hz, 9.2 Hz, 1H), 8.75 (s, 1H), 8.04~8.01 (d, |
| | J = 8.8 Hz, 2H), 7.81~7.78 (d, J = 10.0 Hz, 1H), 6.74~6.71 (dd, J = 2.8 Hz, 8.8 Hz, 2H), 4.88~4.72 |
| | (m, 2H), 4.69~4.39 (dd, J = 13.2 Hz, 106.8 Hz, 1H), 4.37~4.33 (dd, J = 6.0 Hz, 12.4 Hz, 1H), |
| | 4.12~4.05 (m, 1H), 4.04~4.00 (dd, J = 3.6 Hz, 11.6 Hz, 1H), 3.96~3.58 (m, 4H), 3.41~3.23 (m, |
| | 6H), 2.93~2.77 (m, 1H), 2.11~2.10 (d, J = 6.4 Hz, 3H). |
| 115 | 1H NMR (400 MHz, cdcl3) δ 8.89~8.88 (d, J = 1.6 Hz, 1H), 8.74~8.73 (d, J = 1.6 Hz, 1H), |
| | 8.10~8.08 (d, J = 8.8 Hz, 2H), 7.80 (s, 1H), 6.82~6.80 (d, J = 8.8 Hz, 2H), 6.01 (s, 1H), |
| | 4.83~4.68 (m, 2H), 4.26~4.20 (m, 1H), 3.99~3.96 (d, J = 11.2 Hz, 1H), 3.78~3.72 (m, 1H), |
| | 3.39~3.34 (dd, J = 5.2 Hz, 11.6 Hz, 2H), 3.06~3.03 (m, 7H), 2.73~2.70 (d, J = 11.6 Hz, 1H), |
| | 2.54~2.51 (t, J = 6.0 Hz, 2H), 2.30~2.24 (m, 1H), 2.20~2.15 (t, J = 10.8 Hz, 1H), 1.97 (s, 3H). |
| 116 | 1H NMR (400 MHz, cdcl3) δ 8.89~8.88 (d, J = 1.2 Hz, 1H), 8.74~8.73 (d, J = 2.0 Hz, 1H), |
| | 8.10~8.08 (d, J = 8.4 Hz, 2H), 7.80 (s, 1H), 6.83~6.81 (d, J = 8.8 Hz, 2H), 4.93~4.90 (t, J = 5.6 |
| | Hz, 1H), 4.86~4.66 (m, 2H), 4.22~4.16 (m, 1H), 3.99~3.97 (d, J = 11.2 Hz, 1H), 3.78~3.71 (m, |
| | 1H), 3.25~3.20 (m, 2H), 3.06~3.02 (m, 7H), 2.95 (s, 3H), 2.72~2.70 (d, J = 10.0 Hz, 1H), |
| | 2.61~2.58 (t, J = 6.0 Hz, 2H), 2.35~2.29 (m, 1H), 2.26~2.21 (t, J = 10.8 Hz, 1H). |
| 117 | 1H NMR (400 MHz, cdcl3) δ 8.88~8.87 (d, J = 1.6 Hz, 1H), 8.74~8.73 (d, J = 1.6 Hz, 1H), |
| | 8.10~8.07 (d, J = 9.2 Hz, 2H), 7.80 (s, 1H), 6.82~6.80 (d, J = 8.8 Hz, 2H), 5.02 (s, 1H), |
| | 4.83~4.67 (m, 2H), 4.25~4.19 (m, 1H), 3.99~3.96 (d, J = 11.2 Hz, 1H), 3.76~3.70 (m, 1H), |
| | 3.36~3.32 (dd, J = 5.6 Hz, 10.8 Hz, 2H), 3.05~3.02 (m, 7H), 2.86 (s, 6H), 2.73~2.70 (d, J = 11.6 |
| | Hz, 1H), 2.56~2.53 (m, 2H), 2.31~2.25 (m, 1H), 2.22~2.16 (t, J = 10.8 Hz, 1H). |
| 118 | 1H NMR (400 MHz, cdcl3) δ 8.89~8.88 (d, J = 2.0 Hz, 1H), 8.74~8.73 (d, J = 1.6 Hz, 1H), |
| | 8.10~8.08 (d, J = 8.8 Hz, 2H), 7.80 (s, 1H), 6.83~6.81 (d, J = 9.2 Hz, 2H), 4.87~4.65 (m, 3H), |
| | 4.22~4.16 (m, 1H), 3.99~3.97 (d, J = 10.0 Hz, 1H), 3.78~3.72 (m, 1H), 3.16~3.13 (dd, J = 5.6 |
| | Hz, 9.2 Hz, 2H), 3.06~3.01 (m, 7H), 2.78 (s, 6H), 2.73~2.70 (d, J = 12.8 Hz, 1H), 2.59~2.56 (t, |
| | J = 5.6 Hz, 2H), 2.34~2.27 (m, 1H), 2.25~2.20 (t, J = 10.4 Hz, 1H). |
| 119 | 1H NMR (400 MHz, cdcl3) δ 8.88~8.87 (d, J = 2.0 Hz, 1H), 8.73~8.72 (d, J = 2.0 Hz, 1H), |
| | 8.06~8.04 (d, J = 9.2 Hz, 2H), 7.79 (s, 1H), 6.79~6.77 (d, J = 8.8 Hz, 2H), 4.90~4.73 (m, 2H), |
| | 4.29~4.23 (m, 1H), 4.11~4.08 (d, J = 11.6 Hz, 1H), 3.91~3.88 (d, J = 12.0 Hz, 1H), 3.83~3.77 |
| | (m, 1H), 3.62~3.59 (d, J = 11.6 Hz, 1H), 3.47~3.42 (dd, J = 7.2 Hz, 14.0 Hz, 4H), 2.97~2.84 (m, |
| | 2H), 2.81 (s, 3H), 1.24~1.21 (t, J = 6.8 Hz, 6H). |
| 120 | 1H NMR (400 MHz, dmso) δ 10.96 (s, 1H), 9.07 (s, 1H), 8.90 (s, 1H), 8.28 (s, 1H), 8.03 (s, 1H), |
| | 7.90 (d, J = 9.2, 1H), 7.58 (d, J = 7.0, 1H), 7.22 (s, 1H), 4.73 (d, J = 23.6, 2H), 4.05 (dd, J = 21.4, |
| | 7.6, 2H), 3.67 (dd, J = 29.9, 9.2, 2H), 2.89 (dd, J = 21.4, 10.9, 5H), 2.28 (s, 3H). |
| 121 | 1H NMR (400 MHz, dmso) δ 9.12 (d, J = 1.5, 1H), 8.95 (d, J = 1.6, 1H), 8.21 (s, 1H), 7.58 (s, |
| | 2H), 4.73 (d, J = 5.1, 2H), 4.11 (s, 1H), 4.02 (d, J = 11.1, 1H), 3.93 (s, 6H), 3.72 (d, J = 16.6, |
| | 3H), 3.64 (dd, J = 25.1, 10.9, 2H), 3.40 (d, J = 11.6, 1H), 2.94 (s, 5H), 2.92-2.82 (m, 2H). |
| 122 | 1H NMR (400 MHz, dmso) δ 9.07 (d, J = 1.7, 1H), 8.89 (d, J = 1.8, 1H), 8.16 (d, J = 8.9, 2H), |
| | 7.96 (s, 1H), 7.08 (d, J = 8.9, 2H), 4.69 (qd, J = 11.5, 5.4, 2H), 4.02 (d, J = 13.0, 2H), 3.73-3.58 |
| | (m, 6H), 3.41 (d, J = 11.8, 1H), 3.33 (s, 1H), 3.28-3.23 (m, 2H), 2.95 (s, 3H), 2.92-2.82 (m, |
| | 2H), 2.06 (s, 3H). |
| 123 | 1H NMR (400 MHz, dmso) δ 9.00 (d, J = 1.8, 1H), 8.80 (d, J = 1.8, 1H), 8.09 (d, J = 8.9, 2H), |
| | 7.84 (s, 1H), 6.81 (d, J = 9.0, 2H), 5.93-5.80 (m, 1H), 4.11 (dd, J = 10.4, 4.9, 1H), 3.99-3.88 |
| | (m, 2H), 3.82 (td, J = 8.2, 4.9, 1H), 2.95 (s, 6H), 2.40 (dt, J = 14.7, 7.4, 1H), 2.24-2.15 (m, 1H). |
| 124 | 1H NMR (400 MHz, dmso) δ 9.11 (d, J = 1.5, 1H), 8.93 (d, J = 1.5, 1H), 8.21 (d, J = 8.2, 2H), |
| | 8.07 (s, 1H), 7.45 (d, J = 8.2, 2H), 5.91 (s, 1H), 4.14 (dd, J = 10.4, 4.8, 1H), 4.01-3.92 (m, 2H), |
| | 3.85 (dt, J = 13.1, 6.5, 1H), 3.71 (d, J = 11.7, 2H), 2.91 (s, 3H), 2.85 (t, J = 11.2, 2H), 2.74 (t, J = |
| | 12.3, 1H), 2.42 (dd, J = 14.2, 7.3, 1H), 2.27-2.19 (m, 1H), 1.93 (d, J = 12.9, 2H), 1.80-1.69 |
| | (m, 2H). |
| 125 | 1H NMR (400 MHz, dmso) δ 9.03 (s, 1H), 8.83 (s, 1H), 8.11 (d, J = 8.7, 2H), 7.90 (s, 1H), 7.04 |
| | (d, J = 8.9, 2H), 5.87 (s, 1H), 4.11 (dd, J = 10.3, 4.8, 1H), 3.93 (dd, J = 18.1, 9.6, 2H), 3.86- |
| | 3.79 (m, 1H), 3.26 (d, J = 4.2, 4H), 2.45 (d, J = 4.9, 4H), 2.39 (d, J = 6.9, 1H), 2.21 (s, 3H), 2.20- |
| | 2.15 (m, 1H). |
| 126 | 1H NMR (400 MHz, dmso) δ 9.08 (s, 1H), 8.90 (s, 1H), 8.08 (s, 1H), 7.88-7.76 (m, 2H), 7.10 |
| | (d, J = 8.5, 1H), 4.69 (s, 2H), 4.05 (dd, J = 39.3, 8.7, 4H), 3.89 (s, 3H), 3.63 (dd, J = 24.4, 11.1, |
| | 2H), 3.37 (s, 2H), 2.92 (s, 6H), 2.90-2.82 (m, 2H), 2.66 (s, 2H), 2.23 (s, 6H). |
| 127 | 1H NMR (400 MHz, dmso) δ 9.08 (d, J = 1.9, 1H), 8.88 (d, J = 1.8, 1H), 8.15 (d, J = 8.9, 2H), |
| | 7.91 (s, 1H), 6.90 (d, J = 9.0, 2H), 5.50 (dt, J = 10.7, 3.5, 1H), 4.12 (dd, J = 11.2, 3.1, 1H), 3.77 |
| | (ddd, J = 17.7, 11.2, 6.3, 2H), 3.66 (ddd, J = 11.1, 7.8, 3.1, 1H), 3.06 (s, 6H), 2.28 (dd, J = 9.0, |
| | 4.4, 1H), 2.06-1.92 (m, 2H), 1.79-1.66 (m, 1H). |
| 128 | 1H NMR (400 MHz, cdcl3) δ 8.89~8.88 (d, J = 2.0 Hz, 1H), 8.74~8.73 (d, J = 2.0 Hz, 1H), |
| | 8.09~8.07 (d, J = 8.8 Hz, 2H), 7.81 (s, 1H), 6.83~6.81 (d, J = 8.8 Hz, 2H), 44.87~66 (m, 3H), |
| | 4.20~4.14 (m, 1H), 4.02~3.99 (d, J = 12.0 Hz, 1H), 3.75~3.69 (m, 1H), 3.19~3.15 (m, 2H), |
| | 3.11~3.06 (m, 7H), 2.92~2.88 (dd, J = 6.0 Hz, 12.8 Hz, 2H), 2.78~2.72 (m, 4H), 2.39~2.32 (m, |
| | 1H), 2.25~2.20 (t, J = 10.8 Hz, 1H). |
| 129 | 1H NMR (400 MHz, cdcl3) ¦Ä 8.89 (d, J = 1.8, 1H), 8.77 (d, J = 1.8, 1H), 8.07 (dd, J = 9.2, 2.4, |
| | 2H), 7.83 (s, 1H), 6.55 (s, 1H), 4.81 (dd, J = 11.6, |
| | 5.6, 1H), 4.68 (dd, J = 11.5, 5.2, 1H), 4.52 (s, 2H), 4.19 (dtd, J = 10.4, 5.3, 2.6, 1H), 4.03 (dd, |
| | J = 11.7, 1.7, 1H), 3.84 (d, J = 11.5, 1H), 3.74 (td, J = 11.5, 2.6, 1H), 3.55 (d, J = 11.6, 1H), 3.42 |
| | (t, J = 1.1, 1H), 2.92 \ddot{ }C 2.84 (m, 4H), 2.79 (d, J = 10.5, 1H), 2.75 (s, 3H). |
| 130 | 1H NMR (400 MHz, cdcl3) δ 8.97 (d, J = 1.8, 1H), 8.85 (d, J = 1.8, 1H), 7.96 (s, 1H), 7.3-7.71 |
| | (m, 2H), 7.42 (t, J = 8.2, 1H), 7.01 (dd, J = 8.0, 2.4, 1H), 4.87 (dd, J = 11.6, 5.7, 1H), 4.76 (dd, |
| | J = 11.6, 5.0, 1H), 4.29-4.23 (m, 1H), 4.21 (t, J = 5.6, 2H), 4.13--4.05 (m, 1H), 3.92-3.85 (m, |
| | 1H), 3.80 (td, J = 11.5, 2.7, 1H), 3.60 (d, J = 12.0, 1H), 2.95 (dd, J = 11.5, 3.3, 1H), 2.91-2.88 |
| | (m, 3H), 2.86 (d, J = 2.8, 1H), 2.81 (s, 3H), 2.42 (s, 6H). |
| 131 | 1H NMR (400 MHz, cdcl3) δ 8.87 (d, J = 7.8, 1H), 8.72 (s, 1H), 8.05 (d, J = 8.7, 2H), 7.77 (d, J = |
| | 8.9, 1H), 6.77 (d, J = 5.9, 2H), 4.99-4.29 (m, 3H), 4.09 (s, 1H), 4.02 (d, J = 11.6, 1H), 3.73 (m, |
| | 2H), 3.53-3.37 (m, 5H), 3.37 (m, 1H), 2.85 (m, 1H), 2.05 (d, 3H), 1.22 (t, J = 6.9, 6H). |
| 132 | 1H NMR (400 MHz, cdcl3) δ 8.94 (dd, J = 9.2, 1.5, 1H), 8.81 (s, 1H), 7.88 (d, J = 9.8, 1H), 7.77 |
| | (dd, J = 8.4, 1H), 7.71 (d, J = 2.0, 1H), 7.00 (dd, J = 8.4 1H), 4.80 (m, 2H), 4.54 (m, 1H), 4.12- |
| | 4.05 (m, 1H), 4.05-3.91 (m, 8H), 3.69-3.56 (m, 2H), 3.33 (m, 1H), 2.96-2.74 (m, 1H), 2.10 |
| | (d, J = 5.5, 3H). |
| 133 | 1H NMR (400 MHz, cdcl3) δ 8.82 (d, J = 1.7, 1H), 8.69 (d, J = 1.8, 1H), 7.97 (d, J = 8.7, 2H), |
| | 7.73 (s, 1H), 6.66 (d, J = 8.7, 2H), 5.91-5.83 (m, 1H), 4.30 (M, 1H), 4.11-4.03 (m, 2H), 3.96- |
| | 3.89 (m, 1H), 2.86 (s, 3H), 2.39 (M, 2H). |
| 134 | 1H NMR (400 MHz, cdcl3) δ 8.94~8.93 (d, J = 2.0 Hz, 1H), 8.80~8.79 (d, J = 2.0 Hz, 1H), |
| | 7.96~7.94 (m, 2H), 7.88 (s, 1H), 7.13~7.11 (d, J = 9.2 Hz, 1H), 4.90~4.74 (m, 2H), 4.29~4.23 |
| | (m, 1H), 4.12~4.08 (m, 1H), 3.92~3.88 (m, 1H), 3.84~3.77 (m, 1H), 3.62~3.60 (d, J = 9.2 Hz, |
| | 1H), 2.98~2.85 (m, 2H), 2.81 (s, 3H), 2.79 (s, 6H), 2.44 (s, 3H). |
| 135 | 1H NMR (400 MHz, cdcl3) δ 8.91~8.88 (dd, J = 1.6 Hz, 10.8 Hz, 1H), 8.76~8.74 (dd, J = 1.6 Hz, |
| | 6.4 Hz, 1H), 8.05~8.03 (d, J = 8.8 Hz, 2H), 7.81~7.79 (d, J = 8.8 Hz, 1H), 6.72~6.70 (d, J = 8.8 |
| | Hz, 2H), 4.88~4.41 (m, 3H), 4.10~3.71 (m, 4H), 3.64~3.58 (t, J = 12.0 Hz, 1H), 3.38~3.16 (m, |
| | 2H), 2.95~2.67 (m, 6H), 2.66~2.62 (t, J = 8.4 Hz, 1H), 2.49~2.40 (m, 1H), 2.38~2.34 (d, J = |
| | 18 Hz, 3H), 2.13~2.02 (m, 2H). |
| 136 | 1H NMR (400 MHz, cdcl3) δ 8.90~8.89 (d, J = 2.0 Hz, 1H), 8.73~8.72 (d, J = 2.0 Hz, 1H), |
| | 8.10~8.07 (d, J = 9.2 Hz, 2H), 7.81 (s, 1H), 6.83~6.81 (d, J = 8.8 Hz, 2H), 4.89~4.70 (m, 2H), |
| | 4.55 (s, 2H), 4.17~4.11 (m, 1H), 4.04~3.99 (m, 2H), 3.81~3.78 (d, J = 12.4 Hz, 1H), 3.73~3.66 |
| | (m, 1H), 3.15~3.03 (m, 8H). |
| 137 | 1H NMR (400 MHz, cdcl3) δ 8.89~8.88 (d, J = 1.6 Hz, 1H), 8.73~8.72 (d, J = 1.6 Hz, 1H), |
| | 8.09~8.07 (d, J = 8.8 Hz, 2H), 7.80 (s, 1H), 6.83~6.81 (d, J = 8.8 Hz, 2H), 4.88~4..68 (m, 2H), |
| | 4.65~4.64 (d, J = 4.0 Hz, 1H), 4.14~4.08 (m, 1H), 4.03~3.97 (m, 2H), 3.82~3.79 (d, J = 12.4 Hz, |
| | 1H), 3.71~3.64 (m, 1H), 3.08~2.96 (m, 8H), 2.80~2.79 (d, J = 4.4 Hz, 3H). |
| 138 | 1H NMR (400 MHz, cdcl3) δ 8.99~8.96 (dd, J = 1.6 Hz, 8.4 Hz, 1H), 8.87~8.86 (t, J = 2.0 Hz, |
| | 1H), 7.96~7.94 (d, J = 10.4 Hz, 1H), 7.32~7.31 (d, J = 2.0 Hz, 2H), 6.58~6.56 (dd, J = 2.4 Hz, |
| | 4.4 Hz, 1H), 4.87~4.73 (m, 2H), 4.68~4.40 (m, 1H), 4.13~4.00 (m, 2H), 3.96~3.90 (m, 7H), |
| | 3.69~3.57 (m, 1H), 3.40~3.25 (m, 1H), 2.92~2.78 (m, 1H), 2.12~2.11 (d, J = 2.4 Hz, 3H). |
| 139 | 1H NMR (400 MHz, cdcl3) δ 8.95~8.92 (dd, J = 2.0 Hz, 8.8 Hz, 1H), 8.81~8.80 (t, J = 1.6 Hz, |
| | 1H), 7.88~7.83 (m, 3H), 6.99~6.93 (m, 1H), 4.86~4.73 (m, 2H), 4.70~4.41 (m, 1H), 4.13~4.02 |
| | (m, 2H), 3.96~3.58 (m, 2H), 3.41~3.26 (m, 1H), 2.97~2.96 (d, J = 1.6 Hz, 6H), 2.93~2.79 (m, |
| | 1H), 2.12 (s, 3H). |
| 140 | 1H NMR (400 MHz, cdcl3) δ 8.95~8.93 (dd, J = 1.6 Hz, 8.8 Hz, 1H), 8.82 (s, 1H), 8.18~8.16 (dd, |
| | J = 2.0 Hz, 8.4 Hz, 1H), 8.00~7.96 (m, 1H), 7.88~7.85 (d, J = 11.2 Hz, 1H), 7.17~7.14 (dd, J = |
| | 2.8 Hz, 8.4 Hz, 1H), 4.87~4.73 (m, 2H), 4.69~4.40 (m, 1H), 4.12~4.02 (m, 2H), 3.96~3.58 (m, |
| | 2H), 3.40~3.27 (m, 1H), 2.91~2.80 (m, 7H), 2.11 (s, 3H). |
| 141 | 1H NMR (400 MHz, cdcl3) δ 8.90~8.88 (dd, J = 2.0 Hz, 9.2 Hz, 1H), 8.75 (s, 1H), 8.03~8.01 (d, |
| | J = 8.8 Hz, 2H), 7.80~7.78 (d, J = 9.6 Hz, 1H), 6.69~6.66 (dd, J = 3.6 Hz, 8.8 Hz, 2H), 4.88~4.72 |
| | (m, 2H), 4.70~4.40 (m, 1H), 4.13~4.01 (m, 2H), 3.96~3.58 (m, 4H), 3.40~3.25 (m, 1H), |
| | 2.94~2.76 (m, 1H), 2.12~2.10 (d, J = 6.8 Hz, 3H), 1.27~1.25 (d, J = 6.4 Hz, 6H). |
| 142 | 1H NMR (400 MHz, cdcl3) δ 8.94~8.93 (d, J = 1.6 Hz, 1H), 8.80~8.79 (d, J = 2.0 Hz, 1H), 7.88 |
| | (s, 1H), 7.79~7.76 (dd, J = 2.0 Hz, 8.4 Hz, 1H), 7.72~7.71 (d, J = 2.0 Hz, 1H), 7.02~6.99 (d, J = |
| | 8.4 Hz, 1H), 4.90~4.69 (m, 2H), 4.61 (s, 2H), 4.17~4.11 (m, 1H), 4.06~4.00 (m, 5H), 3.96 (s, |
| | 3H), 3.77~3.65 (m, 2H), 3.16~3.02 (m, 2H). |
| 143 | 1H NMR (400 MHz, cdcl3) δ 8.94~8.93 (d, J = 1.6 Hz, 1H), 8.81~8.80 (d, J = 2.0 Hz, 1H), 7.88 |
| | (s, 1H), 7.79~7.76 (dd, J = 2.0 Hz, 8.4 Hz, 1H), 7.73~7.72 (d, J = 2.4 Hz, 1H), 7.02~7.00 (d, J = |
| | 8.4 Hz, 1H), 4.90~4.68 (m, 2H), 4.58~4.55 (m, 1H), 4.16~4.10 (m, 1H), 4.02~3.97 (m, 8H), |
| | 3.75~3.64 (m, 2H), 3.10~2.96 (m, 2H), 2.81~2.79 (d, J = 4.8 Hz, 3H). |
| 144 | 1H NMR (400 MHz, cdcl3) δ 8.89~8.86 (dd, J = 2.0 Hz, 9.2 Hz, 1H), 8.74~8.73 (d, J = 1.6 Hz, |
| | 1H), 8.08~8.05 (m, 2H), 7.81~7.79 (d, J = 9.6 Hz, 1H), 6.89~6.86 (dd, J = 2.8 Hz, 9.2 Hz, 2H), |
| | 4.89~4.73 (m, 2H), 4.69~4.39 (m, 1H), 4.25~4.19 (m, 1H), 4.13~4.00 (m, 2H), 3.97~3.58 (m, |
| | 2H), 3.40~3.25 (m, 1H), 2.94~2.77 (m, 4H), 2.11~2.09 (d, J = 7.6 Hz, 3H), 1.23~1.22 (d, J = 6.4 |
| | Hz, 6H). |
| 145 | 1H NMR (400 MHz, cdcl3) δ 8.97~8.94 (dd, J = 2.0, 9.2 Hz, 1H), 8.85~8.84 (t, J = 1.6 Hz, 1H), |
| | 7.97~7.94 (d, J = 10.0 Hz, 1H), 7.74~7.72 (m, 2H), 7.44~7.40 (t, J = 8.0 Hz, 1H), 7.01~6.99 (d, |
| | J = 8.4 Hz, 1H), 4.88~4.73 (m, 2H), 4.68~4.39 (m, 1H), 4.12~4.00 (m, 2H), 3.96~3.57 (m, 5H), |
| | 3.39~3.26 (m, 1H), 2.93~2.79 (m, 1H), 2.10~2.09 (d, J = 2.8 Hz, 3H). |
| 146 | 1H NMR (400 MHz, cdcl3) δ 8.97~8.94 (dd, J = 1.6 Hz, 9.6 Hz, 1H), 8.84 (s, 1H), 7.98~7.96 (d, |
| | J = 9.2 Hz, 1H), 7.55~7.50 (m, 2H), 7.40~7.36 (t, J = 8.0 Hz, 1H), 6.86~6.84 (d, J = 8.4 Hz, 1H), |
| | 4.92~4.73 (m, 2H), 4.68~4.39 (m, 1H), 4.13~4.07 (m, 1H), 4.03~4.00 (d, J = 12.4 Hz, 1H), |
| | 3.70~3.57 (m, 2H), 3.39~3.22 (m, 1H), 3.05 (s, 6H), 2.95~2.78 (m, 1H), 2.11~2.08 (d, J = 11.6 |
| | Hz, 3H). |
| 147 | 1H NMR (400 MHz, cdcl3) δ 8.94~8.91 (dd, J = 1.6 Hz, 9.2 Hz, 1H), 8.80 (s, 1H), 7.89~7.86 (d, |
| | J = 9.2 Hz, 1H), 7.75~7.71 (m, 2H), 7.02~7.00 (d, J = 8.4 Hz, 1H), 4.91~4.38 (m, 4H), 4.11~4.06 |
| | (m, 1H), 4.03~3.57 (m, 6H), 3.39~3.26 (m, 1H), 2.94~2.77 (m, 1H), 2.10~2.09 (d, J = 6.0 Hz, |
| | 3H), 1.43~1.41 (d, J = 6.0 Hz, 6H). |
| 148 | 1H NMR (400 MHz, dmso) δ 9.01 (d, J = 1.7, 1H), 8.83 (d, J = 1.8, 1H), 7.84 (d, J = 3.2, 1H), |
| | 7.72 (d, J = 8.5, 1H), 7.57 (d, J = 7.6, 1H), 6.78 (d, J = 8.5, 1H), 4.71-4.56 (m, 2H), 4.46-4.14 |
| | (m, 1H), 3.97-3.66 (m, 3H), 3.56-3.39 (m, 1H), 3.34-3.31 (m, 2H), 3.23-3.16 (m, 1H), 2.91 |
| | (s, 3H), 2.76-2.66 (m, 1H), 2.01 (d, J = 4.5, 3H). |
| 149 | 1H NMR (400 MHz, dmso) δ 9.02 (d, J = 1.8, 1H), 8.83 (d, J = 1.8, 1H), 8.13 (d, J = 8.9, 2H), |
| | 7.91 (s, 1H), 7.05 (d, J = 9.0, 2H), 5.91-5.82 (m, 1H), 4.14-4.08 (m, 1H), 3.97-3.89 (m, 2H), |
| | 3.85-3.80 (m, 1H), 3.77-3.73 (m, 4H), 3.23-3.1 9 (m, 4H), 2.45-2.36 (m, 7.3, 1H), 2.24- |
| | 2.16 (m, 1H). |
| 150 | 1H NMR (400 MHz, dmso) δ 9.06 (d, J = 1.9, 1H), 8.88 (d, J = 1.9, 1H), 8.05 (s, 1H), 7.87- |
| | 7.78 (m, 2H), 7.08 (d, J = 8.5, 1H), 5.90-5.84 (m, 1H), 4.14-4.08 (m, 1H), 4.00-3.89 (m, 2H), |
| | 3.89 (s, 3H), 3.86-3.81 (m, 4H), 2.45-2.36 (m, 1H), 2.27-2.18 (m, 1H). |
| 151 | 1H NMR (400 MHz, cdcl3) δ 8.83-8.76 (m, 1H), 8.71-8.60 (m, 1H), 8.05-7.91 (m, 2H), 7.70 (s, |
| | 1H), 6.79-6.61 (m, 2H), 5.91-5.82 (m, 1H), 4.31-4.27 (m, 1H), 4.08-4.01 (m, 2H), 3.94-3.88 (m, |
| | 1H), 3.42-3.33 (m, 4H), 2.41-2.35 (m, 2H), 1.16 (t, J = 7.1, 6H). |
| 152 | 1H NMR (400 MHz, dmso) δ 9.02 (s, 1H), 8.83 (s, 1H), 8.12 (d, J = 8.5, 2H), 7.90 (s, 1H), 7.05 |
| | (d, J = 8.5, 2H), 5.92-5.80 (m, 1H), 4.14-4.07 (m, 1H), 3.97-3.88 (m, 2H), 3.85-3.79 (m, |
| | 1H), 3.62-3.54 (m, 4H), 3.26-3.13 (m, 4H), 2.43-2.35 (m, 1H), 2.23-2.15 (m, 1H), 2.03 (s, |
| | 3H). |
| 153 | 1H NMR (400 MHz, dmso) δ 9.04 (s, 1H), 8.84 (s, 1H), 8.14 (d, J = 8.3, 2H), 7.93 (s, 1H), 7.10 |
| | (d, J = 8.2, 2H), 5.94-5.78 (m, 1H), 4.17-4.08 (m, 1H), 3.99-3.90 (m, 2H), 3.86-3.80 (m, 1H), |
| | 3.41-3.37 (m, 4H), 3.26-3.23 (m, 4H), 2.92 (s, 3H), 2.45-2.37 (m, 1H), 2.28-2.15 (m, 1H). |
| 154 | 1H NMR (400 MHz, dmso) δ 9.07 (d, J = 1.8, 1H), 8.89 (d, J = 1.8, 1H), 8.22 (d, J = 2.0, 1H), |
| | 8.14 (d, J = 2.1, 1H), 8.03 (s, 1H), 7.24 (d, J = 8.5, 1H), 5.9 1-5.82 (m, 1H), 4.13-4.07 (m, 1H), |
| | 4.00-3.88 (m, 2H), 3.99-3.88 (m, 1H), 2.81 (s, 6H), 2.46-2.36 (m, 1H), 2.25-2.15 (m, 1H). |
| 155 | 1H NMR (400 MHz, cdcl3) δ 8.90 (d, J = 1.8, 1H), 8.78 (d, J = 1.7, 1H), 7.97-7.89 ( m, 2H), 7.85 |
| | (s, 1H), 7.12 (s, 1H), 5.96-5.88 (m, 1H), 4.36-4.31 (m, 1H), 4.14-4.06 (m, 2H), 3.99-3.93 (m, |
| | 1H), 2.79 (s, 6H), 2.49-2.38 (m, 5H). |
| 156 | 1H NMR (400 MHz, dmso) δ 9.07 (s, 1H), 8.90 (s, 1H), 8.16 (d, J = 6.2, 2H), 8.02 (s, 1H), 7.38 |
| | (d, J = 6.2, 2H), 5.94-5.82 (m, 1H), 4.53 (d, J = 10.7, 1H), 4.16-4.06 (m, 1H), 4.00-3.87 (m, 3H), |
| | 3.85-3.77 (m, 1H), 3.17-3.09 (m, 2H), 2.87-2.77 (m, 1H), 2.63-2.54 (m, 1H), 2.45-2.36 (m, 1H), |
| | 2.24-2.16 (m, 1H), 2.02 (s, 3H), 1.87-1.74 (m, 2H), 1.67-1.57 (m, 1H), 1.53-1.32 (m, 1H). |
| 157 | 1H NMR (400 MHz, cdcl3) δ 8.94~8.91 (dd, J = 1.6 Hz, 8.8 Hz, 1H), 8.80 (s, 1H), 7.86~7.84 (d, |
| | J = 10.0 Hz, 1H), 7.77~7.73 (m, 2H), 7.01~6.99 (dd, J = 1.6 Hz, 8.4 Hz, 1H), 4.91~4.38 (m, 4H), |
| | 4.10~3.99 (m, 2H), 3.95~3.57 (m, 5H), 3.39~3.26 (m, 1H), 2.93~2.78 (m, 1H), 2.10~2.08 (d, J = |
| | 6.4 Hz, 3H), 1.44~1.42 (d, J = 6.0 Hz, 6H). |
| 158 | 1H NMR (400 MHz, cdcl3) δ 8.93~8.90 (dd, J = 1.6 Hz, 8.8 Hz, 1H), 8.79 (s, 1H), 7.96~7.94 (m, |
| | 2H), 7.88~7.86 (d, J = 10.0 Hz, 1H), 7.13~7.10 (dd, J = 2.0 Hz, 8.8 Hz, 1H), 4.89~4.73 (m, 2H), |
| | 4.70~4.39 (m, 1H), 4.13~3.58 (m, 4H), 3.40~3.26 (m, 1H), 2.95~2.78 (m, 7H), 2.43 (s, 3H), |
| | 2.11~2.09 (d, J = 7.2 Hz, 3H). |
| 159 | 1H NMR (400 MHz, cdcl3) δ 8.88~8.87 (d, J = 1.6 Hz, 1H), 8.74~8.73 (d, J = 1.6 Hz, 1H), |
| | 8.10~8.08 (d, J = 9.2 Hz, 2H), 7.80 (s, 1H), 6.83~6.80 (d, J = 9.2 Hz, 2H), 4.86~4.72 (m, 2H), |
| | 4.21~4.15 (m, 1H), 4.00~3.96 (m, 1H), 3.77~3.69 (m, 2H), 3.49~3.46 (d, J = 13.2 Hz, 1H), |
| | 3.10~3.03 (m, 7H), 3.01~2.95 (dd, J = 10.8 Hz, 13.2 Hz, 1H), 2.83 (s, 6H). |
| 160 | 1H NMR (400 MHz, cdcl3) δ 8.96~8.94 (dd, J = 1.6 Hz, 9.6 Hz, 1H), 8.83 (s, 1H), 8.11~8.08 (d, |
| | J = 8.4 Hz, 2H), 7.95~7.93 (d, J = 10.0 Hz, 1H), 7.39~7.37 (d, J = 8.4 Hz, 2H), 4.90~4.75 (m, 2H), |
| | 4.70~4.40 (m, 1H), 4.11~4.01 (m, 2H), 3.73~3.59 (m, 2H), 3.40~3.26 (m, 1H), 3.03~2.79 (m, |
| | 2H), 2.12~2.09 (d, J = 9.2 Hz, 3H), 1.32~1.31 (d, J = 7.2 Hz, 6H). |
| 161 | 1H NMR (400 MHz, cdcl3) δ 8.94~8.92 (d, J = 8.8 Hz, 1H), 8.81 (s, 1H), 7.90~7.87 (d, J = 10.0 |
| | Hz, 1H), 7.76 (s, 2H), 4.89~4.73 (m, 2H), 4.70~4.40 (m, 1H), 4.11~4.02 (m, 2H), 3.97~3.59 (m, |
| | 2H), 3.40~3.26 (m, 1H), 2.93~2.79 (m, 7H), 2.40 (s, 6H), 2.11~2.09 (d, J = 8.0 Hz, 3H). |
| 162 | 1H NMR (400 MHz, cdcl3) δ 8.96~8.83 (d, J = 9.2 Hz, 1H), 8.83 (s, 1H), 8.11~8.09 (d, J = 8.0 |
| | Hz, 2H), 7.95~7.92 (d, J = 10.4 Hz, 1H), 7.38~7.36 (d, J = 8.0 Hz, 2H), 4.88~4.73 (m, 2H), |
| | 4.69~4.39 (m, 1H), 4.09~4.01 (m, 2H), 3.96~3.58 (m, 2H), 3.39~3.25 (m, 3H), 2.93~2.69 (m, |
| | 4H), 2.56 (s, 2H), 2.11~2.09 (d, J = 7.6 Hz, 3H), 1.92~1.89 (m, 1H), 1.80~1.70 (m, 2H). |
| 163 | 1H NMR (400 MHz, cdcl3) δ 8.90 (s, 1H), 8.76 (s, 1H), 7.95~7.94 (m, 2H), 7.85 (s, 1H), |
| | 7.12~7.10 (d, J = 9.2 Hz, 1H), 4.87~4.69 (m, 4H), 4.16~4.00 (m, 3H), 3.80~3.77 (d, J = 12.8 Hz, |
| | 1H), 3.71~3.66 (t, J = 11.2 Hz, 1H), 3.13~3.02 (m, 2H), 2.79 (s, 6H), 2.43 (s, 3H). |
| 164 | 1H NMR (400 MHz, cdcl3) δ 8.93 (s, 1H), 8.80 (s, 1H), 7.97~7.96 (m, 2H), 7.88 (s, 1H), |
| | 7.14~7.11 (d, J = 9.2 Hz, 1H), 4.89~4.72 (m, 2H), 4.50~4.46 (m, 1H), 4.16~4.10 (m, 1H), |
| | 4.04~3.95 (m, 2H), 3.79~3.76 (d, J = 13.6 Hz, 1H), 3.71~3.65 (m, 1H), 3.10~3.98 (m, 2H), |
| | 2.81~2.80 (m, 9H), 2.44 (s, 3H). |
| 165 | 1H NMR (400 MHz, cdcl3) δ 8.93 (s, 1H), 8.80 (s, 1H), 8.16 (s, 1H), 7.98~7.96 (d, J = 8.4 Hz, |
| | 1H), 7.85 (s, 1H), 7.16~7.14 (d, J = 8.4 Hz, 1H), 4.87~4.67 (m, 4H), 4.15~4.01 (m, 3H), |
| | 3.80~3.77 (d, J = 12.8 Hz, 1H), 3.72~3.66 (t, J = 11.2 Hz, 1H), 3.15~3.03 (m, 2H), 2.91 (s, 6H). |
| 166 | 1H NMR (400 MHz, cdcl3) δ 8.93 (s, 1H), 8.80 (s, 1H), 8.16 (s, 1H), 7.98~7.96 (d, J = 8.4 Hz, |
| | 1H), 7.85 (s, 1H), 7.16~7.14 (d, J = 8.4 Hz, 1H), 4.86~4.65 (m, 3H), 4.14~4.08 (m, 1H), |
| | 4.02~3.97 (t, J = 11.2 Hz, 2H), 3.79~2.76 (d, J = 13.6 Hz, 1H), 3.71~3.67 (m, 1H), 3.09~2.97 |
| | (m, 2H), 2.91 (s, 6H), 2.81~2.80 (d, J = 4.4 Hz, 3H). |
| 167 | 1H NMR (400 MHz, cdcl3) δ 8.86 (s, 1H), 8.74 (s, 1H), 8.10 (d, J = 8.2, 2H), 7.78 (s, 1H), 6.94- |
| | 6.80 (m, 2H), 4.82-4.72 (m, 1H), 4.67-4.57 (m, 1H), 4.10-3.96 (m, 2H), 3.54 (t, J = 10.6, 1H), |
| | 3.05 (s, 6H), 1.95-1.82 (m, 2H), 1.68-1.48 (m, 4H). |
| 168 | 1H NMR (400 MHz, cdcl3) δ 8.79 (s, 1H), 8.66 (s, 1H), 8.02 (d, J = 8.5, 2H), 7.71 (s, 1H), 6.77 |
| | (d, J = 8.5, 2H), 4.75-4.67 (m, 1H), 4.65-4.57 (m, 1H), 4.54-4.46 (m, 1H), 3.94-3.87 (m, 1H), |
| | 3.81-3.74 (m, 1H), 2.98 (s, 6H), 2.14-2.03 (m, 1H), 2.02-1.93 (m, 1H), 1.93-1.86 (m, 1H), 1.85- |
| | 1.78 (m, 1H). |
| 169 | 1H NMR (400 MHz, cdcl3) δ 8.93 (s, 1H), 8.85 (s, 1H), 8.13 (d, J = 7.8, 2H), 7.92 (s, 1H), 7.35 |
| | (d, J = 7.8, 2H), 4.82-4.74 (m, 1H), 4.66-4.58 (m, 1H), 4.07-3.94 (m, 4H), 3.55 (t, J = 11.3, |
| | 1H), 2.88-2.77 (m, 5H), 2.70 (t, J = 12.1, 1H), 2.03-1.99 (m, 1H), 1.95-1.82 (m, 4H), 1.68- |
| | 1.49 (m, 4H). |
| 170 | 1H NMR (400 MHz, cdcl3) δ 8.93 (s, 1H), 8.84 (s, 1H), 8.13 (d, J = 7.6, 2H), 7.92 (s, 1H), 7.34 |
| | (d, J = 7.6, 2H), 4.81-4.73 (m, 1H), 4.72-4.65 (m, 1H), 4.59-4.51 (m, 1H), 4.02-3.92 (m, 3H), |
| | 3.88-3.79 (m, 1H), 2.87-2.76 (m, 5H), 2.74-2.64 (m, 1H), 2.22-2.11 (m, 1H), 2.02-1.97 (m, 3H), |
| | 1.96-1.83 (m, 4H). |
| 171 | 1H NMR (400 MHz, cdcl3) δ 8.96~8.94 (d, J = 8.0 Hz, 1H), 8.82 (s, 1H), 8.14~8.12 (d, J = 8.4 |
| | Hz, 1H), 8.08 (s, 1H), 7.91~7.88 (d, J = 11.6 Hz, 1H), 6.96~6.94 (d, J = 8.0 Hz, 1H), 4.91~4.41 |
| | (m, 3H), 4.12~4.02 (m, 2H), 3.98~3.60 (m, 4H), 3.41~3.28 (m, 4H), 2.96~2.79 (m, 1H), 2.12 (s, |
| | 3H). |
| 172 | 1H NMR (400 MHz, cdcl3) δ 8.88 (s, 1H), 8.74 (s, 1H), 8.09~8.07 (d, J = 8.8 Hz, 2H), 7.80 (s, |
| | 1H), 6.83~6.81 (d, J = 6.8 Hz, 2H), 4.89~4.75 (m, 2H), 4.62~4.40 (m, 1H), 4.37~4.15 (m, 2H), |
| | 4.08~4.01 (m, 1H), 3.71~3.65 (t, J = 11.6 Hz, 1H), 3.44~3.32 (m, 1H), 3.05~2.95 (m, 7H), |
| | 1.89~1.78 (m, 3H). |
| 173 | 1H NMR (400 MHz, cdcl3) δ 8.90~8.88 (d, J = 7.2 Hz, 1H), 8.74 (s, 1H), 8.08~8.06 (d, J = 8.4 |
| | Hz, 2H), 7.82~7.81 (d, J = 5.2 Hz, 1H), 6.83~6.81 (d, J = 8.0 Hz, 2H), 4.90~4.72 (m, 2H), |
| | 4.67~4.36 (m, 1H), 4.25~4.03 (m, 4H), 3.70~3.48 (m, 3H), 3.31~3.21 (m, 1H), 3.06~2.95 (m, |
| | 7H). |
| 174 | 1H NMR (400 MHz, cdcl3) δ 8.93~8.90 (m, 2H), 8.79 (s, 1H), 8.03 (s, 1H), 7.82~7.80 (d, J = 7.6 |
| | Hz, 1H), 4.85~4.69 (m, 2H), 4.67~4.38 (m, 1H), 4.07~3.99 (m, 2H), 3.93~3.56 (m, 2H), |
| | 3.38~3.24 (m, 1H), 2.97 (s, 6H), 2.92~2.76 (m, 1H), 2.40 (s, 3H), 2.09 (s, 3H). |
| 175 | 1H NMR (400 MHz, cdcl3) δ 8.94~8.93 (d, J = 2.0 Hz, 1H), 8.81~8.80 (d, J = 1.6 Hz, 1H), |
| | 8.10~8.08 (d, J = 8.4 Hz, 2H), 7.93 (s, 1H), 7.39~7.37 (d, J = 8.4 Hz, 2H), 4.89~4.67 (m, 4H), |
| | 4.16~4.10 (m, 1H), 4.05~4.00 (m, 2H), 3.79~3.76 (d, J = 13.2 Hz, 1H), 3.72~3.65 (m, 1H), |
| | 3.15~2.96 (m, 3H), 1.32~1.30 (d, J = 6.8 Hz, 6H). |
| 176 | 1H NMR (400 MHz, cdcl3) δ 8.94~8.93 (d, J = 1.6 Hz, 1H), 8.80~8.79 (d, J = 1.6 Hz, 1H), |
| | 8.10~8.07 (d, J = 8.4 Hz, 2H), 7.92 (s, 1H), 7.38~7.36 (d, J = 8.0 Hz, 2H), 4.88~4.67 (m, 3H), |
| | 4.14~4.08 (m, 1H), 4.02~3.98 (d, J = 13.2 Hz, 2H), 3.79~3.76 (d, J = 12.8 Hz, 1H), 3.70~3.64 |
| | (m, 1H), 3.09~2.96 (m, 3H), 2.80~2.78 (d, J = 4.8 Hz, 3H), 1.32~1.30 (d, J = 6.8 Hz, 6H). |
| 177 | 1H NMR (400 MHz, cdcl3) δ 8.90 (s, 1H), 8.78 (s, 1H), 8.12 (d, J = 8.7, 2H), 7.83 (s, 1H), 7.02 |
| | (d, J = 8.7, 2H), 4.83-4.65 (m, 2H), 4.62-4.51 (m, 1H), 4.04-3.94 (m, 1H), 3.89-3.83 (m, |
| | 1H), 3.83 (s, 2H), 3.66 (s, 2H), 3.37-3.25 (m, 4H), 2.16 (s, 3H), 2.09-1.85 (m, 4H). |
| 178 | 1H NMR (400 MHz, dmso) δ 9.01 (s, 1H), 8.82 (s, 1H), 8.09 (d, J = 8.2, 2H), 7.85 (s, 1H), 6.87 |
| | (s, 2H), 6.81 (d, J = 8.3, 2H), 4.77-4.59 (m, 2H), |
| | 4.07-3.92 (m, 2H), 3.67-3.51 (m, 2H), 3.25 (s, 1H), 2.98 (s, 6H), 2.73-2.62 (m, 2H). |
| 179 | 1H NMR (400 MHz, cdcl3) δ 8.90 (s, 1H), 8.79 (s, 1H), 8.12 (d, J = 7.9, 2H), 7.84 (s, 1H), 7.04 |
| | (d, J = 8.1, 2H), 4.87-4.47 (m, 3H), 4.06-3.79 (m, 2H), 3.42 (s, 8H), 2.84 (s, 3H), 2.22-2.12 |
| | (m, 1H), 2.07-1.86 (m, 3H). |
| 180 | 1H NMR (400 MHz, cdcl3) δ 8.94 (s, 1H), 8.84 (s, 1H), 8.14 (d, J = 8.0, 2H), 7.92 (s, 1H), 7.36 |
| | (d, J = 8.1, 2H), 4.85-4.65 (m, 2H), 4.63-4.52 (m, 1H), 4.04-3.92 (m, 3H), 3.91-3.81 (m, |
| | 1H), 2.91-2.76 (m, 5H), 2.75-2.65 (m, 1H), 2.24-2.11 (m, 1H), 2.11-1.84 (m, 7H). |
| 181 | 1H NMR (400 MHz, cdcl3) δ 8.97-8.93 (m, 1H), 8.85-8.81 (m, 1H), 8.09 (d, J = 8.4, 2H), 7.95 |
| | (S, 1H), 7.38 (d, J = 8.2, 2H), 4.95-4.86 (m, 1H), 4.81-4.71 (m, 1H), 4.32-4.19 (m, 1H), |
| | 4.12-4.05 (m, 1H), 3.94-3.86 (m, 1H), 3.84-3.76 (m, 1H), 3.64-3.58 (m, 1H), 3.04-2.83 |
| | (m, 3H), 2.83-2.78 (s, 3H), 1.32 (d, J = 6.9, 1.0, 6H). |
| 182 | 1H NMR (400 MHz, cdcl3) δ 8.94~8.91 (d, J = 8.8 Hz, 1H), 8.80 (s, 1H), 7.97 (s, 2H), 7.90~7.87 |
| | (d, J = 10.0 Hz, 1H), 7.13~7.11 (d, J = 8.4 Hz, 1H), 4.89~4.39 (m, 3H), 4.10~4.01 (m, 2H), |
| | 3.97~3.59 (m, 2H), 3.47~3.26 (m, 2H), 3.06~2.78 (m, 9H), 2.42 (s, 3H), 2.11~2.09 (d, J = 6.4 |
| | Hz, 3H). |
| 183 | 1H NMR (400 MHz, cdcl3) δ 8.93~8.92 (t, J = 1.2 Hz, 1H), 8.78~8.77 (t, J = 1.6 Hz, 1H), |
| | 7.86~7.85 (d, J = 5.6 Hz, 1H), 7.83 (s, 2H), 6.97~6.93 (t, J = 8.8 Hz, 1H), 4.88~4.68 (m, 2H), |
| | 4.63 (s, 2H), 4.16~4.10 (m, 1H), 4.04~4.01 (d, J = 12.8 Hz, 2H), 3.81~3.78 (d, J = 12.8 Hz, 1H), |
| | 3.73~3.66 (m, 1H), 3.16~3.02 (m, 2H), 2.97 (s, 6H). |
| 184 | 1H NMR (400 MHz, cdcl3) δ 8.92~8.91 (t, J = 1.6 Hz, 1H), 8.77~8.76 (d, J = 1.2 Hz, 1H), |
| | 7.85~7.84 (d, J = 3.2 Hz, 1H), 7.82~7.81 (d, J = 1.6 Hz, 2H), 6.97~6.92 (t, J = 8.8 Hz, 1H), |
| | 4.86~4.67 (m, 3H), 4.13~4.07 (m, 1H), 4.03~3.97 (m, 2H), 3.80~3.77 (d, J = 12.8 Hz, 1H), |
| | 3.71~3.64 (m, 1H), 3.09~2.96 (m, 8H), 2.81~2.80 (d, J = 4.4 Hz, 3H). |
| 185 | 1H NMR (400 MHz, cdcl3) δ 8.88~8.86 (d, J = 8.8 Hz, 1H), 8.72 (s, 1H), 7.93~7.91 (d, J = 8.4 |
| | Hz, 1H), 7.78~7.76 (m, 2H), 6.69~6.66 (dd, J = 3.6 Hz, 9.2 Hz, 1H), 4.89~4.72 (m, 2H), |
| | 4.70~4.39 (m, 1H), 4.11~4.01 (m, 2H), 3.97~3.59 (m, 2H), 3.40~3.25 (m, 3H), 2.99 (s, 3H), |
| | 2.94~2.78 (m, 3H), 2.11~2.10 (d, J = 7.2 Hz, 3H), 2.06~2.00 (m, 2H). |
| 186 | 1H NMR (400 MHz, cdcl3) δ 8.95~8.92 (d, J = 8.8 Hz, 1H), 8.81 (s, 1H), 7.97 (s, 2H), 7.90~7.88 |
| | (d, J = 10.0 Hz, 1H), 7.16~7.14 (d, J = 8.8 Hz, 1H), 4.90~4.74 (m, 2H), 4.70~4.40 (m, 1H), |
| | 4.12~4.02 (m, 2H), 3.97~3.59 (m, 2H), 3.41~3.27 (m, 1H), 3.05~3.03 (t, J = 4.4 Hz, 4H), |
| | 2.95~2.80 (m, 1H), 2.63 (s, 4H), 2.42~2.39 (m, 6H), 2.12~2.10 (d, J = 7.6 Hz, 3H). |
| 187 | 1H NMR (400 MHz, cdcl3) δ 8.88 (d, J = 1.6, 1H), 8.79 (d, J = 1.6, 1H), 8.07 (d, J = 8.2, 2H), |
| | 7.90 (s, 1H), 7.39 (d, J = 8.2, 2H), 4.94 (dd, J = 11.6, 5.3, 1H), 4.78-4.63 (m, 3H), 4.29-4.20 |
| | (m, 1H), 4.12-4.05 (m, 1H), 3.87-3.77 (m, 2H), 3.57-3.49 (m, 1H), 3.06-2.83 (m, 3H), |
| | 1.32 (d, J = 6.9, 6H). |
| 188 | 1H NMR (400 MHz, cdcl3) δ 8.79 (s, 1H), 8.72 (s, 1H), 7.96-7.87 (m, 2H), 7.79 (s, 1H), 7.12 |
| | (d, J = 8.9, 1H), 5.06 (s, 2H), 5.02-4.91 (m, 1H), 4.70-4.61 (m, 1H), 4.26-4.17 (m, 1H), 4.15- |
| | 4.05 (m, 1H), 3.88-3.74 (m, 2H), 3.58-3.46 (m, 1H), 3.00-2.92 (m, 1H), 2.92-2.84 (m, |
| | 1H), 2.77 (s, 6H), 2.44 (s, 3H). |
| 189 | 1H NMR (400 MHz, cdcl3) δ 8.89~8.87 (d, J = 8.8 Hz, 1H), 8.74~8.73 (d, J = 2.0 Hz, 1H), |
| | 7.97~7.95 (d, J = 8.4 Hz, 1H), 7.89 (s, 1H), 7.79~7.77 (d, J = 9.2 Hz, 1H), 6.56~6.53 (dd, J = 4.4 |
| | Hz, 8.0 Hz, 1H), 4.89~4.40 (m, 3H), 4.11~4.01 (m, 2H), 3.98~3.59 (s, 2H), 3.47~3.42 (m, 2H), |
| | 3.40~3.26 (m, 1H), 3.10~3.05 (m, 2H), 2.94~2.78 (m, 4H), 2.12~2.10 (d, J = 5.2 Hz, 3H). |
| 190 | 1H NMR (400 MHz, cdcl3) δ 8.94 (s, 1H), 8.83 (s, 1H), 8.09 (d, J = 8.1, 2H), 7.93 (s, 1H), 7.37 |
| | (d, J = 8.3, 2H), 5.95 (s, 1H), 4.38-4.31 (m, 1H), 4.18-4.07 (m, 2H), 4.02-3.94 (m, 1H), 3.43- |
| | 2.91 (m, 3H), 2.69-2.56 (m, 1H), 2.51-2.40 (m, 2H), 2.36-2.03 (m, 2H), 2.01-1.77 (m, |
| | 4H). |
| 191 | 1 H NMR (400 MHz, cdcl3) δ 8.92 (s, 1H), 8.83 (s, 1H), 8.13 (d, J = 8.1, 2H), 7.91 (s, 1H), 7.34 |
| | (d, J = 8.1, 2H), 4.86-4.67 (m, 3H), 4.63-4.49 (m, |
| | 1H), 4.06-3.92 (m, 2H), 3.90-3.80 (m, 1H), 3.21 (t, J = 12.2, 1H), 2.83 (t, J = 12.0, 1H), 2.67 |
| | (t, J = 12.4, 1H), 2.15 (s, 3H), 2.01-1.91 (m, 6H), |
| | 1.76-1.62 (m, 2H). |
| 192 | 1H NMR (400 MHz, cdcl3) δ 8.92 (d, J = 1.5, 1H), 8.81 (s, 1H), 7.97 (d, J = 6.0, 2H), 7.88 (d, J = |
| | 1.3, 1H), 7.13 (d, J = 9.0, 1H), 6.01-5.88 (m, 1H), 4.39-4.32 (m, 1H), 4.15-4.08 (m, 2H), |
| | 4.02-3.95 (m, 1H), 3.92-3.85 (m, 4H), 3.04-2.97 (m, 4H), 2.50-2.44 (m, 5H), 2.44 (s, 3H). |
| 193 | 1H NMR (400 MHz, cdcl3) δ 8.93 (s, 1H), 8.82 (s, 1H), 7.90-7.78 (m, 3H), 7.10-6.94 (m, 1H), |
| | 5.93 (s, 1H), 4.38-4.30 (m, 1H), 4.16-4.07 (m, 2H), 4.02-3.95 (m, 1H), 3.93-3.85 (m, 4H), |
| | 3.24-3.15 (m, 4H), 2.51-2.41 (m, 2H). |
| 194 | 1H NMR (400 MHz, cdcl3) δ 8.97-8.89 (m, 1H), 8.88-8.79 (m, 1H), 8.1 7 (d, J = 2.0, 1H), 8.05- |
| | 7.94 (m, 1H), 7.86 (s, 1H), 7.14 (d, J = 8.4, 1H), 6.03-5.86 (m, 1H), 4.37-4.30 (m, 1H), 4.15- |
| | 4.09 (m, 2H), 4.01-3.95 (m, 1H), 3.95-3.85 (m, 4H), 3.22-3.07 (m, 4H), 2.52-2.37 (m, |
| | 2H). |
| 195 | 1H NMR (400 MHz, cdcl3) δ 8.95 (s, 1H), 8.83~3.82(d, J = 3.6 Hz, 1H), 8.09~8.08 (m, 2H), |
| | 7.96~7.92 (m, 1H), 7.40~7.36 (m, 2H), 4.89~4.73 (m, 2H), 4.67~4.36 (m, 1H), 4.25~4.02 (m, |
| | 4H), 3.69~3.21 (m, 4H), 3.09~2.97 (m, 2H), 1.33~1.29 (t, J = 6.8 Hz, 6H). |
| 196 | 1H NMR (400 MHz, cdcl3) δ 8.90~8.89 (d, J = 4.8 Hz, 1H), 8.75~8.74 (d, J = 2.0 Hz, 1H), |
| | 8.08~8.06 (d, J = 8.0 Hz, 2H), 7.82~7.81 (d, J = 3.6 Hz, 1H), 6.83~6.81 (d, J = 7.2 Hz, 2H), |
| | 4.93~4.36 (m, 4H), 4.16~4.03 (m, 2H), 3.90~3.54 (m, 3H), 3.39~3.23 (m, 1H), 3.06 (s, 6H), |
| | 3.03~2.94 (m, 1H), 1.34~1.26 (m, 3H). |
| 197 | 1H NMR (400 MHz, cdcl3) δ 8.90~8.88 (d, J = 6.0 Hz, 1H), 8.74 (s, 1H), 8.08~8.06 (d, J = 8.4 |
| | Hz, 2H), 7.82 (s, 1H), 6.84~6.81 (d, J = 8.8 Hz, 2H), 4.92~4.71 (m, 2H), 4.66~4.44 (m, 2H), |
| | 4.14~4.03 (m, 2H), 3.91~3.49 (m, 3H), 3.40~3.23 (m, 1H), 3.06 (s, 6H), 3.01~2.93 (m, 1H), |
| | 1.37~1.25 (m, 3H). |
| 198 | 1H NMR (400 MHz, cdcl3) δ 8.90~8.88 (d, J = 7.2 Hz, 1H), 8.74 (s, 1H), 8.09~8.07 (d, J = 8.8 |
| | Hz, 2H), 7.82~7.80 (d, J = 8.4 Hz, 1H), 6.83~6.81 (d, J = 8.0 Hz, 2H), 4.89~4.41 (m, 3H), |
| | 4.11~3.59 (m, 4H), 3.36~3.21 (m, 1H), 3.06 (s, 6H), 2.94~2.79 (m, 1H), 2.40~2.30 (m, 2H), |
| | 1.18~1.10 (m, 3H). |
| 199 | 1H NMR (400 MHz, cdcl3) δ 8.88 (s, 1H), 8.74 (s, 1H), 8.10~8.07 (d, J = 8.8 Hz, 2H), 7.80 (s, |
| | 1H), 6.83~6.81 (d, J = 8.8 Hz, 2H), 4.86~4.71 (m, 2H), 4.13~4.07 (m, 1H), 4.02~3.92 (m, 6H), |
| | 3.71~3.63 (m, 2H), 3.09~2.94 (m, 8H), 2.24~2.17 (m, 2H). |
| 200 | 1H NMR (400 MHz, cdcl3) δ 8.94~8.93 (d, J = 5.2 Hz, 1H), 8.80~8.79 (d, J = 1.6 Hz, 1H), |
| | 7.96~7.94 (m, 2H), 7.89~7.88 (d, J = 4.4 Hz, 1H), 7.14~7.11 (d, J = 9.2 Hz, 1H), 4.94~4.36 (m, |
| | 4H), 4.17~4.03 (m, 2H), 3.91~3.55 (m, 3H), 3.40~3.24 (m, 1H), 3.07~2.95 (m, 1H), 2.80 (s, 6H), |
| | 2.44 (s, 3H), 1.34~1.26 (m, 3H). |
| 201 | 1H NMR (400 MHz, cdcl3) δ 8.94~8.93 (d, J = 5.6 Hz, 1H), 8.80 (s, 1H), 7.96~7.95 (m, 2H), |
| | 7.90~7.89 (d, J = 3.6 Hz, 1H), 7.14~7.12 (d, J = 8.8 Hz, 1H), 4.93~4.72 (m, 2H), 4.67~4.42 (m, |
| | 2H), 4.14~4.05 (m, 2H), 3.91~3.55 (m, 3H), 3.40~3.26 (m, 1H), 3.07~2.94 (m, 1H), 2.80 (s, 6H), |
| | 2.44 (s, 3H), 1.37~1.26 (m, 3H). |
| 202 | 1H NMR (400 MHz, cdcl3) δ 8.94~8.92 (d, J = 8.0 Hz, 1H), 8.80 (s, 1H), 7.97~7.95 (m, 2H), |
| | 7.89~7.87 (d, J = 8.8 Hz, 1H), 7.13~7.11 (d, J = 9.2 Hz, 1H), 4.89~4.42 (m, 3H), 4.11~3.59 (m, |
| | 4H), 3.36~3.22 (m, 1H), 2.96~2.79 (m, 7H), 2.44~2.28 (m, 5H), 1.18~1.10 (m, 3H). |
| 203 | 1H NMR (400 MHz, dmso) δ 13.18 (s, 1H), 9.07 (d, J = 1.9, 1H), 8.90 (d, J = 1.8, 1H), 8.69 (d, |
| | J = 0.8, 1H), 8.27-8.21 (m, 1H), 8.19 (d, J = 0.8, 1H), 8.08 (s, 1H), 7.66-7.61 (m, 1H), 4.79- |
| | 4.67 (m, 2H), 4.10-4.04 (m, 1H), 4.03-3.98 (m, 1H), 3.74-3.68 (m, 1H), 3.67-3.59 (m, |
| | 1H), 3.44-3.39 (m, 1H), 2.92 (s, 3H), 2.91-2.83 (m, 2H). |
| 204 | 1H NMR (400 MHz, dmso) δ 13.18 (s, 1H), 9.07 (d, J = 1.8, 1H), 8.89 (d, J = 1.8, 1H), 8.69 (s, |
| | 1H), 8.24 (d, J = 8.9, 1H), 8.20 (s, 1H), 8.07 (s, 1H), 7.64 (d, J = 8.9, 1H), 6.53-6.45 (m, 1H), |
| | 4.75-4.61 (m, 2H), 4.09-3.99 (m, 1H), 3.95-3.83 (m, 2H), 3.76-3.63 (m, 1H), 3.50-3.44 |
| | (m, 1H), 2.88-2.73 (m, 2H), 2.56 (d, J = 4.3, 3H). |
| 205 | 1H NMR (400 MHz, dmso) δ 9.04 (s, 1H), 8.86 (s, 1H), 8.12-7.89 (m, 3H), 7.10 (d, J = 8.0, |
| | 1H), 5.95-5.78 (m, 1H), 4.15-4.07 (m, 1H), 3.99-3.87 (m, 2H), 3.85-3.77 (m, 1H), 3.62- |
| | 3.54 (m, 4H), 2.93-2.79 (m, 4H), 2.45-2.39 (m, 1H), 2.36 (s, 3H), 2.23-2.16 (m, 1H), 2.03 |
| | (s, 3H). |
| 206 | 1H NMR (400 MHz, cdcl3) δ 8.90~8.89 (d, J = 1.6 Hz, 1H), 8.74~8.73 (d, J = 1.6 Hz, 1H), |
| | 8.08~8.06 (d, J = 8.8 Hz, 2H), 7.81 (s, 1H), 6.84~6.82 (d, J = 9.2 Hz, 2H), 4.79~4.76 (m, 2H), |
| | 3.70~3.66 (dd, J = 7.6 Hz, 9.6 Hz, 1H), 3.51~3.47 (m, 1H), 3.35~3.28 (m, 1H), 3.07 (s, 6H), |
| | 2.68~2.61 (dd, J = 9.2 Hz, 17.2 Hz, 1H), 2.38~2.32 (dd, J = 6.0 Hz, 17.2 Hz, 1H). |
| 207 | 1H NMR (400 MHz, cdcl3) δ 8.88~8.87 (d, J = 2.0 Hz, 1H), 8.73~8.72 (d, J = 2.0 Hz, 1H), |
| | 7.92~7.90 (dd, J = 2.4 Hz, 9.2 Hz, 1H), 7.78~7.76 (m, 2H), 6.68~6.66 (d, J = 8.4 Hz, 1H), |
| | 4.89~4.73 (m, 2H), 4.29~4.23 (m, 1H), 4.12~4.08 (m, 1H), 3.91~3.88 (m, 1H), 3.84~3.77 (m, |
| | 1H), 3.63~3.59 (d, J = 13.6 Hz, 1H), 3.35~3.32 (t, J = 5.6 Hz, 2H), 2.99 (s, 3H), 2.94~2.81 (m, |
| | 7H), 2.06~2.00 (m, 2H). |
| 208 | 1H NMR (400 MHz, cdcl3) δ 8.76 (s, 1H), 8.66 (s, 1H), 7.90~7.88 (dd, J = 2.0 Hz, 8.8 Hz, 1H), |
| | 7.74 (s, 1H), 7.70 (s, 1H), 6.68~6.66 (d, J = 8.8 Hz, 1H), 4.98~4.65 (m, 4H), 4.27~4.21 (m, 1H), |
| | 4.12~4.09 (d, J = 12.0 Hz, 1H), 3.86~3.78 (m, 2H), 3.54~3.52 (d, J = 10.8 Hz, 1H), 3.36~3.33 |
| | (t, J = 5.6 Hz, 2H), 3.00~2.84 (m, 7H), 2.07~2.01 (m, 2H). |
| 209 | 1H NMR (400 MHz, cdcl3) δ 8.88 (s, 1H), 8.71 (s, 1H), 7.93~7.91 (d, J = 8.4 Hz, 1H), 7.78 (s, |
| | 2H), 6.68~6.66 (d, J = 8.8 Hz, 1H), 4.88~4.68 (m, 2H), 4.58 (s, 2H), 4.17~4.11 (m, 1H), |
| | 4.04~4.01 (d, J = 12.0 Hz, 2H), 3.83~3.80 (d, J = 12.8 Hz, 1H), 3.73~3.66 (m, 1H), 3.35~3.33 (t, |
| | J = 5.2 Hz, 2H), 3.14~3.03 (m, 2H), 2.99 (s, 3H), 2.89~2.86 (t, J = 6.4 Hz, 2H), 2.06~2.00 (m, |
| | 2H). |
| 210 | 1H NMR (400 MHz, dmso) δ 9.07 (d, J = 1.8, 1H), 8.89 (d, J = 1.8, 1H), 8.10-8.04 (m, 2H), |
| | 7.99 (s, 1H), 7.18 (d, J = 8.2, 1H), 5.96-5.84 (m, 1H), 4.18-4.10 (m, 1H), 4.00-3.92 (m, 2H), |
| | 3.88-3.82 (m, 1H), 3.35-3.31 (m, 4H), 3.05-3.00 (m, 4H), 2.96 (s, 3H), 2.47-2.41 (m, 1H), |
| | 2.39 (s, 3H), 2.28-2.17 (m, 1H). |
| 211 | 1H NMR (400 MHz, dmso) δ 9.00 (d, J = 1.9, 1H), 8.81 (d, J = 1.9, 1H), 8.09 (d, J = 9.1, 2H), |
| | 7.85 (s, 1H), 7.22-7.15 (m, 1H), 6.80 (d, J = 9.1, 2H), 4.72-4.61 (m, 2H), 4.04-3.91 (m, 2H), |
| | 3.61-3.54 (m, 2H), 3.34-3.30 (m, 1H), 2.98 (s, 6H), 2.84-2.77 (m, 2H), 2.54 (d, J = 4.8, 3H). |
| 212 | 1H NMR (400 MHz, cdcl3) δ 8.98 (d, J = 1.6, 1H), 8.86 (d, J = 1.6, 1H), 8.16-8.18 (m, 2H), |
| | 7.98 (s, 1H), 7.62-7.64 (m, 2H), 4.87-4.92 (m, 1H), 4.75-4.78 (m, 1H), 4.23-4.27 (m, 1H), |
| | 4.08-4.10 (m, 1H), 3.89-3.92 (m, 1H), 3.77-3.83 (m, 1H), 3.59-3.62 (m, 1H), 2.99-2.87 |
| | (m, 2H), 2.81 (s, 3H), 1.79 (s, 6H). |
| 213 | 1H NMR (400 MHz, cdcl3) δ 8.96 (d, J = 1.8, 1H), 8.84 (d, J = 1.8, 1H), 8.12-8.14 (m, 2H), |
| | 7.96 (s, 1H), 7.52-7.54 (m, 2H), 4.97-5.02 (m, 1H), 4.87-4.91 (m, 1H), 4.74-4.78 (m, 1H), |
| | 4.23-4.26 (m, 1H), 4.07-4.10 (m, 1H), 3.89-3.92 (m, 1H), 3.77-3.80 (m, 1H), 3.59-3.62 |
| | (m, 1H), 2.85-2.96 (m, 2H), 2.81 (s, 3H), 1.56 (d, J = 6.5, 3H). |
| 214 | 1H NMR (400 MHz, cdcl3) δ 8.88 (s, 1H), 8.73 (s, 1H), 7.96~7.94 (d, J = 8.0 Hz, 1H), 7.88 (s, |
| | 1H), 7.78 (s, 1H), 6.55~6.53 (d, J = 7.6 Hz, 1H), 4.89~4.73 (m, 2H), 4.28~4.23 (m, 1H), |
| | 4.11~4.07 (m, 1H), 3.92~3.89 (d, J = 11.6 Hz, 1H), 3.83~3.76 (m, 1H), 3.62~3.59 (d, J = 11.2 |
| | Hz, 1H), 3.47~3.43 (t, J = 7.6 Hz, 2H), 3.10~3.05 (t, J = 8.4 Hz, 2H), 2.97~2.80 (m, 8H). |
| 215 | 1H NMR (400 MHz, cdcl3) δ 8.80~8.79 (d, J = 1.6 Hz, 1H), 8.69~8.68 (d, J = 1.6 Hz, 1H), |
| | 7.94~7.92 (dd, J = 1.6 Hz, 8.4 Hz, 1H), 7.86 (s, 1H), 7.73 (s, 1H), 6.55~6.53 (d, J = 8.0 Hz, 1H), |
| | 4.95~4.68 (m, 4H), 4.27~4.21 (m, 1H), 4.11~4.07 (m, 1H), 3.85~3.79 (m, 2H), 3.55~3.44 (m, |
| | 3H), 310~3.068 (t, J = 8.0 Hz, 2H), 2.98~2.86 (m, 5H). |
| 216 | 1H NMR (400 MHz, cdcl3) δ 8.88~8.87 (d, J = 2.0 Hz, 1H), 8.72~8.71 (d, J = 1.6 Hz, 1H), |
| | 7.97~7.94 (dd, J = 2.0 Hz, 8.4 Hz, 1H), 7.89 (s, 1H), 7.78 (s, 1H), 6.55~6.53 (d, J = 8.0 Hz, 1H), |
| | 4.88~4.70 (m, 2H), 4.56 (s, 2H), 4.17~4.11 (m, 1H), 4.04~4.00 (m, 2H), 3.82~3.78 (d, J = 13.6 |
| | Hz, 1H), 3.72~3.66 (m, 1H), 3.47~3.43 (t, J = 8.4 Hz, 2H), 3.15~3.04 (m, 4H), 2.85 (s, 3H). |
| 217 | 1H NMR (400 MHz, dmso) δ 9.09 (s, 1H), 8.92 (s, 1H), 8.19 (d, J = 8.0, 2H), 8.04 (s, 1H), 7.42 |
| | (d, J = 8.0, 2H), 5.91 (s, 1H), 4.17-4.11 (m, 1H), 4.01-3.91 (m, 4H), 3.88-3.81 (m, 1H), 3.51- |
| | 3.43 (m, 2H), 2.90-2.80 (m, 1H), 2.47-2.39 (m, 1H), 2.27-2.19 (m, 1H), 1.78-1.66 (m, |
| | 4H). |
| 218 | 1H NMR (400 MHz, cdcl3) δ 9.04 (d, J = 2.3, 1H), 8.90 (d, J = 1.4, 1H), 8.77 (s, 1H), 8.20 (d, J = |
| | 8.7, 1H), 7.77 (s, 1H), 6.65 (d, J = 9.0, 1H), 4.84-4.88 (m, 1H), 4.69-4.73 (m, 1H), 4.18-4.23 |
| | (m, 1H), 4.05-4.08 (m, 1H), 3.84-3.87 (m, 1H), 3.74-3.81 (m, 1H), 3.57-3.60 (m, 1H), 3.21 |
| | (s, 6H), 2.82-2.95 (m, 2H), 2.80 (s, 3H) |
| 219 | 1H NMR (400 MHz, cdcl3) δ 8.95 (d, J = 4.5, 1H), 8.85 (d, J = 4.5, 1H), 8.12 (d, J = 8.4, 2H), |
| | 7.97 (s, 1H), 7.64 (d, J = 8.3, 2H), 4.86-4.91 (m, 1H), 4.74-4.78 (m, 1H), 4.22-4.27 (m, 1H), |
| | 4.06-4.12 (m, 1H), 3.88-3.90 (m, 1H), 3.76-3.79 (m, 1H), 3.58-3.61 (m, 1H), 2.80-2.96 |
| | (m, 2H), 2.80 (s, 3H), 1.64 (s, 6H). |
| 220 | 1H NMR (400 MHz, dmso) δ 9.09 (d, J = 1.8, 1H), 8.92 (d, J = 1.8, 1H), 8.07 (s, 1H), 7.90- |
| | 7.84 (m, 1H), 7.84-7.79 (m, 1H), 7.25-7.17 (m, 1H), 7.14-7.06 (m, 1H), 4.77-4.69 (m, |
| | 2H), 4.08-4.02 (m, 1H), 4.01-3.95 (m, 1H), 3.91 (s, 3H), 3.84 (s, 3H), 3.64-3.55 (m, 2H), |
| | 3.34-3.32 (m, 1H), 2.88-2.80 (m, 2H), 2.56 (d, J = 4.8, 3H). |
| 221 | 1H NMR (400 MHz, dmso) δ 9.07 (d, J = 1.9, 1H), 8.91 (d, J = 1.9, 1H), 8.05-8.01 (m, 2H), |
| | 7.97 (s, 1H), 7.23-7.16 (m, 1H), 7.14-7.10 (m, 1H), 4.75-4.68 (m, 2H), 4.07-3.96 (m, 2H), |
| | 3.65-3.57 (m, 2H), 3.34-3.32 (m, 1H), 2.88-2.80 (m, 2H), 2.72 (s, 6H), 2.57 (d, J = 4.6, 3H), |
| | 2.38 (s, 3H). |
| 222 | 1H NMR (400 MHz, cdcl3) δ 8.95-8.96 (m, 1H), 8.82-8.83 (m, 1H), 8.08 (d, J = 7.6, 2H), 7.94 |
| | (s, 1H), 7.34 (d, J = 7.7, 2H), 4.86-4.89 (m, 1H), 4.72-4.73 (m, 1H), 4.12-4.15 (m, 1H), 3.96- |
| | 4.02 (m, 2H), 3.75-3.80 (m, 2H), 3.48 (s, 3H), 3.01-3.10 (m, 2H), 2.70-2.76 (m, 2H), 1.29 (t, |
| | J = 7.6, 3H) |
| 223 | 1H NMR (400 MHz, cdcl3) δ 8.92~8.91 (d, J = 2.0 Hz, 1H), 8.78~8.77 (d, J = 1.6 Hz, 1H), |
| | 8.10~8.08 (d, J = 8.8 Hz, 2H), 7.86 (s, 1H), 7.03~7.01 (d, J = 8.8 Hz, 2H), 5.66 (s, 1H), |
| | 4.81~4.73 (m, 2H), 3.91~3.88 (t, J = 4.8 Hz, 4H), 3.71~3.66 (dd, J = 8.0 Hz, 9.6 Hz, 1H), |
| | 3.51~3.47 (dd, J = 5.2 Hz, 9.6 Hz, 1H), 3.34~3.22 (m, 5H), 2.67~2.61 (m, 1H), 2.39~2.33 (m, |
| | 1H). |
| 224 | 1H NMR (400 MHz, cdcl3) δ 8.94~8.93 (d, J = 2.0 Hz, 1H), 8.81~8.80 (d, J = 1.6 Hz, 1H), |
| | 7.99~7.96 (m, 2H), 7.89 (s, 1H), 7.15~7.13 (d, J = 8.4 Hz, 1H), 5.66 (s, 1H), 4.82~4.73 (m, 2H), |
| | 3.3.90~3.88 (t, J = 4.4 Hz, 4H), 3.71~3.67 (t, J = 9.6 Hz, 1H), 3.51~3.47 (m, 1H), 3.34~3.27 (m, |
| | 1H), 3.01~2.99 (t, J = 4.4 Hz, 4H), 2.67~2.61 (m, 1H), 2.44 (s, 3H), 2.40~2.34 (m, 1H). |
| 225 | 1H NMR (400 MHz, cdcl3) δ 8.91~8.90 (d, J = 1.6 Hz, 1H), 8.77~8.76 (d, J = 2.0 Hz, 1H), |
| | 8.08~8.06 (d, J = 8.8 Hz, 2H), 7.84 (s, 1H), 7.04~7.02 (d, J = 8.8 Hz, 2H), 5.67 (s, 1H), |
| | 4.81~4.73 (m, 2H), 3.70~3.64 (dd, J = 8.0 Hz, 10.0 Hz, 1H), 3.51~3.47 (m, 1H), 3.37~3.26 (m, |
| | 5H), 2.67~2.60 (m, 5H), 2.39~2.33 (m, 4H). |
| 226 | 1H NMR (400 MHz, cdcl3) δ 8.93~8.92 (d, J = 1.6 Hz, 1H), 8.79~8.78 (d, J = 1.6 Hz, 1H), |
| | 7.96~7.94 (m, 2H), 7.88 (s, 1H), 7.14~7.12 (d, J = 8.0 Hz, 1H), 5.66 (s, 1H), 4.82~4.73 (m, 2H), |
| | 3.71~3.66 (dd, J = 7.6 Hz, 9.6 Hz, 1H), 3.51~3.47 (m, 1H), 3.36~3.26 (m, 1H), 2.80 (s, 6H), |
| | 2.67~2.61 (m, 1H), 2.44 (s, 3H), 2.40~2.34 (m, 1H). |
| 227 | 1 H NMR (400 MHz, cdcl 3) δ 9.00-8.91 (m, 1H), 8.89-8.80 (m, 1H), 8.11 (d, J = 8.1, 2H), |
| | 7.94 (s, 1H), 7.39 (d, J = 8.2, 2H), 6.01-5.90 (m, 1H), 4.38-4.32 (m, 1H), 4.17-4.09 (m, 2H), |
| | 4.03-3.96 (m, 1H), 3.71 (t, J = 5.2, 2H), 3.17 (d, J = 11.2, 2H), 2.68 (m, 4H), 2.49-2.44 (m, |
| | 2H), 2.37-2.29 (m, 2H), 1.99-1.90 (m, 4H). |
| 228 | 1 H NMR (400 MHz, cdcl 3) δ 9.00-8.84 (m, 1H), 8.83-8.67 (m, 1H), 8.07 (d, J = 8.7, 2H), |
| | 7.82 (s, 1H), 7.03 (d, J = 8.3, 2H), 5.95 (s, 1H), 4.40-4.31 (m, 1H), 4.18-4.08 (m, 2H), 4.02- |
| | 3.90 (m, 3H), 2.85-2.73 (m, 2H), 2.49-2.40 (m, 2H), 1.93-1.84 (m, 2H), 1.52-1.46 (m, |
| | 2H), 1.34-1.14 (m, 8H). |
| 229 | 1 H NMR (400 MHz, cdcl 3) δ 8.91 (d, J = 1.5, 1H), 8.79 (d, J = 1.6, 1H), 8.09 (d, J = 8.7, 2H), |
| | 7.84 (s, 1H), 7.02 (d, J = 8.6, 2H), 5.99-5.90 (m, 1H), 4.39-4.32 (m, 1H), 4.18-4.09 (m, 2H), |
| | 4.03-3.96 (m, 1H), 3.89-3.79 (m, 2H), 3.63-3.56 (m, 2H), 2.56-2.43 (m, 4H), 1.29 (d, J = |
| | 6.2, 6H). |
| 230 | 1H NMR (400 MHz, cdcl3) δ 8.98 (s, 1H), 8.87 (s, 1H), 8.19 (d, J = 8.1, 2H), 7.99 (s, 1H), 7.65 |
| | (d, J = 8.1, 2H), 4.86-4.91 (m, 1H), 4.74-4.78 (m, 1H), 4.22-4.27 (m, 1H), 4.07-4.10 (m, |
| | 1H), 3.82-3.88 (m, 1H), 3.77-3.79 (m, 1H), 3.59-3.61 (m, 1H), 2.87-2.96 (m, 2H), 2.81 (s, |
| | 3H), 1.97 (t, J = 18.1, 3H) |
| 231 | 1H NMR (400 MHz, cdcl3) δ 8.96 (s, 1H), 8.85 (d, J = 1.7, 1H), 8.14 (d, J = 8.4, 2H), 7.97 (s, |
| | 1H), 7.56 (d, J = 8.4, 2H), 5.27 (s, 2H), 4.86-4.89 (m, 1H), 4.73-4.78 (m, 1H), 4.23-4.25 (m, |
| | 1H), 4.06-4.09 (m, 1H), 3.88-3.91 (m, 1H), 3.76-3.82 (m, 1H), 3.58-3.61 (m, 1H), 2.84- |
| | 2.96 (m, 2H), 2.80 (s, 3H), 1.65 (s, 6H) |
| 232 | 1H NMR (400 MHz, cdcl3) δ 8.89~8.88 (d, J = 2.0 Hz, 1H), 8.74~8.73 (d, J = 2.0 Hz, 1H), |
| | 8.08~8.06 (d, J = 9.2 Hz, 2H), 7.81 (s, 1H), 6.84~6.82 (d, J = 8.8 Hz, 2H), 5.46 (s, 1H), |
| | 4.82~4.74 (m, 2H), 3.70~3.66 (dd, J = 8.0 Hz, 10.0 Hz, 1H), 3.51~3.48 (m, 1H), 3.35~3.28 (m, |
| | 1H), 3.06 (s, 6H), 2.67~2.61 (dd, J = 9.2 Hz, 17.2 Hz, 1H), 2.36 (dd, J = 6.4 Hz, 17.2 Hz, 1H). |
| 233 | 1H NMR (400 MHz, dmso) δ 9.01 (d, J = 1.8, 1H), 8.83 (d, J = 1.7, 1H), 8.09 (d, J = 8.5, 2H), |
| | 7.85 (s, 1H), 6.83 (d, J = 8.8, 2H), 5.70-5.57 (m, 1H), 4.55-4.14 (m, 1H), 4.03-3.74 (m, 2H), |
| | 3.71-3.60 (m, 1H), 3.56-3.40 (m, 1H), 3.24-3.14 (m, 1H), 3.00 (s, 6H), 2.77-2.63 (m, 1H), |
| | 2.00 (s, 3H), 1.54-1.44 (m, 3H). |
| 234 | 1H NMR (400 MHz, dmso) δ 9.02 (d, J = 1.9, 1H), 8.83 (d, J = 1.9, 1H), 8.10 (d, J = 9.0, 2H), |
| | 7.85 (s, 1H), 6.84 (d, J = 9.1, 2H), 6.02 (s, 2H), 5.71-5.59 (m, 1H), 4.12-4.05 (m, 1H), 3.92- |
| | 3.85 (m, 1H), 3.77-3.70 (m, 1H), 3.69-3.62 (m, 1H), 3.51-3.41 (m, 1H), 3.01 (s, 6H), 2.85- |
| | 2.74 (m, 2H), 1.47 (d, J = 6.4, 3H). |
| 235 | 1H NMR (400 MHz, dmso) δ 9.02 (d, J = 1.9, 1H), 8.83 (d, J = 1.8, 1H), 8.10 (d, J = 8.8, 2H), |
| | 7.85 (s, 1H), 6.84 (d, J = 8.9, 2H), 6.46 (m, 1H), 5.71-5.59 (m, 1H), 4.09-4.02 (m, 1H), 3.94- |
| | 3.86 (m, 1H), 3.75-3.69 (m, 1H), 3.69-3.62 (m, 1H), 3.50-3.43 (m, 1H), 3.01 (s, 6H), 2.83- |
| | 2.74 (m, 2H), 2.56 (d, J = 4.3, 3H), 1.47 (d, J = 6.4, 3H). |
| 236 | 1H NMR (400 MHz, dmso) δ 9.02 (d, J = 1.8, 1H), 8.83 (d, J = 1.8, 1H), 8.09 (d, J = 8.9, 2H), |
| | 7.86 (s, 1H), 6.84 (d, J = 9.0, 2H), 5.62-5.55 (m, 1H), 4.06-4.01 (m, 1H), 3.88-3.83 (m, 1H), |
| | 3.75-3.70 (m, 1H), 3.67-3.59 (m, 1H), 3.43-3.37 (m, 1H), 3.00 (s, 6H), 2.90-2.78 (m, 5H), |
| | 1.50 (d, J = 6.3, 3H). |
| 237 | 1H NMR (400 MHz, dmso) δ 9.01 (d, J = 1.9, 1H), 8.83 (d, J = 1.8, 1H), 8.17-8.05 (m, 2H), |
| | 7.85 (s, 1H), 6.84 (d, J = 9.0, 2H), 5.81-5.71 (m, 1H), 4.39-4.14 (m, 1H), 3.98-3.65 (m, 3H), |
| | 3.54-3.38 (m, 1H), 3.25-3.17 (m, 1H), 3.01 (s, 6H), 2.78-2.64 (m, 1H), 2.00 (d, J = 3.6, 3H), |
| | 1.47 (dd, J = 14.0, 6.3, 3H). |
| 238 | 1H NMR (400 MHz, dmso) δ 9.01 (d, J = 1.8, 1H), 8.83 (d, J = 1.8, 1H), 8.10 (d, J = 8.9, 2H), |
| | 7.84 (s, 1H), 6.84 (d, J = 9.0, 2H), 6.01 (s, 2H), 5.76-5.69 (m, 1H), 4.07-4.01 (m, 1H), 3.95- |
| | 3.87 (m, 2H), 3.75-3.65 (m, 2H), 3.48-3.41 (m, 1H), 3.01 (s, 6H), 2.85-2.75 (m, 2H), 1.45 |
| | (d, J = 6.5, 3H). |
| 239 | 1H NMR (400 MHz, dmso) δ 9.01 (d, J = 1.9, 1H), 8.83 (d, J = 1.8, 1H), 8.10 (d, J = 9.0, 2H), |
| | 7.84 (s, 1H), 6.84 (d, J = 9.0, 2H), 6.45 (s, 1H), 5.77-5.68 (m, 1H), 3.94-3.87 (m, 2H), 3.74- |
| | 3.65 (m, 2H), 3.48-3.40 (m, 1H), 3.01 (s, 7H), 2.84-2.76 (m, 2H), 2.56 (d, J = 4.3, 3H), 1.45 |
| | (d, J = 6.5, 3H). |
| 240 | 1H NMR (400 MHz, dmso) δ 9.02 (d, J = 1.9, 1H), 8.83 (d, J = 1.9, 1H), 8.10 (d, J = 9.0, 2H), |
| | 7.85 (s, 1H), 6.84 (d, J = 9.1, 2H), 5.84-5.76 (m, 1H), 4.06-4.01 (m, 1H), 3.90-3.85 (m, 1H), |
| | 3.64-3.54 (m, 2H), 3.41-3.36 (m, 1H), 3.01 (s, 6H), 2.93-2.87 (m, 5H), 1.46 (d, J = 6.5, 3H). |
| 241 | 1 H NMR (400 MHz, dmso) δ 9.11 (d, J = 1.7, 1H), 8.95 (d, J = 1.7, 1H), 8.21 (d, J = 8.2, 2H), |
| | 8.06 (s, 1H), 7.43 (d, J = 8.2, 2H), 6.04 (s, 2H), 4.76-4.60 (m, 2H), 4.09-4.02 (m, 1H), 4.02- |
| | 3.95 (m, 2H), 3.94-3.85 (m, 2H), 3.79-3.70 (m, 1H), 3.52-3.43 (m, 3H), 2.94-2.74 (m, |
| | 3H), 1.83-1.64 (m, 4H). |
| 242 | 1 H NMR (400 MHz, dmso) δ 13.39-12.95 (m, 1H), 9.11 (d, J = 1.9, 1H), 8.94 (d, J = 1.9, 1H), |
| | 8.74 (s, 1H), 8.30-8.27 (m, 1H), 8.23 (d, J = 0.9, 1H), 8.11 (s, 1H), 7.68 (d, J = 8.9, 1H), 6.06 |
| | (s, 2H), 4.78-4.69 (m, 2H), 4.14-4.07 (m, 1H), 3.97-3.89 (m, 2H), 3.80-3.72 (m, 1H), 3.55- |
| | 3.47 (m, 1H), 2.94-2.79 (m, 2H). |
| 243 | 1 H NMR (400 MHz, dmso) δ 9.06 (s, 1H), 8.85 (s, 1H), 8.14 (d, J = 8.2, 2H), 7.97 (s, 1H), 7.05 |
| | (d, J = 8.3, 2H), 5.97 (s, 1H), 3.80-3.70 (m, 5H), 3.62-3.55 (m, 1H), 3.43-3.38 (m, 2H), 3.24- |
| | 3.21 (m, 4H), 3.15 (s, 1H), 2.46-2.33 (m, 2H). |
| 244 | 1H NMR (400 MHz, cd3od) δ 9.05 (d, J = 1.3, 1H), 8.88 (d, J = 1.3, 1H), 8.32 (d, J = 8.3, 2H), |
| | 8.05 (s, 1H), 7.67 (d, J = 8.4, 2H), 4.78-4.81 (m, 2H), 4.14-4.17 (m, 1H), 4.04-4.07 (m, 1H), |
| | 3.83-3.86 (m, 1H), 3.69-3.75 (m, 1H), 3.51-3.54 (m, 1H), 2.96-3.02 (m, 2H), 2.89 (s, 3H), |
| | 1.79 (s, 6H). |
| 245 | 1H NMR (400 MHz, cd3od) δ 9.03-9.05 (m, 1H), 8.86-8.88 (m, 1H), 8.29-8.33 (m, 2H), 8.03- |
| | 8.06 (m, 1H), 7.67 (d, J = 8.5, 2H), 4.60-4.63 (m, 1H), 4.30-4.33 (m, 1H), 4.06-4.13 (m, |
| | 1H), 3.97-4.00 (m, 2H), 3.77-3.80 (m, 1H), 3.56-3.64 (m, 1H), 3.34-3.40 (m, 1H), 2.85- |
| | 2.95 (m, 1H), 2.13 (d, J = 4.7, 3H), 1.79 (s, 6H). |
| 246 | 1H NMR (400 MHz, dmso) δ 9.02 (s, 1H), 8.83 (s, 1H), 8.09 (d, J = 8.8, 2H), 7.86 (s, 1H), 6.89- |
| | 6.73 (m, 4H), 5.67-5.58 (m, 1H), 4.06-3.99 (m, 1H), 3.86-3.80 (m, 1H), 3.63 (t, J = 10.2, |
| | 2H), 3.26-3.24 (m, 1H), 3.00 (s, 6H), 2.70-2.63 (m, 1H), 2.62-2.56 (m, 1H), 1.49 (d, J = 6.3, |
| | 3H). |
| 247 | 1H NMR (400 MHz, dmso) δ 9.02 (d, J = 1.9, 1H), 8.83 (d, J = 1.8, 1H), 8.11-8.08 (m, 2H), |
| | 7.85 (s, 1H), 6.86-6.80 (m, 4H), 5.86-5.78 (m, 1H), 4.06-4.01 (m, 1H), 3.90-3.84 (m, 1H), |
| | 3.64-3.58 (m, 1H), 3.46-3.39 (m, 1H), 3.26-3.24 (m, 1H), 3.01 (s, 6H), 2.74-2.65 (m, 2H), |
| | 1.45 (d, J = 6.5, 3H). |
| 248 | 1H NMR (400 MHz, cdcl3) δ 8.97 (d, J = 8.0, 1H), 8.86 (s, 1H), 8.17 (d, J = 8.1, 2H), 7.97 (d, J = |
| | 10.5, 1H), 7.62 (d, J = 8.4, 2H), 4.76-4.85 (m, 2H), 4.39-4.69 (m, 1H), 3.92-4.07 (m, 2H), |
| | 3.61-3.64 (m, 2H), 3.27-3.30 (m, 1H), 2.78-2.91 (m, 1H), 2.10 (d, J = 4.2, 3H), 1.78 (s, 6H). |
| 249 | 1H NMR (400 MHz, cdcl3) δ 8.96 (d, J = 1.6, 1H), 8.84 (d, J = 1.6, 1H), 8.17 (d, J = 8.4, 2H), |
| | 7.95 (s, 1H), 7.62 (d, J = 8.4, 2H), 4.84-4.88 (m, 1H), 4.68-4.71 (m, 1H), 4.09-4.11 (m, 1H), |
| | 3.99-4.03 (m, 2H), 3.71-3.73 (m, 1H), 3.64-3.67 (m, 1H), 3.06-3.10 (m, 1H), 2.95-3.04 |
| | (m, 1H), 2.80 (s, 3H), 1.78 (s, 6H). |
| 250 | 1H NMR (400 MHz, cdcl3) δ 8.95 (s, 1H), 8.85 (s, 1H), 8.16 (d, J = 8.2, 2H), 7.96 (s, 1H), 7.62 |
| | (d, J = 8.2, 2H), 4.88-4.92 (m, 1H), 4.72-4.75 (m, 1H), 4.23-4.28 (m, 1H), 4.07-4.09 (m, |
| | 1H), 3.79-3.81 (m, 2H), 3.51-3.54 (m, 1H), 2.86-2.94 (m, 2H), 1.78 (s, 6H). |
| 251 | 1H NMR (400 MHz, cdcl3) δ 8.92 (s, 1H), 8.81 (s, 1H), 8.11 (d, J = 8.4, 2H), 7.92 (s, 1H), 7.56 |
| | (d, J = 8.4, 2H), 4.78-4.82 (m, 1H), 4.66-4.70 (m, 1H), 4.14-4.16 (m, 1H), 3.97-4.00 (m, |
| | 1H), 3.67-3.72 (m, 2H), 3.41-3.45 (m, 1H), 2.88-2.99 (m, 2H), 2.69 (s, 3H), 1.72 (s, 6H). |
| 252 | 1H NMR (400 MHz, cdcl3) δ 8.97 (s, 1H), 8.85 (s, 1H), 8.17 (d, J = 6.6, 2H), 7.96 (s, 1H), 7.62 |
| | (d, J = 6.7, 2H), 4.86-4.89 (m, 1H), 4.69-4.73 (m, 1H), 4.09-4.1 9 (m, 2H), 4.01-4.06 (m, |
| | 1H), 3.68-3.84 (m, 2H), 3.09-3.23 (m, 2H), 1.78 (s, 6H). |
| 253 | 1H NMR (400 MHz, cdcl3) δ 8.96~8.94 (d, J = 7.6 Hz, 1H), 8.83 (s, 1H), 8.13~8.11 (d, J = 8.4 |
| | Hz, 2H), 7.97~7.95 (d, J = 10.0 Hz, 1H), 7.47~7.45 (d, J = 8.4 Hz, 2H), 4.90~4.76 (m, 2H), |
| | 4.70~4.39 (m, 1H), 4.10~4.01 (m, 2H), 3.97~3.58 (m, 2H), 3.50 (s, 2H), 3.40~3.27 (m, 1H), |
| | 2.94~2.80 (m, 1H), 2.28 (s, 6H), 2.11~2.09 (d, J = 6.8 Hz, 3H). |
| 254 | 1H NMR (400 MHz, cdcl3) δ 8.96~8.94 (d, J = 7.6 Hz, 1H), 8.83 (s, 1H), 8.11~8.09 (d, J = 8.8 |
| | Hz, 2H), 7.96~7.94 (d, J = 10.0 Hz, 1H), 7.56~7.54 (d, J = 8.8 Hz, 2H), 4.90~4.75 (m, 2H), |
| | 4.70~4.39 (m, 1H), 4.11~4.01 (m, 2H), 3.97~3.59 (m, 2H), 3.41~3.27 (m, 1H), 2.94~2.79 (m, |
| | 1H), 2.12~2.09 (d, J = 9.6 Hz, 3H), 1.39 (s, 9H). |
| 255 | 1H NMR (400 MHz, cdcl3) δ 8.95~8.94 (d, J = 1.6 Hz, 1H), 8.81~8.80 (d, J = 2.0 Hz, 1H), |
| | 8.11~8.09 (d, J = 8.4 Hz, 2H), 7.94 (s, 1H), 7.56~7.53 (d, J = 8.8 Hz, 2H), 4.90~4.70 (m, 2H), |
| | 4.64 (s, 2H), 4.16~4.10 (m, 1H), 4.06~4.00 (m, 2H), 3.80~3.77 (d, J = 13.6 Hz, 1H), 3.72~3.66 |
| | (m, 1H), 3.15~3.03 (m, 2H), 1.39 (s, 9H). |
| 256 | 1H NMR (400 MHz, cdcl3) δ 8.96~8.95 (d, J = 1.6 Hz, 1H), 8.83~8.82 (d, J = 2.0 Hz, 1H), |
| | 8.11~8.08 (d, J = 8.8 Hz, 2H), 7.95 (s, 1H), 7.56~7.54 (d, J = 8.8 Hz, 2H), 4.91~4.75 (m, 2H), |
| | 4.28~4.22 (m, 1H), 4.11~4.07 (m, 1H), 3.92~3.88 (m, 1H), 3.84~3.77 (m, 1H), 3.62~3.59 (d, J = |
| | 12.8 Hz, 1H), 2.97~2.85 (m, 2H), 2.81 (s, 3H), 1.39 (s, 9H). |
| 257 | 1H NMR (400 MHz, cdcl3) δ 8.86~8.85 (d, J = 1.6 Hz, 1H), 8.79~8.78 (d, J = 1.6 Hz, 1H), |
| | 8.09~8.07 (d, J = 8.4 Hz, 2H), 7.90 (s, 1H), 7.56~7.54 (d, J = 8.4 Hz, 2H), 4.97~4.69 (m, 4H), |
| | 4.27~4.21 (m, 1H), 4.11~4.08 (d, J = 14.0 Hz, 1H), 3.86~3.78 (m, 2H), 3.54~3.51 (d, J = 12.4 |
| | Hz, 1H), 2.99~2.86 (m, 2H), 1.39 (s, 9H). |
| 258 | 1 H NMR (400 MHz, dmso) δ 9.09 (d, J = 1.5, 1H), 8.93 (d, J = 1.5, 1H), 8.20 (d, J = 8.1, 2H), |
| | 8.06 (s, 1H), 7.44 (d, J = 8.1, 2H), 4.74-4.65 (m, 2H), 4.07-3.98 (m, 2H), 3.71-3.62 (m, 2H), |
| | 3.59-3.54 (m, 4H), 3.52 (s, 2H), 3.39 (d, J = 11.6, 1H), 2.91 (s, 3H), 2.90-2.81 (m, 2H), 2.37 |
| | (s, 4H). |
| 259 | 1 H NMR (400 MHz, cdcl 3) δ 8.92-8.88 (m, 1H), 8.80-8.75 (m, 1H), 8.12-8.01 (m, 2H), |
| | 7.92-7.86 (m, 1H), 7.36-7.23 (m, 2H), 4.88-4.76 (m, 1H), 4.74-4.63 (m, 1H), 4.64-4.52 |
| | (m, 2H), 4.24-4.12 (m, 1H), 4.07-3.96 (m, 1H), 3.82 (d, J = 11.5, 1H), 3.78-3.68 (m, 1H), |
| | 3.53 (d, J = 11.0, 1H), 2.94-2.90 (m, 3H), 2.91-2.78 (m, 2H), 2.74 (s, 3H), 2.12 (s, 3H). |
| 260 | 1 H NMR (400 MHz, cdcl 3) δ 8.80 (s, 1H), 8.72 (s, 1H), 8.01 (d, J = 8.2, 2H), 7.82 (s, 1H), 7.30 |
| | (d, J = 8.3, 2H), 4.92-4.75 (m, 3H), 4.67-4.60 (m, 1H), 4.19-4.12 (m, 1H), 4.08-3.99 (m, |
| | 3H), 3.79-3.70 (m, 2H), 3.55-3.44 (m, 3H), 2.92-2.74 (m, 3H), 1.87-1.73 (m, 4H). |
| 261 | 1H NMR (400 MHz, cdcl3) δ 8.97 (s, 1H), 8.84 (s, 1H), 8.12~8.10 (d, J = 8.0 Hz, 2H), 7.95 (s, |
| | 1H), 7.38~7.36 (d, J = 8.0 Hz, 2H), 4.82~4.74 (m, 2H), 4.00~3.97 (d, J = 11.6 Hz, 2H), |
| | 3.72~3.67 (t, J = 7.6 Hz, 1H), 3.51~3.47 (m, 2H), 3.36~3.27 (m, 1H), 2.84~2.80 (m, 5H), |
| | 2.76~2.61 (m, 2H), 2.40~2.34 (m, 1H), 2.04~1.86 (m, 4H). |
| 262 | 1H NMR (400 MHz, cdcl3) δ 8.95 (s, 1H), 8.83 (s, 1H), 8.12 (d, J = 7.7, 2H), 7.95 (s, 1H), 7.63 |
| | (d, J = 7.5, 2H), 7.55 (s, 1H), 4.83-4.88 (m, 1H), 4.72-4.77 (m, 1H), 4.19-4.24 (m, 1H), 4.03- |
| | 4.06 (m, 1H), 3.72-3.80 (m, 2H), 3.48-4.51 (m, 1H), 3.01-3.06 (m, 1H), 2.93-2.98 (m, 1H), |
| | 2.73 (s, 3H), 1.63 (s, 6H). |
| 263 | 1H NMR (400 MHz, cdcl3) δ 8.91 (d, J = 1.7, 1H), 8.81 (d, J = 1.6, 1H), 8.10 (d, J = 8.4, 2H), |
| | 7.93 (s, 1H), 7.63 (d, J = 8.4, 2H), 4.88-4.92 (m, 1H), 4.71-4.74 (m, 1H), 4.23-4.25 (m, 1H), |
| | 4.06-4.09 (m, 1H), 3.78-3.84 (m, 2H), 3.50-3.53 (m, 1H), 2.85-2.97 (m, 2H), 1.64 (s, 6H). |
| 264 | 1H NMR (400 MHz, cdcl3) δ 8.91 (d, J = 1.5, 1H), 8.76 (d, J = 1.5, 1H), 8.08 (d, J = 8.4, 2H), |
| | 7.89 (s, 1H), 7.60 (d, J = 8.4, 2H), 4.80-4.84 (m, 1H), 4.63-4.67 (m, 1H), 3.96-4.08 (m, 3H), |
| | 3.74-3.77 (m, 1H), 3.62-3.68 (m, 1H), 2.92-3.03 (m, 2H), 2.78 (s, 3H), 1.62 (s, 6H). |
| 265 | 1H NMR (400 MHz, cdcl3) δ 8.95 (s, 1H), 8.84 (s, 1H), 8.12 (d, J = 8.3, 2H), 7.96 (s, 1H), 7.63 |
| | (d, J = 8.3, 2H), 4.67-4.86 (m, 2H), 4.36-4.63 (m, 1H), 3.91-4.10 (m, 3H), 3.58-3.63 (m, |
| | 1H), 3.25-3.29 (m, 1H), 2.78-2.99 (m, 1H), 2.10 (s, 3H), 1.77 (s, 6H). |
| 266 | 1H NMR (400 MHz, cdcl3) δ 8.96 (s, 1H), 8.82 (s, 1H), 8.13 (d, J = 8.3, 2H), 7.96 (s, 1H), 7.64 |
| | (d, J = 8.4, 2H), 7.55 (s, 1H), 4.86-4.90 (m, 1H), 4.69-4.72 (m, 1H), 4.10-4.12 (m, 1H), 4.00- |
| | 4.07 (m, 2H), 3.76-3.79 (m, 1H), 3.66-3.71 (m, 1H), 3.04-3.13 (m, 2H), 1.64 (s, 6H). |
| 267 | 1H NMR (400 MHz, cdcl3) δ 8.97~8.94 (dd, J = 1.6 Hz, 8.8 Hz, 1H), 8.84 (s, 1H), 8.14~8.12 (d, |
| | J = 8.4 Hz, 2H), 7.97~7.94 (d, J = 10.0 Hz, 1H), 7.52~7.49 (d, J = 8.8 Hz, 2H), 4.90~4.74 (m, |
| | 2H), 4.70~4.39 (m, 1H), 4.12~4.01 (m, 2H), 3.96~3.59 (m, 2H), 3.40~3.27 (m, 1H), 2.95~2. (m, |
| | 3H), 2.21 (br, 2H), 2.11~2.10 (d, J = 6.8 Hz, 3H), 1.37 (s, 6H). |
| 268 | 1H NMR (400 MHz, cdcl3) δ 8.95 (s, 1H), 8.83 (s, 1H), 8.12 (d, J = 7.5, 2H), 7.96 (s, 1H), 7.55 |
| | (d, J = 7.8, 2H), 4.86-4.89 (m, 1H), 4.75-4.78 (m, 1H), 4.21-4.25 (m, 1H), 4.06-4.09 (m, |
| | 1H), 3.87-3.90 (m, 1H), 3.76-3.82 (m, 1H), 3.58-3.61 (m, 1H), 3.12 (s, 3H), 2.84-2.95 (m, |
| | 2H), 2.80 (s, 3H), 1.57 (s, 6H). |
| 269 | 1H NMR (400 MHz, cdcl3) δ 8.94 (s, 1H), 8.82 (s, 1H), 8.12 (d, J = 8.2, 2H), 7.95 (s, 1H), 7.54 |
| | (d, J = 8.2, 2H), 4.83-4.86 (m, 2H), 4.35-4.78 (m, 1H), 3.93-4.08 (m, 2H), 3.56-3.61 (m, |
| | 1H), 3.28-3.36 (m, 1H), 3.11 (s, 3H), 2.78-2.91 (m, 1H), 2.09 (s, 3H), 1.96-2.04 (m, 1H), |
| | 1.56 (s, 6H). |
| 270 | 1H NMR (400 MHz, cdcl3) δ 8.97~8.95 (d, J = 8.8 Hz, 1H), 8.84 (s, 1H), 8.15~8.13 (d, J = 8.4 |
| | Hz, 2H), 7.99~7.96 (d, J = 10.0 Hz, 1H), 7.54~7.52 (d, J = 8.4 Hz, 2H), 4.91~4.76 (m, 2H), |
| | 4.70~4.40 (m, 1H), 4.15~4.02 (m, 2H), 3.97~3.59 (m, 2H), 3.41~3.27 (m, 1H), 2.95~2.86 (m, |
| | 1H), 2.61 (s, 1H), 2.12~2.10 (d, J = 8.4 Hz, 3H), 1.98~1.82 (m, 4H), 0.83~0.80 (t, J = 7.6 Hz, |
| | 6H). |
| 271 | 1H NMR (400 MHz, cdcl3) δ 8.97~8.95 (d, J = 8.0 Hz, 1H), 8.84 (s, 1H), 8.13~8.11 (d, J = 8.4 |
| | Hz, 2H), 7.98~7.95 (d, J = 10.4 Hz, 1H), 7.47~7.45 (d, J = 8.4 Hz, 2H), 4.90~4.75 (m, 2H), |
| | 4.70~4.40 (m, 1H), 4.11~4.01 (m, 2H), 3.97~3.59 (m, 2H), 3.40~3.27 (m, 2H), 2.94~2.80 (m, |
| | 1H), 2.25 (s, 6H), 2.11~2.09 (d, J = 8.4 Hz, 3H), 1.43~1.41 (d, J = 6.4 Hz, 3H). |
| 272 | 1 H NMR (400 MHz, cdcl3) δ 8.94 (d, J = 1.5, 1H), 8.82 (d, J = 1.4, 1H), 8.07 (d, J = 8.1, 2H), |
| | 7.93 (s, 1H), 7.36 (d, J = 8.1, 2H), 4.92-4.83 (m, 1H), 4.79-4.72 (m, 1H), 4.28-4.18 (m, 1H), |
| | 4.11-4.05 (m, 1H), 3.89 (d, J = 11.5, 1H), 3.83-3.75 (m, 1H), 3.59 (d, J = 12.2, 1H), 2.96- |
| | 2.83 (m, 4H), 2.80 (s, 3H), 2.76-2.68 (m, 2H), 2.41 (s, 6H). |
| 273 | 1 H NMR (400 MHz, dmso) δ 9.14-9.08 (m, 1H), 8.97-8.91 (m, 1H), 8.20-8.14 (m, 2H), |
| | 8.08 (s, 1H), 7.39-7.34 (m, 2H), 4.77-4.67 (m, 2H), 4.34 (s, 1H), 4.11-4.05 (m, 1H), 4.05- |
| | 4.00 (m, 1H), 3.73-3.61 (m, 2H), 3.41 (d, J = 12.1, 1H), 2.94 (s, 3H), 2.93-2.84 (m, 2H), 2.74 |
| | (s, 2H), 1.11 (s, 3H), 1.07 (s, 3H). |
| 274 | 1 H NMR (400 MHz, dmso) δ 9.10 (d, J = 1.9, 1H), 8.94 (d, J = 1.8, 1H), 8.27-8.23 (m, 2H), |
| | 8.11 (d, J = 2.7, 1H), 7.47-7.42 (m, 2H), 4.74-4.64 (m, 2H), 4.07-3.97 (m, 2H), 3.70-3.59 |
| | (m, 2H), 3.38 (d, J = 11.2, 1H), 2.91-2.82 (m, 5H), 1.85-1.76 (m, 2H), 1.61-1.55 (m, 2H). |
| 275 | 1 H NMR (400 MHz, dmso) δ 9.11 (d, J = 1.8, 1H), 8.95 (d, J = 1.9, 1H), 8.27 (d, J = 8.5, 2H), |
| | 8.11 (s, 1H), 7.47 (d, J = 8.6, 2H), 6.03 (s, 2H), 4.75-4.61 (m, 2H), 4.08-3.99 (m, 1H), 3.93- |
| | 3.84 (m, 2H), 3.73 (d, J = 13.3, 1H), 3.52-3.42 (m, 1H), 2.92-2.73 (m, 2H), 1.86-1.77 (m, |
| | 2H), 1.62-1.54 (m, 2H). |
| 276 | 1 H NMR (400 MHz, dmso) δ 9.10 (d, J = 1.8, 1H), 8.94 (d, J = 1.8, 1H), 8.30-8.22 (m, 2H), |
| | 8.10 (s, 1H), 7.46 (d, J = 8.5, 2H), 4.76-4.64 (m, 2H), 4.50-4.10 (m, 1H), 3.99-3.66 (m, 3H), |
| | 3.58-3.38 (m, 1H), 3.23-3.21 (m, 1H), 2.77-2.65 (m, 1H), 2.00 (s, 3H), 1.83-1.76 (m, 2H), |
| | 1.61-1.53 (m, 2H). |
| 277 | 1H NMR (400 MHz, cdcl3) δ 8.95 (d, J = 1.9, 1H), 8.83 (d, J = 1.9, 1H), 8.10-8.13 (m, 2H), |
| | 7.97 (d, J = 2.7, 1H), 7.60-7.62 (m, 2H), 5.48 (s, 1H), 5.16 (s, 1H), 4.86-4.89 (m, 1H), 4.74- |
| | 4.78 (m, 1H), 4.22-4.25 (m, 1H), 4.07-4.10 (m, 1H), 3.88-3.91 (m, 1H), 3.77-3.82 (m, 1H), |
| | 3.58-3.61 (m, 1H), 2.84-2.96 (m, 2H), 2.80 (s, 3H), 2.20 (d, J = 0.7, 3H). |
| 278 | 1H NMR (400 MHz, cdcl3) δ 8.95~8.92 (dd, J = 1.6 Hz, 10.0 Hz, 1H), 8.81 (s, 1H), 8.11~8.09 (d, |
| | J = 8.4 Hz, 2H), 7.92~7.89 (d, J = 11.6 Hz, 1H), 7.59~7.57 (d, J = 8.4 Hz, 2H), 4.86~4.70 (m, |
| | 2H), 4.69~4.37 (m, 1H), 4.08~3.57 (m, 8H), 3.39~3.26 (m, 1H), 2.94~2.78 (m, 1H), 2.70~2.60 |
| | (m, 2H), 2.10~2.09 (d, J = 3.2 Hz, 3H), 1.33 (s, 3H). |
| 279 | 1H NMR (400 MHz, cdcl3) δ 8.96~8.95 (d, J = 2.0 Hz, 1H), 8.84~8.83 (d, J = 2.0 Hz, 1H), |
| | 8.14~8.12 (d, J = 8.8 Hz, 2H), 7.98 (s, 1H), 7.54~7.52 (d, J = 8.8 Hz, 2H), 4.92~4.75 (m, 2H), |
| | 4.28~4.22 (m, 1H), 4.11~4.07 (m, 1H), 3.92~3.88 (m, 1H), 3.84~3.77 (m, 1H), 3.62~3.59 (d, J = |
| | 11.2 Hz, 1H), 2.97~2.86 (m, 2H), 2.81 (s, 3H), 1.98~1.82 (m, 4H), 0.83~0.80 (t, J = 7.2 Hz, 6H). |
| 280 | 1H NMR (400 MHz, cdcl3) δ 8.96~8.94 (dd, J = 1.6 Hz, 9.6 Hz, 1H), 8.83 (s, 1H), 8.15~8.13 (d, |
| | J = 8.4 Hz, 2H), 7.97~7.94 (d, J = 10.8 Hz, 1H), 7.46~7.44 (d, J = 8.4 Hz, 2H), 4.89~4.74 (m, |
| | 2H), 4.69~4.35 (m, 2H), 4.11~4.00 (m, 2H), 3.96~3.57 (m, 2H), 3.39~3.27 (m, 4H), 2.93~2.79 |
| | (m, 1H), 2.10~2.09 (d, J = 6.8 Hz, 3H), 1.48~1.47 (d, J = 6.4 Hz, 3H). |
| 281 | 1H NMR (400 MHz, cdcl3) δ 8.96~8.95 (d, J = 2.0 Hz, 1H), 8.83~8.82 (d, J = 2.0 Hz, 1H), |
| | 8.15~8.13 (d, J = 8.4 Hz, 2H), 7.97 (s, 1H), 7.54~7.52 (d, J = 8.4 Hz, 2H), 4.90~4.72 (m, 2H), |
| | 4.60 (s, 2H), 4.17~4.11 (m, 1H), 4.06~4.00 (m, 2H), 3.79~3.76 (d, J = 13.2 Hz, 1H), 3.73~3.66 |
| | (m, 1H), 3.16~3.04 (m, 2H), 1.98~1.82 (s, 4H), 0.3~0.802 (t, J = 7.6 Hz, 6H). |
| 282 | 1 H NMR (400 MHz, dmso) δ 9.14-9.08 (m, 1H), 8.99-8.92 (m, 1H), 8.31 (d, J = 8.2, 2H), |
| | 8.13 (s, 1H), 7.61 (d, J = 8.3, 2H), 4.78-4.62 (m, 2H), 4.09-3.98 (m, 2H), 3.72-3.59 (m, 2H), |
| | 3.39 (d, J = 11.3, 1H), 2.92 (s, 3H), 2.91-2.82 (m, 2H), 2.82-2.74 (m, 2H), 2.71-2.62 (m, |
| | 2H), 2.37-2.20 (m, 1H), 2.11-1.97 (m, 1H). |
| 283 | 1H NMR (400 MHz, cdcl3) δ 8.86~8.85 (d, J = 1.6 Hz, 1H), 8.79~8.78 (d, J = 2.0 Hz, 1H), |
| | 8.11~8.09 (d, J = 8.4 Hz, 2H), 7.90 (s, 1H), 7.54~7.52 (d, J = 8.4 Hz, 2H), 4.94~4.68 (m, 2H), |
| | 4.26~4.20 (m, 1H), 4.09~4.07 (d, J = 11.6 Hz, 1H), 3.84~3.78 (m, 2H), 3.53~3.51 (d, J = 11.2 |
| | Hz, 1H), 2.97~2.84 (m, 3H), 1.98~1.82 (m, 4H), 0.83~0.80 (t, J = 7.6 Hz, 6H). |
| 284 | 1H NMR (400 MHz, cdcl3) δ 8.89 (dd, J = 1.8, 0.9, 1H), 8.77 (dd, J = 1.8, 1.0, 1H), 8.03 (d, J = |
| | 8.4, 2H), 7.90 (s, 1H), 7.39 (d, J = 8.2, 2H), 4.80-4.84 (m, 1H), 4.68-4.72 (m, 1H), 4.42 (s, |
| | 1H), 4.15-4.19 (m, 1H), 4.01-4.04 (m, 1H), 3.82-3.84 (m, 1H), 3.70-3.77 (m, 1H), 3.52- |
| | 3.55 (m, 1H), 2.79-2.90 (m, 2H), 2.74 (s, 3H), 0.91 (s, 9H). |
| 285 | 1H NMR (400 MHz, cdcl3) δ 8.95 (d, J = 9.4, 1H), 8.83 (s, 1H), 8.11 (d, J = 8.3, 2H), 7.96 (d, J = |
| | 11.2, 1H), 7.46 (d, J = 8.4, 2H), 4.74-4.82 (m, 2H), 4.39-4.69 (m, 1H), 4.48 (s, 1H), 4.04- |
| | 4.12 (m, 1H), 3.93-4.03 (m, 1H), 3.61-3.67 (m, 2H), 3.26-3.39 (m, 1H), 2.79-2.93 (m, 1H), |
| | 2.10 (d, J = 5.9, 3H), 0.97 (s, 9H). |
| 286 | 1H NMR (400 MHz, cdcl3) δ 8.95 (d, J = 7.8, 1H), 8.84 (s, 1H), 8.13 (d, J = 8.2, 2H), 7.96 (d, J = |
| | 11.4, 1H), 7.46 (d, J = 8.3, 2H), 4.74-4.90 (m, 2H), 4.39-4.69 (m, 2H), 3.93-4.12 (m, 3H), |
| | 3.59-3.68 (m, 1H), 3.26-3.40 (m, 1H), 2.79-2.93 (m, 1H), 2.10 (d, J = 5.7, 3H), 1.99-2.04 |
| | (m, 1H), 1.03 (d, J = 6.7, 3H), 0.87 (d, J = 6.8, 3H). |
| 287 | 1H NMR (400 MHz, cdcl3) δ 8.89 (d, J = 1.8, 1H), 8.77 (d, J = 1.8, 1H), 8.06 (d, J = 8.4, 2H), |
| | 7.90 (s, 1H), 7.40 (d, J = 8.3, 2H), 4.80-4.84 (m, 1H), 4.68-4.72 (m, 1H), 4.40-4.42 (m, 1H), |
| | 4.17-4.19 (m, 1H), 4.01-4.06 (m, 1H), 3.82-3.85 (m, 1H), 3.70-3.76 (m, 1H), 3.52-3.54 |
| | (m, 1H), 2.78-2.90 (m, 2H), 2.74 (s, 3H), 1.93-1.98 (m, 1H), 0.96 (d, J = 6.7, 3H), 0.81 (d, J = |
| | 6.8, 3H). |
| 288 | 1 H NMR (400 MHz, cdcl 3) δ 8.89 (d, J = 1.8, 1H), 8.77 (d, J = 1.8, 1H), 8.11-8.01 (m, 2H), |
| | 7.89 (s, 1H), 7.53-7.43 (m, 2H), 4.86-4.65 (m, 2H), 4.21-4.13 (m, 1H), 4.05-3.98 (m, 1H), |
| | 3.86-3.79 (m, 1H), 3.77-3.68 (m, 1H), 3.62 (s, 2H), 3.56-3.49 (m, 1H), 2.92-2.77 (m, 2H), |
| | 2.74 (s, 3H), 1.33 (s, 6H). |
| 289 | 1 H NMR (400 MHz, cdcl 3) δ 8.91 (d, J = 8.3, 1H), 8.80 (s, 1H), 8.12 (d, J = 8.1, 2H), 7.91 (d, |
| | J = 10.6, 1H), 7.51 (d, J = 8.4, 2H), 4.88-4.29 (m, 3H), 4.05-3.50 (m, 4H), 3.36-3.17 (m, 1H), |
| | 2.93-2.70 (m, 3H), 2.68-2.54 (m, 2H), 2.50-2.31 (m, 1H), 2.15-1.97 (m, 4H). |
| 290 | 1H NMR (400 MHz, cdcl 3) δ 8.97 (d, J = 1.8, 1H), 8.84 (d, J = 1.8, 1H), 8.21-8.15 (m, 2H), |
| | 7.97 (s, 1H), 7.61-7.53 (m, 2H), 4.90-4.85 (m, 1H), 4.73-4.68 (m, 1H), 4.16-3.99 (m, 3H), |
| | 3.79-3.63 (m, 2H), 3.18-3.02 (m, 2H), 2.93-2.84 (m, 2H), 2.73-2.62 (m, 2H), 2.53-2.41 |
| | (m, 1H), 2.18-2.08 (m, 1H). |
| 291 | 1H NMR (400 MHz, cdcl3) δ 8.96~8.94 (d, J = 8.8 Hz, 1H), 8.83 (s, 1H), 8.13~8.11 (d, J = 7.6 |
| | Hz, 2H), 7.97~7.95 (d, J = 10.4 Hz, 1H), 7.45~7.43 (d, J = 8.0 Hz, 2H), 4.90~4.75 (m, 2H), |
| | 4.70~4.39 (m, 1H), 4.12~4.01 (m, 2H), 3.97~3.58 (m, 3H), 3.40~3.27 (m, 1H), 2.94~2.79 (m, |
| | 1H), 2.11~2.10 (d, J = 6.8 Hz, 3H), 1.96~1.87 (m, 1H), 1.02~1.00 (d, J = 6.8 Hz, 3H), 0.85~0.83 |
| | (d, J = 6.8 Hz, 3H). |
| 292 | 1H NMR (400 MHz, dmso) δ 9.09 (d, J = 1.8, 1H), 8.91 (d, J = 1.8, 1H), 8.07 (s, 1H), 7.88- |
| | 7.84 (m, 1H), 7.82 (d, J = 2.0, 1H), 7.10 (d, J = 8.5, 1H), 6.88 (s, 2H), 4.80-4.65 (m, 2H), 4.13- |
| | 3.99 (m, 2H), 3.91 (s, 3H), 3.84 (s, 3H), 3.69-3.52 (m, 2H), 3.28-3.26 (m, 1H), 2.75-2.64 |
| | (m, 2H). |
| 293 | 1 H NMR (400 MHz, cd 3 od) δ 8.93 (d, J = 1.8, 1H), 8.78 (d, J = 1.8, 1H), 8.09 (d, J = 8.5, 2H), |
| | 7.89 (s, 1H), 7.40-7.35 (m, 2H), 4.86-4.62 (m, 2H), 4.20-4.11 (m, 1H), 4.05-4.01 (m, 1H), |
| | 3.76-3.66 (m, 2H), 3.39 (d, J = 11.6, 1H), 2.90-2.74 (m, 2H), 1.77-1.71 (m, 2H), 1.49-1.43 |
| | (m, 2H). |
| 294 | 1 H NMR (400 MHz, cd 3 od) δ 8.95 (d, J = 1.9, 1H), 8.80 (d, J = 1.9, 1H), 8.18-8.14 (m, 2H), |
| | 7.93 (s, 1H), 7.56-7.51 (m, 2H), 4.84-4.69 (m, 2H), 4.23-4.15 (m, 1H), 4.04-3.99 (m, 1H), |
| | 3.80-3.68 (m, 2H), 3.46-3.37 (m, 1H), 2.91-2.79 (m, 4H), 2.71-2.62 (m, 2H), 2.50-2.33 |
| | (m, 1H), 2.15-2.06 (m, 1H). |
| 295 | 1H NMR (400 MHz, dmso) δ 9.12 (d, J = 1.5, 1H), 8.96 (d, J = 1.7, 1H), 8.27 (s, 2H), 8.11 (s, |
| | 1H), 7.53 (d, J = 8.2, 2H), 4.77-4.67 (m, 2H), 4.58-4.52 (m, 1H), 4.50-4.45 (m, 0.5H), 4.31- |
| | 4.25 (m, 1H), 4.21-3.84 (m, 6H), 3.75-3.69 (m, 0.5H), 3.59-3.42 (m, 1H), 3.25-3.16 (m, |
| | 1H), 2.79-2.68 (m, 1H), 2.03 (s, 3H), 1.82 (s, 3H). |
| 296 | 1H NMR (400 MHz, dmso) δ 9.12 (s, 1H), 8.96 (s, 1H), 8.27 (d, J = 8.0, 2H), 8.10 (s, 1H), 7.53 |
| | (d, J = 8.1, 2H), 6.05 (s, 2H), 4.72-4.65 (m, 2H), 4.58-4.52 (m, 1H), 4.32-4.25 (m, 1H), 4.21- |
| | 4.16 (m, 1H), 4.09-4.03 (m, 1H), 3.94-3.85 (m, 4H), 3.77-3.71 (m, 1H), 3.52-3.45 (m, |
| | 1H), 2.91-2.76 (m, 2H), 1.82 (s, 3H). |
| 297 | 1H NMR (400 MHz, dmso) δ 9.12 (d, J = 1.8, 1H), 8.96 (d, J = 1.8, 1H), 8.20-8.13 (m, 2H), |
| | 8.08 (s, 1H), 7.41 (d, J = 7.9, 1H), 5.06 (s, 2H), 4.76-4.67 (m, 2H), 4.52-4.16 (m, 1H), 4.00- |
| | 3.70 (m, 3H), 3.58-3.43 (m, 1H), 3.25-3.16 (m, 1H), 2.78-2.67 (m, 1H), 2.03 (s, 3H), 1.47 |
| | (s, 6H). |
| 298 | 1H NMR (400 MHz, dmso) δ 9.12 (d, J = 1.9, 1H), 8.96 (d, J = 1.9, 1H), 8.18 (d, J = 8.0, 1H), |
| | 8.15 (s, 1H), 8.08 (s, 1H), 7.41 (d, J = 7.9, 1H), 6.05 (s, 2H), 5.06 (s, 2H), 4.74-4.61 (m, 2H), |
| | 4.11-4.02 (m, 1H), 3.94-3.84 (m, 2H), 3.79-3.69 (m, 1H), 3.53-3.45 (m, 1H), 2.93-2.75 |
| | (m, 2H), 1.47 (s, 6H). |
| 299 | 1H NMR (400 MHz, dmso) δ 9.12 (d, J = 1.9, 1H), 8.96 (d, J = 1.9, 1H), 8.20-8.16 (m, 2H), |
| | 8.14 (d, J = 0.8, 1H), 8.09 (s, 1H), 7.41 (d, J = 8.0, 1H), 5.06 (s, 2H), 4.76-4.68 (m, 2H), 4.11- |
| | 3.97 (m, 2H), 3.74-3.68 (m, 1H), 3.68-3.60 (m, 1H), 3.45-3.38 (m, 1H), 2.94 (s, 3H), 2.92- |
| | 2.84 (m, 2H), 1.47 (s, 6H). |
| 300 | 1H NMR (400 MHz, cd3od) δ 9.01 (dd, J = 8.7, 1.6, 1H), 8.83 (dd, J = 6.7, 1.7, 1H), 8.03-7.97 |
| | (m, 1H), 7.97-7.91 (m, 2H), 7.32 (d, J = 8.2, 1H), 4.75-4.26 (m, 2H), 4.17-3.88 (m, 5H), |
| | 3.83-3.54 (m, 2H), 3.41-3.33 (m, 1H), 2.97-2.88 (m, 3H), 2.14 (d, J = 5.3, 3H), 1.56 (s, 6H). |
| 301 | 1H NMR (400 MHz, cd3od) δ 9.02 (d, J = 1.9, 1H), 8.83 (d, J = 1.9, 1H), 8.00 (dd, J = 8.3, 2.0, |
| | 1H), 7.95 (s, 1H), 7.93 (d, J = 1.7, 1H), 7.32 (d, J = 8.3, 1H), 4.67-4.61 (m, 1H), 4.21-4.14 |
| | (m, 1H), 4.07-3.96 (m, 4H), 3.89-3.84 (m, 1H), 3.68-3.60 (m, 1H), 3.52-3.41 (m, 1H), |
| | 3.09-2.98 (m, 2H), 2.95-2.89 (m, 2H), 2.65 (s, 2H), 1.56 (s, 6H). |
| 302 | 1H NMR (400 MHz, cd3od) δ 9.02 (d, J = 1.9, 1H), 8.83 (d, J = 1.9, 1H), 7.99 (d, J = 8.2, 1H), |
| | 7.95 (s, 1H), 7.93 (s, 1H), 7.32 (d, J = 8.3, 1H), 4.20-4.12 (m, 1H), 4.09-4.04 (m, 1H), 4.01- |
| | 3.96 (m, 2H), 3.89-3.82 (m, 1H), 3.78-3.70 (m, 1H), 3.56-3.51 (m, 1H), 3.04-2.96 (m, |
| | 2H), 2.95-2.88 (m, 5H), 1.56 (s, 6H). comment: 4.71-4.83 (m, 2H) |
| 303 | 1H NMR (400 MHz, cdcl3) δ 8.94~8.93 (d, J = 2.0 Hz, 1H), 8.80~8.79 (d, J = 1.6 Hz, 1H), |
| | 8.11~8.09 (d, J = 9.2 Hz, 2H), 7.88 (s, 1H), 7.03~7.01 (d, J = 9.2 Hz, 2H), 4.98~4.76 (m, 2H), |
| | 4.51~4.45 (m, 1H), 4.39~4.25 (m, 2H), 3.91~3.89 (t, J = 4.8 Hz, 4H), 3.68~3.62 (t, J = 12.0 Hz, |
| | 1H), 3.49~3.45 (m, 1H), 3.30~3.28 (t, J = 4.8 Hz, 4H), 3.02 (s, 3H). |
| 304 | 1H NMR (400 MHz, cdcl3) δ 8.93~8.92 (d, J = 2.0 Hz, 1H), 8.79~8.78 (d, J = 2.0 Hz, 1H), |
| | 8.09~8.07 (d, J = 8.8 Hz, 2H), 7.86 (s, 1H), 7.04~7.02 (d, J = 8.8 Hz, 2H), 4.97~4.76 (m, 2H), |
| | 4.51~4.44 (m, 1H), 4.38~4.25 (m, 2H), 3.67~3.62 (t, J = 11.6 Hz, 1H), 3.49~3.45 (m, 1H), |
| | 3.37~3.34 (t, J = 5.2 Hz, 4H), 3.02 (s, 3H), 2.61~2.59 (t, J = 4.8 Hz, 4H), 2.37 (s, 3H). |
| 305 | 1H NMR (400 MHz, cdcl3) δ 8.96~8.95 (d, J = 2.0 Hz, 1H), 8.83~8.82 (d, J = 2.0 Hz, 1H), 7.91 |
| | (s, 1H), 7.79~7.77 (dd, J = 2.4 Hz, 8.8 Hz, 1H), 7.77~7.70 (d, J = 2.0 Hz, 1H), 7.02~7.00 (d, J = |
| | 8.4 Hz, 1H), 4.98~4.75 (m, 2H), 4.51~4.45 (m, 1H), 4.38~4.24 (m, 2H), 4.01 (s, 3H), 3.97 (s, |
| | 3H), 3.68~3.63 (t, J = 12.0 Hz, 1H), 3.49~3.45 (m, 1H), 3.02 (s, 3H). |
| 306 | 1H NMR (400 MHz, cdcl3) δ 8.98~8.97 (d, J = 2.0 Hz, 1H), 8.86~8.85 (d, J = 1.6 Hz, 1H), |
| | 8.13~8.11 (d, J = 8.4 Hz, 2H), 7.97 (s, 1H), 7.38~7.36 (d, J = 8.4 Hz, 2H), 4.97~4.77 (m, 2H), |
| | 4.51~4.45 (m, 1H), 4.39~4.25 (m, 2H), 4.00~3.97 (d, J = 12.0 Hz, 2H), 3.68~3.63 (t, J = 11.6 |
| | Hz, 1H), 3.49~3.45 (dd, J = 3.6 Hz, 12.0 Hz, 1H), 3.02 (s, 3H), 2.85~2.79 (m, 5H), 2.75~2.69 |
| | (m, 1H), 2.04~1.86 (m, 4H). |
| 307 | 1H NMR (400 MHz, cdcl3) δ 9.00~8.99 (d, J = 1.6 Hz, 1H), 8.89~8.88 (d, J = 1.6 Hz, 1H), |
| | 8.19~8.17 (d, J = 8.8 Hz, 2H), 8.00 (s, 1H), 7.65~7.63 (d, J = 8.8 Hz, 2H), 4.98~4.77 (m, 2H), |
| | 4.51~4.45 (m, 1H), 4.39~4.25 (m, 2H), 3.69~3.64 (t, J = 11.6 Hz, 1H), 3.49~3.45 (dd, J = 3.2 |
| | Hz, 11.6 Hz, 1H), 3.03 (s, 3H), 1.80 (s, 6H). |
| 308 | 1H NMR (400 MHz, cdcl3) δ 8.90 (d, J = 1.9, 1H), 8.78 (d, J = 1.9, 1H), 8.09 (d, J = 8.2, 2H), |
| | 7.91 (s, 1H), 7.59 (d, J = 8.2, 2H), 4.85-4.81 (m, 1H), 4.72-4.68 (m, 1H), 4.22-4.15 (m, 1H), |
| | 4.04-4.01 (m, 1H), 3.85-3.82 (m, 1H), 3.74-3.70 (m, 1H), 3.56-3.53 (m, 1H), 2.91-2.85 |
| | (m, 1H), 2.84-2.81 (m, 1H), 2.74 (s, 3H), 2.60-2.53 (m, 2H), 2.40-2.33 (m, 2H), 2.05-1.97 |
| | (m, 2H), 1.74-1.67 (m, 1H). |
| 309 | 1H NMR (400 MHz, cdcl3) δ 8.89 (d, J = 9.2, 1H), 8.78 (s, 1H), 8.10 (d, J = 8.4, 2H), 7.90 (d, J = |
| | 11.2, 1H), 7.62-7.55 (m, 2H), 4.85-4.66 (m, 2H), 4.63-4.32 (m, 1H), 4.02-3.86 (m, 2H), |
| | 3.61-3.52 (m, 2H), 3.33-3.20 (m, 1H), 2.87-2.78 (m, 1H), 2.59-2.53 (m, 2H), 2.40-2.33 |
| | (m, 2H), 2.10-2.08 (m, 1H), 2.04 (d, J = 5.1, 3H), 1.73-1.66 (m, 1H). |
| 310 | 1H NMR (400 MHz, cdcl3) δ 8.97 (s, 1H), 8.85 (s, 1H), 8.14-8.12 (d, J = 8.4 Hz, 2H), 7.98 (s, |
| | 1H), 7.66-7.64 (d, J = 8.4 Hz, 2H), 4.97-4.77 (m, 2H), 4.51-4.44 (m, 1H), 4.38-4.25 (m, 2H), |
| | 3.68-3.63 (t, J = 11.6 Hz, 1H), 3.48-3.45 (dd, J = 2.8 Hz, 11.6 Hz, 1H), 3.02 (s, 3H), 1.65 (s, |
| | 6H). |
| 311 | 1H NMR (400 MHz, cdcl3) δ 8.88 (d, J = 1.8, 1H), 8.73 (d, J = 1.8, 1H), 8.04 (d, J = 8.6, 2H), |
| | 7.85 (d, J = 0.9, 1H), 7.53 (d, J = 8.0, 2H), 4.79-4.75 (m, 1H), 4.67 (s, 2H), 4.65-4.59 (m, 1H), |
| | 4.06-4.03 (m, 2H), 4.00-3.94 (m, 1H), 3.74-3.71 (m, 1H), 3.62-3.59 (m, 1H), 3.10-2.94 (m, |
| | 2H), 2.59-2.51 (m, 3H), 2.39-2.33 (m, 2H), 2.06-1.96 (m, 1H). |
| 312 | 1H NMR (400 MHz, cdcl3) δ 8.85 (d, J = 1.8, 1H), 8.75 (d, J = 1.8, 1H), 8.06 (d, J = 8.2, 2H), |
| | 7.86 (s, 1H), 7.58 (d, J = 8.2, 2H), 4.85-4.81 (m, 1H), 4.70-4.60 (m, 3H), 4.18-4.17 (m, 1H), |
| | 4.03-4.01 (m, 1H), 3.78-3.72 (m, 2H), 3.47-3.42 (m, 1H), 2.90-2.79 (m, 2H), 2.59-2.52 |
| | (m, 2H), 2.40-2.33 (m, 2H), 2.07-1.97 (m, 1H), 1.73-1.66 (m, 1H). |
| 313 | 1H NMR (400 MHz, cdcl3) δ 8.96~8.95 (d, J = 1.6 Hz, 1H), 8.84~8.86 (d, J = 1.6 Hz, 1H), |
| | 8.12~8.10 (d, J = 8.4 Hz, 2H), 7.95 (s, 1H), 7.55~7.52 (d, J = 8.8 Hz, 2H), 5.78 (s, 1H), |
| | 4.90~4.75 (m, 2H), 4.28~4.22 (m, 1H), 4.11~4.07 (m, 1H), 3.91~3.88 (m, 1H), 3.83~3.77 (m, |
| | 1H), 3.62~3.59 (d, J = 11.2 Hz, 1H), 2.97~2.84 (m, 2H), 2.81 (s, 3H), 2.01 (s, 3H), 1.75 (s, 6H). |
| 314 | 1H NMR (400 MHz, cdcl3) δ 8.97~8.96 (d, J = 2.0 Hz, 1H), 8.86~8.85 (d, J = 2.0 Hz, 1H), |
| | 8.17~8.15 (d, J = 8.8 Hz, 2H), 7.98 (s, 1H), 7.67~7.65 (d, J = 8.4 Hz, 2H), 4.92~4.75 (m, 2H), |
| | 4.28~4.22 (m, 1H), 4.12~4.07 (m, 1H), 4.01~3.89 (m, 5H), 3.84~3.77 (m, 1H), 3.63~3.60 (d, J = |
| | 11.6 Hz, 1H), 2.98~2.85 (m, 2H), 2.82 (s, 3H), 2.30~2.22 (m, 2H), 1.76~1.71 (m, 3H). |
| 315 | 1H NMR (400 MHz, cdcl3) δ 8.93 (s, 1H), 8.83 (d, J = 1.6, 1H), 8.16 (d, J = 8.2, 2H), 7.93 (s, |
| | 1H), 7.34 (d, J = 8.3, 2H), 5.28-5.25 (m, 1H), 5.05-5.02 (m, 1H), 4.84-4.82 (m, 1H), 4.19- |
| | 4.16 (m, 1H), 3.99-3.89 (m, 3H), 3.69-3.68 (m, 1H), 3.30-3.27 (m, 1H), 3.05 (s, 3H), 2.83 (s, |
| | 3H), 2.81-2.78 (m, 2H), 2.72-2.66 (m, 1H), 2.03-2.00 (m, 2H), 1.94-1.88 (m, 2H). |
| 316 | 1H NMR (400 MHz, cdcl3) δ 8.93 (s, 1H), 8.82 (s, 1H), 8.17 (d, J = 6.6, 2H), 7.93 (s, 1H), 7.62 |
| | (d, J = 6.6, 2H), 5.27-5.24 (m, 1H), 5.08-5.03 (m, 1H), 4.84-4.82 (m, 1H), 4.18-4.15 (m, |
| | 1H), 3.93-3.88 (m, 1H), 3.70-3.65 (m, 1H), 3.29-3.27 (m, 1H), 3.05 (s, 3H), 1.63 (s, 6H). |
| 317 | 1H NMR (400 MHz, cdcl3) δ 8.96 (d, J = 1.8, 1H), 8.84 (d, J = 1.8, 1H), 8.14 (d, J = 8.3, 2H), |
| | 7.96 (s, 1H), 7.35 (d, J = 8.4, 2H), 5.02 (d, J = 5.6, 2H), 4.90-4.86 (m, 1H), 4.78-4.74 (m, 1H), |
| | 4.68 (d, J = 5.6, 2H), 4.29-4.21 (m, 1H), 4.09-4.07 (m, 1H), 3.91-3.88 (m, 1H), 3.82-3.76 |
| | (m, 1H), 3.65-3.58 (m, 1H), 2.95-2.84 (m, 2H), 2.80 (s, 3H), 1.78 (s, 3H). |
| 318 | 1H NMR (400 MHz, cdcl3) δ 8.78 (s, 1H), 8.59 (s, 1H), 7.69 (d, J = 8.1, 2H), 7.55 (s, 1H), 7.34 |
| | (d, J = 8.2, 2H), 4.82-4.74 (m, 1H), 4.59-4.55 (m, 2H), 4.12-3.99 (m, 2H), 3.77-3.70 (m, |
| | 2H), 3.53-3.50 (m, 1H), 2.93-2.85 (m, 1H), 2.77 (s, 3H), 1.55 (s, 3H), 1.54 (s, 3H). |
| 319 | 1H NMR (400 MHz, cdcl3) δ 8.93 (d, J = 1.9, 1H), 8.82 (d, J = 1.9, 1H), 8.19-8.14 (m, 2H), |
| | 7.94 (s, 1H), 7.64-7.60 (m, 2H), 6.17 (s, 1H), 5.25-5.21 (m, 1H), 5.11-5.08 (m, 1H), 4.85- |
| | 4.83 (m, 1H), 4.20-4.15 (m, 1H), 3.90-3.83 (m, 1H), 3.72-3.65 (m, 1H), 3.39 (dt, J = 8.4, |
| | 3.4, 1H), 1.63 (s, 6H). |
| 320 | 1H NMR (400 MHz, cdcl3) δ 8.93 (d, J = 1.9, 1H), 8.82 (d, J = 1.9, 1H), 8.15 (d, J = 8.4, 2H), |
| | 7.93 (s, 1H), 7.34 (d, J = 8.5, 2H), 6.06 (s, 1H), 5.25-5.22 (m, 1H), 5.12-5.07 (m, 1H), 4.86- |
| | 4.83 (m, 1H), 4.19-4.16 (m, 1H), 3.98-3.95 (m, 2H), 3.87-3.85 (m, 1H), 3.69-3.65 (m, 1H), |
| | 3.41-3.37 (m, 1H), 2.83-2.78 (m, 5H), 2.69-2.66 (m, 1H), 2.03-1.99 (m, 2H), 1.93-1.84 |
| | (m, 2H). |
| 321 | 1H NMR (400 MHz, dmso) δ 9.07 (d, J = 1.4, 1H), 8.91 (d, J = 1.4, 1H), 8.19 (d, J = 8.3, 2H), |
| | 8.04 (s, 1H), 7.40 (d, J = 8.3, 2H), 4.72-4.62 (m, 2H), 4.36 (d, J = 8.2, 1H), 4.13 (d, J = 8.2, |
| | 1H), 4.08-3.96 (m, 3H), 3.87 (d, J = 9.3, 1H), 3.71-3.58 (m, 2H), 3.40-3.37 (m, 1H), 2.91 (s, |
| | 3H), 2.89-2.80 (m, 2H), 1.78 (s, 3H), 1.57 (s, 3H). |
| 322 | 1H NMR (400 MHz, cdcl3) δ 8.96 (d, J = 1.7, 1H), 8.85 (d, J = 1.7, 1H), 8.20 (d, J = 1.5, 1H), |
| | 8.12-8.10 (m, 1H), 7.96 (d, J = 1.2, 1H), 7.30 (d, J = 8.2, 1H), 5.54-5.40 (m, 1H), 4.88-4.83 |
| | (m, 1H), 4.82-4.71 (m, 1H), 4.26-4.22 (m, 1H), 4.19-4.06 (m, 3H), 3.90-3.87 (m, 1H), 3.83- |
| | 3.75 (m, 1H), 3.60-3.57 (m, 1H), 2.95-2.83 (m, 2H), 2.82-2.76 (m, 3H), 1.64 (s, 3H), 1.53 |
| | (s, 3H). |
| 323 | 1H NMR (400 MHz, cdcl3) δ 8.96 (d, J = 8.5, 1H), 8.85 (s, 1H), 8.20-8.112 (m, 2H), 7.96 (d, |
| | J = 9.3, 1H), 7.30 (d, J = 7.8, 1H), 5.53-5.40 (m, 1H), 4.81-4.76 (m, 2H), 4.69-4.38 (m, 1H), |
| | 4.15-3.91 (m, 4H), 3.63-3.60 (m, 2H), 3.38-3.28 (m, 1H), 2.89-2.77 (m, 1H), 2.10 (s, 3H), |
| | 1.64 (s, 3H), 1.53 (s, 3H). |

### Example 2

### Enzymatic Assay

### SYK Enzymatic Assay:

Syk kinase assay are performed in vitro using Kit-Tyr 2 Peptide (Invitrogen, Cat. No. PV3191) and in a 384-well assay plate. All reactions (40 μL) are started by adding 0.8 μL of the testing compound in 100% DMSO solution, 10 μL of Kinase/Peptide substrate mixture or Phospho-Peptide solution (Invitrogen, Cat. No. PV3192, diluted with 1.33× Kinase Buffer), 5 μL ATP solution (100×M) or 1.33× kinase buffer (Invitrogen, Cat. No. PV3189, 5× diluted with distilled water), 4.2 μL distilled water. The 384-well assay plate (Corning, Cat. No. 3575) is mixed and incubated at room temperature for 1 hour. 10 μL of the Development Solution (prepared by diluting Development Reagent A (Cat. No. PV3297) to 1/32 with Development Buffer (Cat. No. PV3127)) is then added to each well, mixed and incubated at room temperature for another 1 hour. The reactions are then stopped by adding 10 μL of the Stop Reagent (Invitrogen, Cat. No. PV3094), and the plate is read with Wallac 1420 VICTOR³ Multilabel Counter (PerkinElmer™) at 445 nm and 520 nm fluorescence. All compounds are tested at 8 concentrations (1 μM down to 0.0003 μM) using a 1:3 serial dilution scheme.

Below are the IC₅₀ values of some compounds.

| Cmpd | IC₅₀ (μM) |
|---|---|
| 1 | 0.270 |
| 2 | 0.552 |
| 3 | 0.459 |
| 4 | 0.160 |
| 5 | 0.172 |
| 6 | 0.034 |
| 7 | 0.071 |
| 8 | 0.048 |
| 9 | 0.098 |
| 10 | 0.018 |
| 11 | 0.052 |
| 12 | 0.024 |
| 13 | 0.025 |
| 14 | 0.025 |
| 15 | 0.096 |
| 16 | 0.036 |
| 17 | 0.032 |
| 18 | 0.023 |
| 19 | 0.029 |
| 20 | 0.041 |
| 21 | 0.030 |
| 22 | 0.016 |
| 23 | 0.062 |
| 24 | 0.076 |
| 25 | 0.067 |
| 26 | 0.018 |
| 27 | 0.021 |
| 28 | 0.040 |
| 29 | 0.066 |
| 30 | 0.043 |
| 31 | 0.017 |
| 32 | 0.060 |
| 33 | 0.027 |
| 34 | 0.227 |
| 35 | 0.131 |
| 36 | 0.055 |
| 37 | 0.040 |
| 38 | 0.083 |
| 39 | 0.033 |
| 40 | 0.338 |
| 41 | 0.138 |
| 42 | 0.139 |
| 43 | 0.013 |
| 44 | 0.324 |
| 45 | 0.846 |
| 46 | 0.192 |
| 47 | 0.122 |
| 48 | 0.087 |
| 49 | 0.087 |
| 50 | 0.064 |
| 51 | 0.094 |
| 52 | 0.042 |
| 53 | 0.032 |
| 54 | 0.073 |
| 55 | 0.065 |
| 56 | 0.121 |
| 57 | 0.014 |
| 58 | 0.031 |
| 59 | 0.030 |
| 60 | 0.588 |
| 61 | 0.027 |
| 62 | 0.024 |
| 63 | 0.037 |
| 64 | 0.020 |
| 65 | 0.053 |
| 66 | 0.058 |
| 67 | 0.052 |
| 68 | 0.063 |
| 69 | 0.039 |
| 70 | 0.099 |
| 71 | 0.127 |
| 72 | 0.109 |
| 73 | 0.204 |
| 74 | 0.127 |
| 75 | 0.095 |
| 76 | 0.040 |
| 77 | 0.085 |
| 78 | 0.061 |
| 79 | 0.231 |
| 80 | 0.107 |
| 81 | 0.166 |
| 82 | 0.113 |
| 83 | 0.074 |
| 84 | 0.081 |
| 85 | 0.283 |
| 86 | 0.110 |
| 87 | 0.060 |
| 88 | 0.032 |
| 89 | 0.096 |
| 90 | 0.046 |
| 91 | 0.474 |
| 92 | 0.076 |
| 93 | 0.808 |
| 94 | 0.015 |
| 95 | 0.031 |
| 96 | 0.134 |
| 97 | 0.144 |
| 98 | 0.055 |
| 99 | 0.023 |
| 100 | 0.031 |
| 101 | 0.052 |
| 102 | 0.038 |
| 103 | 0.032 |
| 104 | 0.038 |
| 105 | 0.039 |
| 106 | 0.13 |
| 107 | 0.919 |
| 108 | 0.019 |
| 109 | 0.097 |
| 110 | 0.211 |
| 111 | 0.117 |
| 114 | 0.122 |
| 119 | 0.016 |
| 120 | 0.061 |
| 122 | 0.015 |
| 123 | 0.03 |
| 124 | 0.033 |
| 125 | 0.031 |
| 126 | 0.041 |
| 127 | 0.058 |
| 128 | 0.477 |
| 129 | 0.050 |
| 130 | 0.454 |
| 131 | 0.028 |
| 132 | 0.109 |
| 133 | 0.045 |
| 134 | 0.003 |
| 136 | 0.025 |
| 137 | 0.028 |
| 139 | 0.095 |
| 140 | 0.052 |
| 141 | 0.098 |
| 142 | 0.068 |
| 143 | 0.080 |
| 144 | 0.057 |
| 147 | 0.115 |
| 148 | 0.095 |
| 149 | 0.030 |
| 150 | 0.092 |
| 151 | 0.044 |
| 152 | 0.033 |
| 153 | 0.033 |
| 154 | 0.043 |
| 155 | 0.032 |
| 156 | 0.038 |
| 158 | 0.030 |
| 160 | 0.102 |
| 161 | 0.151 |
| 162 | 0.151 |
| 163 | 0.036 |
| 164 | 0.066 |
| 165 | 0.048 |
| 166 | 0.052 |
| 167 | 0.405 |
| 168 | 0.211 |
| 169 | 0.272 |
| 170 | 0.182 |
| 171 | 0.458 |
| 172 | 0.050 |
| 173 | 0.032 |
| 174 | 0.816 |
| 175 | 0.055 |
| 176 | 0.059 |
| 177 | 0.091 |
| 178 | 0.019 |
| 179 | 0.190 |
| 180 | 0.177 |
| 181 | 0.091/0.068 |
| 182 | 0.110 |
| 183 | 0.076 |
| 184 | 0.084 |
| 185 | 0.040 |
| 186 | 0.081 |
| 187 | 0.109 |
| 188 | 0.011 |
| 189 | 0.061 |
| 190 | 0.050 |
| 191 | 0.115 |
| 192 | 0.030 |
| 193 | 0.066 |
| 194 | 0.136 |
| 195 | 0.063 |
| 196 | 0.067 |
| 197 | 0.085 |
| 198 | 0.040 |
| 199 | 0.088 |
| 200 | 0.051 |
| 201 | 0.049 |
| 202 | 0.026 |
| 203 | 0.060 |
| 204 | 0.126 |
| 205 | 0.027 |
| 206 | 0.072 |
| 207 | 0.011 |
| 208 | 0.013 |
| 209 | 0.017 |
| 210 | 0.030 |
| 211 | 0.058 |
| 212 | 0.017 |
| 213 | 0.057 |
| 214 | 0.016 |
| 215 | 0.019 |
| 216 | 0.049 |
| 217 | 0.045 |
| 218 | 0.026 |
| 219 | 0.032/0.028 |
| 220 | 0.086 |
| 221 | 0.044 |
| 222 | 0.084 |
| 223 | 0.057 |
| 224 | 0.048 |
| 225 | 0.047 |
| 226 | 0.029 |
| 227 | 0.037 |
| 228 | 0.028 |
| 229 | 0.049 |
| 230 | 0.152 |
| 231 | 0.042 |
| 232 | 0.067 |
| 233 | 0.386 |
| 234 | 0.193 |
| 235 | 0.212 |
| 236 | 0.109 |
| 238 | 0.275 |
| 239 | 0.451 |
| 240 | 0.262 |
| 241 | 0.047 |
| 242 | 0.111 |
| 243 | 0.300 |
| 244 | 0.136 |
| 245 | 0.449 |
| 246 | 0.095 |
| 248 | 0.088 |
| 249 | 0.042 |
| 250 | 0.028 |
| 251 | 0.075 |
| 252 | 0.031 |
| 254 | 0.055 |
| 255 | 0.027 |
| 256 | 0.036 |
| 257 | 0.063 |
| 258 | 0.081 |
| 259 | 0.054 |
| 260 | 0.024 |
| 261 | 0.052 |
| 262 | 0.079 |
| 263 | 0.023 |
| 264 | 0.038 |
| 265 | 0.088 |
| 266 | 0.044 |
| 267 | 0.477 |
| 268 | 0.018 |
| 269 | 0.091 |
| 270 | 0.179 |
| 271 | 0.741 |
| 272 | 0.178 |
| 273 | 0.085 |
| 274 | 0.040 |
| 275 | 0.076 |
| 276 | 0.112 |
| 277 | 0.192 |
| 278 | 0.175 |
| 279 | 0.035 |
| 280 | 0.113 |
| 281 | 0.065 |
| 282 | 0.026 |
| 283 | 0.044 |
| 284 | 0.097 |
| 285 | 0.258 |
| 286 | 0.403 |
| 287 | 0.105 |
| 288 | 0.025 |
| 289 | 0.115 |
| 290 | 0.050 |
| 292 | 0.014 |
| 293 | 0.050 |
| 294 | 0.051 |
| 295 | 0.128 |
| 296 | 0.072 |
| 297 | 0.058 |
| 298 | 0.028 |
| 299 | 0.014 |
| 300 | 0.030 |
| 301 | 0.024 |
| 302 | 0.009 |
| 308 | 0.035 |
| 309 | 0.127 |
| 310 | 0.588 |
| 311 | 0.035 |
| 312 | 0.069 |
| 313 | 0.104 |
| 314 | 0.043 |
| 315 | 0.103 |
| 316 | 0.115 |
| 317 | 0.033 |
| 318 | 0.017 |
| 319 | 0.208 |
| 320 | 0.119 |
| 321 | 0.014 |

### Transcreener Kinase Assay of VEGFR-2 (KDR)

### 1. Solution Preparation

1) Transcreenen™ KINASE Assy kit: Bellbrook Labs., 3003-10K;
2) Recombinant human KDR: Invitrogen, PV3660;
3) Poly E4Y (substrate): Sigma, P0275; 5 mg/mL, dissolved in MilliQ water;
4) Assay buffer: 67 mM HEPES, 0.013% Triton X-100, 27 mM MgCl₂, 0.67 mM MnCl₂, 1.25 mM DTT, PH 7.4;
5) 10 mM ATP: Invitrogen, PV3227;
6) 500 mM EDTA: Invitrogen, 15575-038;
7) 96 well black Greiner plate: Greiner, 675076.

### 2. Prepare Solution

1) Dilute the compound to 5 folds of final concentrations, keeping the DMSO concentration at 5%. The final concentrations are 1, 0.33, 0.11, 0.037, 0.012, 0.004, 0.0014, 0.0005 μM; and the final concentration of DMSO is 1%.
2) Prepare Enzyme/Substrate stock, Recombinant human KDR and Poly E4Y are both diluted in assay buffer. The final concentration is KDR (0.3 ng/μL), Poly E4Y (62.5 ng/μL). The mixture is keeping on ice surface before use;
3) Prepare ATP Diluents, 10 mM ATP is diluted in assay buffer, the final concentration is 25 μM;
4) Prepare ADP Diluents: diluted ADP (500 μM) in assay buffer, the final concentration is 25 μM;
5) Prepare ATP standard curve stock as following:

| Column | ADP diluents (μL) | ATP diluents (μL) |
|---|---|---|
| 1 | 50 | 0 |
| 2 | 25 | 25 |
| 3 | 10 | 40 |
| 4 | 5 | 45 |
| 5 | 5 | 95 |
| 6 | 5 | 195 |
| 7 | 5 | 495 |
| 8 | 4 | 496 |
| 9 | 3 | 497 |
| 10 | 2 | 498 |
| 11 | 1 | 499 |
| 12 | 1 | 999 |

### 3. Enzymatic Reaction

1) Add 5 μL of compound or control. (positive control, 5 μL of 5% DMSO; negative control, 5 μL of 500 mM EDTA);
2) Add 10 μL of Enzyme/Substrate stock;
3) Add 10 μL of ATP Diluents to begin the enzyme reaction and mix on plate shaker;
4) Add 5 μL of 5% DMSO, 10 μL of assay buffer and 10 μL of ATP standard curve stock into standard curve wells;
5) Incubate at 28° C. for 45 min, keeping plate in gently shaking.

### 4. Stop Reaction and Detect ADP

1) Prepare Detection Mix: diluted ADP Alexa633 tracer (1:100), ADP antibody (1:158), and stop & detect buffer (1:10) by MilliQ water;
2) Prepare Tracer Only control: diluted ADP Alexa633 tracer (1:100) and stop & detect buffer (1:10) by MilliQ water;
3) Prepare No Tracer control: diluted stop & detect buffer (1:10) by MilliQ water;
4) Add 25 μL of detection mix, Tracer Only control and No Tracer control into corresponding wells, respectively;
5) Incubate at 28° C. for 1 h, keeping plate in gently shaking;
6) Measure florescence polarization (FP) on TECAN F500. Excitation wavelength: 610 nm, Emission wavelength: 670 nm.

### 5. Data Analysis

$$Inhibition ( \% ) = 100 - \frac{Compound well [ ADP ]}{Positive control well [ ADP ]} \times 100$$

Wherein:
1) Compound well [ADP] represents the ADP concentration of compound well.
2) Positive control well [ADP] represents the ADP concentration of 5% DMSO well
3) Conversion of mP value to ADP concentration based on the formula which determined by standard curve. And measurement of mP value is following the suggestion of instruction which provided by BellBrook Labs. (www.bellbrooklabs.com).
   IC₅₀: calculated using XL-Fit 2.0 software.

Below are the IC₅₀ values of some compounds.

| | VEGFR-2 (KDR) |
|---|---|
| Compound | IC₅₀ (μM) |
| 26 | 1.598 |
| 33 | 1.328 |
| 43 | >3 |
| 53 | 2.962 |
| 57 | 1.56 |
| 108 | >3 |
| 219 | 0.748 |
| 300 | 1.133 |

### Z-Lyte Kinase Assay of Flt-3:

### Materials and Reagents:

| | Vender | Cat Number |
|---|---|---|
| Z-lyte assay kit-TYR2 | Invitrogen | PV3191 |
| Z-LYTE Tyr 2 Peptide | Invitrogen | PV3261 |
| Z-LYTE Tyr 2 Phospho-peptide | Invitrogen | PV3262 |
| 5X Kinase Buffer | Invitrogen | PV3189 |
| 10 mM ATP | Invitrogen | PV3227 |
| Development Reagent A | Invitrogen | PV3297 |
| Development Buffer | Invitrogen | P3127 |
| Stop Reagent | Invitrogen | P3094 |
| Flt3 kinase | Invitrogen | PV3182 |
| 384-well plate(black) | Corning | 3575 |
| Victor3 | PerkinElmer ™ | |

### Reaction Steps

### 1. Plate Map

| | Ref cpd Cons | Cpd 1 Cons | Cpd 2 Cons | | Cpd N Cons |
|---|---|---|---|---|---|
| 1 | (μM) | (μM) | (μM) | . . . | (μM) |
| C1 | 3.00E−01 | 3.00E+00 | 3.00E+00 | | 3.00E+00 |
| | 3.00E−01 | 3.00E+00 | 3.00E+00 | | 3.00E+00 |
| | 1.00E−01 | 1.00E+00 | 1.00E+00 | | 1.00E+00 |
| | 1.00E−01 | 1.00E+00 | 1.00E+00 | | 1.00E+00 |
| C2 | 3.33E−02 | 3.33E−01 | 3.33E−01 | | 3.33E−01 |
| | 3.33E−02 | 3.33E−01 | 3.33E−01 | | 3.33E−01 |
| | 1.11E−02 | 1.11E−01 | 1.11E−01 | | 1.11E−01 |
| | 1.11E−02 | 1.11E−01 | 1.11E−01 | | 1.11E−01 |
| C3 | 3.70E−03 | 3.70E−02 | 3.70E−02 | | 3.70E−02 |
| | 3.70E−03 | 3.70E−02 | 3.70E−02 | | 3.70E−02 |
| | 1.23E−03 | 1.23E−02 | 1.23E−02 | | 1.23E−02 |
| | 1.23E−03 | 1.23E−02 | 1.23E−02 | | 1.23E−02 |
| | 4.12E−04 | 4.12E−03 | 4.12E−03 | | 4.12E−03 |
| | 4.12E−04 | 4.12E−03 | 4.12E−03 | | 4.12E−03 |
| | 1.37E−04 | 1.37E−03 | 1.37E−03 | | 1.37E−03 |
| | 1.37E−04 | 1.37E−03 | 1.37E−03 | | 1.37E−03 |

### 2. Solution Preparation

### 1) 1.33× Kinase Buffer

Dilute 5× Kinase Buffer to 1.33× with ddH₂O

### 2) 4× Test Compounds

Serially dilute the test compounds to 4 folds of the concentrations desired, keeping the DMSO concentration at 8%. The final concentrations were 3, 1, 0.33, 0.11, 0.037, 0.012, 0.004, 0.0014 μM, and the final concentration of DMSO was 2%.

### 3) Kinase/Peptide Mixture (P/K Solution)

Prepare Kinase/Peptide Mixture by diluting the kinase to 0.12 μg/mL and the Z-LYTE™ Tyr 2 peptide to 4 μM in 1.33× Kinase Buffer. Mix gently by pipetting.

### 4) Phospho-Peptide Solution (PP Solution)

Add 0.4 μL of Z-LYTE™ Tyr 2 Phospho-peptide to 99.6 μL of 1.33× Kinase Buffer.

### 5) ATP Solution

Prepare ATP Solution by diluting the 10 mM of ATP in 1.33× Kinase Buffer to 1.88 mM.

6) Development Solution Dilute Development Reagent A with Development Buffer as 1:64.

### 3. Reaction

### 1) Kinase Reaction (10 μL of Volume)

a. Add 2.5 μL of 4× test Cpds to each well except C1, C2, C3 wells
   Add 2.5 μL of 8% DMSO to C1, C2, C3 wells
b. Put the 384-plate on ice
c. Add 5 μL of P/K mixture to each test Cpd wells and C1, C2 wells
d. Add 5 μL of PP Solution to C3 well
e. Add 2.5 μL of 1.33× kinase buffer to C1 and C3 wells
f. Add 2.5 μL of 4×ATP Solution to each test Cpd wells and C2 well, respectively.
   Shake the plate for 30 Sec and centrifuge (1500 rpm, 1 min)
g. Seal the plate to protect from the light and incubate the plate for 1 hour at RT (25-30° C.).

### 2) Development Reaction

a. Add 5 μL of the Development solution to all wells
b. Shake the plate for 30 Sec and centrifuge (1500 rpm, 1 min)
c. Seal the plate to protect from the light and incubate the plate for 1 hour at RT (25-30° C.).

### 3) Stop and Read

a. Add 5 μL of the Stop reagent to all wells
b. Shake the plate for 30 Sec and centrifuge (1500 rpm, 1 min)
c. Measure the value of Coumarin (Ex400 nm, Em445 nm) and fluorescein (Ex400 nm, Em520 nm), respectively.

### 4. Data Analysis

Emission Ratio(ER)=Coumarin Emission(445 nm)/Fluorescein Emission(520 nm)

% Phosphorylation=1−[ER×C3₅₂₀ₙₘ−C3₄₄₅ₙₘ]/[(C1₄₄₅ₙₘ−C3₄₄₅ₙₘ)+ER×(C3₅₂₀ₙₘ−C1₅₂₀ₙₘ)]

inhibition ratio(IR)=1−% Pho_{test Cpd}/% Pho_{C2}

IC₅₀ Value: determined with add-in software for Microsoft Excel, XLfit™ (version 2.0) from ID Business Solutions (Guildford, UK)

Below are the IC₅₀ values of some compounds.

| Compound | Flt-3 IC₅₀ (μM) |
|---|---|
| 32 | >3 |
| 43 | 1.602 |
| 57 | 1.64 |
| 67 | 2.523 |
| 69 | >3 |
| 90 | >3 |
| 94 | 2.009 |
| 95 | 2.821 |
| 100 | >3 |
| 108 | 2.423 |
| 219 | 0.880 |
| 300 | 0.885 |

### Example 3

### Cellular Assays

For the determination of IgE-induced Beta-hexosaminidase secretion, RBL-2H3 cells (SIBS) are seeded in 96 well plates at 4×10⁴ cells per well and incubated in MEM media with 15% FBS and Glutamine (2 nM) for 4 hours and sensitized with 0.5 ug/ml of SPE-7 overnight. Cells are washed 3 times with Tyrode's buffer and incubated in the presence or absence of various concentrations of the testing compound for 20 min at 37° C., 5% CO₂. Cells are stimulated by adding 10 uL of DNP-BSA solution (150 ng/mL) to each well and incubating for 45 minutes at 37° C., 5% CO₂. Then, 45 μL of the supernatant is taken and incubated with 100 μL of 1 mM 4-Nitrophenyl N-acetyl-β-D-glucosaminide (Sigma, Cat. No. N9376), which is diluted in 0.05 M citrate buffer (pH 4.5), for 1.5 hr at 37° C. The reactions are quenched by adding 185 μL of 0.05 M sodium carbonate buffer (pH 10.0). Plates are read at 405 nm on Multiskan (MK 3).

Below are the IC₅₀ values of some compounds.

| Cmpd | IC₅₀ (μM) |
|---|---|
| 6 | 0.260 |
| 7 | 0.122 |
| 8 | 0.113 |
| 9 | 0.127 |
| 11 | 0.048 |
| 12 | 0.021 |
| 13 | 0.040 |
| 14 | 0.631 |
| 15 | 0.102 |
| 16 | 0.033 |
| 17 | 0.056 |
| 18 | 0.062 |
| 19 | 0.110 |
| 20 | 0.066 |
| 21 | 0.088 |
| 22 | 0.073 |
| 23 | 0.109 |
| 24 | 0.127 |
| 25 | 0.155 |
| 26 | 0.113 |
| 27 | 0.222 |
| 28 | 0.142 |
| 29 | 0.106 |
| 30 | 0.277 |
| 31 | 0.056 |
| 32 | 0.076 |
| 33 | 0.064 |
| 36 | 0.097 |
| 38 | 0.278 |
| 39 | 0.076 |
| 43 | 0.037 |
| 48 | 0.136 |
| 49 | 0.315 |
| 50 | 0.108 |
| 51 | 0.159 |
| 52 | 0.047 |
| 53 | 0.037 |
| 54 | 0.257 |
| 55 | 0.144 |
| 57 | 0.082 |
| 58 | 0.990 |
| 59 | 0.117 |
| 61 | 0.029 |
| 62 | 0.097 |
| 63 | 0.342 |
| 64 | 0.487 |
| 65 | 0.181 |
| 66 | 0.230 |
| 67 | 0.089 |
| 68 | 0.110 |
| 69 | 0.062 |
| 70 | 0.213 |
| 71 | 0.335 |
| 72 | 0.188 |
| 73 | 0.142 |
| 74 | 0.186 |
| 75 | 0.131 |
| 76 | 0.088 |
| 77 | 0.202 |
| 78 | 0.074 |
| 83 | 0.092 |
| 84 | 0.316 |
| 86 | 0.616 |
| 87 | 0.200 |
| 88 | 0.098 |
| 89 | 0.207 |
| 90 | 0.061 |
| 92 | 0.072 |
| 94 | 0.022/0.040 |
| 95 | 0.049/0.071 |
| 98 | 0.23 |
| 99 | 0.106 |
| 100 | 0.068 |
| 101 | 0.503 |
| 102 | 0.108 |
| 103 | 0.325 |
| 104 | 0.127 |
| 105 | 0.077 |
| 108 | 0.052 |
| 109 | 0.131 |
| 110 | 0.163 |
| 111 | 0.119 |
| 119 | 0.034 |
| 120 | 0.041 |
| 122 | 0.073 |
| 123 | 0.035 |
| 124 | 0.045 |
| 125 | 0.043 |
| 126 | 0.366 |
| 127 | 0.077 |
| 129 | 0.230 |
| 131 | 0.039 |
| 133 | 0.024 |
| 134 | 0.015 |
| 136 | 0.099 |
| 137 | 0.045 |
| 139 | 0.119/0.194 |
| 140 | 0.031 |
| 141 | 0.081 |
| 142 | 0.950 |
| 143 | 0.244 |
| 144 | 0.080 |
| 148 | 0.137 |
| 149 | 0.060 |
| 150 | 0.097 |
| 151 | 0.043 |
| 152 | 0.048 |
| 153 | 0.026 |
| 154 | 0.078 |
| 155 | 0.029 |
| 156 | 0.032 |
| 158 | 0.032 |
| 161 | 0.108 |
| 163 | 0.053 |
| 164 | 0.082 |
| 165 | 0.095 |
| 166 | 0.071 |
| 172 | 0.055 |
| 173 | 0.054 |
| 175 | 0.069 |
| 176 | 0.067 |
| 177 | 0.079 |
| 178 | 0.063 |
| 181 | 0.057/0.069 |
| 183 | 0.095 |
| 184 | 0.062 |
| 185 | 0.064 |
| 186 | 0.114 |
| 188 | 0.039 |
| 189 | 0.078 |
| 190 | 0.645 |
| 192 | 0.049 |
| 193 | 0.143 |
| 195 | 0.129 |
| 196 | 0.128 |
| 197 | 0.081 |
| 198 | 0.086 |
| 199 | 0.211 |
| 200 | 0.133 |
| 201 | 0.100 |
| 202 | 0.090 |
| 203 | 0.107 |
| 205 | 0.019 |
| 206 | 0.052 |
| 207 | 0.008/0.014 |
| 208 | 0.020 |
| 209 | 0.057 |
| 210 | 0.032 |
| 211 | 0.078 |
| 212 | 0.030 |
| 213 | 0.071 |
| 214 | 0.020 |
| 215 | 0.040 |
| 216 | 0.122 |
| 217 | 0.068 |
| 218 | 0.027 |
| 219 | 0.037 |
| 220 | 0.069 |
| 221 | 0.038 |
| 222 | 0.105 |
| 223 | 0.121 |
| 224 | 0.103 |
| 225 | 0.302 |
| 226 | 0.06 |
| 227 | 0.07 |
| 228 | 0.039 |
| 229 | 0.094 |
| 230 | 0.429 |
| 231 | 0.087 |
| 232 | 0.056 |
| 241 | 0.124 |
| 246 | 0.044 |
| 248 | 0.054 |
| 249 | 0.069 |
| 250 | 0.112 |
| 251 | 0.083 |
| 252 | 0.381 |
| 254 | 0.099 |
| 255 | 0.097 |
| 256 | 0.092 |
| 257 | 0.16 |
| 258 | 0.187 |
| 259 | 0.297 |
| 260 | 0.135 |
| 261 | 0.418 |
| 262 | 0.269 |
| 263 | 0.4 |
| 264 | 0.339 |
| 265 | 0.166 |
| 266 | 1.092 |
| 267 | 1.256 |
| 268 | 0.036 |
| 269 | 0.088 |
| 270 | 0.185 |
| 273 | 0.176 |
| 274 | 0.059 |
| 275 | 1.416/0.613 |
| 279 | 0.087 |
| 281 | 0.663 |
| 282 | 0.092 |
| 293 | 0.241 |
| 283 | 0.248 |
| 284 | 0.194 |
| 288 | 0.085/0.048 |
| 290 | 0.374/0.467 |
| 294 | 0.218/0.132 |
| 292 | 0.232 |
| 296 | >3.333 |
| 297 | 0.088 |
| 298 | 0.191 |
| 299 | 0.047 |
| 300 | 0.052 |
| 301 | 0.107 |
| 302 | 0.03 |
| 308 | 0.097 |
| 311 | 0.62 |
| 312 | 0.873 |
| 314 | 0.117 |
| 315 | 0.123 |
| 316 | 0.113 |
| 317 | 0.094 |
| 318 | 0.174 |
| 321 | 0.127 |

1. A compound of formula (I): 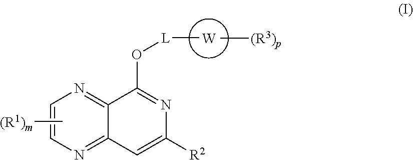
and/or its racemic mixture, enantiomers, diastereomers, tautomers, or mixtures of optional ratio, or at least one pharmaceutically acceptable salt, or solvate thereof, wherein
R¹ is independently chosen from hydrogen, halo, —CN, —OH, optionally substituted C₁-C₆alkyl, optionally substituted C₁-C₆alkoxy, —NH₂, —NH(C₁-C₄alkyl), and —N(C₁-C₄alkyl)(C₁-C₄ alkyl);
R² is aryl, or heteroaryl, each of which is optionally substituted by one or more groups selected from halo, —NR⁵R⁶, —OR⁷, —S(O)ₙR⁸, —C(O)R⁹, —C(O)OR⁷, —CN, —C(O)NR⁵R⁶, —NR⁵C(O)R⁹, —NR⁵S(O)ₙR⁸, —NR⁵S(O)ₙNR¹⁰R¹¹, —NR⁵C(O)OR⁷, —NR⁵C(O)NR¹⁰R¹¹, —NO₂, —S(O)ₙNR⁵R⁶, optionally substituted lower alkyl, optionally substituted cycloalkyl, optionally substituted heterocycle, optionally substituted heteroaryl, optionally substituted aryl, optionally substituted alkenyl, and optionally substituted alkynyl;
L is a bond, or optionally substituted C₁-C₆alkylene;
W is cycloalkyl, heterocycle, aryl, or heteroaryl;
R³ is independently selected from hydrogen, -Lx-halo, -Lx-R⁴, -Lx-NR⁵R⁶, -Lx-OR⁷, -Lx-S(O)ₙR⁸, -Lx-C(O)R⁹, —S(O)ₙ-Lx-R⁸, —C(O)-Lx-R⁹, -Lx-CN, -Lx-NR⁵C(O)R⁹, -Lx-NR⁵S(O)ₙR⁸, -Lx-NR⁵C(O)NR¹⁰R¹¹, Lx-NR⁵S(O)ₙNR¹⁰R¹¹, -Lx-NR⁵C(O)OR⁷, -Lx-NR⁵S(O)ₙOR⁷, —NO₂, -Lx-C(O)NR⁵R⁶, -Lx-S(O)ₙNR⁵R⁶, oxo(═O), optionally substituted cycloalkyl, optionally substituted heterocycle, optionally substituted heteroaryl, and optionally substituted aryl,
provided when L is methylene and W is 5- or 6-membered heterocycle, R³ is independently selected from -Lx-NR⁵R⁶, -Lx-OR⁷, -Lx-S(O)ₙR⁸, -Lx-C(O)R⁹, —S(O)ₙ-Lx-R⁸, —C(O)-Lx-R⁹, -Lx-CN, -Lx-NR⁵C(O)R⁹, -Lx-NR⁵S(O)ₙR⁸, -Lx-NR⁵C(O)NR¹⁰R¹¹, -Lx-NR⁵S(O)ₙNR¹⁰R¹¹, -Lx-NR⁵C(O)OR⁷, -Lx-NR⁵S(O)ₙOR⁷, —NO₂, -Lx-C(O)NR⁵R⁶, -Lx-S(O)ₙNR⁵R⁶, oxo(═O), optionally substituted cycloalkyl, optionally substituted heterocycle, optionally substituted heteroaryl, and optionally substituted aryl;
R⁴ is C₁-C₆alkyl, C₂-C₆alkenyl, or C₂-C₆alkynyl, each of which is optionally substituted;
R⁵, R⁶, R⁷, R⁸, R⁹, R¹⁰, and R¹¹ are independently selected from hydrogen, alkyl, cycloalkyl, aryl, heteroaryl, and heterocycle, each of which except for hydrogen, is optionally substituted with one or more groups selected from halo, —OH, —O(C₁-C₄alkyl), —CN, C₁-C₄alkyl, —NH₂, —NH(C₁-C₄alkyl), —N(C₁-C₄alkyl)(C₁-C₄alkyl), —C(O)NH₂, —C(O)NH(C₁-C₄alkyl), —C(O)N(C₁-C₄alkyl)(C₁-C₄alkyl), —C(O)(C₁-C₄alkyl), —NHC(O)(C₁-C₄alkyl), —N(C₁-C₄alkyl)C(O)(C₁-C₄alkyl), —S(O)ₙNH₂, —S(O)ₙNH(C₁-C₄alkyl), —S(O)ₙN(C₁-C₄alkyl)(C₁-C₄alkyl), —S(O)ₙ(C₁-C₄alkyl), —NHS(O)ₙ(C₁-C₄alkyl), —N(C₁-C₄alky)S(O)ₙ(C₁-C₄alkyl), optionally substituted C₃-C₈cycloalkyl, and optionally substituted 3-8 membered heterocycle, wherein C₁-C₄alkyl is optionally substituted by halo, —OH, —OMe, —CN;
or R⁵ and R⁶, R⁵ and R⁷, R⁵ and R⁸, R⁵ and R⁹, and R⁵ and R¹⁰ together with the atom(s) to which they are attached can form a ring, which is optionally substituted with one or more groups selected from halo, —OH, —O(C₁-C₄alkyl), —CN, C₁-C₄alkyl, —NH₂, —NH(C₁-C₄alkyl), —N(C₁-C₄alkyl)(C₁-C₄alkyl), —C(O)NH₂, —C(O)NH(C₁-C₄alkyl), —C(O)N(C₁-C₄alkyl)(C₁-C₄alkyl), —C(O)(C₁-C₄alkyl), —NHC(O)(C₁-C₄alkyl), —N(C₁-C₄alkyl)C(O)(C₁-C₄alkyl), —S(O), NH₂, —S(O)ₙNH(C₁-C₄alkyl), —S(O)ₙN(C₁-C₄alkyl)(C₁-C₄alkyl), —S(O)ₙ(C₁-C₄alkyl), —NHS(O)ₙ(C₁-C₄alkyl), —N(C₁-C₄alky)S(O)ₙ(C₁-C₄alkyl), optionally substituted C₃-C₈cycloalkyl, and optionally substituted 3-8 membered heterocycle, wherein C₁-C₄alkyl is optionally substituted by halo, —OH, —OMe, or CN;
Lx is a bond, or optionally substituted C₁-C₆alkylene;
m is 0, 1 or 2,
n is 1 or 2,
p is 1, 2 or 3.

2. (canceled)

3. The compound of claim 1, and/or its racemic mixture, enantiomers, diastereomers, tautomers, or mixtures of optional ratio, or at least one pharmaceutically acceptable salt, or solvate thereof, wherein R¹ is independently chosen from hydrogen, halo, —CN, —OH; or is chosen from methyl, ethyl, n-propyl, i-propyl, —NH₂, N-methylamino, N,N-dimethylamino, N-ethylamino, N-n-propylamino, N-i-propylamino, methoxy, ethoxy, propoxy, isopropoxy, each of which is optionally substituted.

4. (canceled)

5. (canceled)

6. The compound of claim 1, and/or its racemic mixture, enantiomers, diastereomers, tautomers, or mixtures of optional ratio, or at least one pharmaceutically acceptable salt, or solvate thereof, wherein R² is C₅-C₁₀aryl, or 5-10 membered heteroaryl, each of which is optionally substituted by one or more groups selected from halo, —NR⁵R⁶, —OR⁷, —S(O)ₙR⁸, —C(O)R⁹, —C(O)OR⁷, —CN, —C(O)NR⁵R⁶, —NR⁵C(O)R⁹, —NR⁵S(O)ₙR⁸, —NR⁵S(O)ₙNR¹⁰R¹¹, —NR⁵C(O)OR⁷, —NR⁵C(O)NR¹⁰R¹¹, —NO₂, —S(O)ₙNR⁵R⁶, optionally substituted C₁-C₆ alkyl, optionally substituted C₃-C₈ cycloalkyl, optionally substituted 3-8 membered heterocycle, optionally substituted 5-10 membered heteroaryl, optionally substituted C₅-C₁₀ aryl, optionally substituted C₂-C₆ alkenyl, and optionally substituted C₂-C₆ alkynyl,
R⁵, R⁶, R⁷, R⁸, R⁹, R¹⁰, and R¹¹ are independently selected from hydrogen, alkyl, cycloalkyl, aryl, heteroaryl, and heterocycle, each of which except for hydrogen, is optionally substituted with one or more groups selected from halo, —OH, —O(C₁-C₄alkyl), —CN, C₁-C₄alkyl, —NH₂, —NH(C₁-C₄alkyl), —N(C₁-C₄alkyl)(C₁-C₄alkyl), —C(O)NH₂, —C(O)NH(C₁-C₄alkyl), —C(O)N(C₁-C₄alkyl)(C₁-C₄alkyl), —C(O)(C₁-C₄alkyl), —NHC(O)(C₁-C₄alkyl), —N(C₁-C₄alkyl)C(O)(C₁-C₄alkyl), —S(O)ₙNH₂, —S(O)ₙNH(C₁-C₄alkyl), —S(O)ₙN(C₁-C₄alkyl)(C₁-C₄alkyl), —S(O)ₙ(C₁-C₄alkyl), —NHS(O)ₙ(C₁-C₄alkyl), —N(C₁-C₄alky)S(O)ₙ(C₁-C₄alkyl), optionally substituted C₃-C₈cycloalkyl, and optionally substituted 3-8 membered heterocycle, wherein C₁-C₄alkyl is optionally substituted by halo, —OH, —OMe, —CN;
or R⁵ and R⁶, R⁵ and R⁷, R⁵ and R⁸, R⁵ and R⁹, and R⁵ and R¹⁰ together with the atom(s) to which they are attached can form a ring, which is optionally substituted with one or more groups selected from halo, —OH, —O(C₁-C₄alkyl), —CN, C₁-C₄alkyl, —NH₂, —NH(C₁-C₄alkyl), —N(C₁-C₄alkyl)(C₁-C₄alkyl), —C(O)NH₂, —C(O)NH(C₁-C₄alkyl), —C(O)N(C₁-C₄alkyl)(C₁-C₄alkyl), —C(O)(C₁-C₄alkyl), —NHC(O)(C₁-C₄alkyl), —N(C₁-C₄alkyl)C(O)(C₁-C₄alkyl), —S(O)ₙNH₂, —S(O)ₙNH(C₁-C₄alkyl), —S(O)ₙN(C₁-C₄alkyl)(C₁-C₄alkyl), —S(O)ₙ(C₁-C₄alkyl), —NHS(O)ₙ(C₁-C₄alkyl), —N(C₁-C₄alky)S(O)ₙ(C₁-C₄alkyl), optionally substituted C₃-C₈cycloalkyl, and optionally substituted 3-8 membered heterocycle, wherein C₁-C₄alkyl is optionally substituted by halo, —OH, —OMe, or CN.

7. The compound of claim 6, and/or its racemic mixture, enantiomers, diastereomers, tautomers, or mixtures of optional ratio, or at least one pharmaceutically acceptable salt, or solvate thereof, wherein R² is independently chosen from phenyl, naphthyl, pyridyl, pyrimidinyl, pyrazinyl, pyridazinyl, pyrrolyl pyrazolyl, imidazolinyl, oxazolyl, isoxazolyl, thiazolyl, thienyl, furyl, benzofuryl, benzothienyl, benzoimidazolinyl, indolyl, indazolyl, quinolinyl, and, indanyl, indolinyl, indolin-2-one, 2,3-dihydrobenzofuryl, benzo[d][1,3]dioxolyl, and 1,2,3,4-tetrahydroquinolinyl, chroman, 2,3-dihydrobenzo[b][1,4]dioxinyl, 3,4-dihydro-2H-benzo[b][1,4]oxazinyl, isochroman, 1,3-dihydroisobenzofuryl, 1H-benzo[d][1,3]oxazin-2(4H)-onyl, each of which is optionally substituted by one or more groups selected from halo, —NR⁵R⁶, —OR⁷, —S(O)ₙR⁸, —C(O)R⁹, —C(O)OR⁷, —CN, —C(O)NR⁵R⁶, —NR⁵C(O)R⁹, —NR⁵S(O)ₙR⁸, —NR⁵S(O)ₙNR¹⁰R¹¹, —NR⁵C(O)OR⁷, —NR⁵C(O)NR¹⁰R¹¹, —NO₂, and —S(O)ₙNR⁵R⁶; or selected from methyl, ethyl, n-propyl, i-propyl, n-butyl, i-butyl, and t-butyl, cyclopropyl, cyclobutyl, cyclopentyl, cyclopentenyl, cyclohexyl, cyclohexenyl, pyrrolidinyl, tetrahydrofuryl, tetrahydropyranyl, piperidinyl, piperazinyl, morpholinyl, homomorpholinyl, thiomorpholinyl, diazepanyl, oxazepanyl, pyridyl, pyrimidinyl, pyrazinyl, pyridazinyl, pyrroly, l pyrazolyl, imidazolinyl, oxazolyl, isoxazolyl, thiazolyl, thienyl, furyl, benzofuryl, benzothienyl, benzoimidazolinyl, indolyl, indazolyl, quinolinyl, phenyl, and naphthyl, each of which is optionally substituted by one or more groups selected from halo, —NR⁵R⁶, —OR⁷, —S(O)ₙR⁸, —C(O)R⁹, —C(O)OR⁷, —CN, —C(O)NR⁵R⁶, —NR⁵C(O)R⁹, —NR⁵S(O)ₙR⁸, —NR⁵S(O)ₙNR¹⁰R¹¹, —NR⁵C(O)OR⁷, —NR⁵C(O)NR¹⁰R¹¹, —NO₂, —S(O)ₙNR⁵R⁶, optionally substituted lower alkyl, optionally substituted cycloalkyl, optionally substituted heterocycle, optionally substituted heteroaryl, optionally substituted aryl, optionally substituted alkenyl, and optionally substituted alkynyl,
R⁵, R⁶, R⁷, R⁸, R⁹, R¹⁰, and R¹¹ are independently selected from hydrogen, alkyl, cycloalkyl, aryl, heteroaryl, and heterocycle, each of which except for hydrogen, is optionally substituted with one or more groups selected from halo, —OH, —O(C₁-C₄alkyl), —CN, C₁-C₄alkyl, —NH₂, —NH(C₁-C₄alkyl), —N(C₁-C₄alkyl)(C₁-C₄alkyl), —C(O)NH₂, —C(O)NH(C₁-C₄alkyl), —C(O)N(C₁-C₄alkyl)(C₁-C₄alkyl), —C(O)(C₁-C₄alkyl), —NHC(O)(C₁-C₄alkyl), —N(C₁-C₄alkyl)C(O)(C₁-C₄alkyl), —S(O)ₙNH₂, —S(O)NH(C₁-C₄alkyl), —S(O)ₙN(C₁-C₄alkyl)(C₁-C₄alkyl), —S(O)ₙ(C₁-C₄alkyl), —NHS(O)ₙ(C₁-C₄alkyl), —N(C₁-C₄alky)S(O)ₙ(C₁-C₄alkyl), optionally substituted C₃-C₈cycloalkyl, and optionally substituted 3-8 membered heterocycle, wherein C₁-C₄alkyl is optionally substituted by halo, —OH, —OMe, —CN;
or R⁵ and R⁶, R⁵ and R⁷, R⁵ and R⁸, R⁵ and R⁹, and R⁵ and R¹⁰ together with the atom(s) to which they are attached can form a ring, which is optionally substituted with one or more groups selected from halo, —OH, —O(C₁-C₄alkyl), —CN, C₁-C₄alkyl, —NH₂, —NH(C₁-C₄alkyl), —N(C₁-C₄alkyl)(C₁-C₄alkyl), —C(O)NH₂, —C(O)NH(C₁-C₄alkyl), —C(O)N(C₁-C₄alkyl)(C₁-C₄alkyl), —C(O)(C₁-C₄alkyl), —NHC(O)(C₁-C₄alkyl), —N(C₁-C₄alkyl)C(O)(C₁-C₄alkyl), —S(O)ₙNH₂, S(O)ₙNH(C₁-C₄alkyl), —S(O)ₙN(C₁-C₄alkyl)(C₁-C₄alkyl), —S(O)ₙ(C₁-C₄alkyl), —NHS(O)ₙ(C₁-C₄alkyl), —N(C₁-C₄alky)S(O)ₙ(C₁-C₄alkyl), optionally substituted C₃-C₈cycloalkyl, and optionally substituted 3-8 membered heterocycle, wherein C₁-C₄alkyl is optionally substituted by halo, —OH, —OMe, or CN.

8. The compound of claim 7, and/or its racemic mixture, enantiomers, diastereomers, tautomers, or mixtures of optional ratio, or at least one pharmaceutically acceptable salt, or solvate thereof, wherein R² is independently chosen from 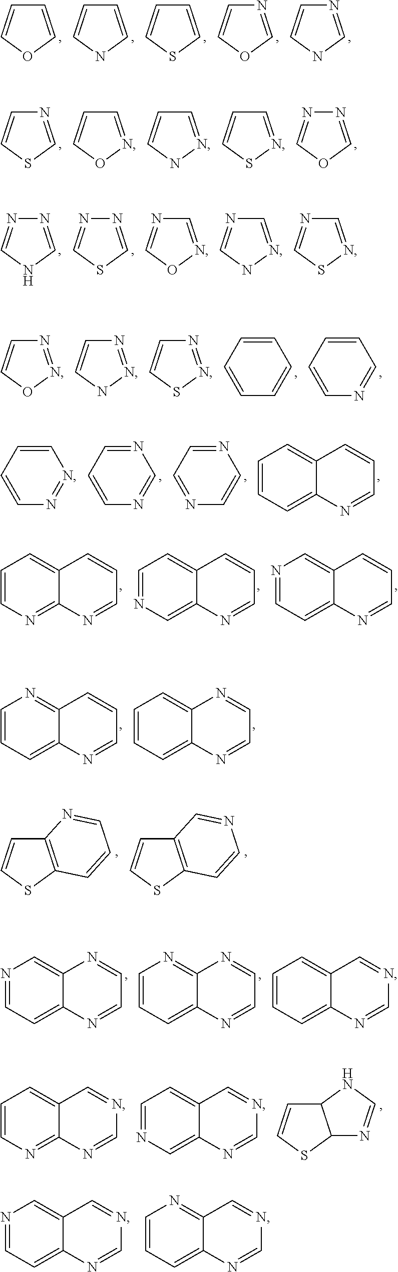 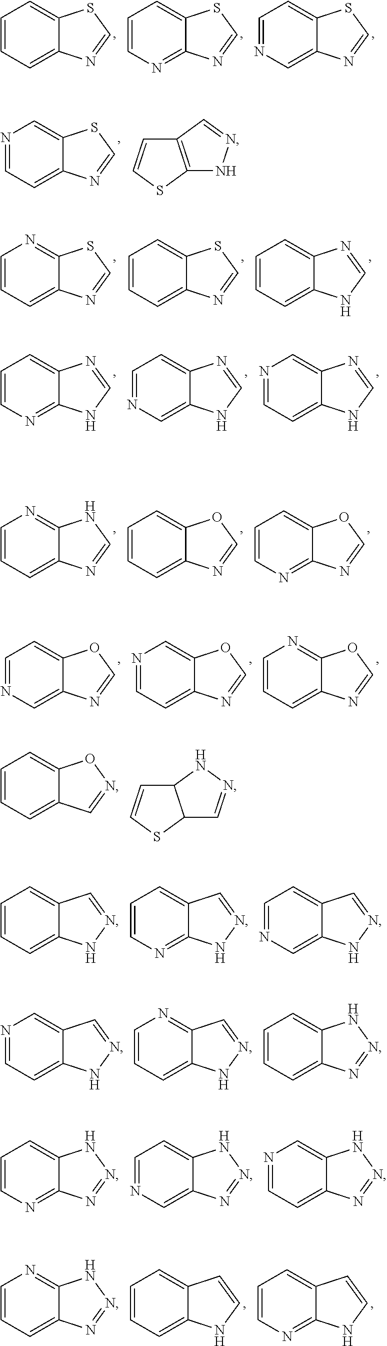 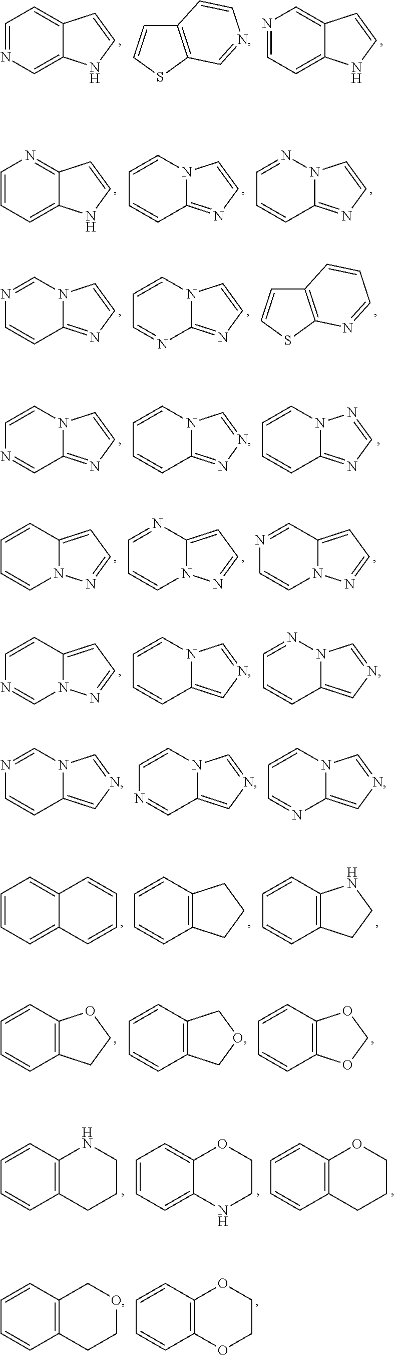 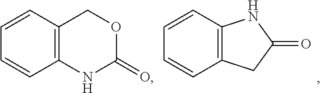
each of which is optionally substituted by one or more groups selected from halo, —NR⁵R⁶, —OR⁷, —S(O)ₙR⁸, —C(O)R⁹, —C(O)OR⁷, —CN, —C(O)NR⁵R⁶, —NR⁵C(O)R⁹, —NR⁵S(O)ₙR⁸, —NR⁵S(O)ₙNR¹⁰R¹¹, —NR⁵C(O)OR⁷, —NR⁵C(O)NR¹⁰R¹¹, —NO₂, and —S(O)ₙNR⁵R⁶; or selected from methyl, ethyl, n-propyl, i-propyl, n-butyl, i-butyl, and t-butyl, cyclopropyl, cyclobutyl, cyclopentyl, cyclopentenyl, cyclohexyl, cyclohexenyl, pyrrolidinyl, tetrahydrofuryl, tetrahydropyranyl, piperidinyl, piperazinyl, morpholinyl, homomorpholinyl, thiomorpholinyl, diazepanyl, oxazepanyl, pyridyl, pyrimidinyl, pyrazinyl, pyridazinyl, pyrroly, l pyrazolyl, imidazolinyl, oxazolyl, isoxazolyl, thiazolyl, thienyl, furyl, benzofuryl, benzothienyl, benzoimidazolinyl, indolyl, indazolyl, quinolinyl, phenyl, and naphthyl, each of which is optionally substituted by one or more groups selected from halo, —NR⁵R⁶, —OR⁷, —S(O)ₙR⁸, —C(O)R⁹, —C(O)OR⁷, —CN, —C(O)NR⁵R⁶, —NR⁵C(O)R⁹, —NR⁵S(O)ₙR⁸, —NR⁵S(O)ₙNR¹⁰R¹¹, —C(O)OR⁷, —NR⁵C(O)NR¹⁰R¹¹, —NO₂, —S(O)ₙNR⁵R⁶, optionally substituted lower alkyl, optionally substituted cycloalkyl, optionally substituted heterocycle, optionally substituted heteroaryl, optionally substituted aryl, optionally substituted alkenyl, and optionally substituted alkynyl,
R⁵, R⁶, R⁷, R⁸, R⁹, R¹⁰, and R¹¹ are independently selected from hydrogen, alkyl, cycloalkyl, aryl, heteroaryl, and heterocycle, each of which except for hydrogen, is optionally substituted with one or more groups selected from halo, —OH, —O(C₁-C₄ alkyl), —CN, C₁-C₄ alkyl, —NH₂, —NH(C₁-C₄ alkyl), —N(C₁-C₄ alkyl)(C₁-C₄ alkyl), —C(O)NH₂, —C(O)NH(C₁-C₄ alkyl), —C(O)N(C₁-C₄ alkyl)(C₁-C₄ alkyl), —C(O)(C₁-C₄ alkyl), —NHC(O)(C₁-C₄ alkyl), —N(C₁-C₄ alkyl)C(O)(C₁-C₄ alkyl), —S(O)ₙNH₂, —S(O)ₙNH(C₁-C₄ alkyl), —S(O)ₙN(C₁-C₄ alkyl)(C₁-C₄ alkyl), —S(O)ₙ(C₁-C₄ alkyl), —NHS(O)ₙ(C₁-C₄ alkyl), —N(C₁-C₄ alky)S(O)ₙ(C₁-C₄ alkyl), optionally substituted C₃-C₈ cycloalkyl, and optionally substituted 3-8 membered heterocycle, wherein C₁-C₄ alkyl is optionally substituted by halo, —OH, —OMe, or —CN,
or R⁵ and R⁶, R⁵ and R⁷, R⁵ and R⁸, R⁵ and R⁹, and Wand R¹⁰ together with the atom(s) to which they are attached can form a ring, which is optionally substituted with one or more groups selected from halo, —OH, —O(C₁-C₄ alkyl), —CN, C₁-C₄ alkyl, —NH₂, —NH(C₁-C₄ alkyl), —N(C₁-C₄ alkyl)(C₁-C₄ alkyl), —C(O)NH₂, —C(O)NH(C₁-C₄ alkyl), —C(O)N(C₁-C₄ alkyl)(C₁-C₄ alkyl), —C(O)(C₁-C₄ alkyl), —NHC(O)(C₁-C₄ alkyl), —N(C₁-C₄ alkyl)C(O)(C₁-C₄ alkyl), —S(O)ₙNH₂, —S(O)ₙNH(C₁-C₄ alkyl), —S(O)N(C₁-C₄ alkyl)(C₁-C₄ alkyl), —S(O)ₙ(C₁-C₄ alkyl), —NHS(O)ₙ(C₁-C₄ alkyl), —N(C₁-C₄ alky)S(O)ₙ(C₁-C₄ alkyl), optionally substituted C₃-C₈ cycloalkyl, and optionally substituted 3-8 membered heterocycle, wherein C₁-C₄ alkyl is optionally substituted by halo, —OH, —OMe, or —CN.

9. The compound of claim 8, and/or its racemic mixture, enantiomers, diastereomers, tautomers, or mixtures of optional ratio, or at least one pharmaceutically acceptable salt, or solvate thereof, wherein R² is independently chosen from 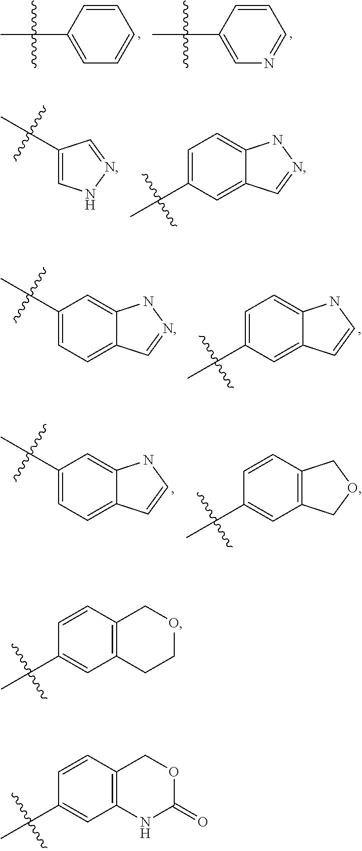
each of which is optionally substituted by one or more groups selected from halo, —NR⁵R⁶, —OR⁷, —S(O)ₙR⁸, —C(O)R⁹, —C(O)OR⁷, —CN, —C(O)NR⁵R⁶, —NR⁵C(O)R⁹, —NR⁵S(O)ₙR⁸, —NR⁵S(O)ₙNR¹⁰R¹¹, —NR⁵C(O)OR⁷, —NR⁵C(O)NR¹⁰R¹¹, —NO₂, and —S(O)ₙNR⁵R⁶; or selected from methyl, ethyl, n-propyl, i-propyl, n-butyl, i-butyl, and t-butyl, cyclopropyl, cyclobutyl, cyclopentyl, cyclopentenyl, cyclohexyl, cyclohexenyl, pyrrolidinyl, tetrahydrofuryl, tetrahydropyranyl, piperidinyl, piperazinyl, morpholinyl, homomorpholinyl, thiomorpholinyl, diazepanyl, oxazepanyl, pyridyl, pyrimidinyl, pyrazinyl, pyridazinyl, pyrroly, l pyrazolyl, imidazolinyl, oxazolyl, isoxazolyl, thiazolyl, thienyl, furyl, benzofuryl, benzothienyl, benzoimidazolinyl, indolyl, indazolyl, quinolinyl, phenyl, and naphthyl, each of which is optionally substituted by one or more groups selected from halo, —NR⁵R⁶, —OR⁷, —S(O)ₙR⁸, —C(O)R⁹, —C(O)OR⁷, —CN, —C(O)NR⁵R⁶, —NR⁵C(O)R⁹, —NR⁵S(O)ₙR⁸, —NR⁵S(O)ₙNR¹⁰R¹¹, —NR⁵C(O)OR⁷, —NR⁵C(O)NR¹⁰R¹¹, —NO₂, —S(O)ₙNR⁵R⁶, optionally substituted lower alkyl, optionally substituted cycloalkyl, optionally substituted heterocycle, optionally substituted heteroaryl, optionally substituted aryl, optionally substituted alkenyl, and optionally substituted alkynyl,
R⁵, R⁶, R⁷, R⁸, R⁹, R¹⁰, and R¹¹ are independently selected from hydrogen, alkyl, cycloalkyl, aryl, heteroaryl, and heterocycle, each of which except for hydrogen, is optionally substituted with one or more groups selected from halo, —OH, —O(C₁-C₄ alkyl), —CN, C₁-C₄ alkyl, —NH₂, —NH(C₁-C₄ alkyl), —N(C₁-C₄ alkyl)(C₁-C₄ alkyl), —C(O)NH₂, —C(O)NH(C₁-C₄ alkyl), —C(O)N(C₁-C₄ alkyl)(C₁-C₄ alkyl), —C(O)(C₁-C₄ alkyl), —NHC(O)(C₁-C₄ alkyl), —N(C₁-C₄ alkyl)C(O)(C₁-C₄ alkyl), —S(O)NH₂, —S(O)NH(C₁-C₄ alkyl), —S(O)ₙN(C₁-C₄ alkyl)(C₁-C₄ alkyl), —S(O)ₙ(C₁-C₄ alkyl), —NHS(O)ₙ(C₁-C₄ alkyl), —N(C₁-C₄ alky)S(O)ₙ(C₁-C₄ alkyl), optionally substituted C₃-C₈ cycloalkyl, and optionally substituted 3-8 membered heterocycle, wherein C₁-C₄ alkyl is optionally substituted by halo, —OH, —OMe, or —CN,
or R⁵ and R⁶, R⁵ and R⁷, R⁵ and R⁸, R⁵ and R⁹, and R⁵ and R¹⁰ together with the atom(s) to which they are attached can form a ring, which is optionally substituted with one or more groups selected from halo, —OH, —O(C₁-C₄ alkyl), —CN, C₁-C₄ alkyl, —NH₂, —NH(C₁-C₄ alkyl), —N(C₁-C₄ alkyl)(C₁-C₄ alkyl), —C(O)NH₂, —C(O)NH(C₁-C₄ alkyl), —C(O)N(C₁-C₄ alkyl)(C₁-C₄ alkyl), —C(O)(C₁-C₄ alkyl), —NHC(O)(C₁-C₄ alkyl), —N(C₁-C₄ alkyl)C(O)(C₁-C₄ alkyl), —S(O)ₙNH₂, —S(O)ₙNH(C₁-C₄ alkyl), —S(O)ₙN(C₁-C₄ alkyl)(C₁-C₄ alkyl), —S(O)ₙ(C₁-C₄ alkyl), —NHS(O)ₙ(C₁-C₄ alkyl), —N(C₁-C₄ alky)S(O)ₙ(C₁-C₄ alkyl), optionally substituted C₃-C₈ cycloalkyl, and optionally substituted 3-8 membered heterocycle, wherein C₁-C₄ alkyl is optionally substituted by halo, —OH, —OMe, or —CN.

10. (canceled)

11. (canceled)

12. The compound of claim 11, and/or its racemic mixture, enantiomers, diastereomers, tautomers, or mixtures of optional ratio, or at least one pharmaceutically acceptable salt, or solvate thereof, wherein L is a bond, or —CH₂—, or —CH₂—CH₂—.

13. The compound of claim 1, and/or its racemic mixture, enantiomers, diastereomers, tautomers, or mixtures of optional ratio, or at least one pharmaceutically acceptable salt, or solvate thereof, wherein W is C₃-C₈cycloalkyl, 3-8 membered heterocycle, C₅-C₁₀aryl, or 5-10 membered heteroaryl.

14. The compound of claim 13, and/or its racemic mixture, enantiomers, diastereomers, tautomers, or mixtures of optional ratio, or at least one pharmaceutically acceptable salt, or solvate thereof, wherein W is cyclopropyl, cyclobutyl, cyclopentyl, cyclopentenyl, cyclohexyl, cyclohexenyl, pyrrolidinyl, tetrahydrofuryl, tetrahydropyranyl, piperidinyl, piperazinyl, morpholinyl, homomorpholinyl, thiomorpholinyl, phenyl, naphthyl pyridyl, pyrimidinyl, pyrazinyl, pyridazinyl, pyrrolyl, pyrazolyl, imidazolinyl, oxazolyl, isoxazolyl, thiazolyl, thienyl, furyl, benzofuryl, benzothienyl, benzoimidazolinyl, indolyl, indazolyl, or quinolinyl.

15. (canceled)

16. The compound of claim 14, and/or its racemic mixture, enantiomers, diastereomers, tautomers, or mixtures of optional ratio, or at least one pharmaceutically acceptable salt, or solvate thereof, wherein W is tetrahydrofuryl, tetrahydropyranyl, or morpholinyl.

17.-19. (canceled)

20. The compound of claim 1, and/or its racemic mixture, enantiomers, diastereomers, tautomers, or mixtures of optional ratio, or at least one pharmaceutically acceptable salt, or solvate thereof, wherein R³ is independently selected from hydrogen, -Lx-halo, -Lx-R⁴, -Lx-NR⁵R⁶, -Lx-OR⁷, -Lx-S(O)ₙR⁸, -Lx-C(O)R⁹, —S(O)ₙ-Lx-R⁸, —C(O)-Lx-R⁹, -Lx-CN, -Lx-NR⁵C(O)R⁹, -Lx-NR⁵S(O)ₙR⁸, -Lx-NR⁵C(O)NR¹⁰R¹¹, -Lx-NR⁵S(O)ₙNR¹⁰R¹¹, -Lx-NR⁵C(O)OR⁷, -Lx-NR⁵S(O)ₙOR⁷, —NO₂, -Lx-C(O)NR⁵R⁶, -Lx-S(O)ₙNR⁵R⁶, oxo(═O), optionally substituted C₃-C₈cycloalkyl, optionally substituted 3-8 membered heterocycle, optionally substituted C₅-C₁₀aryl, and optionally substituted 5-10 membered heteroaryl, provided when L is methylene and W is 5- or 6-membered heterocycle, R³ is independently selected from -Lx-NR⁵R⁶, -Lx-OR⁷, -Lx-S(O)ₙR⁸, -Lx-C(O)R⁹, —S(O)ₙ-Lx-R⁸, —C(O)-Lx-R⁹, -Lx-CN, -Lx-NR⁵C(O)R⁹, -Lx-NR⁵S(O)ₙR⁸, -Lx-NR⁵C(O)NR¹⁰R¹¹, -Lx-NR⁵S(O)ₙNR¹⁰R¹¹, -Lx-NR⁵C(O)OR⁷, -Lx-NR⁵S(O)ₙOR⁷, —NO₂, -Lx-C(O)NR⁵R⁶, -Lx-S(O)ₙNR⁵R⁶, oxo(═O), optionally substituted C₃-C₈cycloalkyl, optionally substituted 3-8 membered heterocycle, optionally substituted C₅-C₁₀aryl, and optionally substituted 5-10 membered heteroaryl
R⁴ is optionally substituted C₁-C₄alkyl,
R⁵, R⁶, R⁷, R⁸, R⁹, R¹⁰, and R¹¹ are independently selected from hydrogen, alkyl, cycloalkyl, aryl, heteroaryl, and heterocycle, each of which except for hydrogen, is optionally substituted with one or more groups selected from halo, —OH, —O(C₁-C₄ alkyl), —CN, C₁-C₄ alkyl, —NH₂, —NH(C₁-C₄ alkyl), —N(C₁-C₄ alkyl)(C₁-C₄ alkyl), —C(O)NH₂, —C(O)NH(C₁-C₄ alkyl), —C(O)N(C₁-C₄ alkyl)(C₁-C₄ alkyl), —C(O)(C₁-C₄ alkyl), —NHC(O)(C₁-C₄ alkyl), —N(C₁-C₄ alkyl)C(O)(C₁-C₄ alkyl), —S(O)NH₂, —S(O)NH(C₁-C₄ alkyl), —S(O)N(C₁-C₄ alkyl)(C₁-C₄ alkyl), —S(O)ₙ(C₁-C₄ alkyl), —NHS(O)ₙ(C₁-C₄ alkyl), —N(C₁-C₄ alky)S(O)ₙ(C₁-C₄ alkyl), optionally substituted C₃-C₈ cycloalkyl, and optionally substituted 3-8 membered heterocycle, wherein C₁-C₄ alkyl is optionally substituted by halo, —OH, —OMe, or —CN,
or R⁵ and R⁶, R⁵ and R⁷, R⁵ and R⁸, R⁵ and R⁹, and R⁵ and R¹⁰ together with the atom(s) to which they are attached can form a ring, which is optionally substituted with one or more groups selected from halo, —OH, —O(C₁-C₄ alkyl), —CN, C₁-C₄ alkyl, —NH₂, —NH(C₁-C₄ alkyl), —N(C₁-C₄ alkyl)(C₁-C₄ alkyl), —C(O)NH₂, —C(O)NH(C₁-C₄ alkyl), —C(O)N(C₁-C₄ alkyl)(C₁-C₄ alkyl), —C(O)(C₁-C₄ alkyl), —NHC(O)(C₁-C₄ alkyl), —N(C₁-C₄ alkyl)C(O)(C₁-C₄ alkyl), —S(O)ₙNH₂, —S(O)NH(C₁-C₄ alkyl), —S(O)N(C₁-C₄ alkyl)(C₁-C₄ alkyl), —S(O)ₙ(C₁-C₄ alkyl), —NHS(O)ₙ(C₁-C₄ alkyl), —N(C₁-C₄ alky)S(O)ₙ(C₁-C₄ alkyl), optionally substituted C₃-C₈ cycloalkyl, and optionally substituted 3-8 membered heterocycle, wherein C₁-C₄ alkyl is optionally substituted by halo, —OH, —OMe, or CN;
Lx is a bond, or optionally substituted C₁-C₆ alkylene.

21. The compound of claim 20, and/or its racemic mixture, enantiomers, diastereomers, tautomers, or mixtures of optional ratio, or at least one pharmaceutically acceptable salt, or solvate thereof, wherein R³ is independently selected from hydrogen, -Lx-halo, -Lx-R⁴, -Lx-NR⁵R⁶, -Lx-OR⁷, -Lx-S(O)ₙR⁸, -Lx-C(O)R⁹, —S(O)ₙ-Lx-R⁸, —C(O)-Lx-R⁹, -Lx-CN, -Lx-NR⁵C(O)R⁹, -Lx-NR⁵S(O)ₙR⁸, -Lx-NR⁵C(O)NR¹⁰R¹¹, -Lx-NR⁵S(O)ₙNR¹⁰R¹¹, -Lx-NR⁵C(O)OR⁷, -Lx-NR⁵S(O)ₙOR⁷, —NO₂, -Lx-C(O)NR⁵R⁶, -Lx-S(O)ₙNR⁵R⁶, oxo(═O), or selected from cyclopropyl, cyclobutyl, cyclopentyl, cyclopentenyl, cyclohexyl, cyclohexenyl, pyrrolidinyl, tetrahydrofuryl, tetrahydropyranyl, piperidinyl, piperazinyl, morpholinyl, homomorpholinyl, thiomorpholinyl, diazepanyl, oxazepanyl, phenyl, naphthyl pyridyl, pyrimidinyl, pyrazinyl, pyridazinyl, pyrrolyl, pyrazolyl, imidazolinyl, oxazolyl, isoxazolyl, thiazolyl, thienyl, furyl, benzofuryl, benzothienyl, benzoimidazolinyl, indolyl, indazolyl, and quinolinyl, each of which is optionally substituted, provided when L is methylene and W is 5- or 6-membered heterocycle, R³ is independently selected from -Lx-NR⁵R⁶, -Lx-OR⁷, -Lx-S(O)ₙR⁸, -Lx-C(O)R⁹, —S(O)ₙ-Lx-R⁸, —C(O)-Lx-R⁹, -Lx-CN, -Lx-NR⁵C(O)R⁹, -Lx-NR⁵S(O)ₙR⁸, -Lx-NR⁵C(O)NR¹⁰R¹¹, -Lx-NR⁵S(O)ₙNR¹⁰R¹¹, -Lx-NR⁵C(O)OR⁷, -Lx-NR⁵S(O)ₙOR⁷, —NO₂, -Lx-C(O)NR⁵R⁶, -Lx-S(O)ₙNR⁵R⁶, oxo(═O),
R⁴ is methyl, ethyl, n-propyl, i-propyl, n-butyl, i-butyl, and t-butyl, each of which is optionally substituted
R⁵, R⁶, R⁷, R⁸, R⁹, R¹⁰, and R¹¹ are independently selected from hydrogen, alkyl, cycloalkyl, aryl, heteroaryl, and heterocycle, each of which except for hydrogen, is optionally substituted with one or more groups selected from halo, —OH, —O(C₁-C₄ alkyl), —CN, C₁-C₄ alkyl, —NH₂, —NH(C₁-C₄ alkyl), —N(C₁-C₄ alkyl)(C₁-C₄ alkyl), —C(O)NH₂, —C(O)NH(C₁-C₄ alkyl), —C(O)N(C₁-C₄ alkyl)(C₁-C₄ alkyl), —C(O)(C₁-C₄ alkyl), —NHC(O)(C₁-C₄ alkyl), —N(C₁-C₄ alkyl)C(O)(C₁-C₄ alkyl), —S(O)NH₂, —S(O)NH(C₁-C₄ alkyl), —S(O)ₙN(C₁-C₄ alkyl)(C₁-C₄ alkyl), —S(O)ₙ(C₁-C₄ alkyl), —NHS(O)ₙ(C₁-C₄ alkyl), —N(C₁-C₄ alky)S(O)ₙ(C₁-C₄ alkyl), optionally substituted C₃-C₈ cycloalkyl, and optionally substituted 3-8 membered heterocycle, wherein C₁-C₄ alkyl is optionally substituted by halo, —OH, —OMe, or —CN,
or R⁵ and R⁶, R⁵ and R⁷, R⁵ and R⁸, R⁵ and R⁹, and R⁵ and R¹⁰ together with the atom(s) to which they are attached can form a ring, which is optionally substituted with one or more groups selected from halo, —OH, —O(C₁-C₄ alkyl), —CN, C₁-C₄ alkyl, —NH₂, —NH(C₁-C₄ alkyl), —N(C₁-C₄ alkyl)(C₁-C₄ alkyl), —C(O)NH₂, —C(O)NH(C₁-C₄ alkyl), —C(O)N(C₁-C₄ alkyl)(C₁-C₄ alkyl), —C(O)(C₁-C₄ alkyl), —NHC(O)(C₁-C₄ alkyl), —N(C₁-C₄ alkyl)C(O)(C₁-C₄ alkyl), —S(O)ₙNH₂, —S(O)₁NH(C₁-C₄ alkyl), —S(O)ₙN(C₁-C₄ alkyl)(C₁-C₄ alkyl), —S(O)ₙ(C₁-C₄ alkyl), —NHS(O)ₙ(C₁-C₄ alkyl), —N(C₁-C₄ alky)S(O)ₙ(C₁-C₄ alkyl), optionally substituted C₃-C₈ cycloalkyl, and optionally substituted 3-8 membered heterocycle, wherein C₁-C₄ alkyl is optionally substituted by halo, —OH, —OMe, or CN;
Lx is a bond, or optionally substituted C₁-C₄alkylene.

22. (canceled)

23. The compound of claim 1, and/or its racemic mixture, enantiomers, diastereomers, tautomers, or mixtures of optional ratio, or at least one pharmaceutically acceptable salt, or solvate thereof, wherein R⁵, R⁶, R⁷, R⁸, R⁹, R¹⁰, and R¹¹ are independently selected from hydrogen, C₁-C₄alkyl, C₃-C₈cycloalkyl, C₅-C₁₀aryl, 5-10 membered heteroaryl, and 3-8 membered heterocycle, each of which except for hydrogen, is optionally substituted with one or more groups selected from halo, —OH, —O(C₁-C₄ alkyl), —CN, C₁-C₄ alkyl, —NH₂, —NH(C₁-C₄ alkyl), —N(C₁-C₄ alkyl)(C₁-C₁ alkyl), —C(O)NH₂, —C(O)NH(C₁-C₄ alkyl), —C(O)N(C₁-C₄ alkyl)(C₁-C₁ alkyl), —C(O)(C₁-C₄ alkyl), —NHC(O)(C₁-C₄ alkyl), —N(C₁-C₄ alkyl)C(O)(C₁-C₄ alkyl), —S(O)ₙNH₂, —S(O)ₙNH(C₁-C₄ alkyl), —S(O)ₙN(C₁-C₄ alkyl)(C₁-C₄ alkyl), —S(O)ₙ(C₁-C₄ alkyl), —NHS(O)ₙ(C₁-C₄ alkyl), —N(C₁-C₄ alky)S(O)ₙ(C₁-C₄ alkyl), optionally substituted C₃-C₈ cycloalkyl, and optionally substituted 3-8 membered heterocycle, wherein C₁-C₄ alkyl is optionally substituted by halo, —OH, —OMe, or —CN.

24. (canceled)

25. The compound of claim 1, and/or its racemic mixture, enantiomers, diastereomers, tautomers, or mixtures of optional ratio, or at least one pharmaceutically acceptable salt, or solvate thereof, wherein R⁵ and R⁶, R⁵ and R⁷, R⁵ and R⁸, R⁵ and R⁹, and R⁵ and R¹⁰ together with the atom(s) to which they are attached can form a ring, which is optionally substituted with one or more groups selected from halo, —OH, —O(C₁-C₄ alkyl), —CN, C₁-C₄ alkyl, —NH₂, —NH(C₁-C₄ alkyl), —N(C₁-C₄ alkyl)(C₁-C₁ alkyl), —C(O)NH₂, —C(O)NH(C₁-C₄ alkyl), —C(O)N(C₁-C₄ alkyl)(C₁-C₄ alkyl), —C(O)(C₁-C₄ alkyl), —NHC(O)(C₁-C₄ alkyl), —N(C₁-C₄ alkyl)C(O)(C₁-C₄ alkyl), —S(O)ₙNH₂, —S(O)ₙNH(C₁-C₄ alkyl), —S(O)ₙN(C₁-C₄ alkyl)(C₁-C₄ alkyl), —S(O)ₙ(C₁-C₄ alkyl), —NHS(O)ₙ(C₁-C₄ alkyl), —N(C₁-C₄ alky)S(O)ₙ(C₁-C₄ alkyl), optionally substituted C₃-C₈ cycloalkyl, and optionally substituted 3-8 membered heterocycle, wherein C₁-C₄ alkyl is optionally substituted by halo, —OH, —OMe, or CN.

26. (canceled)

27. (canceled)

28. The compound of formula (I) according to claim 1, and/or its racemic mixture, enantiomers, diastereomers, tautomers, or mixtures of optional ratio, or at least one pharmaceutically acceptable salt, or solvate thereof, wherein
R¹ is independently chosen from hydrogen, halo, —CN, —OH; or is chosen from methyl, ethyl, n-propyl, i-propyl, —NH₂, N-methylamino, N,N-dimethylamino, N-ethylamino, N-n-propylamino, N-i-propylamino, methoxy, ethoxy, propoxy, isopropoxy, each of which is optionally substituted,
R² is independently chosen from phenyl, naphthyl, pyridyl, pyrimidinyl, pyrazinyl, pyridazinyl, pyrrolyl pyrazolyl, imidazolinyl, oxazolyl, isoxazolyl, thiazolyl, thienyl, furyl, benzofuryl, benzothienyl, benzoimidazolinyl, indolyl, indazolyl, quinolinyl, and, indanyl, indolinyl, indolin-2-one, 2,3-dihydrobenzofuryl, benzo[d][1,3]dioxolyl, and 1,2,3,4-tetrahydroquinolinyl, chroman, 2,3-dihydrobenzo[b][1,4]dioxinyl, 3,4-dihydro-2H-benzo[b][1,4]oxazinyl, isochroman, 1,3-dihydroisobenzofuryl, 1H-benzo[d][1,3]oxazin-2(4H)-onyl, each of which is optionally substituted by one or more groups selected from halo, —NR⁵R⁶, —OR⁷, —S(O)ₙR⁸, —C(O)R⁹, —C(O)OR⁷, —CN, —C(O)NR⁵R⁶, —NR⁵C(O)R⁹, —NR⁵S(O)ₙR⁸, —NR⁵S(O)ₙNR¹⁰R¹¹, —NR⁵C(O)OR⁷, —NR⁵C(O)NR¹⁰R¹¹, —NO₂, and —S(O)ₙNR⁵R⁶; or selected from methyl, ethyl, n-propyl, i-propyl, n-butyl, i-butyl, and t-butyl, cyclopropyl, cyclobutyl, cyclopentyl, cyclopentenyl, cyclohexyl, cyclohexenyl, pyrrolidinyl, tetrahydrofuryl, tetrahydropyranyl, piperidinyl, piperazinyl, morpholinyl, homomorpholinyl, thiomorpholinyl, diazepanyl, oxazepanyl, pyridyl, pyrimidinyl, pyrazinyl, pyridazinyl, pyrroly, l pyrazolyl, imidazolinyl, oxazolyl, isoxazolyl, thiazolyl, thienyl, furyl, benzofuryl, benzothienyl, benzoimidazolinyl, indolyl, indazolyl, quinolinyl, phenyl, and naphthyl, each of which is optionally substituted by one or more groups selected from halo, —NR⁵R⁶, —OR⁷, —S(O)ₙR⁸, —C(O)R⁹, —C(O)OR⁷, —CN, —C(O)NR⁵R⁶, —NR⁵C(O)R⁹, —NR⁵S(O)ₙR⁸, —NR⁵S(O)ₙNR¹⁰R¹¹, —NR⁵C(O)OR⁷, —NR⁵C(O)NR¹⁰R¹¹, S(O)ₙNR⁵R⁶, optionally substituted lower alkyl, optionally substituted cycloalkyl, optionally substituted heterocycle, optionally substituted heteroaryl, optionally substituted aryl, optionally substituted alkenyl, and optionally substituted alkynyl,
L is a bond, or optionally substituted C₁-C₆ alkylene,
W is cyclopropyl, cyclobutyl, cyclopentyl, cyclopentenyl, cyclohexyl, cyclohexenyl, pyrrolidinyl, tetrahydrofuryl, tetrahydropyranyl, piperidinyl, piperazinyl, morpholinyl, homomorpholinyl, thiomorpholinyl, phenyl, naphthyl pyridyl, pyrimidinyl, pyrazinyl, pyridazinyl, pyrrolyl, pyrazolyl, imidazolinyl, oxazolyl, isoxazolyl, thiazolyl, thienyl, furyl, benzofuryl, benzothienyl, benzoimidazolinyl, indolyl, indazolyl, or quinolinyl,
R³ is independently selected from hydrogen, -Lx-halo, -Lx-R⁴, -Lx-NR⁵R⁶, -Lx-OR⁷, -Lx-S(O)ₙR⁸, -Lx-C(O)R⁹, —S(O)ₙ-Lx-R⁸, —C(O)-Lx-R⁹, -Lx-CN, -Lx-NR⁵C(O)R⁹, -Lx-NR⁵S(O)ₙR⁸, -Lx-NR⁵C(O)NR¹⁰R¹¹, -Lx-NR⁵S(O)ₙNR¹⁰R¹¹, -Lx-NR⁵C(O)OR⁷, -Lx-NR⁵S(O)ₙOR⁷, —NO₂, -Lx-C(O)NR⁵R⁶, -Lx-S(O)ₙNR⁵R⁶, and oxo(═O), provided when L is methylene and W is 5- or 6-membered heterocycle, R³ is independently selected from -Lx-NR⁵R⁶, -Lx-OR⁷, -Lx-S(O)ₙR⁸, -Lx-C(O)R⁹, —S(O)ₙ-Lx-R⁸, —C(O)-Lx-R⁹, -Lx-CN, -Lx-NR⁵C(O)R⁹, -Lx-NR⁵S(O)ₙR⁸, -Lx-NR⁵C(O)NR¹⁰R¹¹, -Lx-NR⁵S(O)₁₇NR¹⁰R¹¹, -Lx-NR⁵C(O)OR⁷, -Lx-NR⁵S(O)ₙOR⁷, —NO₂, -Lx-C(O)NR⁵R⁶, -Lx-S(O)ₙNR⁵R⁶, oxo(═O),
R⁴ is methyl, ethyl, n-propyl, i-propyl, n-butyl, i-butyl, and t-butyl, each of which is optionally substituted,
R⁵, R⁶, R⁷, R⁸, R⁹, R¹⁰, and R¹¹ are independently selected from hydrogen, methyl, ethyl, n-propyl, i-propyl, n-butyl, i-butyl, and t-butyl, cyclopropyl, cyclobutyl, cyclopentyl, cyclopentenyl, cyclohexyl, cyclohexenyl, phenyl, naphthyl, pyridyl, pyrimidinyl, pyrazinyl, pyridazinyl, pyrrolyl pyrazolyl, imidazolinyl, oxazolyl, isoxazolyl, thiazolyl, thienyl, furyl, benzofuryl, benzothienyl, benzoimidazolinyl, indolyl, indazolyl, quinolinyl, pyrrolidinyl, tetrahydrofuryl, tetrahydropyranyl, piperidinyl, piperazinyl, morpholinyl, homomorpholinyl, thiomorpholinyl, diazepanyl, and oxazepanyl, each of which except for hydrogen, is optionally substituted with one or more groups selected from halo, —OH, —O(C₁-C₄ alkyl), —CN, C₁-C₄ alkyl, —NH₂, —NH(C₁-C₄ alkyl), —N(C₁-C₄ alkyl)(C₁-C₄ alkyl), —C(O)NH₂, —C(O)NH(C₁-C₄ alkyl), —C(O)N(C₁-C₄ alkyl)(C₁-C₄ alkyl), —C(O)(C₁-C₄ alkyl), —NHC(O)(C₁-C₄ alkyl), —N(C₁-C₄ alkyl)C(O)(C₁-C₄ alkyl), —S(O)ₙNH₂, —S(O)NH(C₁-C₄ alkyl), —S(O)ₙN(C₁-C₄ alkyl)(C₁-C₄ alkyl), —S(O)ₙ(C₁-C₄ alkyl), —NHS(O)ₙ(C₁-C₄ alkyl), —N(C₁-C₄ alky)S(O)ₙ(C₁-C₄ alkyl), optionally substituted C₃-C₈ cycloalkyl, and optionally substituted 3-8 membered heterocycle, wherein C₁-C₄ alkyl is optionally substituted by halo, —OH, —OMe, or CN,
or R⁵ and R⁶, R⁵ and R⁷, R⁵ and R⁸, R⁵ and R⁹, and R⁵ and R¹⁰ together with the atom(s) to which they are attached can form a ring, which is optionally substituted with one or more groups selected from halo, —OH, —O(C₁-C₄ alkyl), —CN, C₁-C₄ alkyl, —NH₂, —NH(C₁-C₄ alkyl), —N(C₁-C₄ alkyl)(C₁-C₄ alkyl), —C(O)NH₂, —C(O)NH(C₁-C₄ alkyl), —C(O)N(C₁-C₄ alkyl)(C₁-C₄ alkyl), —C(O)(C₁-C₄ alkyl), —NHC(O)(C₁-C₄ alkyl), —N(C₁-C₄ alkyl)C(O)(C₁-C₄ alkyl), —S(O)ₙNH₂, —S(O)₁NH(C₁-C₄ alkyl), —S(O)ₙN(C₁-C₄ alkyl)(C₁-C₄ alkyl), —S(O)ₙ(C₁-C₄ alkyl), —NHS(O)ₙ(C₁-C₄ alkyl), —N(C₁-C₄ alky)S(O)ₙ(C₁-C₄ alkyl), optionally substituted C₃-C₈ cycloalkyl, and optionally substituted 3-8 membered heterocycle, wherein C₁-C₄ alkyl is optionally substituted by halo, —OH, —OMe, or CN,
Lx is a bond, or optionally substituted C₁-C₄alkylene,
m is 0, 1 or 2,
n is 1 or 2,
p is 1, 2 or 3.

29. (canceled)

30. The compound of claim 1, chosen from compounds 1 to 323 and/or its racemic mixture, enantiomers, diastereomers, tautomers, or mixtures of optional ratio, or at least one pharmaceutically acceptable salt, or solvate thereof.

31. A composition comprising the compound of claim 1, and/or its racemic mixture, enantiomers, diastereomers, tautomers, or mixtures of optional ratio, or at least one pharmaceutically acceptable salt, or solvate thereof and at least one pharmaceutically acceptable carrier.

32.-34. (canceled)

35. A method for inhibiting a Syk kinase, comprising administering to a system or a subject in need thereof a therapeutically effective amount of a compound of Formula (I) of claim 1.

36. A method for treating a Syk-mediated disease comprising administering to a subject in need thereof a therapeutically effective amount of a compound of Formula (I) of claim 1.

37. (canceled)

38. The method of claim 36, wherein the disease is allergic asthma, allergic rhinitis, rheumatoid arthritis, multiple sclerosis, lupus, systemic lupus erythematosus, lymphoma, B cell lymphoma, T cell lymphoma, leukemia, myelodysplasic syndrome, anemia, leucopenia, neutropenia, thrombocytopenia, granuloctopenia, pancytoia or idiopathic thrombocytopenic purpura.

39. (canceled)
